(19) United States
(12) Patent Application Publication
(10) Pub. No.: US 2019/0152992 A1
(43) Pub. Date: May 23, 2019

(54) BENZODIOXANE DERIVATIVES AND THEIR PHARMACEUTICAL USE

(71) Applicant: Orion Corporation, Espoo (FI)

(72) Inventors: Anssi HAIKARAINEN, Järvenpää (FI); Esa KUMPULAINEN, Espoo (FI); Antti POHJAKALLIO, Espoo (FI); Jarmo PYSTYNEN, Espoo (FI); Shouming WANG, Espoo (FI)

(21) Appl. No.: 16/313,956

(22) PCT Filed: Jun. 28, 2017
(86) PCT No.: PCT/FI2017/050484

§ 371 Date: Dec. 28, 2018

(30) Foreign Application Priority Data
Jun. 29, 2016 (FI) 20165536

(51) Int. Cl. C07D 513/04; C07D 413/14; C07D 405/14; C07D 417/14; C07D 471/04; C07D 473/00; C07D 487/04; C07D 487/10; A61P 25/08; A61P 25/16; A61P 25/18; A61P 25/24; A61P 25/28

(52) U.S. Cl. CPC C07D 513/04; C07D 413/14; C07D 405/14; C07D 417/14; C07D 471/04; C07D 473/00; A61P 25/28; C07D 487/10; A61P 25/08; A61P 25/16; A61P 25/18; A61P 25/24; C07D 487/04

(57) ABSTRACT

Compounds of formula I,

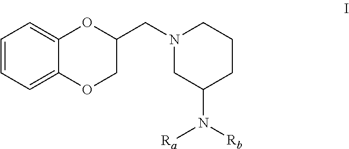

wherein Rₐ and R_{b} are as defined in the claims, exhibit alpha2C antagonistic activity and are thus useful as alpha2C antagonists.

### TECHNICAL FIELD

The present disclosure relates to pharmacologically active 1-((2,3-dihydrobenzo[b][1,4]-dioxin-2-yl)methyl)piperidine derivatives, or pharmaceutically acceptable salts and esters thereof, as well as to pharmaceutical compositions comprising them and to their use as alpha2C antagonists.

### BACKGROUND OF THE INVENTION

It is generally known and accepted in the art that compounds exhibiting alpha adrenergic activity may be used for the treatment of a wide variety of diseases and conditions of the peripheric system and the central nervous system (CNS).

The alpha adrenergic receptors can be divided on a pharmacological basis into alpha1 and alpha2 adrenoceptors, which can both be further divided into subtypes. Three genetically encoded subtypes, namely alpha2A, alpha2B, and alpha2C adrenoceptors, have been discovered in human. A fourth pharmacologically defined subtype, namely alpha2D adrenoceptor, is known in some other mammals and in rodents. It corresponds to the genetically defined alpha2A adrenoceptor.

The alpha2 adrenoceptor subtypes have distinct tissue distributions and functional roles. For instance, while alpha2A adrenoceptors are widely expressed in various tissues, alpha2C adrenoceptors are concentrated in the CNS and appear to play a role in the modulation of specific CNS mediated behavioral and physiological responses.

Some compounds that are non-specific for any of the above-mentioned alpha2 subtypes and some compounds that are specific for certain alpha2 subtypes are known in the art. For example, atipamezole disclosed in EP 183 492 is a non-specific alpha2 antagonist. Compounds that are selective antagonists for the alpha2C subtype and are thus useful for the treatment of diseases of the central nervous system, are described, for example in WO 2009/013390 and WO 2010/058060.

In order to be able to reduce the risk of adverse events during treatment, an enhanced selectivity of the alpha2 antagonists would be desirable. For example, the use of non-selective alpha2 antagonists is attributed with side effects, such as increases in blood pressure, heart rate, salivary secretion, gastrointestinal secretion, and anxiety. Also an enhanced potency of the alpha2C antagonists would be desirable, in order to be able to reduce the dose needed.

### SUMMARY OF THE INVENTION

An object of the present disclosure is to provide novel alpha2C antagonists that can be used for the treatment of diseases or conditions of the peripheric or central nervous system wherein alpha2C antagonists are indicated to be useful. Accordingly, an object of the present disclosure is to provide further compounds to be used as alpha2C antagonists in the treatment of mammals. Furthermore, pharmaceutical compositions comprising the presently disclosed compounds are also provided.

The alpha2 antagonists of the present disclosure have an improved selectivity for the alpha2C adrenoceptor subtype, an enhanced potency, improved metabolic stability, and/or improved solubility, moreover, more desirable pharmacokinetic and pharmacodynamics.

### DETAILED DESCRIPTION OF THE INVENTION

The present disclosure relates to novel compounds having the general formula I,

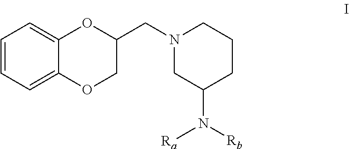

wherein;
Rₐ and R_{b} form, together with the nitrogen atom to which they are attached, a 5 or 6 membered saturated or unsaturated heterocyclic ring, containing, in addition to the nitrogen atom to which Rₐ and R_{b} are attached, 0, 1 or 2 ring heteroatom(s) each independently selected from N, O and S, wherein said heterocyclic ring is substituted with 1 substituent R₁, or said heterocyclic ring is substituted with 2 substituents R₁ and R₂, or said heterocyclic ring is substituted with 3 substituents R₁, R₂, and R₃, or said heterocyclic ring is substituted with 4 substituents R₁, R₂, R₃, and R₄, or said heterocyclic ring is substituted with 5 substituents R₁, R₂, R₃, R₄, and R₅;
R₁, R₂, R₃, R₄ and R₅ are independently oxo, (C₁-C₆)alkyl, (C₁-C₆)alkoxy(C₁-C₆)alkyl, (R₆)₂N—, (R₆)₂N—(C₁-C₆)alkyl, (R₆)₂N—(C═O)—(C₁-C₆)alkyl, (C₁-C₆)alkyl-(C═O)—NR₆—(C₁-C₆)alkyl, cyclo(C₃-C₆)alkyl, cyclo(C₃-C₆)alkyl(C₁-C₆)alkyl, phenyl, phenyl(C₁-C₆)alkyl, heterocyclyl, or heterocyclyl(C₁-C₆)alkyl, wherein said phenyl, cyclo(C₃-C₆)alkyl, or heterocyclyl is optionally substituted with 1 or 2 substituent(s) each independently being halogen, (C₁-C₆)alkoxy, or (C₁-C₆)alkyl-(C═O);
or two of R₃, R₄ and R₅, both attached to the same carbon ring atom form, together with the carbon ring atom to which they are attached, a 3 membered unsubstituted carbocyclic ring;
or two of R₁, R₂, R₃, R₄ and R₅, attached to the adjacent carbon ring atoms form, together with the carbon ring atoms to which they are attached, a phenyl ring, a 3 to 6 membered saturated or unsaturated carbocyclic ring, or a 5 or 6 membered saturated or unsaturated heterocyclic ring containing 1 or 2 ring heteroatom(s) each independently selected from N and S, wherein said phenyl ring, carbocyclic ring, or heterocyclic ring is unsubstituted, or said phenyl ring, carbocyclic ring, or heterocyclic ring is substituted with 1 substituent R₇, or said phenyl ring, carbocyclic ring, or heterocyclic ring is substituted with 2 substituents R₇ and R₈;
R₆ is H or (C₁-C₆)alkyl;
R₇ and R₈ are independently halogen, (C₁-C₆)alkyl, (C₁-C₆)alkoxy, (C₁-C₆)alkyl-S—, CN, or (C₁-C₆)alkyl-(C═O)—NR₆—(C₁-C₆)alkyl;
or R₇ and R₈, attached to the non-adjacent carbon ring atoms, form a bridge;
or a pharmaceutically acceptable salt or ester thereof.

In one embodiment the present disclosure relates to compounds of formula I, wherein the compound is a compound of formula Ia,

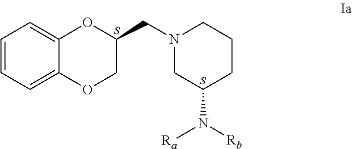

In one embodiment the present disclosure relates to compounds of formula I, wherein; Rₐ and R_{b} form, together with the nitrogen atom to which they are attached, a 5 or 6 membered saturated or unsaturated heterocyclic ring, containing, in addition to the nitrogen atom to which Rₐ and R_{b} are attached, 0, 1 or 2 ring heteroatom(s) each independently selected from N, O and S, wherein said heterocyclic ring is substituted with 1 substituent R₁, or said heterocyclic ring is substituted with 2 substituents R₁ and R₂, or said heterocyclic ring is substituted with 3 substituents R₁, R₂, and R₃, or said heterocyclic ring is substituted with 4 substituents R₁, R₂, R₃, and R₄, or said heterocyclic ring is substituted with 5 substituents R₁, R₂, R₃, R₄, and R₅;

R₁ is oxo;
R₂ is oxo, (C₁-C₆)alkyl, (C₁-C₆)alkoxy(C₁-C₆)alkyl, (R₆)₂N—(C₁-C₆)alkyl, (R₆)₂N—(C═O)—(C₁-C₆)alkyl, (C₁-C₆)alkyl-(C═O)—NR₆—(C₁-C₆)alkyl, cyclo(C₃-C₆)alkyl, cyclo(C₃-C₆)alkyl(C₁-C₆)alkyl, phenyl, phenyl(C₁-C₆)alkyl, heterocyclyl, or heterocyclyl(C₁-C₆)alkyl, wherein said phenyl, cyclo(C₃-C₆)alkyl, or heterocyclyl is optionally substituted with 1 or 2 substituent(s) each independently being halogen, (C₁-C₆)alkoxy, or (C₁-C₆)alkyl-(C═O);
R₃ is oxo, (C₁-C₆)alkyl, or phenyl;
R₄ is oxo or (C₁-C₆)alkyl;
R₅ is (C₁-C₆)alkyl;
or two of R₃, R₄ and R₅, both attached to the same carbon ring atom form, together with the carbon ring atom to which they are attached, a 3 membered unsubstituted carbocyclic ring;
or two of R₁, R₂, R₃, R₄ and R₅ attached to the adjacent carbon ring atoms form, together with the carbon ring atoms to which they are attached, a phenyl ring, a 3 to 6 membered saturated or unsaturated carbocyclic ring, or a 5 or 6 membered saturated or unsaturated heterocyclic ring containing 1 or 2 ring heteroatom(s) each independently selected from N and S, wherein said phenyl ring, carbocyclic ring, or heterocyclic ring is unsubstituted, or said phenyl ring, carbocyclic ring, or heterocyclic ring is substituted with 1 substituent R₇, or said phenyl ring, carbocyclic ring, or heterocyclic ring is substituted with 2 substituents R₇ and R₈;
R₆ is H or (C₁-C₆)alkyl;
R₇ is halogen, (C₁-C₆)alkyl, (C₁-C₆)alkoxy, (C₁-C₆)alkyl-S—, CN, or (C₁-C₆)alkyl-(C═O)—NR₆—(C₁-C₆)alkyl;
R₈ is halogen or (C₁-C₆)alkoxy;
or R₇ and R₈, attached to the non-adjacent carbon ring atoms, form a bridge.

In one embodiment the present disclosure relates to compounds of formula I, wherein Rₐ and R_{b} form, together with the nitrogen atom to which they are attached any one of the following groups

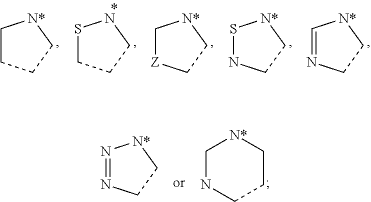

wherein;

### Z is N or O;

atom marked with * is bonded to the parent molecular moiety, and the dotted line is a single or a double bond.

In one embodiment the present disclosure relates to compounds of formula I, wherein Rₐ and R_{b} form, together with the nitrogen atom to which they are attached any one of the following groups

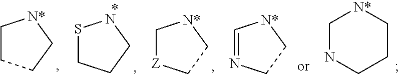

wherein Z is N or O, and the dotted line is a single or a double bond.

In one embodiment the present disclosure relates to compounds of formula I, wherein Rₐ and R_{b} form, together with the nitrogen atom to which they are attached any one of the following groups

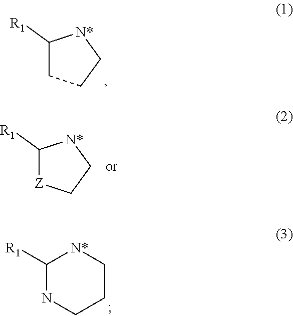

wherein;
group (1), (2), or (3) is optionally further substituted with R₂, R₃, and/or R₄;

### Z is N or O;

R₁ is oxo;
R₂ is (C₁-C₆)alkyl, (R₆)₂N—(C═O)—(C₁-C₆)alkyl, (C₁-C₆)alkyl-(C═O)—NR₆—(C₁-C₆)alkyl, phenyl, or phenyl(C₁-C₆)alkyl, wherein said phenyl is optionally substituted with 1 substituent being halogen or (C₁-C₆)alkoxy;
R₃ is oxo, (C₁-C₆)alkyl, or phenyl;
R₄ is (C₁-C₆)alkyl;
R₆ is H or (C₁-C₆)alkyl;
the atom marked with * is bonded to the parent molecular moiety, and the dotted line is a single or a double bond.

In one embodiment the present disclosure relates to compounds of formula I, wherein Rₐ and R_{b} form, together with the nitrogen atom to which they are attached any one of the following groups

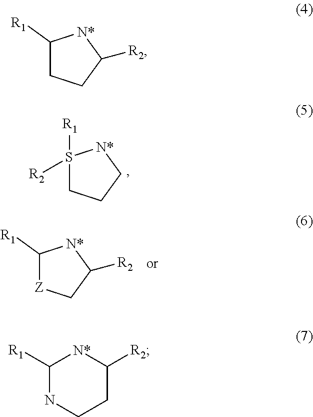

wherein;
group (4), (5), (6), or (7) is optionally further substituted with R₃, R₄, and/or R₅;

### Z is N or O;

R₁ is oxo;
R₂ is oxo;
R₃ is (C₁-C₆)alkyl, (R₆)₂N—(C₁-C₆)alkyl, (R₆)₂N—(C═O)—(C₁-C₆)alkyl, cyclo(C₃-C₆)alkyl, cyclo(C₃-C₆)alkyl(C₁-C₆)alkyl, phenyl, phenyl(C₁-C₆)alkyl, heterocyclyl, or heterocyclyl(C₁-C₆)alkyl, wherein said phenyl, cyclo(C₃-C₆)alkyl, or heterocyclyl is optionally substituted with 1 or 2 substituent(s) each independently being halogen or (C₁-C₆)alkyl-(C═O);
R₄ is oxo or (C₁-C₆)alkyl;
R₅ is (C₁-C₆)alkyl;
R₆ is (C₁-C₆)alkyl;
or two of R₃, R₄ and R₅, both attached to the same carbon ring atom form, together with the carbon ring atom to which they are attached, a 3 membered unsubstituted carbocyclic ring; and the atom marked with * is bonded to the parent molecular moiety.

In one embodiment the present disclosure relates to compounds of formula I, wherein Rₐ and R_{b} form, together with the nitrogen atom to which they are attached any one of the following groups

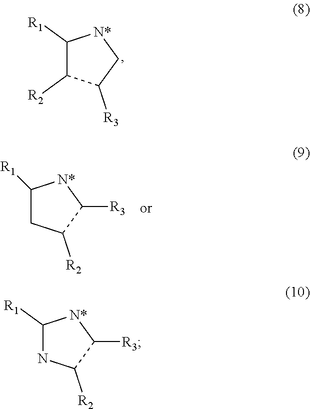

wherein;
group (8), (9), or (10) is optionally further substituted with R₄;
R₁ is oxo;
R₂ and R₃ form, together with the carbon ring atoms to which they are attached, a phenyl ring or a 5 or 6 membered unsaturated heterocyclic ring containing 1 or 2 ring heteroatom(s) each independently selected from N and S, wherein said phenyl ring or heterocyclic ring is unsubstituted, or said phenyl ring or heterocyclic ring is substituted with 1 substituent R₇, or said phenyl ring is substituted with 2 substituents R₇ and R₈;
R₄ is (C₁-C₆)alkyl or phenyl;
R₇ is halogen, (C₁-C₆)alkyl, (C₁-C₆)alkoxy, (C₁-C₆)alkyl-S—, CN, or (C₁-C₆)alkyl-(C═O)—NH—(C₁-C₆)alkyl;
R₈ is halogen or (C₁-C₆)alkoxy;
the atom marked with * is bonded to the parent molecular moiety, and the dotted line is a single or a double bond.

In one embodiment the present disclosure relates to compounds of formula I, wherein Rₐ and R_{b} form, together with the nitrogen atom to which they are attached any one of the following groups

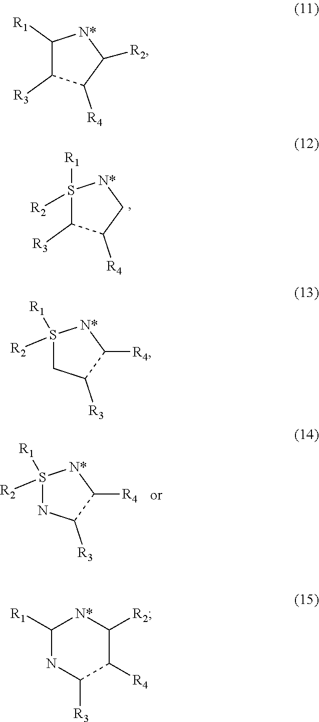

wherein;
group (11), (12), (13), (14), or (15) is optionally further substituted with R₅;
R₁ is oxo;
R₂ is oxo;
R₃ and R₄ form, together with the carbon ring atoms to which they are attached, a phenyl ring, a 3 to 6 membered saturated or unsaturated carbocyclic ring, or a 6 membered unsaturated heterocyclic ring containing 1 ring heteroatom being N, wherein said phenyl ring, carbocyclic ring, or heterocyclic ring is unsubstituted, or said phenyl ring is substituted with 1 substituent R₇, or said phenyl ring or carbocyclic ring is substituted with 2 substituents R₇ and R₈;
R₅ is phenyl;
R₇ is halogen or (C₁-C₆)alkoxy;
R₈ is (C₁-C₆)alkoxy;
or R₇ and R₈, attached to the non-adjacent carbon ring atoms, form a bridge;
the atom marked with * is bonded to the parent molecular moiety, and the dotted line is a single or a double bond.

In one embodiment the present disclosure relates to compounds of formula I, wherein Rₐ and R_{b} form, together with the nitrogen atom to which they are attached any one of the following groups

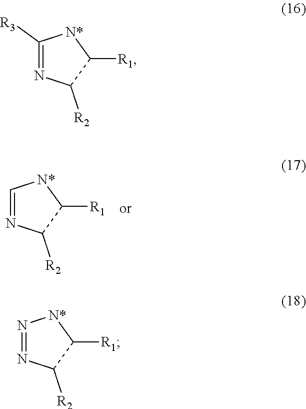

wherein;
R₁ and R₂ form, together with the carbon ring atoms to which they are attached, a phenyl ring, or a 6 membered saturated or unsaturated heterocyclic ring containing 1 or 2 ring heteroatom(s) each independently selected from N, wherein said phenyl ring, or heterocyclic ring is unsubstituted, or said phenyl ring, or heterocyclic ring is substituted with 1 substituent R₇, or said phenyl ring is substituted with 2 substituents R₇ and R₈;
R₃ is (C₁-C₆)alkyl, (C₁-C₆)alkoxy(C₁-C₆)alkyl, or (R₆)₂N—(C₁-C₆)alkyl;
R₆ is H or (C₁-C₆)alkyl;
R₇ is halogen or (C₁-C₆)alkoxy;
R₈ is halogen; and
the atom marked with * is bonded to the parent molecular moiety, and the dotted line is a single or a double bond.

In one embodiment the present disclosure relates to compounds of formula I, wherein the compound is 1-((S)-1-(((S)-2,3-dihydrobenzo[b][1,4]dioxin-2-yl)methyl)piperidin-3-yl)-4,4-dimethylpyrrolidin-2-one, 1-((S)-1-(((S)-2,3-dihydrobenzo[b][1,4]dioxin-2-yl)methyl)-piperidin-3-yl)-4,4-diphenylimidazolidin-2-one, 1-((S)-1-(((S)-2,3-dihydrobenzo[b][1,4]-dioxin-2-yl)methyl)piperidin-3-yl)-4-phenylimidazolidin-2-one, (3R,4R)-1-((S)-1-(((S)-2,3-dihydrobenzo[b][1,4]dioxin-2-yl)methyl)piperidin-3-yl)-3,4-dimethylpyrrolidine-2,5-dione, 3-((S)-1-(((S)-2,3-dihydrobenzo[b][1,4]dioxin-2-yl)methyl)piperidin-3-yl)-5,5-diethyloxazolidine-2,4-dione, (R)-1-((S)-1-(((S)-2,3-dihydrobenzo[b][1,4]dioxin-2-yl)methyl)-piperidin-3-yl)-3-isopropyl-4-phenylimidazolidin-2-one, (S)-1-((S)-1-(((S)-2,3-dihydrobenzo[b][1,4]dioxin-2-yl)methyl)piperidin-3-yl)-3-isopropyl-4-phenylimidazolidin-2-one, (R)-1-((S)-1-(((S)-2,3-dihydrobenzo[b][1,4]dioxin-2-yl)methyl)piperidin-3-yl)-4-phenylimidazolidin-2-one, (S)-1-((S)-1-(((S)-2,3-dihydrobenzo[b][1,4]dioxin-2-yl)methyl)piperidin-3-yl)-4-phenylimdin-2-one, 6-((S)-1-(((S)-2,3-dihydrobenzo[b][1,4]dioxin-2-yl)methyl)piperidin-3-yl)-6,7-dihydro-5H-pyrrolo[3,4-b]pyridin-5-one, 2-((S)-1-(((S)-2,3-dihydrobenzo[b][1,4]dioxin-2-yl)methyl)piperidin-3-yl)-1H-pyrrolo[3,4-c]pyridin-3(2H)-one, 6-((S)-1-(((S)-2,3-dihydrobenzo[b][1,4]dioxin-2-yl)methyl)piperidin-3-yl)-2-(methylthio)-5H-pyrrolo[3,4-d]pyrimidin-7(6H)-one, 5-((S)-1-(((S)-2,3-dihydrobenzo[b][1,4]dioxin-2-yl)methyl)piperidin-3-yl)-5,6-dihydro-4H-pyrrolo[3,4-d]thiazol-4-one, (R)-1-((S)-1-(((S)-2,3-dihydrobenzo[b][1,4]dioxin-2-yl)methyl)piperidin-3-yl)-3-methyl-3-phenylpyrrolidin-2-one, (S)-1-((S)-1-(((S)-2,3-dihydrobenzo[b][1,4]dioxin-2-yl)methyl)-piperidin-3-yl)-3-methyl-3-phenylpyrrolidin-2-one, 1-((S)-1-(((S)-2,3-dihydrobenzo[b][1,4]dioxin-2-yl)methyl)piperidin-3-yl)-3-ethylimidazolidin-2-one, 2-(3-((S)-1-(((S)-2,3-dihydrobenzo[b][1,4]dioxin-2-yl)methyl)-piperidin-3-yl)-2-oxoimidazolidin-1-yl)-N,N-dimethylacetamide, 5-tert-butyl-3-((S)-1-(((S)-2,3-dihydrobenzo[b][1,4]dioxin-2-yl)methyl)piperidin-3-yl)oxazolidin-2-one, 3-((S)-1-(((S)-2,3-dihydrobenzo[b][1,4]dioxin-2-yl)methyl)piperidin-3-yl)-5-isopropyloxazolidin-2-one, N-((3-((S)-1-(((S)-2,3-dihydrobenzo[b][1,4]dioxin-2-yl)methyl)piperidin-3-yl)-2-oxooxazolidin-5-yl)methyl)acetamide, 3-((S)-1-(((S)-2,3-dihydrobenzo[b][1,4]dioxin-2-yl)methyl)piperidin-3-yl)-5-(4-fluorophenyl)oxazolidin-2-one, 6-((S)-1-(((S)-2,3-dihydrobenzo[b][1,4]dioxin-2-yl)methyl)piperidin-3-yl)-2-(methylthio)-6,7-dihydro-5H-pyrrolo[3,4-d]pyrimidin-5-one, 6-((S)-1-(((S)-2,3-dihydrobenzo[b][1,4]dioxin-2-yl)methyl)piperidin-3-yl)-6,7-dihydro-5H-pyrrolo[3,4-d]pyrimidin-5-one, 5-((S)-1-(((S)-2,3-dihydrobenzo[b][1,4]dioxin-2-yl)methyl)piperidin-3-yl)-1-methyl-4,5-dihydropyrrolo[3,4-c]pyrazol-6(1H)-one, 1-((S)-1-(((S)-2,3-dihydrobenzo[b][1,4]dioxin-2-yl)methyl)piperidin-3-yl)-1,3-dihydrobenzo[c]isothiazole 2,2-dioxide, 1-((S)-1-(((S)-2,3-dihydrobenzo[b][1,4]-dioxin-2-yl)methyl)piperidin-3-yl)indolin-2-one, 1-((S)-1-(((S)-2,3-dihydrobenzo[b][1,4]-dioxin-2-yl)methyl)piperidin-3-yl)-4,6-difluoro-2-methyl-1H-benzo[d]imidazole, 1-((S)-1-(((S)-2,3-dihydrobenzo[b][1,4]dioxin-2-yl)methyl)piperidin-3-yl)-5,6-difluoro-2-methyl-1H-benzo[d]imidazole, 1-((S)-1-(((S)-2,3-dihydrobenzo[b][1,4]dioxin-2-yl)methyl)piperidin-3-yl)pyrrolidin-2-one, 1-((S)-1-(((S)-2,3-dihydrobenzo[b][1,4]dioxin-2-yl)methyl)piperidin-3-yl)-5-methylpyrrolidin-2-one, 1-((S)-1-(((S)-2,3-dihydrobenzo[b][1,4]dioxin-2-yl)methyl)-piperidin-3-yl)-3-phenylpyrrolidin-2-one, 1-((S)-1-(((S)-2,3-dihydrobenzo[b][1,4]dioxin-2-yl)methyl)piperidin-3-yl)-4-phenylpyrrolidin-2-one, 1-((S)-1-(((S)-2,3-dihydrobenzo-[b][1,4]dioxin-2-yl)methyl)piperidin-3-yl)pyrrolidine-2,5-dione, 2-((S)-1-(((S)-2,3-dihydrobenzo[b][1,4]dioxin-2-yl)methyl)piperidin-3-yl)-3a,4,7,7a-tetrahydro-1H-4,7-methanoisoindole-1,3(2H)-dione, 1-((S)-1-(((S)-2,3-dihydrobenzo[b][1,4]dioxin-2-yl)methyl)piperidin-3-yl)-3-methyl-3-phenylpyrrolidine-2,5-dione, (R)-1-((S)-1-(((S)-2,3-dihydrobenzo[b][1,4]dioxin-2-yl)methyl)piperidin-3-yl)-3-methyl-3-phenylpyrrolidine-2,5-dione, (S)-1-((S)-1-(((S)-2,3-dihydrobenzo[b][1,4]dioxin-2-yl)methyl)piperidin-3-yl)-3-methyl-3-phenylpyrrolidine-2,5-dione, 3-((S)-1-(((S)-2,3-dihydrobenzo[b][1,4]dioxin-2-yl)methyl)piperidin-3-yl)-1-phenyl-3-azabicyclo[3.1.0]hexane-2,4-dione, 1-((S)-1-(((S)-2,3-dihydrobenzo[b][1,4]dioxin-2-yl)methyl)piperidin-3-yl)-4-phenylpyrrolidine-2,3-dione, 1-((S)-1-(((S)-2,3-dihydrobenzo[b][1,4]dioxin-2-yl)methyl)piperidin-3-yl)-4-phenyl-1H-pyrrol-2(5H)-one, 1-((S)-1-(((S)-2,3-dihydrobenzo[b][1,4]dioxin-2-yl)methyl)piperidin-3-yl)-4-methyl-1H-pyrrol-2(5H)-one, 1-((S)-1-(((S)-2,3-dihydrobenzo[b][1,4]dioxin-2-yl)methyl)piperidin-3-yl)-4-(4-fluorophenyl)-1H-pyrrol-2(5H)-one, 1-((S)-1-(((S)-2,3-dihydrobenzo[b][1,4]dioxin-2-yl)methyl)piperidin-3-yl)-4-methyl-3-phenyl-1H-pyrrol-2(5H)-one, 1-((S)-1-(((S)-2,3-dihydrobenzo[b][1,4]dioxin-2-yl)methyl)piperidin-3-yl)-4-(2-methoxyphenyl)-1H-pyrrol-2(5H)-one formate, 1-((S)-1-(((S)-2,3-dihydrobenzo[b][1,4]dioxin-2-yl)methyl)piperidin-3-yl)imidazolidin-2-one, 1-((S)-1-(((S)-2,3-dihydrobenzo[b][1,4]dioxin-2-yl)methyl)piperidin-3-yl)-4,4-dimethylimidazolidin-2-one, (R)-1-((S)-1-(((S)-2,3-dihydrobenzo[b][1,4]dioxin-2-yl)methyl)piperidin-3-yl)-4-methylimidazolidin-2-one, 1-((S)-1-(((S)-2,3-dihydrobenzo[b][1,4]dioxin-2-yl)methyl)piperidin-3-yl)-5-methylimidazolidin-2-one, 1-((S)-1-(((S)-2,3-dihydrobenzo[b][1,4]dioxin-2-yl)methyl)piperidin-3-yl)-3-phenylimidazolidin-2-one, 1-((S)-1-(((S)-2,3-dihydrobenzo[b][1,4]dioxin-2-yl)methyl)piperidin-3-yl)-3,4-dimethylimidazolidin-2-one, 1-benzyl-3-((S)-1-(((S)-2,3-dihydrobenzo[b][1,4]dioxin-2-yl)methyl)piperidin-3-yl)imidazolidin-2-one, 1-((S)-1-(((S)-2,3-dihydrobenzo[b][1,4]dioxin-2-yl)methyl)piperidin-3-yl)-3,4,4-trimethylimidazolidin-2-one, 1-((S)-1-(((S)-2,3-dihydrobenzo[b][1,4]dioxin-2-yl)methyl)piperidin-3-yl)-3-(1-phenylethyl)imidazolidin-2-one hydrochloride, 1-((S)-1-(((S)-2,3-dihydrobenzo[b][1,4]dioxin-2-yl)methyl)piperidin-3-yl)tetrahydropyrimidin-2(1H)-one, 1-((S)-1-(((S)-2,3-dihydrobenzo[b][1,4]dioxin-2-yl)methyl)piperidin-3-yl)-3-methyltetrahydropyrimidin-2(1H)-one, 2-((S)-1-(((S)-2,3-dihydrobenzo[b][1,4]dioxin-2-yl)methyl)piperidin-3-yl)-4-methoxyisoindolin-1-one, 2-((S)-1-(((S)-2,3-dihydrobenzo[b][1,4]dioxin-2-yl)methyl)piperidin-3-yl)-4,5-difluoroisoindolin-1-one hydrochloride, 2-((S)-1-(((S)-2,3-dihydrobenzo[b][1,4]dioxin-2-yl)methyl)piperidin-3-yl)-4-fluoroisoindolin-1-one hydrochloride, 2-((S)-1-(((S)-2,3-dihydrobenzo[b][1,4]-dioxin-2-yl)methyl)piperidin-3-yl)-5-fluoroisoindolin-1-one hydrochloride, 2-((S)-1-(((S)-2,3-dihydrobenzo[b][1,4]dioxin-2-yl)methyl)piperidin-3-yl)-5-methylisoindolin-1-one formate, 5-chloro-2-((S)-1-(((S)-2,3-dihydrobenzo[b][1,4]dioxin-2-yl)methyl)piperidin-3-yl)isoindolin-1-one, 2-((S)-1-(((S)-2,3-dihydrobenzo[b][1,4]dioxin-2-yl)methyl)piperidin-3-yl)isoindoline-1,3-dione, 2-((S)-1-(((S)-2,3-dihydrobenzo[b][1,4]dioxin-2-yl)methyl)-piperidin-3-yl)-5-fluoroisoindoline-1,3-dione, 2-((S)-1-(((S)-2,3-dihydrobenzo[b][1,4]-dioxin-2-yl)methyl)piperidin-3-yl)-4-fluoroisoindoline-1,3-dione hydrochloride, 4-chloro-2-((S)-1-(((S)-2,3-dihydrobenzo[b][1,4]dioxin-2-yl)methyl)piperidin-3-yl)isoindoline-1,3-dione hydrochloride, 2-((S)-1-(((S)-2,3-dihydrobenzo[b][1,4]dioxin-2-yl)methyl)piperidin-3-yl)-4-methoxyisoindoline-1,3-dione, 2-((S)-1-(((S)-2,3-dihydrobenzo[b][1,4]dioxin-2-yl)methyl)piperidin-3-yl)-4,5-dimethoxyisoindoline-1,3-dione hydrochloride, 2-((S)-1-(((S)-2,3-dihydrobenzo[b][1,4]dioxin-2-yl)methyl)piperidin-3-yl)-1-oxoisoindoline-5-carbonitrile, 2-((S)-1-(((S)-2,3-dihydrobenzo[b][1,4]dioxin-2-yl)methyl)piperidin-3-yl)-5,6-dimethoxyisoindolin-1-one, 2-((S)-1-(((S)-2,3-dihydrobenzo[b][1,4]dioxin-2-yl)methyl)-piperidin-3-yl)-5-methoxyisoindolin-1-one, 2-((S)-1-(((S)-2,3-dihydrobenzo[b][1,4]dioxin-2-yl)methyl)piperidin-3-yl)-6-methoxyisoindolin-1-one, N-((2-((S)-1-(((S)-2,3-dihydrobenzo[b][1,4]dioxin-2-yl)methyl)piperidin-3-yl)-1-oxoisoindolin-5-yl)methyl)-acetamide, 3-((S)-1-(((S)-2,3-dihydrobenzo[b][1,4]dioxin-2-yl)methyl)-piperidin-3-yl)quinazoline-2,4(1H,3H)-dione, 3-((S)-1-(((S)-2,3-dihydrobenzo[b][1,4]dioxin-2-yl)methyl)-piperidin-3-yl)-5-ethyl-1-methyl-5-phenylimidazolidine-2,4-dione, 3-((S)-1-(((S)-2,3-dihydrobenzo[b][1,4]dioxin-2-yl)methyl)piperidin-3-yl)-5-methyl-5-phenylimidazolidine-2,4-dione, 6-((S)-1-(((S)-2,3-dihydrobenzo[b][1,4]dioxin-2-yl)methyl)-piperidin-3-yl)-5H-pyrrolo[3,4-b]pyridine-5,7(6H)-dione, 2-((S)-1-(((S)-2,3-dihydrobenzo[b][1,4]dioxin-2-yl)methyl)piperidin-3-yl)-2,3-dihydro-1H-pyrrolo[3,4-c]pyridin-1-one, 6-((S)-1-(((S)-2,3-dihydrobenzo[b][1,4]dioxin-2-yl)methyl)piperidin-3-yl)-5H-pyrrolo[3,4-b]pyridin-7(6H)-one, 6-((S)-1-(((S)-2,3-dihydrobenzo[b][1,4]dioxin-2-yl)methyl)piperidin-3-yl)-5H-pyrrolo[3,4-d]pyrimidin-7(6H)-one, 5-((S)-1-(((S)-2,3-dihydrobenzo[b][1,4]dioxin-2-yl)methyl)piperidin-3-yl)-4H-pyrrolo[3,4-d]thiazol-6(5H)-one, 5-((S)-1-(((S)-2,3-dihydrobenzo[b][1,4]dioxin-2-yl)methyl)piperidin-3-yl)-1-methyl-5,6-dihydropyrrolo[3,4-c]pyrazol-4(1H)-one, 2-((S)-1-(((S)-2,3-dihydrobenzo[b][1,4]dioxin-2-yl)methyl)piperidin-3-yl)-2,3-dihydrobenzo[d]isothiazole-1,1-dioxide, 1-((S)-1-(((S)-2,3-dihydrobenzo[b][1,4]dioxin-2-yl)methyl)piperidin-3-yl)-6-fluoro-1H-benzo[d]imidazole, 1-((S)-1-(((S)-2,3-dihydrobenzo[b][1,4]dioxin-2-yl)methyl)piperidin-3-yl)-6-fluoro-2-methyl-1H-benzo[d]imidazole, 1-((S)-1-(((S)-2,3-dihydrobenzo[b][1,4]dioxin-2-yl)methyl)-piperidin-3-yl)-2-ethyl-6-fluoro-1H-benzo[d]imidazole, 1-((S)-1-(((S)-2,3-dihydrobenzo-[b][1,4]dioxin-2-yl)methyl)piperidin-3-yl)-6-fluoro-2-isopropyl-1H-benzo[d]imidazole, 1-((S)-1-(((S)-2,3-dihydrobenzo[b][1,4]dioxin-2-yl)methyl)piperidin-3-yl)-6-fluoro-1H-benzo[d]imidazol-2(3H)-one, 1-((S)-1-(((S)-2,3-dihydrobenzo[b][1,4]dioxin-2-yl)methyl)-piperidin-3-yl)-5-fluoro-1H-benzo[d]imidazole, 1-((S)-1-(((S)-2,3-dihydrobenzo[b][1,4]-dioxin-2-yl)methyl)piperidin-3-yl)-5-fluoro-1H-benzo[d]imidazol-2(3H)-one, 6-chloro-1-((S)-1-(((S)-2,3-dihydrobenzo[b][1,4]dioxin-2-yl)methyl)piperidin-3-yl)-1H-benzo[d]-imidazole, 6-chloro-1-((S)-1-(((S)-2,3-dihydrobenzo[b][1,4]dioxin-2-yl)methyl)piperidin-3-yl)-2-methyl-1H-benzo[d]imidazole, 5-chloro-1-((S)-1-(((S)-2,3-dihydrobenzo[b][1,4]-dioxin-2-yl)methyl)piperidin-3-yl)-1H-benzo[d]imidazole, 2-((S)-1-(((S)-2,3-dihydrobenzo-[b][1,4]dioxin-2-yl)methyl)piperidin-3-yl)tetrahydrocyclopenta[c]pyrrole-1,3(2H,3aH)-dione hydrochloride, (3 aR,7aS)-2-((S)-1-(((S)-2,3-dihydrobenzo[b][1,4]dioxin-2-yl)methyl)piperidin-3-yl)hexahydro-1H-isoindole-1,3 (2H)-dione hydrochloride, 2-((S)-1-(((S)-2,3-dihydrobenzo[b][1,4]dioxin-2-yl)methyl)-piperidin-3-yl)-4,5,6,7-tetrahydro-1H-isoindole-1,3(2H)-dione hydrochloride, 3-((S)-1-(((S)-2,3-dihydrobenzo[b][1,4]dioxin-2-yl)methyl)piperidin-3-yl)-3-azabicyclo[3.1.0]hexane-2,4-dione, 1-((S)-1-(((S)-2,3-dihydrobenzo[b][1,4]dioxin-2-yl)methyl)-piperidin-3-yl)-3-ethylpyrimidine-2,4,6(1H,3H,5H)-trione, 1-((S)-1-(((S)-2,3-dihydrobenzo[b][1,4]dioxin-2-yl)methyl)-piperidin-3-yl)-3-methylpyrrolidin-2-one, 1-((S)-1-(((S)-2,3-dihydrobenzo[b][1,4]dioxin-2-yl)methyl)piperidin-3-yl)-3-methylpyrrolidin-2-one diastereomer 1,1-((S)-1-(((S)-2,3-dihydrobenzo[b][1,4]dioxin-2-yl)methyl)piperidin-3-yl)-3-methylpyrrolidin-2-one diastereomer 2,1-((S)-1-(((S)-2,3-dihydrobenzo[b][1,4]dioxin-2-yl)methyl)piperidin-3-yl)-3,3-dimethylpyrrolidin-2-one, 1-((S)-1-(((S)-2,3-dihydrobenzo[b][1,4]dioxin-2-yl)methyl)piperidin-3-yl)-3-methylimidazolidin-2-one, 3-((S)-1-(((S)-2,3-dihydrobenzo[b][1,4]dioxin-2-yl)methyl)piperidin-3-yl)imidazolidine-2,4-dione, 3-((S)-1-(((S)-2,3-dihydrobenzo[b][1,4]dioxin-2-yl)methyl)piperidin-3-yl)-1-methylimidazolidine-2,4-dione, 3-((S)-1-(((S)-2,3-dihydrobenzo[b][1,4]dioxin-2-yl)methyl)piperidin-3-yl)-1-isopropylimidazolidine-2,4-dione, 3-((S)-1-(((S)-2,3-dihydrobenzo[b][1,4]dioxin-2-yl)methyl)-piperidin-3-yl)-1-ethylimidazolidine-2,4-dione, 1-cyclopentyl-3-((S)-1-(((S)-2,3-dihydrobenzo[b][1,4]dioxin-2-yl)methyl)piperidin-3-yl)imidazolidine-2,4-dione, 3-((S)-1-(((S)-2,3-dihydrobenzo[b][1,4]dioxin-2-yl)methyl)piperidin-3-yl)-1-isobutylimidazolidine-2,4-dione, 1-(cyclopropylmethyl)-3-((S)-1-(((S)-2,3-dihydrobenzo[b][1,4]dioxin-2-yl)methyl)piperidin-3-yl)imidazolidine-2,4-dione, 2-(3-((S)-1-(((S)-2,3-dihydrobenzo-[b][1,4]dioxin-2-yl)methyl)piperidin-3-yl)-2,4-dioxoimidazolidin-1-yl)-N,N-dimethyl-acetamide, 3-((S)-1-(((S)-2,3-dihydrobenzo[b][1,4]dioxin-2-yl)methyl)piperidin-3-yl)-5,5-dimethylimidazolidine-2,4-dione, 3-((S)-1-(((S)-2,3-dihydrobenzo[b][1,4]dioxin-2-yl)methyl)-piperidin-3-yl)-1,5,5-trimethylimidazolidine-2,4-dione, (R)-3-((S)-1-(((S)-2,3-dihydrobenzo[b][1,4]dioxin-2-yl)methyl)piperidin-3-yl)-5-methylimidazolidine-2,4-dione, (S)-3-((S)-1-(((S)-2,3-dihydrobenzo[b][1,4]dioxin-2-yl)methyl)piperidin-3-yl)-5-methylimidazolidine-2,4-dione, 3-((S)-1-(((S)-2,3-dihydrobenzo[b][1,4]dioxin-2-yl)methyl)-piperidin-3-yl)-5-phenylimidazolidine-2,4-dione, 3-((S)-1-(((S)-2,3-dihydrobenzo[b][1,4]-dioxin-2-yl)methyl)-piperidin-3-yl)-1,5-dimethylimidazolidine-2,4-dione, 3-((S)-1-(((S)-2,3-dihydrobenzo[b][1,4]dioxin-2-yl)methyl)-piperidin-3-yl)-l-isopropyl-5,5-dimethyl-imidazolidine-2,4-dione, 1-tert-butyl-3-((S)-1-(((S)-2,3-dihydrobenzo[b][1,4]dioxin-2-yl)methyl)piperidin-3-yl)imidazolidine-2,4-dione, 1-benzyl-3-((S)-1-(((S)-2,3-dihydrobenzo-[b][1,4]dioxin-2-yl)methyl)piperidin-3-yl)imidazolidine-2,4-dione, 1-cyclopropyl-3-((S)-1-(((S)-2,3-dihydrobenzo[b][1,4]dioxin-2-yl)methyl)piperidin-3-yl)imidazolidine-2,4-dione, 3-((S)-1-(((S)-2,3-dihydrobenzo[b][1,4]dioxin-2-yl)methyl)-piperidin-3-yl)-1-(oxetan-3-yl)imidazolidine-2,4-dione, 1-(3,3-difluorocyclobutyl)-3-((S)-1-(((S)-2,3-dihydrobenzo-[b][1,4]dioxin-2-yl)methyl)piperidin-3-yl)imidazolidine-2,4-dione, 6-((S)-1-(((S)-2,3-dihydrobenzo[b][1,4]dioxin-2-yl)methyl)-piperidin-3-yl)-4,6-diazaspiro[2.4]heptane-5,7-dione, 6-((S)-1-(((S)-2,3-dihydrobenzo[b][1,4]dioxin-2-yl)methyl)-piperidin-3-yl)-4-methyl-4,6-diazaspiro[2.4]heptane-5,7-dione, 2-(6-((S)-1-(((S)-2,3-dihydrobenzo[b][1,4]dioxin-2-yl)methyl)piperidin-3-yl)-5,7-dioxo-4,6-diazaspiro[2.4]heptan-4-yl)-N,N-dimethyl-acetamide, 1-((S)-1-(((S)-2,3-dihydrobenzo[b][1,4]dioxin-2-yl)methyl)-piperidin-3-yl)-3-ethylimidazolidine-2,4,5-trione, 1-cyclohexyl-3-((S)-1-(((S)-2,3-dihydrobenzo[b][1,4]-dioxin-2-yl)methyl)piperidin-3-yl)imidazolidine-2,4,5-trione, 1-cyclopentyl-3-((S)-1-(((S)-2,3-dihydrobenzo[b][1,4]dioxin-2-yl)methyl)piperidin-3-yl)imidazolidine-2,4,5-trione, 1-((S)-1-(((S)-2,3-dihydrobenzo[b][1,4]dioxin-2-yl)methyl)-piperidin-3-yl)-3-((R)-1-phenylethyl)imidazolidine-2,4,5-trione, 1-((S)-1-(((S)-2,3-dihydrobenzo[b][1,4]dioxin-2-yl)methyl)-piperidin-3-yl)-3-phenylimidazolidine-2,4,5-trione, 1-((S)-1-(((S)-2,3-dihydrobenzo[b][1,4]dioxin-2-yl)methyl)-piperidin-3-yl)-3-isopropylimidazolidine-2,4,5-trione, 1-benzyl-3-((S)-1-(((S)-2,3-dihydrobenzo[b][1,4]dioxin-2-yl)methyl)piperidin-3-yl)imidazolidine-2,4,5-trione, 1-((S)-1-(((S)-2,3-dihydrobenzo[b][1,4]dioxin-2-yl)methyl)-piperidin-3-yl)-3-propylimidazolidine-2,4,5-trione, 1-((S)-1-(((S)-2,3-dihydrobenzo[b][1,4]-dioxin-2-yl)methyl)-piperidin-3-yl)-3-((S)-1-phenylethyl)imidazolidine-2,4,5-trione, 1-tert-butyl-3-((S)-1-(((S)-2,3-dihydrobenzo[b][1,4]dioxin-2-yl)methyl)piperidin-3-yl)-imidazolidine-2,4,5-trione, 1-((S)-1-(((S)-2,3-dihydrobenzo[b][1,4]dioxin-2-yl)methyl)-piperidin-3-yl)-3-(2-(dimethylamino)ethyl)imidazolidine-2,4,5-trione, 1-((S)-1-(((S)-2,3-dihydrobenzo[b][1,4]dioxin-2-yl)methyl)piperidin-3-yl)-3-(tetrahydro-2H-pyran-4-yl)imidazolidine-2,4,5-trione, 1-((S)-1-(((S)-2,3-dihydrobenzo[b][1,4]dioxin-2-yl)methyl)piperidin-3-yl)-3-(piperidin-4-yl)imidazolidine-2,4,5-trione dihydrochloride, 1-((S)-1-(((S)-2,3-dihydrobenzo[b][1,4]dioxin-2-yl)methyl)piperidin-3-yl)-3-methylimidazolidine-2,4,5-trione, 1-(1-acetylpiperidin-4-yl)-3-((S)-1-(((S)-2,3-dihydrobenzo[b][1,4]dioxin-2-yl)methyl)piperidin-3-yl)imidazolidine-2,4,5-trione, 1-((S)-1-(((S)-2,3-dihydrobenzo[b][1,4]dioxin-2-yl)methyl)piperidin-3-yl)-3-(pyridin-4-ylmethyl)imidazolidine-2,4,5-trione, 1-((S)-1-(((S)-2,3-dihydrobenzo[b][1,4]dioxin-2-yl)methyl)piperidin-3-yl)-3-isobutylimidazolidine-2,4,5-trione, 1-((S)-1-(((S)-2,3-dihydrobenzo[b][1,4]dioxin-2-yl)methyl)piperidin-3-yl)-3-(pyridin-2-ylmethyl)-imidazolidine-2,4,5-trione, 1-((S)-1-(((S)-2,3-dihydrobenzo[b][1,4]dioxin-2-yl)methyl)-piperidin-3-yl)-3-(pyridin-3-ylmethyl)imidazolidine-2,4,5-trione, 1-((S)-1-(((S)-2,3-dihydrobenzo[b][1,4]dioxin-2-yl)methyl)piperidin-3-yl)-1H-benzo[d]imidazol-2(3H)-one, 1-((S)-1-(((S)-2,3-dihydrobenzo[b][1,4]dioxin-2-yl)methyl)piperidin-3-yl)-1H-benzo-[d][1,2,3]triazole, 1-((S)-1-(((S)-2,3-dihydrobenzo[b][1,4]dioxin-2-yl)methyl)piperidin-3-yl)-1,3-dihydrobenzo[c][1,2,5]thiadiazole-2,2-dioxide, 1-((S)-1-(((S)-2,3-dihydrobenzo-[b][1,4]dioxin-2-yl)methyl)piperidin-3-yl)-3-methyl-1H-benzo[d]imidazol-2(3H)-one, 1-((S)-1-(((S)-2,3-dihydrobenzo[b][1,4]dioxin-2-yl)methyl)piperidin-3-yl)-1H-benzo[d]imidazole, 1-((S)-1-(((S)-2,3-dihydrobenzo[b][1,4]dioxin-2-yl)methyl)piperidin-3-yl)-3-phenyl-1H-benzo[d]imidazol-2(3H)-one, 1-((S)-1-(((S)-2,3-dihydrobenzo[b][1,4]-dioxin-2-yl)methyl)piperidin-3-yl)-2-methyl-1H-benzo[d]imidazole, 1-((S)-1-(((S)-2,3-dihydrobenzo[b][1,4]dioxin-2-yl)methyl)piperidin-3-yl)-2-(methoxymethyl)-1H-benzo[d]-imidazole, 1-(6-chloro-1-((S)-1-(((S)-2,3-dihydrobenzo[b][1,4]dioxin-2-yl)methyl)piperidin-3-yl)-1H-benzo[d]imidazol-2-yl)-N,N-dimethylmethanamine, 1-(6-chloro-1-((S)-1-(((S)-2,3-dihydrobenzo[b][1,4]dioxin-2-yl)methyl)piperidin-3-yl)-1H-benzo[d]imidazol-2-yl)-N-methylmethanamine, 1-((S)-1-(((S)-2,3-dihydrobenzo[b][1,4]dioxin-2-yl)methyl)piperidin-3-yl)-4-fluoro-1H-benzo[d]imidazol-2(3H)-one, 3-((S)-1-(((S)-2,3-dihydrobenzo[b][1,4]-dioxin-2-yl)methyl)piperidin-3-yl)-5-methoxy-2-methyl-3H-imidazo[4,5-b]pyridine, 3-((S)-1-(((S)-2,3-dihydrobenzo[b][1,4]dioxin-2-yl)methyl)-piperidin-3-yl)-2-methyl-3H-imidazo[4,5-b]pyridine, 1-((S)-1-(((S)-2,3-dihydrobenzo[b][1,4]dioxin-2-yl)methyl)-piperidin-3-yl)-2-methyl-1H-imidazo[4,5-b]pyridine, 1-((S)-1-(((S)-2,3-dihydrobenzo-[b][1,4]dioxin-2-yl)methyl)piperidin-3-yl)-2-methyl-1H-imidazo[4,5-c]pyridine, 3-((S)-1-(((S)-2,3-dihydrobenzo[b][1,4]dioxin-2-yl)methyl)piperidin-3-yl)-2-ethyl-3H-imidazo[4,5-b]pyridine, 9-((S)-1-(((S)-2,3-dihydrobenzo[b][1,4]dioxin-2-yl)methyl)piperidin-3-yl)-8-methyl-9H-purine, 9-((S)-1-(((S)-2,3-dihydrobenzo[b][1,4]dioxin-2-yl)methyl)-piperidin-3-yl)-9H-purine, 2-((S)-1-(((S)-2,3-dihydrobenzo[b][1,4]dioxin-2-yl)methyl)piperidin-3-yl)-5,5-dimethylisothiazolidine-1,1-dioxide, or 3-((S)-1-(((S)-2,3-dihydrobenzo[b][1,4]dioxin-2-yl)methyl)piperidin-3-yl)oxazolidine-2,4-dione.

The terms employed herein have the meanings indicated below.

The term “at least one”, employed in the meanings below, refers to one or several, such as one.

The term “halo” or “halogen”, as employed herein as such or as part of another group, refers to fluorine, chlorine, bromine, or iodine.

The term “oxo”, as employed herein as such or as part of another group, refers to a ═O group attached as a substituent.

The term “(C₁-C₆)alkyl”, as employed herein as such or as part of another group, refers to a saturated hydrocarbon group having a straight or branched moiety, containing 1, 2, 3, 4, 5 or 6 carbon atom(s). Representative examples of (C₁-C₆)alkyl include, but are not limited to, methyl, ethyl, n-propyl, iso-propyl, n-butyl, iso-butyl, sec-butyl, tert-butyl, n-pentyl, iso-pentyl, and n-hexyl.

The term “cyclo(C₃-C₆)alkyl”, as employed herein as such or as part of another group, refers to a saturated hydrocarbon group having cyclic moiety, containing 3, 4, 5, or 6 carbon atom(s). Representative examples of cyclo(C₃-C₆)alkyl include, but are not limited to cyclopropyl, cyclobutyl, cyclopentyl, and cyclohexyl.

The term “cyclo(C₃-C₆)alkyl(C₁-C₆)alkyl”, as employed herein as such or as part of another group, refers to a cyclo(C₃-C₆)alkyl group, as defined herein, bonded to an (C₁-C₆)alkyl group, as defined herein. Representative examples of cyclo(C₃-C₆)alkyl(C₁-C₆)alkyl include, but are not limited to, cyclopropylmethyl, cyclobutylmethyl, cyclopentylmethyl, and cyclohexylmethyl.

The term “(C₁-C₆)alkoxy”, as employed herein as such or as part of another group, refers to an (C₁-C₆)alkyl group, as defined herein, bonded to an oxygen atom. Representative examples of (C₁-C₆)alkoxy include, but are not limited to, methoxy, ethoxy, n-propoxy, n-butoxy, iso-butoxy, sec-butoxy, tert-butoxy, 2,2-dimethylpropoxy, 3-methylbutoxy, and n-hexoxy.

The term “(C₁-C₆)alkoxy(C₁-C₆)alkyl”, as employed herein as such or as part of another group, refers to at least one (C₁-C₆)alkoxy group, as defined herein, bonded to an (C₁-C₆)alkyl group, as defined herein. When there are several (C₁-C₆)alkoxy groups, the (C₁-C₆)alkoxy groups can be identical or different. Representative examples of (C₁-C₆)alkoxy(C₁-C₆)alkyl include, but are not limited to, methoxymethyl, ethoxymethyl, propoxymethyl, 2-methoxyethyl, 2-ethoxyethyl, 2,2-dimethoxyethyl, 1-methyl-2-propoxyethyl, 1-methoxy-1-methylethyl, and 4-methoxybutyl.

The term “(C₁-C₆)alkyl-(C═O)”, as employed herein as such or as part of another group, refers to a (C₁-C₆)alkyl group, as defined herein, bonded to a carbonyl group. Representative examples of (C₁-C₆)alkyl-(C═O) include, but are not limited to, acetyl, ethylcarbonyl, propylcarbonyl, and isopropylcarbonyl.

The term “(C₁-C₆)alkyl-S-”, as employed herein as such or as part of another group, refer to an (C₁-C₆)alkyl group, as defined herein, bonded to a sulfur atom. Representative examples of (C₁-C₆)alkyl-S— include, but are not limited to, thiomethyl, thioethyl, thiopropyl, and thiobutyl.

The term “phenyl(C₁-C₆)alkyl”, as employed herein as such or as part of another group, refers to a phenyl group, bonded to a (C₁-C₆)alkyl group, as defined herein. Representative examples of phenyl(C₁-C₆)alkoxy include, but are not limited to, benzyl, 2-phenylethyl, 3-phenylpropyl, and 2-phenyl-2-methyl-ethyl.

The term “heterocyclyl” or “heterocyclic ring”, as employed herein as such or as part of another group, refers to a 4, 5 or 6 membered saturated or unsaturated monocyclic group containing 1, 2, or 3 ring heteroatom(s) each independently selected from N, O, and S. Representative examples of heterocyclyl or heterocyclic ring include, but are not limited to pyrrolidin-1-yl, imidazolidin-1-yl, oxazolidin-3-yl, isothiazolidinyl, pyrazol-1-yl hexahydropyrimidin-1-yl, piperidin-1-yl, piperidin-2-yl, piperidin-3-yl, piperidin-4-yl, pyridin-2-yl, pyridin-3-yl, pyridin-4-yl, pyrimidin-2-yl, pyrimidin-4-yl morpholin-4-yl, tetrahydropyran-4-yl, azetidin-1-yl, and oxetan-3-yl.

The term “heterocyclyl(C₁-C₆)alkyl”, as employed herein as such or as part of another group, refers to a heterocyclyl group, as defined herein, bonded to a (C₁-C₆)alkyl group, as defined herein. Representative examples of heterocyclyl(C₁-C₆)alkyl include, but are not limited to, pyridin-2-ylmethyl, pyridin-3-ylmethyl, pyridin-4-ylmethyl, pyridin-2-ylethyl, pyridin-3-ylethyl, and pyridin-4-ylethyl.

The term “bridge”, as employed herein, refers to a valence bond, an atom, or an unbranched chain of atoms connecting two different parts of molecule.

The expression “compounds of the present disclosure”, as employed herein refers to the compounds of formula I or Ia.

The “pharmaceutically acceptable salts” according to the present disclosure include therapeutically active, non-toxic, base and acid salt forms, which the compounds of formula I are able to form with both organic and inorganic bases and acids. Representative examples of pharmaceutically acceptable base addition salt forms, for example, metal or amine salts, include, but are not limited to, ammonium salts, lithium, sodium, potassium, calcium, magnesium, aluminum and zinc salts, salts with organic bases, such as N-methyl-D-glucamine, hydrabamine salts and salts with amino acids, such as arginine, lysine, and the like. Representative examples of pharmaceutically acceptable acid addition salts include, but are not limited to, chlorides, bromides, sulfates, nitrates, phosphates, sulfonates, methane sulfonates, formates, tartrates, maleates, citrates, benzoates, salicylates, ascorbates, acetates and oxalates, fumarates, and succinates.

Pharmaceutically acceptable esters, when applicable, may be prepared by known methods using pharmaceutically acceptable acids that are conventional in the field of pharmaceuticals and that retain the pharmacological properties of the free form. Non-limiting examples of these esters include esters of aliphatic or aromatic alcohols. Representative examples of pharmaceutically acceptable esters include, but are not limited to, methyl, ethyl, n-propyl, iso-propyl, n-butyl, iso-butyl, sec-butyl, tert-butyl, and benzyl esters.

The present disclosure includes all the possible geometric isomers, for example cis and trans isomers, of the compounds of formula I, as well as all the possible optical isomers, such as diastereomers and enantiomers, of the compound of formula I. Furthermore, the present disclosure includes all the individual isomers and any mixtures thereof, such as racemic mixture. The individual isomers may be obtained using the corresponding isomeric forms of the starting materials or they may be separated after the preparation of the end compound according to conventional separation methods. For the separation of optical isomers, such as enantiomers, from the mixture thereof, conventional resolution methods, for example fractional crystallization or preparative chiral chromatography, may be used.

Compounds of the invention can be prepared by a variety of synthetic routes analogously or according to the methods known in the literature using suitable starting materials. The starting materials used in the processes herein are either commercially available or can be prepared via synthetic routes known in the literature.

In general, compounds of formula I can be prepared analogously or according to the following scheme 1:

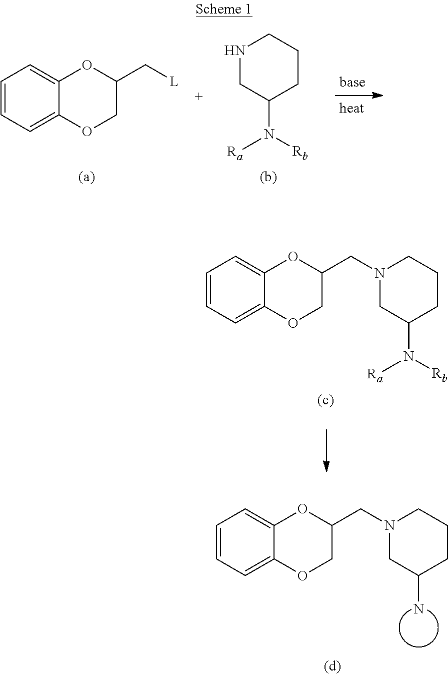

For example, suitable starting materials containing the benzodioxane moiety are compounds of formula (a), wherein L is a leaving group, e.g. halogen, mesylate, or tosylate. Compounds of formula (a) can be prepared according to known methods.

Suitable starting materials containing the piperidine ring are compounds of formula (b), wherein NRₐR_{b} is for example, NH₂, NHBoc-t, or N-containing heterocycle.

The compounds of formula (c) wherein NRₐR_{b} is N-containing heterocycle, can be directly prepared from known compound (a) and suitably substituted piperidine (b) with bases under 25° C. to 150° C. in a suitable solvent.

In addition, compounds of formula (d) can be further synthesized from compounds of formula (c) wherein NRₐR_{b} is NH₂, by alkylation or acetylation with an appropriate leaving group followed by internal cyclizations with known methods.

The following schemes 2 and 3 describe the general synthesis of some more specific classes of compounds exemplified in this disclosure.

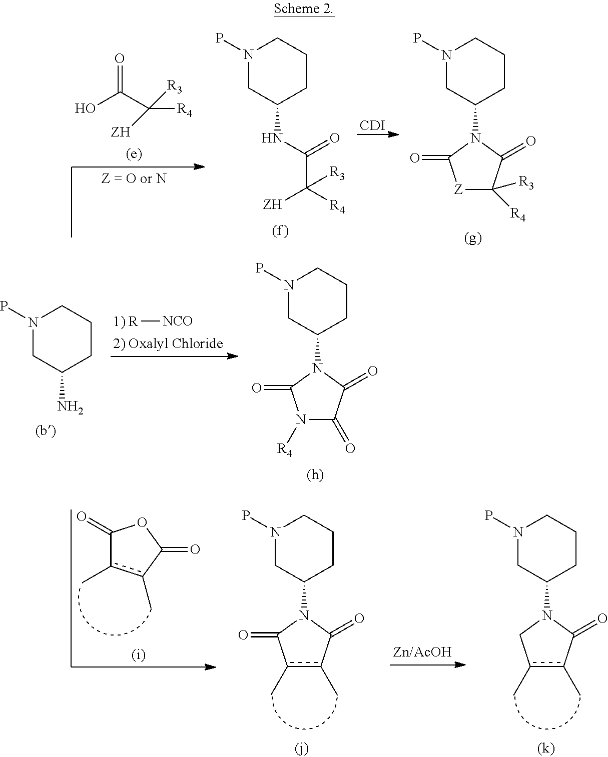

In scheme 2, a suitably N-protected (P is e.g. CBz or t-Boc) 3-amino piperidine (b′) can be acylated with carboxylating reagent (e) under standard amide coupling procedures to form amide (f), which can be cyclized with further acylation, e.g. by CDI, to form five membered heterocycles (g). Compound of formula (b′) can also react with isocyanates and subsequent treatment with oxalyl chloride to yield tricarbonylated compounds (h). Furthermore, compound of formula (b′) can also directly react with various aliphatic substituted or aryl/heteroaryl fused anhydrides (i) to form compounds of formula (j), which can be partially reduced by Zn/AcOH to compounds of formula (k).

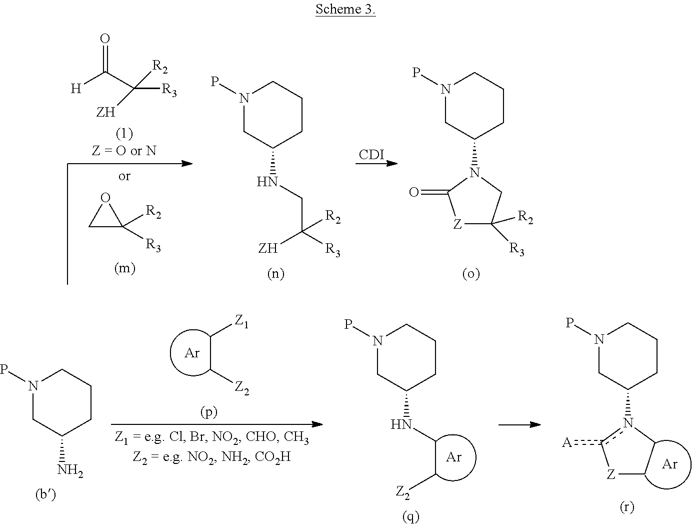

In scheme 3, compound of formula (b′) (P is e.g. compound of formula (a), CBz or t-Boc) can be N-alkylated by reductive amination with aldehydes (1) or epoxides (m) to form compound of formula (n), which can be cyclized by carbonylating reagents, such as CDI, to form compounds of formula (o). In addition, compound of formula (b′) can also react with suitably ortho-substituted aryl/heteroaryl (p) by standard displacement to form functionalized compounds of formula (q), which can be cyclized with carbonylating agents, such as carboxylic acid, acid anhydrides, aldehydes, CDI etc., to form aryl/heteroaryl fused compounds of formula (r), wherein A is e.g. H, O, or alkyl.

A person skilled in the art realizes that any starting material or intermediate in the reactions described above can be protected, if necessary, in a manner known in the art. Any protected functionality can subsequently be deprotected in a manner known in the art.

The synthetic routes described above are meant to illustrate the preparation of the compounds of formula I and the preparation is by no means limited thereto, that is, there are also other possible synthetic methods which are within the general knowledge of a person skilled in the art.

The compounds of formula I may be converted, if desired, into their pharmaceutically acceptable salt or ester form using methods known in the art.

The present disclosure will be explained in more detail by the following examples. The examples are meant for illustrating purposes only and do not limit the scope of the invention defined in the claims.

Normal phase and reverse phase flash chromatography was performed using CombiFlash instruments together with disposable Redisep columns (Teledyne ISCO). Preparative HPLC purifications were performed with a Waters preparative HPLC/MS autopurification system equipped with an XBridge Prep C18 (5 μm, 30×150 mm) column. Typically, a gradient of water/acetonitrile with 0.1% formic acid was used as eluent. Microwave heating was performed using microwave reactors from Biotage. The structures of the products were confirmed by ¹H NMR. The spectra were measured with a Bruker Avance 400 instrument. LC-MS analyses were performed using a Waters Acquity UPLC/MS/MS with a TQ detector. For the chiral HPLC analysis, Agilent 1100-series HPLC instrument equipped with diode array detector was used.

The following general abbreviations are used: EtOAc=ethyl acetate, DCE=1,2-dichloroethane, NaBH(OAc)₃=sodium triacetoxyborohydride, CDI=1,1′-carbonyldiimidazole, TFA=trifluoroacetic acid, ACN=acetonitrile, AcOH=acetic acid, Ac₂O=acetic anhydride, DEA=diethanolamine, IPA=isopropyl alcohol, DMSO-d₆=deuterated dimethyl sulfoxide, D₂O=deuterium oxide, CDCl₃=deuterated chloroform, DIPEA=N,N-disopropylethylamine, DCM=dichloromethane, DMF=N,N-dimethylformamide, THF=tetrahydrofuran, AIBN=azobisisobutyronitrile, NBS=N-bromosuccinimide, HCl=hydrochloric acid, PCC=pyridinium chloro-chromate, MTBE=methyl tert-butyl ether, Pd/C=palladium on carbon, Pd₂(dba)₃=tris-(dibenzylideneacetone)dipalladium(0), LiHMDS=lithium hexamethyldisilazide, DMAP=4-dimethyl-aminopyridine, HOBt=hydroxybenzotriazole, TEA=triethylamine, EDC HCl=1-ethyl-3-(3-dimethylamino-propyl)carbodiimide hydrochloride, PdCl₂(dppf.).CH₂Cl₂=1,1′-bis(diphenylphosphino)-ferrocene-dichloropalladium(II) dichloromethane complex, Pd(OAc)₂=palladium(II) acetate, XPhos=2-dicyclohexylphosphino-2′,4′,6′-triisopropylbiphenyl, p-TsOH=p-toluenesulfonic acid, T3P=propylphosphonic anhydride, KOtBu=potassium tert-butoxide, HBTU=2-(1H-benzotriazol-1-yl)-1,1,3,3-tetramethyluronium hexafluorophosphate, LDA=lithium diisopropylamide, TEMPO=(2,2,6,6-tetramethylpiperidin-1-yl)oxyl, TBTU=2-(1H-benzotriazole-1-yl)-1,1,3,3-tetramethyluronium tetrafluoroborate, HEPES=4-(2-hydroxyethyl)-1-piperazine-ethanesulfonic acid, EDTA=ethylenediaminetetraacetic acid, RT=room temperature, MW=microwave, LC-MS=liquid chromatography-mass spectrometry, SFC=supercritical fluid chromatography, HPLC=high performance liquid chromatography, RP-HPLC=reversed phase HPLC, ¹H NMR=proton nuclear magnetic resonance.

### Preparation of the Compounds of the Present Disclosure

### General Procedure A

1-(Piperidin-3-yl) derivative (1 eq) was dissolved in acetonitrile or DMF (˜1 M) in microvial. DIPEA (0-1.2 eq), K₂CO₃ (1.5-2.5 eq) and benzodioxin derivative (1-1.2 eqv) were added under nitrogen and the vial was sealed. The reaction mixture was heated at 120° C. for 3 hours. The solvents were removed under reduced pressure.

### General Procedure B

To a solution of suitable 4-oxobutanoate derivative (1 eq) and (S)-3-aminopiperidine derivative (1-1.1 eq) in DCE (0.1-0.2 M) was added NaBH(OAc)₃ (1.2-2 eq) at 0° C. then stirred at rt for 6-18 h. The reaction mixture was quenched with water and extracted with DCM. The organic layer was dried and evaporated.

### General Procedure C

To a solution of suitable 2-hydroxy asetamide derivative (1 eq) in DMF (0.1-0.28 M) was added Et₃N or DIPEA (2-3 eq) at 0° C., followed by CDI (1.2-2.5 eq) and the resulting mixture was stirred at RT or heated at 90° C. for 16 h. The solvent was evaporated off and the residue was dissolved in EtOAc, washed with water, dried and evaporated to dryness.

### General Procedure D

The starting material was dissolved in ethyl acetate (50-120 mM) and 10% Pd/C (15-75 wt %) was added. The reaction was hydrogenated 5-16 h. After completion, the reaction mixture was filtered through celite pad and evaporated.

### General Procedure E

(S)-tert-Butyl piperidine-1-carboxylate derivative (1 eq) was taken in HCl in Et₂O or dioxane (10-15 eq) at 0° C. and the resulting mixture was stirred at RT for 2-5 h. The solvents were evaporated.

### General Procedure F

Nitro compound was dissolved in THF/MeOH/H₂O (4:1:1 to 2:1:1 v/v/v). NH₄Cl (10 eq) and Zn dust (10 eq) were added at 0° C. The resulting mixture was stirred at 0° C. for 5 min, allowed to warm to RT and stirred until completion. The mixture was filtered through a Celite pad, diluted with EtOAc, washed with brine, dried, and evaporated to dryness.

### Intermediate 1: (S)-1-(((S)-2,3-Dihydrobenzo[b][1,4]dioxin-2-yl)methyl)piperidin-3-amine

A flask was charged with (2R)-2-(bromomethyl)-2,3-dihydro-1,4-benzodioxin (4.49 g, 19.61 mmol) or (R)-(2,3-dihydrobenzo[b][1,4]dioxin-2-yl)methyl 4-methylbenzenesulfonate (6.28 g, 19.61 mmol, U.S. Pat. No. 5,767,116 A1), (S)-tert-butyl piperidin-3-ylcarbamate (3.57 g, 17.83 mmol), sodium carbonate (2.267 g, 21.39 mmol) and DMF (60 mL). Reaction was heated to 100-110° C. for 6 h. Mixture was allowed to cool to room temperature and acidified by addition of 1 M HCl solution (70 mL). Aqueous mixture was washed with MTBE (2×50 mL), then basified by addition of solid Na₂CO₃. Oily solution was extracted with MTBE (3×50 mL). Organic extracts were washed with brine (50 mL), dried with anhydrous Na₂SO₄ and evaporated to dryness to give 5.94 g of crude tert-butyl (S)-1-(((S)-2,3-dihydrobenzo[b][1,4]dioxin-2-yl)methyl)piperidin-3-ylcarbamate as brown oil. This oil was mixed with 4 M HCl solution (43 mL) and heated to 60° C. for 2 hours. Reaction mixture was allowed to cool to room temperature and washed with EtOAc (15 mL). Aqueous phase was basidified by addition of 6 M NaOH solution to pH 10 and extracted with EtOAc (3×25 mL). Combined organic extracts were washed with brine (25 mL), dried with anhydrous Na₂SO₄ and evaporated to dryness to give 3.52 g (80%) of (S)-1-(((S)-2,3-dihydrobenzo[b][1,4]dioxin-2-yl)methyl)piperidin-3-amine as oil.

LC-MS (ES+) [M+1]: 249.5.

### Intermediate 2: (S)-1-(((S)-2,3-Dihydrobenzo[b][1,4]dioxin-2-yl)methyl)piperidin-3-amine, p-toluenesulfonate

A flask was charged with (S)-1-(((S)-2,3-dihydrobenzo[b][1,4]dioxin-2-yl)methyl)piperidin-3-amine (3.90 g, 15.72 mmol, Intermediate 1) and acetonitrile (100 mL). To this was added p-toluenesulfonic acid monohydrate (2.99 g, 15.72 mmol) and mixture was heated to reflux until became clear. Solution was allowed to cool towards room temperature and seeded with previously obtained product crystals. Once mixture was cooled to room temperature, it was further cooled with ice bath. Solids were filtered and washed with cold acetonitrile. Product was dried in 40° C. vacuum oven to give 5.2 g of (S)-1-(((S)-2,3-dihydrobenzo[b][1,4]dioxin-2-yl)methyl)piperidin-3-amine, p-toluenesulfonate as white solids.

LC-MS (ES+) [M-OTs]: 249.5.

### EXAMPLE 1: 1-((S)-1-(((S)-2,3-Dihydrobenzo[b][1,4]dioxin-2-yl)methyl)piperidin-3-yl)-4,4-dimethylpyrrolidin-2-one

### Step 1: (S)-Benzyl 3-(4,4-dimethyl-2-oxopyrrolidin-1-yl)piperidine-1-carboxylate

(S)-Benzyl 3-(4,4-dimethyl-2-oxopyrrolidin-1-yl)piperidine-1-carboxylate was prepared according to the general procedure B using methyl 3,3-dimethyl-4-oxobutanoate (1.5 g, 10.4 mmol, Organic Syntheses 1993, 71, 189), (S)-benzyl 3-aminopiperidine-1-carboxylate (2.68 g, 11.4 mmol) and NaBH(OAc)₃ (2.64 g, 12.5 mmol) and DCE (100 ml). The product was purified by flash chromatography to obtain 1 g of (S)-benzyl 3-(4,4-dimethyl-2-oxopyrrolidin-1-yl)piperidine-1-carboxylate.

LC-MS (ES+) [M+1]: 331.2.

### Step 2: (S)-4,4-Dimethyl-1-(piperidin-3-yl)pyrrolidin-2-one

The intermediate was prepared according to the general procedure D using (S)-benzyl 3-(4,4-dimethyl-2-oxopyrrolidin-1-yl)piperidine-1-carboxylate from Step 1 (1.2 g, 3.6 mmol), 10% Pd/C (200 mg) and EtOAc (50 ml). The crude product was purified by washing with diethyl ether and pentane to obtain 200 mg of the product.

LC-MS (ES+) [M+1]: 197.2.

### Step 3: 1-((S)-1-(((S)-2,3-Dihydrobenzo[b][1,4]dioxin-2-yl)methyl)piperidin-3-yl)-4,4-dimethylpyrrolidin-2-one

The 1-((S)-1-(((S)-2,3-dihydrobenzo[b][1,4]dioxin-2-yl)methyl)piperidin-3-yl)-4,4-dimethylpyrrolidin-2-one was prepared according to the general procedure A using (S)-4,4-dimethyl-1-(piperidin-3-yl)pyrrolidin-2-one (100 mg, 0.509 mmol), acetonitrile (0.5 ml), DIPEA (0.106 ml, 0.611 mmol), K₂CO₃ (106 mg, 0.764 mmol) and (2R)-2-(bromomethyl)-2,3-dihydro-1,4-benzodioxin (117 mg, 0.509 mmol). The crude product was purified by reversed phase flash chromatography to obtain 98 mg of the product.

¹H NMR (400 MHz, CDCl₃) δ ppm 1.13-1.15 (m, 6H), 1.25-1.45 (m, 1H), 1.63-1.78 (m, 3H), 2.06-2.17 (m, 2H), 2.21 (s, 2H), 2.64 (d, 2H), 2.81 (d, 1H), 2.87-2.94 (m, 1H), 3.05-3.14 (m, 2H), 3.95-4.05 (m, 1H), 4.11-4.21 (m, 1H), 4.25-4.33 (m, 2H), 6.81-6.89 (m, 4H).

### EXAMPLE 2: 1-((S)-1-(((S)-2,3-Dihydrobenzo[b][1,4]dioxin-2-yl)methyl)piperidin-3-yl)-4,4-diphenylimidazolidin-2-one

### Step 1: Benzyl (2-oxo-1,1-diphenylethyl)carbamate

To an ice cold stirred solution of benzyl (2-hydroxy-1,1-diphenylethyl)carbamate (8.0 g, 23.05 mmol, European Journal of Organic Chemistry, 2008 (2), 350) in DCM (200 ml) was added a mixture of PCC (9.9 g, 46.10 mmol) and silica gel (10 g) and stirred at rt for 12 h. The reaction mixture was diluted with DCM and filtered through a pad celite. The filtrate was evaporated to obtain the crude compound, which was purified by flash column using to obtain 4.0 g of the product.

LC-MS (ES+) [M+1]: 346.1.

### Step 2: (S)-tert-Butyl 3-((2-(((benzyloxy)carbonyl)amino)-2,2-diphenylethyl)amino)-piperidine-1-carboxylate

(S)-tert-Butyl 3-((2-(((benzyloxy)carbonyl)amino)-2,2-diphenylethyl)amino)piperidine-1-carboxylate was prepared according to the general procedure B using (S)-tert-butyl 3-aminopiperidine-1-carboxylate (2.32 g, 11.59 mmol), benzyl (2-oxo-1,1-diphenylethyl)-carbamate from Step 1 (4.0 g, 11.59 mmol), DCE (100 ml) and NaBH(OAc)₃ (4.9 g, 23.18 mmol). The evaporation residue was purified by column chromatography to obtain 3.2 g of the product.

LC-MS (ES+) [M+1]: 530.3.

### Step 3: (S)-tert-Butyl 3-((2-amino-2,2-diphenylethyl)amino)piperidine-1-carboxylate

(S)-tert-Butyl 3-((2-amino-2,2-diphenylethyl)amino)piperidine-1-carboxylate was prepared according to the general procedure D using (S)-tert-butyl 3-((2-(((benzyloxy)carbonyl)-amino)-2,2-diphenylethyl)amino)piperidine-1-carboxylate (3.2 g, 6.05 mmol), 10% Pd/C (1.5 g) and EtOAc (50 ml). The evaporation residue was purified by triturating with n-pentane/Et₂O to obtain 2.0 g of the product.

LC-MS (ES+) [M+1]: 396.2.

### Step 4: (S)-tert-Butyl 3-(2-oxo-4,4-diphenylimidazolidin-1-yl)piperidine-1-carboxylate

To an ice cold stirred solution of (S)-tert-butyl 3-((2-amino-2,2-diphenylethyl)amino)-piperidine-1-carboxylate (2.0 g, 5.06 mmol) in DCM (100 ml) was added Et₃N (1.84 ml, 13.16 mmol) and triphosgene (0.49 g, 1.67 mmol). The reaction mixture was stirred at rt for 2 h. 10% aqueous NaHCO₃ was added and the reaction mixture extracted with DCM (2×150 ml). The combined organic layers were washed with water, dried over anhydrous sodium sulfate and concentrated under reduced. The residue was purified riturated with Et₂O/pentane to obtain 1.5 g of the product.

LC-MS (ES+) [M+1]: 422.2.

### Step 5: (S)-4,4-Diphenyl-1-(piperidin-3-yl)imidazolidin-2-one hydrochloride

(S)-4,4-Diphenyl-1-(piperidin-3-yl)imidazolidin-2-one hydrochloride was prepared according to the general procedure E using (S)-tert-butyl 3-(2-oxo-4,4-diphenylimidazolidin-1-yl)piperidine-1-carboxylate (1.0 g, 2.37 mmol) in Et₂O (5 ml) and 1M HCl in Et₂O (30 ml). The residue was triturated with Et₂O/pentane to obtain 380 mg of product as HCl salt.

LC-MS (ES+) [M+1]: 322.2.

### Step 6: 1-((S)-1-(((S)-2,3-Dihydrobenzo[b][1,4]dioxin-2-yl)methyl)piperidin-3-yl)-4,4-diphenylimidazolidin-2-one

1-((S)-1-(((S)-2,3-Dihydrobenzo[b][1,4]dioxin-2-yl)methyl)piperidin-3-yl)-4,4-diphenylimidazolidin-2-one was prepared according to general procedure A using (S)-4,4-diphenyl-1-(piperidin-3-yl)imidazolidin-2-one (100 mg, 0.311 mmol), (2R)-2-(bromomethyl)-2,3-dihydro-1,4-benzodioxin (71.3 mg, 0.311 mmol), DIPEA (0.065 ml, 0.373 mmol), K₂CO₃ (64.5 mg, 0.467 mmol) and ACN (0.5 ml). The product was purified by reversed phase flash chromatography to obtain 70 mg of the product.

¹H NMR (400 MHz, CDCl₃) δ ppm 1.43-1.53 (m, 1H), 1.62-1.78 (m, 2H), 1.79-1.87 (m, 1H), 2.13-2.31 (m, 2H), 2.50-2.71 (m, 2H), 2.78 (d, 1H), 2.98 (dd, 1H), 3.97-4.06 (m, 4H), 4.26-4.32 (m, 2H), 4.94 (s, 1H), 6.81-6.89 (m, 4H), 7.29-7.38 (m, 10H).

### EXAMPLE 3: 1-((S)-1-(((S)-2,3-Dihydrobenzo[b][1,4]dioxin-2-yl)methyl)piperidin-3-yl)-4-phenylimidazolidin-2-one

### Step 1: (3S)-Benzyl 3-((2-((tert-butoxycarbonyl)amino)-2-phenylethyl)amino)-piperidine-1-carboxylate

(3S)-Benzyl 3-((2-((tert-butoxycarbonyl)amino)-2-phenylethyl)amino)piperidine-1-carboxylate was prepared according to the general procedure B using (S)-benzyl 3-aminopiperidine-1-carboxylate (3.0 g, 12.82 mmol), (R)-tert-butyl (2-oxo-1-phenylethyl)carbamate (3.01 g, 12.82 mmol, WO2006/014357), DCE (60 ml) and NaBH(OAc)₃ (4.07 g, 19.23 mmol). The crude product was purified by flash chromatography to obtain 2.0 g of the product.

LC-MS (ES+) [M+1]: 325.1.

### Step 2: (3S)-Benzyl 3-((2-amino-2-phenylethyl)amino)piperidine-1-carboxylate hydrochloride

(3S)-Benzyl 3-((2-amino-2-phenylethyl)amino)piperidine-1-carboxylate hydrochloride was prepared according to the general procedure E using (3S)-benzyl 3-((2-((tert-butoxycarbonyl)amino)-2-phenylethyl)amino)piperidine-1-carboxylate (2.0 g, 4.41 mmol), Et₂O (20 mil) and Et₂O—HCl (1 M, 20 ml). The crude product was purified by triturating with Et₂O/n-pentane to obtain 1.25 g of the product as HCl salt.

LC-MS (ES+) [M+1]: 380.1.

### Step 3: (3S)-Benzyl 3-(2-oxo-4-phenylimidazolidin-1-yl)piperidine-1-carboxylate

(3S)-Benzyl 3-(2-oxo-4-phenylimidazolidin-1-yl)piperidine-1-carboxylate was prepared according to the general procedure C using (3S)-benzyl 3-((2-amino-2-phenylethyl)amino)-piperidine-1-carboxylate hydrochloride (1.25 g, 3.21 mmol), DMF (20 ml), Et₃N (0.985 ml, 7.06 mmol) and CDI (0.624 g, 3.85 mmol). The crude product was purified by flash chromatography to obtain 560 mg of the product.

LC-MS (ES+) [M+1]: 354.1.

### Step 4: 4-Phenyl-1-((S)-piperidin-3-yl)imidazolidin-2-one

4-Phenyl-1-((S)-piperidin-3-yl)imidazolidin-2-one was prepared according to the general procedure D using of (3S)-benzyl 3-(2-oxo-4-phenylimidazolidin-1-yl)piperidine-1-carboxylate (500 mg, 2.11 mmol), 10% Pd/C (400 mg) and EtOAc (20 ml). The product was purified by triturating with Et₂O/n-pentane to obtain 180 mg of the product.

LC-MS (ES+) [M+1]: 245.9.

### Step 5: 1-((S)-1-(((S)-2,3-Dihydrobenzo[b][1,4]dioxin-2-yl)methyl)piperidin-3-yl)-4-phenylimidazolidin-2-one

1-((S)-1-(((S)-2,3-Dihydrobenzo[b][1,4]dioxin-2-yl)methyl)piperidin-3-yl)-4-phenylimidazolidin-2-one was prepared according to the general procedure A using 4-phenyl-1-((S)-piperidin-3-yl)imidazolidin-2-one (100 mg, 0.408 mmol), (2R)-2-(bromomethyl)-2,3-dihydro-1,4-benzodioxin (93 mg, 0.408 mmol), DIPEA (0.085 ml, 0.489 mmol), K₂CO₃ (85 mg, 0.611 mmol) and ACN (0.5 ml). The crude product was purified by reversed phase flash chromatography to obtain 69.2 mg of the product.

¹H NMR (400 MHz, CDCl₃) δ ppm 1.27-1.47 (m, 1H), 1.60-1.74 (m, 2H), 1.74-1.88 (m, 1H), 2.03-2.30 (m, 2H), 2.62 (ddd, 2H), 2.78 (dd, 1H), 2.90-3.04 (m, 1H), 3.20-3.32 (m, 1H), 3.85-3.91 (m, 1H), 3.92-4.04 (m, 2H), 4.21-4.35 (m, 2H), 4.60 (br. s., 1H), 4.69-4.76 (m, 1H), 6.78-6.89 (m, 4H), 7.30-7.41 (m, 5H).

### EXAMPLE 4: (3R,4R)-1-((S)-1-(((S)-2,3-Dihydrobenzo[b][1,4]dioxin-2-yl)methyl)piperidin-3-yl)-3,4-dimethylpyrrolidine-2,5-dione

### Step 1: (S)-tert-Butyl 3-(3,4-dimethyl-2,5-dioxo-2,5-dihydro-1H-pyrrol-1-yl)piperidine-1-carboxylate

To a stirred solution of (S)-tert-butyl 3-(methylamino)piperidine-1-carboxylate (5 g, 25 mmol) in toluene (150 ml) was added triethylamine (5.42 ml, 37.5 mmol) followed by 3,4-dimethylfuran-2,5-dione (3.15 g, 25 mmol) at RT and the resulting reaction mixture was heated at reflux temperature for 16 h. The reaction mixture was cooled to RT and diluted with EtOAc and washed with water. The organic layer was dried and evaporated. The crude product was purified by flash chromatography to obtain 6.0 g of the product.

¹H NMR (300 MHz, CDCl₃) δ ppm 1.45 (s, 9H), 1.65-1.83 (m, 2H), 1.95 (s, 6H), 2.05-2.29 (m, 2H), 2.60-2.75 (m, 1H), 3.22-3.35 (m, 1H), 3.92-4.10 (m, 3H).

### Step 2: (3S)-tert-Butyl 3-((3R,4R)-dimethyl-2,5-dioxopyrrolidin-1-yl)piperidine-1-carboxylate

To a stirred solution of (S)-tert-butyl 3-(3,4-dimethyl-2,5-dioxo-2,5-dihydro-1H-pyrrol-1-yl)piperidine-1-carboxylate from Step 1(800 mg, 2.5 mmol) and NiCl₂.6H₂O (61 mg, 0.25 mmol) in methanol (20 ml) was added NaBH₄ (99 mg, 2.5 mmol) at −10° C. and stirred for 1 h. The reaction mixture was neutralized with 1M HCl and extracted with ethyl acetate. The organic layer was dried evaporated. The crude compound was purified by flash chromatography followed by combi flash to obtain 260 mg of pure isomer.

LC-MS (ES+) [M+1]: 311.2.

### Step 3: (3R,4R)-Dimethyl-1-((S)-piperidin-3-yl)pyrrolidine-2,5-dione hydrochloride

(3R,4R)-Dimethyl-1-((S)-piperidin-3-yl)pyrrolidine-2,5-dione was prepared according to the general procedure E using (3S)-tert-butyl 3-(3,4-dimethyl-2,5-dioxopyrrolidin-1-yl)piperidine-1-carboxylate (700 mg, 2 mmol) and HCl in Et₂O (25 ml) to obtain 550 mg of the product as HCl salt.

LC-MS (ES+) [M+1]: 241.46.

### Step 4: 1-((S)-1-(((S)-2,3-Dihydrobenzo[b][1,4]dioxin-2-yl)methyl)piperidin-3-yl)-4-phenylimidazolidin-2-one

1-((S)-1-(((S)-2,3-Dihydrobenzo[b][1,4]dioxin-2-yl)methyl)piperidin-3-yl)-4-phenylimidazolidin-2-one was prepared according to the general procedure A using (3R,4R)-3,4-dimethyl-1-((S)-piperidin-3-yl)pyrrolidine-2,5-dione (75 mg, 0.357 mmol), (2R)-2-(bromomethyl)-2,3-dihydro-1,4-benzodioxin (82 mg, 0.357 mmol), DIPEA (0.075 ml, 0.428 mmol), K₂CO₃ (123 mg, 0.892 mmol) and ACN (0.5 ml). The crude product was purified by reversed phase flash chromatography to obtain 55.4 mg of the product.

¹H NMR (400 MHz, CDCl₃) δ ppm 1.26-1.39 (m, 6H), 1.59-1.83 (m, 3H), 2.05-2.29 (m, 2H), 2.30-2.42 (m, 2H), 2.57-2.94 (m, 5H), 3.92-4.06 (m, 1H), 4.12-4.37 (m, 3H), 6.78-6.92 (m, 4H).

### EXAMPLE 5: 3-((S)-1-(((S)-2,3-Dihydrobenzo[b][1,4]dioxin-2-yl)methyl)piperidin-3-yl)-5,5-diethyloxazolidine-2,4-dione

### Step 1: (S)-tert-Butyl 3-(2-ethyl-2-hydroxybutanamido)piperidine-1-carboxylate

To a stirred solution of(S)-tert-butyl 3-aminopiperidine-1-carboxylate (2.0 g, 10 mmol) in DCM (100 ml) was added 2-ethyl-2-hydroxybutanoic acid (1.45 g, 11 mmol) followed by EDC, HOBt and DMAP at 0° C., then the reaction mixture was stirred at rt for 16 h. The reaction mixture was diluted with DCM and washed with water. The organic layer was dried evaporated. The crude compound was purified by flash chromatography to obtain 1.4 g of the product.

¹H NMR (400 MHz, CDCl₃) δ ppm 0.75 (t, 6H), 1.36-1.62 (m, 12H), 1.61-1.77 (m, 5H), 2.85-3.10 (m, 2H), 3.45-3.70 (m, 3H), 4.88 (m, 1H), 7.38 (m, 1H).

### Step 2: (S)-tert-Butyl 3-(5,5-diethyl-2,4-dioxooxazolidin-3-yl)piperidine-1-carboxylate

(S)-tert-Butyl 3-(5,5-diethyl-2,4-dioxooxazolidin-3-yl)piperidine-1-carboxylate was prepared according to the general procedure C using (S)-tert-butyl 3-(2-ethyl-2-hydroxybutanamido)piperidine-1-carboxylate (1.0 g, 3.1 mmol), DMF (20 ml), DIPEA (1.76 ml, 9.5 mmol) and CDI 1.3 g, 7.9 mmol). The crude product was purified by flash chromatography to obtain 700 mg of the product.

LC-MS (ES+) [M+1]: 241.0.

### Step 3: (S)-5,5-Diethyl-3-(piperidin-3-yl)oxazolidine-2,4-dione hydrochloride

(S)-5,5-Diethyl-3-(piperidin-3-yl)oxazolidine-2,4-dione hydrochloride was prepared according to the general procedure E using (S)-tert-butyl 3-(5,5-diethyl-2,4-dioxooxazolidin-3-yl)piperidine-1-carboxylate (250 mg, 0.8 mmol) and HCl in dioxane to obtain 170 mg of the product as HCl salt.

LC-MS (ES+) [M+1]: 211.1.

### Step 4: 3-((S)-1-(((S)-2,3-Dihydrobenzo[b][1,4]dioxin-2-yl)methyl)piperidin-3-yl)-5,5-diethyloxazolidine-2,4-dione

3-((S)-1-(((S)-2,3-Dihydrobenzo[b][1,4]dioxin-2-yl)methyl)piperidin-3-yl)-5,5-diethyloxazolidine-2,4-dione was prepared according to the general procedure A using (S)-5,5-diethyl-3-(piperidin-3-yl)oxazolidine-2,4-dione, HCl (150 mg, 0.542 mmol), (2R)-2-(bromomethyl)-2,3-dihydro-1,4-benzodioxin (124 mg, 0.542 mmol), DIPEA (0.113 ml, 0.650 mmol), K₂CO₃ (112 mg, 0.813 mmol) and ACN (1 ml). The product was purified by reversed phase flash chromatography to obtain 55.4 mg of the product.

¹H NMR (400 MHz, CDCl₃) δ ppm 0.70 (t, 6H), 1.36-1.62 (m, 3H), 1.61-1.77 (m, 4H), 1.85-2.09 (m, 2H), 2.35-2.73 (m, 5H), 3.75-3.85 (m, 1H), 3.88-4.01 (m, 1H), 4.02-4.16 (m, 2H), 6.54-6.74 (m, 4H).

### EXAMPLE 6: (R)-1-((S)-1-(((S)-2,3-Dihydrobenzo[b][1,4]dioxin-2-yl)methyl)-piperidin-3-yl)-3-isopropyl-4-phenylimidazolidin-2-one

### Step 1: (3S)-Benzyl 3-((2-((tert-butoxycarbonyl)amino)-2-phenylethyl)amino)-piperidine-1-carboxylate

(3S)-Benzyl 3-((2-((tert-butoxycarbonyl)amino)-2-phenylethyl)amino)piperidine-1-carboxylate was prepared according to the general procedure B using (S)-tert-butyl (2-oxo-1-phenylethyl)carbamate (15.0 g, 64.10 mmol, Synlett, 2005 (13), 2110), (S)-benzyl 3-aminopiperidine-1-carboxylate (15.06 g, 64.10 mmol), NaBH(OAc)₃ (20.38 g, 96.15 mmol) and DCE (300 ml). The crude product was purified by flash chromatography to obtain 13 g of the product.

LC-MS (ES+) [M+1]: 454.3.

### Step 2: (3S)-Benzyl 3-((2-amino-2-phenylethyl)amino)piperidine-1-carboxylate hydrochloride

(3S)-Benzyl 3-((2-amino-2-phenylethyl)amino)piperidine-1-carboxylate was prepared according to the general procedure E using (3S)-benzyl 3-((2-((tert-butoxycarbonyl)amino)-2-phenylethyl)amino)piperidine-1-carboxylate (8.0 g, 17.66 mmol), dioxane (80 ml) and 1M HCl in dioxane (80 ml). The residue was triturated with Et₂O/pentane to obtain 7.5 g of product as HCl salt.

LC-MS (ES+) [M+1]: 354.3.

### Step 3: (3S)-Benzyl 3-(2-oxo-4-phenylimidazolidin-1-yl)piperidine-1-carboxylate

(3S)-Benzyl 3-(2-oxo-4-phenylimidazolidin-1-yl)piperidine-1-carboxylate was prepared according to the general procedure C using the product from Step 2 (12.0 g, 33.99 mmol), DMF (120 ml), Et₃N (10.89 ml, 78.18 mmol) and CDI (6.61 g, 40.79 mmol). The crude product was purified by flash chromatography to obtain 6.8 g of the product.

LC-MS (ES+) [M+1]: 380.2.

### Step 4: (3S)-Benzyl 3-(3-isopropyl-2-oxo-4-phenylimidazolidin-1-yl)piperidine-1-carboxylate

To an ice cold solution of (3S)-benzyl 3-(2-oxo-4-phenylimidazolidin-1-yl)piperidine-1-carboxylate (3.5 g, 9.23 mmol) in DMF (70.0 ml) was added NaH (2.21 g, 92.3 mmol) followed by isopropyl bromide (2.60 ml, 27.70 mmol). The reaction mixture was stirred at RT for 18 h. The reaction mixture was quenched with ice cold water and evaporated. The residue was diluted with EtOAc and washed with water. The organic layer was dried and evaporated. The crude product was purified by flash chromatography to obtain 900 mg the product.

LC-MS (ES+) [M+1]: 422.3.

### Step 5: (R)-3-Isopropyl-4-phenyl-1-((S)-piperidin-3-yl)imidazolidin-2-one and (S)-3-isopropyl-4-phenyl-1-((S)-piperidin-3-yl)imidazolidin-2-one

3-Isopropyl-4-phenyl-1-((S)-piperidin-3-yl)imidazolidin-2-one was prepared according to the general procedure D using the product from Step 4 (2.6 g, 6.17 mmol), 10% Pd/C (2.0 g) and EtOAc (50 ml). The enantiomers were separated by SFC column to obtain 200 mg of enantiomer 1 and 330 mg of enantiomer 2.

LC-MS (ES+) [M+1]: 288.3 for both enantiomers.

### Step 6: (R)-1-((S)-1-(((S)-2,3-Dihydrobenzo[b][1,4]dioxin-2-yl)methyl)piperidin-3-yl)-3-isopropyl-4-phenylimidazolidin-2-one

(R)-1-((S)-1-(((S)-2,3-Dihydrobenzo[b][1,4]dioxin-2-yl)methyl)piperidin-3-yl)-3-isopropyl-4-phenylimidazolidin-2-one was prepared according to the general procedure A using (R)-3-isopropyl-4-phenyl-1-((S)-piperidin-3-yl)imidazolidin-2-one (266 mg, 0.926 mmol), (2R)-2-(bromomethyl)-2,3-dihydro-1,4-benzodioxin (212 mg, 0.926 mmol), DIPEA (0.193 ml, 1.111 mmol), K₂CO₃ (192 mg, 1.388 mmol) and ACN (1.1 ml). The product was purified by reversed phase flash chromatography to obtain 114 mg of the product.

¹H NMR (400 MHz, CDCl₃) δ ppm 0.87 (d, 3H) 1.21 (d, 3H) 1.29-1.41 (m, 1H) 1.68-1.76 (m, 3H) 1.78-1.86 (m, 1H) 2.01-2.17 (m, 2H) 2.60-2.66 (m, 2H) 2.79 (d, 1H) 2.96-3.05 (m, 1H) 3.14 (t, 1H) 3.55-3.70 (m, 1H) 3.83 (m, 1H) 3.92-4.07 (m, 2H) 4.22-4.36 (m, 2H) 4.45-4.60 (m, 1H) 4.22-4.36 (m, 2H) 4.45-4.60 (m, 1H) 6.76-6.91 (m, 4H) 7.28-7.42 (m, 5H).

### EXAMPLE 7: (S)-1-((S)-1-(((S)-2,3-Dihydrobenzo[b][1,4]dioxin-2-yl)methyl)piperidin-3-yl)-3-isopropyl-4-phenylimidazolidin-2-one

(S)-1-((S)-1-(((S)-2,3-Dihydrobenzo[b][1,4]dioxin-2-yl)methyl)piperidin-3-yl)-3-isopropyl-4-phenylimidazolidin-2-one was prepared according to the general procedure A using (S)-3-isopropyl-4-phenyl-1-((S)-piperidin-3-yl)imidazolidin-2-one (266 mg, 0.926 mmol), (2R)-2-(bromomethyl)-2,3-dihydro-1,4-benzodioxin (212 mg, 0.926 mmol), DIPEA (0.193 ml, 1.111 mmol), K₂CO₃ (192 mg, 1.388 mmol) and ACN (1.2 ml). The product was purified by reversed phase flash chromatography to obtain 177.4 mg of the product.

¹H NMR (400 MHz, CDCl₃) δ ppm 0.87 (d, 3H) 1.21 (d, 3H) 1.29-1.41 (m, 1H) 1.61-1.76 (m, 3H) 1.73-1.86 (m, 1H) 2.01-2.17 (m, 2H) 2.60-2.66 (m, 2H) 2.79 (d, 1H) 2.96-3.05 (m, 1H) 3.14 (t, 1H) 3.55-3.70 (m, 1H) 3.83 (m, 1H) 3.92-4.07 (m, 2H) 4.22-4.36 (m, 2H) 4.45-4.60 (m, 1H) 6.76-6.91 (m, 4H) 7.28-7.42 (m, 5H).

### EXAMPLE 8: (R)-1-((S)-1-(((S)-2,3-Dihydrobenzo[b][1,4]dioxin-2-yl)methyl)piperidin-3-yl)-4-phenylimidazolidin-2-one

### Step 1: (R)-4-Phenyl-1-((S)-piperidin-3-yl)imidazolidin-2-one and (S)-4-phenyl-1-((S)-piperidin-3-yl)imidazolidin-2-one

4-Phenyl-1-((S)-piperidin-3-yl)imidazolidin-2-one was prepared according to the general procedure D using (3S)-benzyl 3-(2-oxo-4-phenylimidazolidin-1-yl)piperidine-1-carboxylate (2.1 g, 5.54 mmol), 10% Pd/C (1.0 g) and EtOAc (40 ml). The crude compound was purified by SFC purification to obtain 248 mg of enantiomer 1 and 340 mg of enantiomer 2.

LC-MS (ES+) [M+1]: 246.2 for both enantiomers.

### Step 2: (R)-1-((S)-1-(((S)-2,3-Dihydrobenzo[b][1,4]dioxin-2-yl)methyl)piperidin-3-yl)-4-phenylimidazolidin-2-one

(R)-1-((S)-1-(((S)-2,3-Dihydrobenzo[b][1,4]dioxin-2-yl)methyl)piperidin-3-yl)-4-phenylimidazolidin-2-one was prepared according to the general procedure A using (R)-4-phenyl-1-((S)-piperidin-3-yl)imidazolidin-2-one (266 mg, 1.084 mmol), (2R)-2-(bromomethyl)-2,3-dihydro-1,4-benzodioxin (248 mg, 1.084 mmol), DIPEA (0.227 ml, 1.301 mmol), K₂CO₃ (225 mg, 1.626 mmol) and ACN (1.1 ml). The product was purified by reversed phase flash chromatography using 0.5% HCOOH/ACN as eluent resulting in 19.5 mg of oil.

¹H NMR (400 MHz, CDCl₃) δ ppm 7.28-7.43 (m, 5H) 6.72-6.92 (m, 4H) 4.73 (td, 1H) 4.60 (s, 1H) 4.20-4.34 (m, 2H) 3.80-4.05 (m, 3H) 3.18-3.33 (m, 1H) 2.87-3.01 (m, 1H) 2.77 (d, 1H) 2.47-2.66 (m, 2H) 2.04-2.18 (m, 2H) 1.63-1.89 (m, 3H) 1.33-1.50 (m, 1H) 1.21-1.30 (m, 1H).

### EXAMPLE 9: (S)-1-((S)-1-(((S)-2,3-Dihydrobenzo[b][1,4]dioxin-2-yl)methyl)piperidin-3-yl)-4-phenylimidazolidin-2-one

(S)-1-((S)-1-(((S)-2,3-Dihydrobenzo[b][1,4]dioxin-2-yl)methyl)piperidin-3-yl)-4-phenylimidazolidin-2-one was prepared according general procedure A using (S)-4-phenyl-1-((S)-piperidin-3-yl)imidazolidin-2-one (226 mg, 0.921 mmol), (2R)-2-(bromomethyl)-2,3-dihydro-1,4-benzodioxin (211 mg, 0.921 mmol), DIPEA (0.193 ml, 1.105 mmol), K₂CO₃ (191 mg, 1.382 mmol) and ACN (1.1 ml). The product was purified by reversed phase flash chromatography to obtain 134.9 mg of the product.

¹H NMR (400 MHz, CDCl₃) δ ppm 7.27-7.45 (m, 5H) 6.77-6.93 (m, 4H) 4.67-4.79 (m, 1H) 4.59 (s, 1H) 4.22-4.36 (m, 2H) 3.89-4.09 (m, 2H) 3.73-3.85 (m, 1H) 3.22-3.35 (m, 1H) 2.95-3.08 (m, 1H) 2.78 (d, 1H) 2.51-2.72 (m, 2H) 2.02-2.29 (m, 2H) 1.60-1.83 (m, 3H) 1.28-1.40 (m, 1H).

### EXAMPLE 10: 6-((S)-1-(((S)-2,3-Dihydrobenzo[b][1,4]dioxin-2-yl)methyl)piperidin-3-yl)-6,7-dihydro-5H-pyrrolo[3,4-b]pyridin-5-one

### Step 1: (S)-Benzyl 3-(5,7-dioxo-5H-pyrrolo[3,4-b]pyridin-6(7H)-yl)piperidine-1-carboxylate

To an ice cold stirred solution of (S)-benzyl 3-aminopiperidine-1-carboxylate (1.0 g, 4.268 mmol) in toluene (10 ml) was added Et₃N (0.89 ml, 6.402 mmol) and stirred at RT for 30 mins. Furo[3,4-b]pyridine-5,7-dione (764 mg, 5.122 mmol) was added to the above mixture and then heated to 110° C. for 16 h. The reaction mixture was diluted with EtOAc (60 ml) and washed with water (2×30 ml). The organic layer was dried and evaporated. The crude compound was purified by column chromatography to obtain 250 mg of the product.

LC-MS (ES+) [M+1]: 366.1.

### Step 2: (S)-Benzyl 3-(5-oxo-5H-pyrrolo[3,4-b]pyridin-6(7H)-yl)piperidine-1-carboxylate

A mixture of (S)-benzyl 3-(5,7-dioxo-5H-pyrrolo[3,4-b]pyridin-6(7H)-yl)piperidine-1-carboxylate (700 mg, 1.916 mmol) and zinc dust (550 mg, 8.429 mmol) in acetic acid (14 ml) was heated at 110° C. for 16 h. The reaction mixture was cooled to RT and filtered through a pad of celite and washed with EtOAc (15 ml). Filtrate was concentrated under reduced pressure. The residue was carefully basified with saturated aqueous NaHCO₃ solution and extracted with DCM (2×80 ml). The combined organic layer was dried and evaporated. The crude compound was purified by column chromatography to obtain 320 mg of the product.

LC-MS (ES+) [M+1]: 352.2.

### Step 3: (S)-Benzyl 3-(5-oxo-5H-pyrrolo[3,4-b]pyridin-6(7H)-yl)piperidine-1-carboxylate

(S)-Benzyl 3-(5-oxo-5H-pyrrolo[3,4-b]pyridin-6(7H)-yl)piperidine-1-carboxylate was prepared according to the general procedure D using (S)-benzyl 3-(5-oxo-5H-pyrrolo[3,4-b]pyridin-6(7H)-yl)piperidine-1-carboxylate (600 mg, mmol), 10% Pd/C (300 mg) and EtOAc (40 ml). The crude compound was purified by triturating with Et₂O to obtain 220 mg of the product.

LC-MS (ES+) [M+1]: 218.2.

### Step 4: 6-((S)-1-(((S)-2,3-Dihydrobenzo[b][1,4]dioxin-2-yl)methyl)piperidin-3-yl)-6,7-dihydro-5H-pyrrolo[3,4-b]pyridin-5-one

6-((S)-1-(((S)-2,3-Dihydrobenzo[b][1,4]dioxin-2-yl)methyl)piperidin-3-yl)-6,7-dihydro-5H-pyrrolo[3,4-b]pyridin-5-one was prepared according to the general procedure A using (S)-6-(piperidin-3-yl)-6,7-dihydro-5H-pyrrolo[3,4-b]pyridin-5-one (208 mg, 0.957 mmol), (R)-(2,3-dihydrobenzo[b][1,4]dioxin-2-yl)methyl 4-methylbenzenesulfonate (310 mg, 0.968 mmol), DIPEA (0.200 ml, 1.149 mmol), K₂CO₃ (198 mg, 1.436 mmol) and ACN (1 ml). The product was purified by reversed phase flash chromatography to obtain 68 mg of the product.

¹H NMR (400 MHz, CDCl₃) δ ppm 1.55-1.68 (m, 1H) 1.70-1.87 (m, 2H) 1.88-2.02 (m, 1H) 2.20-2.40 (m, 2H) 2.58-2.77 (m, 2H) 2.79-2.91 (m, 1H) 3.00-3.13 (m, 1H) 3.97-4.08 (m, 1H) 4.25-4.38 (m, 2H) 4.39-4.56 (m, 3H) 6.69-6.97 (m, 4H) 7.33-7.47 (m, 1H) 8.07-8.19 (m, 1H) 8.67-8.78 (m, 1H).

### EXAMPLE 11: 2-((S)-1-(((S)-2,3-Dihydrobenzo[b][1,4]dioxin-2-yl)methyl)piperidin-3-yl)-1H-pyrrolo[3,4-c]pyridin-3(2H)-one

### Step 1: (S)-Benzyl 3-(3-oxo-1H-pyrrolo[3,4-c]pyridin-2(3H)-yl)piperidine-1-carboxylate

A mixture of (S)-benzyl 3-(1,3-dioxo-1H-pyrrolo[3,4-c]pyridin-2(3H)-yl)piperidine-1-carboxylate (1.0 g, 2.73 mmol) and zinc dust (0.9 g, 13.68 mmol) in acetic acid (16 ml) was heated at 110° C. for 6 h. The reaction mixture was cooled to RT and filtered through a pad of Celite and washed with EtOAc (20 ml). Filtrate was concentrated under reduced pressure. The residue was basified with saturated aqueous NaHCO₃ solution and extracted with DCM (2×80 ml). The combined organic layer was dried and evaporated. The crude compound was purified by column chromatography to obtain 800 mg of the product.

LC-MS (ES+) [M+1]: 352.2.

### Step 2: (S)-2-(Piperidin-3-yl)-1H-pyrrolo[3,4-c]pyridin-3(2H)-one

(S)-2-(Piperidin-3-yl)-1H-pyrrolo[3,4-c]pyridin-3(2H)-one was prepared according to the general procedure D using ((S)-benzyl 3-(1,3-dioxo-1H-pyrrolo[3,4-c]pyridin-2(3H)-yl)piperidine-1-carboxylate (0.5 g, 1.42 mmol), 10% Pd/C (0.4 g) and EtOAc (20 ml). The crude compound was purified by triturating with Et₂O to obtain 150 mg of the product.

LC-MS (ES+) [M+1]: 218.1.

### Step 3: 2-((S)-1-(((S)-2,3-Dihydrobenzo[b][1,4]dioxin-2-yl)methyl)piperidin-3-yl)-1H-pyrrolo[3,4-c]pyridin-3(2H)-one

2-((S)-1-(((S)-2,3-Dihydrobenzo[b][1,4]dioxin-2-yl)methyl)piperidin-3-yl)-1H-pyrrolo[3,4-c]pyridin-3(2H)-one was prepared according to the general procedure A using ((S)-2-(piperidin-3-yl)-1H-pyrrolo[3,4-c]pyridin-3(2H)-one (180 mg, 0.828 mmol), (R)-(2,3-dihydrobenzo[b][1,4]dioxin-2-yl)methyl 4-methylbenzenesulfonate (265 mg, 0.828 mmol), DIPEA (0.173 ml, 0.994 mmol), K₂CO₃ (172 mg, 1.243 mmol) and ACN (1 ml). The product was purified by reversed phase flash chromatography to obtain 47 mg of the product.

¹H NMR (400 MHz, CDCl₃) δ ppm 1.55-1.64 (m, 1H) 1.74-1.84 (m, 2H) 1.86-1.95 (m, 1H) 2.26-2.42 (m, 2H) 2.62-2.74 (m, 2H) 2.83 (dt, 1H) 3.03 (dd, 1H) 4.03 (dd, 1H) 4.25-4.34 (m, 2H) 4.41-4.57 (m, 3H) 6.80-6.88 (m, 4H) 7.42 (dd, 1H) 8.73-8.77 (m, 1H) 9.10 (d, 1H).

### EXAMPLE 12: 6-((S)-1-(((S)-2,3-Dihydrobenzo[b][1,4]dioxin-2-yl)methyl)piperidin-3-yl)-2-(methylthio)-5H-pyrrolo[3,4-d]pyrimidin-7(6H)-one

### Step 1: (4-Chloro-2-(methylthio)pyrimidin-5-yl)methyl methanesulfonate

To a cold stirred solution of (4-chloro-2-(methylthio)pyrimidin-5-yl)methanol (4.3 g, 22.63 mmol, WO2008/094602) in DCM (45 ml) were added Et₃N (7.95 ml, 56.57 mmol) and methanesulfonyl chloride (2.1 ml, 27.157 mmol) at 0° C. The resulting solution was stirred at RT for 2 h. The reaction mixture was diluted with DCM (50 ml) and washed with water (1×50 ml) brine (1×50 ml). The organic layer was dried and evaporated to obtain 3.9 g of the product.

LC-MS (ES+) [M+1]: 269.0.

### Step 2: (S)-tert-Butyl 3-(((4-chloro-2-(methylthio)pyrimidin-5-yl)methyl)amino)-piperidine-1-carboxylate

To a stirred solution of (4-chloro-2-(methylthio)pyrimidin-5-yl)methyl methanesulfonate (3.9 g, 14.606 mmol) and (S)-tert-butyl 3-aminopiperidine-1-carboxylate (2.92 g, 14.606 mmol) in ACN (40 ml) was added K₂CO₃ (5.03 g, 36.516 mmol) at RT and stirred for 16 h. The reaction was concentrated under reduced pressure. The obtained residue was dissolved in mixture of EtOAc (100 ml) and water (100 ml). The organic layer was dried and evaporated. The crude product was purified by column chromatography to obtain 2.7 g of the product.

¹H NMR (400 MHz, DMSO) δ ppm 1.21-1.32 (m, 3H) 1.37 (s, 9H) 1.63-1.66 (m, 1H) 1.85-1.99 (m, 1H) 2.28-2.33 (m, 1H) 2.41-2.45 (m, 1H) 2.52 (s, 3H) 2.84-2.90 (m, 1H) 3.02 (dd, 1H) 3.6 (bs, 1H) 3.76 (s, 2H) 8.65 (s, 1H).

### Step 3: (S)-tert-Butyl 3-(2-(methylthio)-7-oxo-5H-pyrrolo[3,4-d]pyrimidin-6(7H)-yl)piperidine-1-carboxylate

To a solution of (S)-tert-butyl 3-(((4-chloro-2-(methylthio)pyrimidin-5-yl)methyl)amino)-piperidine-1-carboxylate (2.7 g, 7.258 mmol) in ethanol (80 ml) were added sodium acetate and PdCl₂(dppf).CH₂Cl₂ (142 mg, 0.362 mmol) in a steel bomb. The resulting reaction mixture was subjected to carbonyl insertion with CO gas (500 psi) and heated to 140° C. for 16 h. The reaction mixture was concentrated under reduced pressure. The residue was dissolved in EtOAc (100 ml), washed with water (2×100 ml) and brine (1×100 ml). The organic layer was dried and evaporated. The crude product was purified by column chromatography to obtain 1.6 g of the product.

¹H NMR (400 MHz, DMSO) δ ppm 1.21-1.32 (m, 3H) 1.40 (s, 9H) 1.35-1.49 (m, 2H) 1.75-1.80 (m, 2H) 1.89 (m, 1H) 2.58 (s, 3H) 2.75 (m, 1H) 2.96 (m, 1H) 3.86-3.90 (m, 1H) 3.99-4.04 (m, 2H) 4.53 (s, 2H) 8.99 (s, 1H).

### Step 4: (S)-2-(Methylthio)-6-(piperidin-3-yl)-5H-pyrrolo[3,4-d]pyrimidin-7(6H)-one

(S)-2-(Methylthio)-6-(piperidin-3-yl)-5H-pyrrolo[3,4-d]pyrimidin-7(6H)-one was prepared according to the general procedure E using (S)-tert-butyl 3-(2-(methylthio)-7-oxo-5H-pyrrolo[3,4-d]pyrimidin-6(7H)-yl)piperidine-1-carboxylate (400 mg, 1.098 mmol) and 1M HCl in dioxane (25 ml). The residue was triturated with pentane to obtain 300 mg of product.

LC-MS (ES+) [M+1]: 265.1.

### Step 5: 6-((S)-1-(((S)-2,3-Dihydrobenzo[b][1,4]dioxin-2-yl)methyl)piperidin-3-yl)-2-(methylthio)-5H-pyrrolo[3,4-d]pyrimidin-7(6H)-one

6-((S)-1-(((S)-2,3-Dihydrobenzo[b][1,4]dioxin-2-yl)methyl)piperidin-3-yl)-2-(methylthio)-5H-pyrrolo[3,4-d]pyrimidin-7(6H)-one was prepared according to the general procedure A using ((S)-2-(piperidin-3-yl)-1H-pyrrolo[3,4-c]pyridin-3(2H)-one (310 mg, 0.968 mmol), (R)-(2,3-dihydrobenzo[b][1,4]dioxin-2-yl)methyl 4-methylbenzenesulfonate (265 mg, 0.968 mmol), DIPEA (0.202 ml, 1.161 mmol), K₂CO₃ (201 mg, 1.451 mmol) and ACN (1 ml). The product was purified by reversed phase flash chromatography to obtain 99 mg of the product.

¹H NMR (400 MHz, CDCl₃) δ ppm 1.67-1.96 (m, 4H) 2.24-2.52 (m, 2H) 2.58-2.74 (m, 2H) 2.66 (s, 3H) 2.77-2.91 (m, 1H) 3.02 (dd, 1H) 3.96-4.11 (m, 1H) 4.23-4.35 (m, 2H) 4.47-4.61 (m, 3H) 6.74-6.95 (m, 4H) 8.72 (s, 1H).

### EXAMPLE 13: 5-((S)-1-(((S)-2,3-Dihydrobenzo[b][1,4]dioxin-2-yl)methyl)piperidin-3-yl)-5,6-dihydro-4H-pyrrolo[3,4-d]thiazol-4-one

### Step 1: (S)-tert-Butyl 3-(5-methylthiazole-4-carboxamido)piperidine-1-carboxylate

To a stirred solution of 5-methylthiazole-4-carboxylic acid (4.0 g, 27.97 mmol, WO2008/016192) and (S)-tert-butyl 3-aminopiperidine-1-carboxylate (6.15 g, 30.76 mmol) in DCM were added EDC.HCl (6.51 g, 41.95 mmol), HOBt (6.42 g, 41.95 mmol) and DMAP (8.53 g, 69.93 mmol) at 0° C. The reaction mixture was stirred for 16 h. The reaction mixture was diluted with DCM, washed with water, brine, dried and evaporated. The crude product was purified by flash column to obtain 6.0 g of the product.

LC-MS (ES+) [M+1]:326.2.

### Step 2: (S)-tert-Butyl 3-(5-(bromomethyl)thiazole-4-carboxamido)piperidine-1-carboxylate

To a stirred solution of (S)-tert-butyl 3-(5-methylthiazole-4-carboxamido)piperidine-1-carboxylate (4.0 g, 12.30 mmol) in CCl₄ (80 ml) was added NBS (3.28 g, 18.46 mmol) followed by AIBN (1.61 g, 9.84 mmol) at RT. The reaction mixture was heated at 80° C. for 2 h. The reaction mixture was diluted with DCM and washed with water. The combined organic layer was dried and evaporated. The crude product was purified by flash column to obtain 1.0 g of the product.

LC-MS (ES+) [M+1]: 406.1.

### Step 3: (S)-tert-Butyl 3-(4-oxo-4H-pyrrolo[3,4-d]thiazol-5(6H)-yl)piperidine-1-carboxylate

To a stirred solution of (S)-tert-butyl 3-(5-(bromomethyl)thiazole-4-carboxamido)-piperidine-1-carboxylate (0.4 g, 0.992 mmol) in THF (10 ml) was added NaH (0.034 g, 1.48 mmol) at −10° C. The reaction mixture was stirred at −10° C. to 0° C. for 30 minutes. The reaction mixture was diluted with EtOAc and quenched with water. The organic layer was dried and evaporated. The crude product was purified by flash column to obtain 200 mg of the product.

LC-MS (ES+) [M+1]: 324.2.

### Step 4: (S)-5-(Piperidin-3-yl)-5,6-dihydro-4H-pyrrolo[3,4-d]thiazol-4-one, HCl

(S)-5-(Piperidin-3-yl)-5,6-dihydro-4H-pyrrolo[3,4-d]thiazol-4-one was prepared according to the general procedure E using (S)-tert-butyl 3-(4-oxo-4H-pyrrolo[3,4-d]thiazol-5(6H)-yl)piperidine-1-carboxylate (300 mg 0.928 mmol) and HCl in dioxane (10 ml). The residue was triturated with Et₂O— pentane to obtain 230 mg of the product.

LC-MS (ES+) [M+1]: 224.1.

### Step 5: 5-((S)-1-(((S)-2,3-Dihydrobenzo[b][1,4]dioxin-2-yl)methyl)piperidin-3-yl)-5,6-dihydro-4H-pyrrolo[3,4-d]thiazol-4-one

5-((S)-1-(((S)-2,3-Dihydrobenzo[b][1,4]dioxin-2-yl)methyl)piperidin-3-yl)-5,6-dihydro-4H-pyrrolo[3,4-d]thiazol-4-one was prepared according to the general procedure A using (S)-5-(piperidin-3-yl)-5,6-dihydro-4H-pyrrolo[3,4-d]thiazol-4-one hydrochloride (220 mg, 0.847 mmol), (R)-(2,3-dihydrobenzo[b][1,4]dioxin-2-yl)methyl 4-methylbenzenesulfonate (271 mg, 0.847 mmol), DIPEA (0.177 ml, 1.016 mmol), K₂CO₃ (293 mg, 2.117 mmol) and ACN (1 ml). The product was purified by reversed phase flash chromatography to obtain 55 mg of the product.

¹H NMR (400 MHz, CDCl3) δ ppm 1.68-1.85 (m, 2H) 1.86-1.97 (m, 1H) 1.97-2.05 (m, 1H) 2.19-2.50 (m, 2H) 2.62-2.74 (m, 2H) 2.74-2.86 (m, 1H) 3.04 (dd, 1H) 3.91-4.08 (m, 1H) 4.23-4.37 (m, 2H) 4.37-4.50 (m, 1H) 4.52-4.68 (m, 2H) 6.75-6.94 (m, 4H) 8.89 (s, 1H).

### EXAMPLE 14: (R)-1-((S)-1-(((S)-2,3-Dihydrobenzo[b][1,4]dioxin-2-yl)methyl)piperidin-3-yl)-3-methyl-3-phenylpyrrolidin-2-one

### Step 1: (S)-Benzyl-3-((R)-3-methyl-2-oxo-3-phenylpyrrolidin-1-yl)piperidine-1-carboxylate and (S)-benzyl-3-((S)-3-methyl-2-oxo-3-phenylpyrrolidin-1-yl)piperidine-1-carboxylate

To and ice cold stirred solution of methyl 2-methyl-4-oxo-2-phenylbutanoate (3.2 g, 15.51 mmol, Organic Letters, 2014, 16(1), 14) in acetic acid (65 ml) was added (S)-benzyl 3-aminopiperidine-1-carboxylate (3.64 g, 15.51 mmol) and zinc metal (10 g, 155 mmol). The reaction mixture was stirred at 120° C. for 6 h. The reaction mixture was cooled to RT and filtered through a pad of celite. Filtrate was concentrated under reduced pressure; the residue was diluted with DCM (150 ml) and washed with saturated aqueous NaHCO₃ (2×60 ml) and brine. The organic layer was dried and evaporated. The crude compound was purified by column chromatography to obtain 710 mg of (S)-benzyl 3-((R)-3-methyl-2-oxo-3-phenylpyrrolidin-1-yl)piperidine-1-carboxylate and 700 mg of (S)-benzyl 3-((S)-3-methyl-2-oxo-3-phenylpyrrolidin-1-yl)piperidine-1-carboxylate.

LC-MS (ES+) [M+1]: 393.2 for both enantiomers.

### Step 2: (R)-3-Methyl-3-phenyl-1-((S)-piperidin-3-yl)pyrrolidin-2-one

(R)-3-Methyl-3-phenyl-1-((S)-piperidin-3-yl)pyrrolidin-2-one was prepared according to the general procedure D using (S)-benzyl 3-((R)-3-methyl-2-oxo-3-phenylpyrrolidin-1-yl)piperidine-1-carboxylate (760 mg, 1.94 mmol), 10% Pd/C (0.4 g) and EtOAc (80 ml). The crude compound was used without further purification.

LC-MS (ES+) [M+1]: 259.3.

### Step 3: (R)-1-((S)-1-(((S)-2,3-Dihydrobenzo[b][1,4]dioxin-2-yl)methyl)piperidin-3-yl)-3-methyl-3-phenylpyrrolidin-2-one

(R)-1-((S)-1-(((S)-2,3-Dihydrobenzo[b][1,4]dioxin-2-yl)methyl)piperidin-3-yl)-3-methyl-3-phenylpyrrolidin-2-one was prepared according to the general procedure A using (R)-3-methyl-3-phenyl-1-((S)-piperidin-3-yl)pyrrolidin-2-one (170 mg, 0.658 mmol), (R)-(2,3-dihydrobenzo[b][1,4]dioxin-2-yl)methyl 4-methylbenzenesulfonate (253 mg, 0.79 mmol), K₂CO₃ (227 mg, 1.645 mmol) and ACN (1 ml). The product was purified by reversed phase flash chromatography to obtain 136 mg of the product.

¹H NMR (400 MHz, CDCl₃) δ ppm 1.35-1.48 (m, 1H) 1.53 (s, 3H) 1.63-1.82 (m, 3H) 2.03-2.27 (m, 3H) 2.41 (ddd, 1H) 2.56-2.71 (m, 2H) 2.74-2.87 (m, 1H) 2.87-3.02 (m, 1H) 3.15-3.39 (m, 2H) 4.02 (dd, 1H) 4.15-4.38 (m, 3H) 6.76-6.93 (m, 4H) 7.12-7.26 (m, 1H) 7.30-7.45 (m, 4H).

### EXAMPLE 15: (S)-1-((S)-1-(((S)-2,3-Dihydrobenzo[b][1,4]dioxin-2-yl)methyl)-piperidin-3-yl)-3-methyl-3-phenylpyrrolidin-2-one

### Step 1: (S)-3-Methyl-3-phenyl-1-((S)-piperidin-3-yl)pyrrolidin-2-one

(S)-3-Methyl-3-phenyl-1-((S)-piperidin-3-yl)pyrrolidin-2-one was prepared according to the general procedure D using (S)-benzyl 3-((S)-3-methyl-2-oxo-3-phenylpyrrolidin-1-yl)piperidine-1-carboxylate (740 mg, 1.89 mmol), 10% Pd/C (0.4 g) and EtOAc (80 ml). The crude compound was used without further purification.

LC-MS (ES+) [M+1]: 259.3.

### Step 2: (S)-1-((S)-1-(((S)-2,3-Dihydrobenzo[b][1,4]dioxin-2-yl)methyl)piperidin-3-yl)-3-methyl-3-phenylpyrrolidin-2-one

(S)-1-((S)-1-(((S)-2,3-Dihydrobenzo[b][1,4]dioxin-2-yl)methyl)piperidin-3-yl)-3-methyl-3-phenylpyrrolidin-2-one was prepared according to the general procedure A using (S)-3-methyl-3-phenyl-1-((S)-piperidin-3-yl)pyrrolidin-2-one (150 mg, 0.581 mmol), (R)-(2,3-dihydrobenzo[b][1,4]dioxin-2-yl)methyl 4-methylbenzenesulfonate (223 mg, 0.697 mmol), K₂CO₃ (201 mg, 1.451 mmol) and ACN (1 ml). The product was purified by reversed phase flash chromatography to obtain 67 mg of the product.

¹H NMR (400 MHz, CDCl₃) δ ppm 1.41-1.50 (m, 1H) 1.52 (s, 3H) 1.63-1.85 (m, 3H) 2.06-2.21 (m, 3H) 2.41 (ddd, 1H) 2.56-2.72 (m, 2H) 2.82 (br d, 1H) 2.88-2.95 (m, 1H) 3.22 (dt, 1H) 3.37 (ddd, 1H) 3.93-4.10 (m, 1H) 4.13-4.33 (m, 3H) 6.80-6.89 (m, 4H) 7.19-7.25 (m, 1H) 7.28-7.44 (m, 4H).

### EXAMPLE 16: 1-((S)-1-(((S)-2,3-Dihydrobenzo[b][1,4]dioxin-2-yl)methyl)piperidin-3-yl)-3-ethylimidazolidin-2-one

### 1-((S)-1-(((S)-2,3-dihydrobenzo[b][1,4]dioxin-2-yl)methyl)piperidin-3-yl)imidazolidin-2-one

(100 mg, 0.315 mmol) was dissolved in DMF (1 ml) and cooled down to 0° C. under nitrogen atmosphere and NaH (25.2 mg, 0.630 mmol) was added. After stirring the reaction mixture for 20 minutes, bromoethane (0.030 ml, 0.410 mmol) was added and the reaction mixture was stirred on an ice-bath for 4 hours and continued at room temperature over night. After completion of the reaction water was added and the reaction mixture was extracted three times with dichloromethane. The organic phase was dried and evaporated to dryness. The evaporation residue was purified by reversed phase flash chromatography followed by normal phase flash chromatography to obtain 23.9 mg of the product.

¹H NMR (400 MHz, CDCl₃): δ ppm 6.78-6.89 (m, 4H) 4.22-4.36 (m, 2H) 3.80-4.06 (m, 2H) 3.17-3.43 (m, 6H) 2.88-3.01 (m, 1H) 2.75-2.85 (m, 1H) 2.50-2.71 (m, 2H) 1.94-2.24 (m, 2H) 1.63-1.83 (m, 3H) 1.31-1.45 (m, 1H) 1.04-1.17 (m, 3H).

### EXAMPLE 17: 2-(3-((S)-1-(((S)-2,3-Dihydrobenzo[b][1,4]dioxin-2-yl)methyl)-piperidin-3-yl)-2-oxoimidazolidin-1-yl)-N,N-dimethylacetamide

2-(3-((S)-1-(((S)-2,3-Dihydrobenzo[b][1,4]dioxin-2-yl)methyl)piperidin-3-yl)-2-oxoimidazolidin-1-yl)-N,N-dimethylacetamide was prepared as compound of Example 16 using 1-((S)-1-(((S)-2,3-dihydrobenzo[b][1,4]dioxin-2-yl)methyl)piperidin-3-yl)imidazolidin-2-one (200 mg, 0.630 mmol) and 2-chloro-N,N-dimethylacetamide (0.084 ml, 0.819 mmol). The evaporation residue was purified by reversed phase flash chromatography followed by normal phase flash chromatography to obtain 12.3 mg of the product.

¹H NMR (400 MHz, CDCl₃): δ ppm 6.76-6.92 (m, 4H) 4.17-4.40 (m, 2H) 3.83-4.07 (m, 4H) 3.30-3.55 (m, 4H) 2.90-3.05 (m, 7H) 2.75-2.86 (m, 1H) 2.50-2.73 (m, 2H) 2.02-2.27 (m, 2H) 1.65-1.86 (m, 3H) 1.32-1.48 (m, 1H).

### EXAMPLE 18: 5-tert-Butyl-3-((S)-1-(((S)-2,3-dihydrobenzo[b][1,4]dioxin-2-yl)methyl)piperidin-3-yl)oxazolidin-2-one

### Step 1: 1-((S)-1-(((S)-2,3-Dihydrobenzo[b][1,4]dioxin-2-yl)methyl)piperidin-3-ylamino)-3,3-dimethylbutan-2-ol

A mixture of (1,1-dimethylethyl)oxirane (4.59 mmol, 0.559 ml), (S)-1-(((S)-2,3-dihydrobenzo[b][1,4]dioxin-2-yl)methyl)piperidin-3-amine (2.295 mmol, 0.57 g) and 2-propanol (4.2 ml) was heated at 170° C. in the microwave for 2.5 hours. The solvents were evaporated. The evaporation residue was purified by reversed phase flash chromatography to obtain 595 mg of the product.

LC-MS (ES+) [M+1]: 349.3.

### Step 2: 5-tert-Butyl-3-((S)-1-(((S)-2,3-dihydrobenzo[b][1,4]dioxin-2-yl)methyl)piperidin-3-yl)oxazolidin-2-one

A mixture of 1-((S)-1-(((S)-2,3-dihydrobenzo[b][1,4]dioxin-2-yl)methyl)piperidin-3-ylamino)-3,3-dimethylbutan-2-ol (0.861 mmol, 300 mg), N,N′-carbonyldiimidazole (1.291 mmol, 209 mg) and DMF (4 ml) was heated in the microwave at 170° C. for 16 hours. The reaction mixture was diluted with DCM and made acidic with 0.5 M HCl. The mixture was filtered through celite, the phases were separated and the aqueous phase was extracted with DCM. The combined organic phases were washed with water and brine, then dried and evaporated. The evaporation residue was purified by flash chromatography to obtain 17 mg of the product.

¹H NMR (400 MHz, CDCl₃) δ ppm 0.96 (s, 9H) 1.32-1.54 (m, 1H) 1.58-1.90 (m, 3H) 2.16-2.23 (m, 2H) 2.55-2.70 (m, 2H) 2.72-3.07 (m, 2H) 3.22-3.59 (m, 2H) 3.79-3.93 (m, 1H) 3.94-4.06 (m, 1H) 4.10-4.20 (m, 1H) 4.22-4.39 (m, 2H) 6.64-6.98 (m, 4H).

### EXAMPLE 19: 3-((S)-1-(((S)-2,3-Dihydrobenzo[b][1,4]dioxin-2-yl)methyl)piperidin-3-yl)-5-isopropyloxazolidin-2-one

### Step 1: 1-(((S)-1-(((S)-2,3-Dihydrobenzo[b][1,4]dioxin-2-yl)methyl)piperidin-3-yl)amino)-3-methylbutan-2-ol

1-(((S)-1-(((S)-2,3-Dihydrobenzo[b][1,4]dioxin-2-yl)methyl)piperidin-3-yl)amino)-3-methylbutan-2-ol was prepared as step 1 in example 18 using 1,2-epoxy-3-methylbutane (2.215 mmol, 0.234 ml) (S)-1-(((S)-2,3-dihydrobenzo[b][1,4]dioxin-2-yl)methyl)piperidin-3-amine (2.215 mmol, 550 mg) and 2-propanol (2 ml). The crude product was purified by combiflash chromatography to obtain 270 mg of the product.

LC-MS (ES+) [M+1]: 335.2.

### Step 2: 3-((S)-1-(((S)-2,3-Dihydrobenzo[b][1,4]dioxin-2-yl)methyl)piperidin-3-yl)-5-isopropyloxazolidin-2-one

1-((S)-1-(((S)-2,3-Dihydrobenzo[b][1,4]dioxin-2-yl)methyl)piperidin-3-ylamino)-3-methylbutan-2-ol (0.276 mmol, 110 mg), N,N′-carbonyldiimidazole (0.414 mmol, 67.2 mg), 4-dimethylaminopyridine (0.028 mmol, 3.38 mg) and ACN was heated at 100° C. in the microwave for 1 h. After evaporation of the solvents the residue was purified by reversed phase flash chromatography to obtain 67 mg of the product.

¹H NMR (400 MHz, CDCl₃) δ ppm 0.92 (d, 3H) 1.00 (dd, 3H) 1.44-1.48 (m, 1H) 1.62-1.93 (m, 4H) 2.06-2.28 (m, 2H) 2.50-2.72 (m, 2H) 2.72-2.84 (m, 1H) 2.90-3.01 (m, 1H) 3.11-3.35 (m, 1H) 3.44-3.67 (m, 1H) 3.77-3.94 (m, 1H) 3.94-4.06 (m, 1H) 4.11-4.39 (m, 3H) 6.74-6.93 (m, 4H).

### EXAMPLE 20: N-((3-((S)-1-(((S)-2,3-Dihydrobenzo[b][1,4]dioxin-2-yl)methyl)piperidin-3-yl)-2-oxooxazolidin-5-yl)methyl)acetamide

### Step 1: tert-Butyl 3-((S)-1-(((S)-2,3-dihydrobenzo[b][1,4]dioxin-2-yl)methyl)piperidin-3-ylamino)-2-hydroxypropylcarbamate

tert-Butyl 3-((S)-1-(((S)-2,3-dihydrobenzo[b][1,4]dioxin-2-yl)methyl)piperidin-3-ylamino)-2-hydroxypropylcarbamate was prepared as step 1 in example 18 using (S)-1-(((S)-2,3-dihydrobenzo[b][1,4]dioxin-2-yl)methyl)piperidin-3-amine (5.24 mmol, 1.30 g), tert-butyl N-(2-oxiranylmethyl carbamate (5.76 mmol, 0.997 g) and 2-propanol (5 ml). The cure product was purified by reversed phase flash chromatography to obtain 1.3 g of the product.

¹H NMR (400 MHz, CDCl₃) δ ppm 1.18-1.30 (m, 1H) 1.45 (s, 9H) 1.63-1.80 (m, 4H) 2.07-2.20 (m, 1H) 2.29-2.40 (m, 1H) 2.45-2.70 (m, 5H) 2.73-2.89 (m, 2H) 3.03-3.10 (m, 1H) 3.23-3.37 (m, 1H) 3.55-3.73 (m, 2H) 3.98 (dd, 1H) 4.27-4.33 (m, 2H) 4.99 (br s, 1H) 6.80-6.93 (m, 4H).

### Step 2: tert-Butyl (3-((S)-1-(((S)-2,3-dihydrobenzo[b][1,4]dioxin-2-yl)methyl)piperidin-3-yl)-2-oxooxazolidin-5-yl)methylcarbamate

tert-Butyl (3-((S)-1-(((S)-2,3-dihydrobenzo[b][1,4]dioxin-2-yl)methyl)piperidin-3-yl)-2-oxooxazolidin-5-yl)methylcarbamate was prepared as step 2 in example 19 using tert-butyl 3-((S)-1-(((S)-2,3-dihydrobenzo[b][1,4]dioxin-2-yl)methyl)piperidin-3-ylamino)-2-hydroxypropylcarbamate (3.08 mmol, 1.30 g), N,N′-carbonyldiimidazole (4.63 mmol, 0.750 g), 4-dimethylaminopyridine (0.308 mmol, 0.038 g) and ACN (3 ml). The crude product was purified by flash chromatography to obtain 1.12 g of the product.

LC-MS (ES+) [M+1]: 449.0.

### Step 3: 5-(Aminomethyl)-3-((S)-1-(((S)-2,3-dihydrobenzo[b][1,4]dioxin-2-yl)methyl)piperidin-3-yl)oxazolidin-2-one, HCl

5-(Aminomethyl)-3-((S)-1-(((S)-2,3-dihydrobenzo[b][1,4]dioxin-2-yl)methyl)piperidin-3-yl)oxazolidin-2-one was prepared according to the general procedure E using tert-butyl (3-((S)-1-(((S)-2,3-dihydrobenzo[b][1,4]dioxin-2-yl)methyl)piperidin-3-yl)-2-oxooxazolidin-5-yl)methylcarbamate (2.503 mmol, 1.12 g), HCl in dioxane (0.626 ml) and DCM (15 ml). The residue was triturated with DCM to obtain 940 mg of product as HCL salt.

LC-MS (ES+) [M+1]: 348.5.

### Step 4: N-((3-((S)-1-(((S)-2,3-Dihydrobenzo[b][1,4]dioxin-2-yl)methyl)piperidin-3-yl)-2-oxooxazolidin-5-yl)methyl)acetamide

5-(Aminomethyl)-3-((S)-1-(((S)-2,3-dihydrobenzo[b][1,4]dioxin-2-yl)methyl)piperidin-3-yl)oxazolidin-2-one (0.288 mmol, 100 mg) and Et₃N (0.345 mmol, 0.048 ml) were dissolved in THF. The reaction mixture was cooled down on ice and acetic anhydride (0.317 mmol, 0.030 ml) was added. The reaction mixture was stirred on ice for 30 minutes. Saturated NaHCO₃ was added and the mixture was extracted with EtOAc. The combine organic phases were washed with brine, dried and evaporated to obtain 48 mg of the product.

¹H NMR (400 MHz, CDCl₃) δ ppm 1.32-1.53 (m, 1H) 1.66-1.89 (m, 3H) 2.02 (s, 3H) 1.99-2.03 (m, 1H) 2.07-2.30 (m, 2H) 2.58-2.72 (m, 2H) 2.79 (br d, 1H) 2.95 (td, 1H) 3.22-3.41 (m, 1H) 3.39-3.54 (m, 1H) 3.55-3.72 (m, 2H) 3.77-3.92 (m, 1H) 3.99 (dd, 1H) 4.19-4.42 (m, 1H) 4.48-4.70 (m, 1H) 6.09 (br d, 1H) 6.73-6.98 (m, 4H).

### EXAMPLE 21: 3-((S)-1-(((S)-2,3-Dihydrobenzo[b][1,4]dioxin-2-yl)methyl)piperidin-3-yl)-5-(4-fluorophenyl)oxazolidin-2-one

### Step 1: 2-((S)-1-(((S)-2,3-Dihydrobenzo[b][1,4]dioxin-2-yl)methyl)piperidin-3-ylamino)-1-(4-fluorophenyl)ethanol

(S)-1-(((S)-2,3-Dihydrobenzo[b][1,4]dioxin-2-yl)methyl)piperidin-3-amine (2.014 mmol, 0.5 g), 2-(4-fluorophenyl)oxirane (2.82 mmol, 0.389 g) and 2-propanol were heated in the microwave at 150° C. for 1 h. The solvents were evaporated and the residue was purified by reversed phase flash chromatography to obtain 330 mg of the product.

LC-MS (ES+) [M+1]: 388.4.

### Step 2: 3-((S)-1-(((S)-2,3-Dihydrobenzo[b][1,4]dioxin-2-yl)methyl)piperidin-3-yl)-5-(4-fluorophenyl)oxazolidin-2-one

3-((S)-1-(((S)-2,3-Dihydrobenzo[b][1,4]dioxin-2-yl)methyl)piperidin-3-yl)-5-(4-fluorophenyl)oxazolidin-2-one was prepared as in step 2 of example 19 using 2-((S)-1-(((S)-2,3-dihydrobenzo[b][1,4]dioxin-2-yl)methyl)piperidin-3-ylamino)-1-(4-fluorophenyl)-ethanol (0.854 mmol, 330 mg), N,N′-carbonyldiimidazole (1.281 mmol, 208 mg), 4-dimethylaminopyridine (0.085 mmol, 10.43 mg) and ACN (8.5 ml). The crude product was purified by reversed phase flash chromatography to obtain 186 mg of the product.

¹H NMR (400 MHz, CDCl₃) δ ppm 1.34-1.55 (m, 1H) 1.62-1.93 (m, 3H) 2.11-2.35 (m, 2H) 2.55-2.70 (m, 2H) 2.70-2.84 (m, 1H) 2.88-3.10 (m, 1H) 3.46 (ddd, 1H) 3.85-4.08 (m, 3H) 4.19-4.35 (m, 2H) 5.35-5.55 (m, 1H) 6.72-6.93 (m, 4H) 7.01-7.15 (m, 2H) 7.27-7.36 (m, 2H).

### EXAMPLE 22: 6-((S)-1-(((S)-2,3-Dihydrobenzo[b][1,4]dioxin-2-yl)methyl)piperidin-3-yl)-2-(methylthio)-6,7-dihydro-5H-pyrrolo[3,4-d]pyrimidin-5-one

### Step 1: (S)-tert-Butyl 3-(2-(methylthio)-5-oxo-5H-pyrrolo[3,4-d]pyrimidin-6(7H)-yl)piperidine-1-carboxylate

To a stirred solution of ethyl 4-(bromomethyl)-2-(methylthio)pyrimidine-5-carboxylate (2.80 g, 9.62 mmol) in DMF (15 ml) were added DIPEA (5.00 ml, 3.71 g, 28.7 mmol) and (S)-tert-butyl 3-aminopiperidine-1-carboxylate (1.93 g, 9.62 mmol) at RT and the resulting mixture was stirred for 67 h. The reaction mixture was diluted with water and extracted with EtOAc. The combined organic layers were dried and evaporated to dryness. The residue was purified by preparative HPLC yielding 0.22 g (S)-tert-butyl 3-(2-(methylthio)-5-oxo-5H-pyrrolo[3,4-d]pyrimidin-6(7H)-yl)piperidine-1-carboxylate.

LC-MS, m/z=365.2 (M+1)⁺.

### Step 2: (S)-2-(Methylthio)-6-(piperidin-3-yl)-6,7-dihydro-5H-pyrrolo[3,4-d]pyrimidin-5-one hydrochloride

A mixture of (S)-tert-butyl 3-(2-(methylthio)-5-oxo-5H-pyrrolo[3,4-d]pyrimidin-6(7H)-yl)piperidine-1-carboxylate (0.40 g, 1.10 mmol) and 4 M HCl in dioxane (10 ml, 40 mmol) was stirred at RT for 16 h. The solvent was evaporated off and the residue was triturated with 1:1 Et₂O/pentane yielding 0.23 g (S)-2-(Methylthio)-6-(piperidin-3-yl)-6,7-dihydro-5H-pyrrolo[3,4-d]pyrimidin-5-one hydrochloride.

LC-MS, m/z=265.2 (M+1)⁺.

### Step 3: 6-((S)-1-(((S)-2,3-Dihydrobenzo[b][1,4]dioxin-2-yl)methyl)piperidin-3-yl)-2-(methylthio)-6,7-dihydro-5H-pyrrolo[3,4-d]pyrimidin-5-one

Prepared using general procedure A from (S)-2-(methylthio)-6-(piperidin-3-yl)-6,7-dihydro-5H-pyrrolo[3,4-d]pyrimidin-5-one hydrochloride (0.10 g, 0.33 mmol), (R)-(2,3-dihydrobenzo[b][1,4]dioxin-2-yl)methyl 4-methylbenzenesulfonate (0.15 g, 0.45 mmol), and K₂CO₃ (0.12 g, 0.83 mmol) in ACN (3 ml) yielding 94 mg 6-((S)-1-(((S)-2,3-dihydrobenzo[b][1,4]dioxin-2-yl)methyl)piperidin-3-yl)-2-(methylthio)-6,7-dihydro-5H-pyrrolo[3,4-d]pyrimidin-5-one.

¹H NMR (400 MHz, CDCl₃) δ ppm 1.51-1.67 (2H, m), 1.68-1.94 (3H, m), 2.26-2.40 (2H, m), 2.63 (3H, s), 2.65-2.71 (1H, m), 2.76-2.86 (1H, m), 2.96-3.05 (1H, m), 3.98-4.06 (1H, m), 4.25-4.33 (2H, m), 4.40-4.51 (3H, m), 6.77-6.90 (4H, m), 8.89 (1H, s).

### EXAMPLE 23: 6-((S)-1-(((S)-2,3-Dihydrobenzo[b][1,4]dioxin-2-yl)methyl)piperidin-3-yl)-6,7-dihydro-5H-pyrrolo[3,4-d]pyrimidin-5-one

### Step 1: (S)-tert-Butyl 3-(5-oxo-5H-pyrrolo[3,4-d]pyrimidin-6(7H)-yl)piperidine-1-carboxylate

To a suspension of 10% Pd/C (0.30 g) in THF (30 ml) was added (S)-tert-butyl 3-(2-(methylthio)-5-oxo-5H-pyrrolo[3,4-d]pyrimidin-6(7H)-yl)piperidine-1-carboxylate (0.60 g, 1.65 mmol) and triethylsilane (1.32 ml, 0.96 g, 8.26 mmol) at RT and the resulting mixture was heated to 35° C. for 18 h. The reaction mixture was filtered through a pad of celite and evaporated to dryness. The residue was purified by flash chromatography yielding 0.48 g (S)-tert-butyl 3-(5-oxo-5H-pyrrolo[3,4-d]pyrimidin-6(7H)-yl)piperidine-1-carboxylate.

LC-MS, m/z=319.2 (M+1)⁺.

### Step 2: (S)-6-(Piperidin-3-yl)-6,7-dihydro-5H-pyrrolo[3,4-d]pyrimidin-5-one hydrochloride

A mixture of (S)-tert-butyl 3-(5-oxo-5H-pyrrolo[3,4-d]pyrimidin-6(7H)-yl)piperidine-1-carboxylate (0.48 g, 1.51 mmol) and 4 M HCl in dioxane (10 ml) was stirred at RT for 16 h. The solvent was evaporated off and the residue was triturated with 1:1 Et₂O/pentane yielding 0.38 g (S)-6-(piperidin-3-yl)-6,7-dihydro-5H-pyrrolo[3,4-d]pyrimidin-5-one hydrochloride.

LC-MS, m/z=219.2 (M+1)⁺.

### Step 3: 6-((S)-1-(((S)-2,3-Dihydrobenzo[b][1,4]dioxin-2-yl)methyl)piperidin-3-yl)-6,7-dihydro-5H-pyrrolo[3,4-d]pyrimidin-5-one

Prepared using general procedure A from (S)-6-(piperidin-3-yl)-6,7-dihydro-5H-pyrrolo[3,4-d]pyrimidin-5-one hydrochloride (0.10 g, 0.39 mmol), (R)-(2,3-dihydrobenzo[b][1,4]dioxin-2-yl)methyl 4-methylbenzenesulfonate (0.15 g, 0.47 mmol), and K₂CO₃ (0.16 g, 1.18 mmol) in ACN (3 ml) yielding 15 mg 6-((S)-1-(((S)-2,3-dihydrobenzo[b][1,4]dioxin-2-yl)methyl)piperidin-3-yl)-6,7-dihydro-5H-pyrrolo[3,4-d]pyrimidin-5-one.

¹H NMR (400 MHz, CDCl₃) δ ppm 1.58-1.70 (1H, m), 1.71-1.87 (2H, m), 1.88-1.97 (1H, m), 2.28-2.43 (2H, m), 2.61-2.76 (2H, m), 2.78-2.88 (1H, m), 2.99-3.07 (1H, m), 3.98-4.06 (1H, m), 4.26-4.34 (2H, m), 4.44-4.59 (3H, m), 6.78-6.92 (4H, m), 9.15 (1H, s), 9.35 (1H, s).

### EXAMPLE 24: 5-((S)-1-(((S)-2,3-Dihydrobenzo[b][1,4]dioxin-2-yl)methyl)piperidin-3-yl)-1-methyl-4,5-dihydropyrrolo[3,4-c]pyrazol-6(1H)-one

### Step 1: (S)-tert-Butyl 3-(((5-bromo-1-methyl-1H-pyrazol-4-yl)methyl)amino)-piperidine-1-carboxylate

To an ice-cold stirred solution of (5-bromo-1-methyl-1H-pyrazol-4-yl)methanol (5.10 g, 26.7 mmol) in CH₂Cl₂ (50 ml) were added TEA (11.23 ml, 8.15 g, 80.6 mmol) and methanesulfonyl chloride (2.50 ml, 3.70 g, 32.3 mmol). The resulting solution was stirred for 2 h at RT. The reaction mixture was diluted with DCM, washed with water and brine, dried and evaporated. The residue (3.10 g, 11.52 mmol) and (S)-tert-butyl 3-aminopiperidine-1-carboxylate (2.30 g, 11.52 mmol) were dissolved in ACN (35 ml) and K₂CO₃ (4.77 g, 34.57 mmol) was added. The resulting mixture was heated to 80° C. for 16 h. The solvent was evaporated off and the residue was dissolved in mixture of EtOAc, washed with water, dried and evaporated. The residue was purified by flash chromatography yielding 1.70 g (S)-tert-butyl 3-(((5-bromo-1-methyl-1H-pyrazol-4-yl)methyl)amino)-piperidine-1-carboxylate.

LC-MS, m/z=373.1 (M+1)⁺.

### Step 2: (S)-tert-Butyl 3-(((5-(ethoxycarbonyl)-1-methyl-1H-pyrazol-4-yl)methyl)-amino)piperidine-1-carboxylate

To a solution of (S)-tert-butyl 3-(((5-bromo-1-methyl-1H-pyrazol-4-yl)methyl)amino)-piperidine-1-carboxylate (1.70 g, 4.02 mmol) in EtOH (30 ml) were added NaOAc (0.66 g, 8.04 mmol) and PdCl₂(dppf).CH₂Cl₂ (0.16 g, 0.20 mmol) in an autoclave. The autoclave was pressurized with CO (500 psi) and stirred at 140° C. for 24 h. The solvent was evaporated off and the residue was dissolved in EtOAc, washed with water and brine, dried and evaporated. The residue was purified by flash chromatography yielding 0.80 g (S)-tert-butyl 3-(((5-(ethoxycarbonyl)-1-methyl-1H-pyrazol-4-yl)methyl)amino)piperidine-1-carboxylate.

LC-MS, m/z=367.3 (M+1)⁺.

### Step 3: (S)-4-(((1-(tert-Butoxycarbonyl)piperidin-3-yl)amino)methyl)-1-methyl-1H-pyrazole-5-carboxylic acid

To a stirred solution of (S)-tert-butyl 3-(((5-(ethoxycarbonyl)-1-methyl-1H-pyrazol-4-yl)methyl)amino)piperidine-1-carboxylate (0.75 g, 2.05 mmol) in a mixture of THF (7.5 ml) and water (7.5 ml) was added LiOH.H₂O (0.43 g, 10.2 mmol) and the resulting mixture was stirred for 5 h at RT. The reaction mixture was concentrated under reduced pressure. The obtained residue was dissolved in water (50 ml). The aqueous layer was washed with EtOAc. The aqueous layer pH was adjusted to 4 with citric acid and extracted with 10% MeOH in CH₂Cl₂. The organic layer was dried and concentrated yielding 0.69 g (S)-4-(((1-(tert-butoxycarbonyl)piperidin-3-yl)amino)methyl)-1-methyl-1H-pyrazole-5-carboxylic acid.

LC-MS, m/z=339.3 (M+1)⁺.

### Step 4: (S)-tert-Butyl 3-(1-methyl-6-oxopyrrolo[3,4-c]pyrazol-5(1H,4H,6H)-yl)piperidine-1-carboxylate

To a solution of (S)-4-(((1-(tert-butoxycarbonyl)piperidin-3-yl)amino)methyl)-1-methyl-1H-pyrazole-5-carboxylic acid (0.69 gg, 2.04 mmol) in DCM (20 ml) were added EDC.HCl (0.69 g, 3.06 mmol), HOBt (0.43 g, 3.06 mmol) and DMAP (0.62 g, 5.10 mmol) at 0° C. The reaction mixture was stirred at RT for 16 h. The mixture was diluted with DCM, washed with water and brine, dried and evaporated. The residue was purified by flash chromatography yielding 0.14 g (S)-tert-butyl 3-(1-methyl-6-oxopyrrolo[3,4-c]pyrazol-5 (1H,4H,6H)-yl)piperidine-1-carboxylate.

LC-MS, m/z=321.2 (M+1)⁺.

### Step 5: (S)-1-Methyl-5-(piperidin-3-yl)-4,5-dihydropyrrolo[3,4-c]pyrazol-6(1H)-one trifluoroacetate

A solution of (S)-tert-butyl 3-(1-methyl-6-oxopyrrolo[3,4-c]pyrazol-5(1H,4H,6H)-yl)piperidine-1-carboxylate (0.14 g, 0.44 mmol) in DCM (10 ml) was treated with TFA (0.35 ml, 0.52 g, 4.57 mmol) at 0° C. The reaction mixture was stirred for 6 h at RT and evaporated to dryness. The residue was triturated with Et₂O yielding 0.12 g (S)-1-methyl-5-(piperidin-3-yl)-4,5-dihydropyrrolo[3,4-c]pyrazol-6(1H)-one trifluoroacetate.

LC-MS, m/z=221.2 (M+1)⁺

### Step 6: 5-((S)-1-(((S)-2,3-Dihydrobenzo[b][1,4]dioxin-2-yl)methyl)piperidin-3-yl)-1-methyl-4,5-dihydropyrrolo[3,4-c]pyrazol-6(1H)-one

Prepared using general procedure A from (S)-1-methyl-5-(piperidin-3-yl)-4,5-dihydropyrrolo[3,4-c]pyrazol-6(1H)-one trifluoroacetate (0.10 g, 0.30 mmol), (R)-(2,3-dihydrobenzo[b][1,4]dioxin-2-yl)methyl 4-methylbenzenesulfonate (0.12 g, 0.36 mmol), and Na₂CO₃ (70 mg, 0.66 mmol) in DMF (3 ml) yielding 51 mg 5-((S)-1-(((S)-2,3-dihydrobenzo[b][1,4]dioxin-2-yl)methyl)piperidin-3-yl)-1-methyl-4,5-dihydropyrrolo[3,4-c]pyrazol-6(1H)-one.

¹H NMR (400 MHz, CDCl₃) δ ppm 1.46-1.64 (1H, m), 1.67-1.83 (2H, m), 1.85-1.94 (1H, m), 2.18-2.36 (2H, m), 2.61-2.73 (2H, m), 2.79-2.88 (1H, m), 2.99-3.07 (1H, m), 3.99-4.05 (1H, m), 4.05 (3H, s), 4.19-4.25 (2H, m), 4.25-4.33 (3H, m), 6.80-6.90 (4H, m), 7.36 (1H, s).

### EXAMPLE 25: 1-((S)-1-(((S)-2,3-Dihydrobenzo[b][1,4]dioxin-2-yl)methyl)piperidin-3-yl)-1,3-dihydrobenzo[c]isothiazole 2,2-dioxide

### Step 1: 1-(2-Bromophenyl)-N—((S)-1-(((S)-2,3-dihydrobenzo[b][1,4]dioxin-2-yl)methyl)piperidin-3-yl)methanesulfonamide

To a solution of (S)-1-(((S)-2,3-dihydrobenzo[b][1,4]dioxin-2-yl)methyl)piperidin-3-amine (0.26 g, 1.03 mmol) and TEA (0.17 ml, 0.13 g, 1.24 mmol) in DCM (5 ml) was added a DCM (2 ml) solution of 2-bromobenzylsulfonyl chloride (0.28 g, 1.03 mmol) at 0° C. The resulting mixture was stirred overnight at RT, diluted with DCM and washed with water and brine. After drying and evaporating to dryness 0.48 g 1-(2-bromophenyl)-N—((S)-1-(((S)-2,3-dihydrobenzo[b][1,4]dioxin-2-yl)methyl)piperidin-3-yl)methanesulfonamide was obtained.

LC-MS, m/z=481.3 (M+1)⁺.

### Step 2: 1-((S)-1-(((S)-2,3-Dihydrobenzo[b][1,4]dioxin-2-yl)methyl)piperidin-3-yl)-1,3-dihydrobenzo[c]isothiazole 2,2-dioxide

To a degassed mixture of 1-(2-bromophenyl)-N—((S)-1-(((S)-2,3-dihydrobenzo[b][1,4]-dioxin-2-yl)methyl)piperidin-3-yl)methanesulfonamide (0.25 g, 0.52 mmol), K₃PO₄ (0.33 g, 1.56 mmol) and toluene (6 ml) was added CuI (20 mg, 0.10 mml) and N,N′-dimethyl-1,2-diaminoethane (11 μl, 9.2 mg, 0.10 mmol). The resulting mixture was heated at microwave reactor at 110° C. for 5 h. After diluting with EOAc the mixture was filtered through a Celite pad and evaporated to dryness. The crude product was purified by flash chromatography yielding 0.15 g 1-((S)-1-(((S)-2,3-dihydrobenzo[b][1,4]dioxin-2-yl)methyl)piperidin-3-yl)-1,3-dihydrobenzo[c]isothiazole 2,2-dioxide.

¹H NMR (400 MHz, CDCl₃) δ ppm 1.63-1.77 (1H, m), 1.80-1.90 (1H, m), 2.01-2.15 (1H, m), 2.16-2.31 (2H, m), 2.61-2.81 (3H, m), 2.88-2.97 (1H, m), 3.23-3.31 (1H, m), 3.95-4.08 (2H, m), 4.26-4.36 (4H, m), 6.79-6.89 (5H, m), 6.94-7.41 (1H, m), 7.19-7.24 (1H, m), 7.28-7.35 (1H, m).

### EXAMPLE 26: 1-((S)-1-(((S)-2,3-Dihydrobenzo[b][1,4]dioxin-2-yl)methyl)piperidin-3-yl)indolin-2-one

### Step 1: 2-(2-Bromophenyl)-N—((S)-1-(((S)-2,3-dihydrobenzo[b][1,4]dioxin-2-yl)methyl)piperidin-3-yl)acetamide

To a solution of (S)-1-(((S)-2,3-dihydrobenzo[b][1,4]dioxin-2-yl)methyl)piperidin-3-amine (0.22 g, 0.89 mmol) and TEA (0.25 ml, 0.18 g, 1.77 mmol) in DCM (10 ml) was added 2-(2-bromophenyl)acetyl chloride (from 0.20 g, 0.93 mmol of 2-bromophenylacetic acid, Chem. Comm., (24), 2874-2875, 2004) at 0° C. The resulting mixture was stirred overnight at RT. The solution was diluted with DCM and washed with NaHCO₃ solution, water and brine. After drying and evaporating to dryness the residue was purified by flash chromatography yielding 0.22 g 2-(2-bromophenyl)-N—((S)-1-(((S)-2,3-dihydrobenzo-[b][1,4]dioxin-2-yl)methyl)piperidin-3-yl)acetamide.

LC-MS, m/z=445.3 (M+1)⁺.

### Step 2: 1-((S)-1-(((S)-2,3-Dihydrobenzo[b][1,4]dioxin-2-yl)methyl)piperidin-3-yl)indolin-2-one

A degassed solution of 2-(2-bromophenyl)-N—((S)-1-(((S)-2,3-dihydrobenzo[b][1,4]dioxin-2-yl)methyl)piperidin-3-yl)acetamide (0.22 g, 0.48 mmol), K₂CO₃ (0.17 g, 1.21 mmol), Pd(OAc)₂ (5.4 mg, 0.024 mmol), phenylboronic acid (7.4 mg, 0.060 mmol) and XPhos (29 mg, 0.060 mmol) in t-BuOH (7 ml) was refluxed overnight. The resulting mixture was diluted with DCM, filtered through a Celite pad and evaporated to dryness. The crude product was purified by flash chromatography yielding 24 mg 1-((S)-1-(((S)-2,3-dihydrobenzo[b][1,4]dioxin-2-yl)methyl)piperidin-3-yl)indolin-2-one.

¹H NMR (400 MHz, CDCl₃) δ ppm 1.66-1.89 (3H, m), 2.19-2.34 (2H, m), 2.63-2.79 (2H, m), 2.86-3.02 (3H, m), 3.50 (2H, s), 3.96-4.05 (1H, m), 4.23-4.36 (3H, m), 6.78-6.89 (4H, m), 6.98-7.05 (2H, m), 7.20-7.28 (2H, m).

### EXAMPLE 27: 1-((S)-1-(((S)-2,3-Dihydrobenzo[b][1,4]dioxin-2-yl)methyl)piperidin-3-yl)-4,6-difluoro-2-methyl-1H-benzo[d]imidazole

### Step 1: (S)—N-(3,5-Difluoro-2-nitrophenyl)-1-(((S)-2,3-dihydrobenzo[b][1,4]dioxin-2-yl)methyl)piperidin-3-amine

A mixture of (S)-1-(((S)-2,3-dihydrobenzo[b][1,4]dioxin-2-yl)methyl)piperidin-3-amine (0.55 g, 2.22 mmol), 2,4,6-trifluoronitrobenzene (0.39 g, 2.22 mmol) and DIPEA (0.46 ml, 0.34 g, 2.66 mmol) in DMF (15 ml) was stirred overnight at RT. The mixture was diluted with EtOAc and washed with water and brine. After drying and evaporating to drynees the residue was purified by flash chromatography yielding 0.68 g (S)—N-(3,5-difluoro-2-nitrophenyl)-1-(((S)-2,3-dihydrobenzo[b][1,4]dioxin-2-yl)methyl)piperidin-3-amine.

LC-MS, m/z=406.8 (M+1)⁺.

### Step 2: N1-((S)-1-(((S)-2,3-dihydrobenzo[b][1,4]dioxin-2-yl)methyl)piperidin-3-yl)-3,5-difluorobenzene-1,2-diamine

Prepared using General procedure F from (S)—N-(3,5-difluoro-2-nitrophenyl)-1-(((S)-2,3-dihydrobenzo[b][1,4]dioxin-2-yl)methyl)piperidin-3-amine (0.68 g, 1.67 mmol), NH₄Cl (0.89 g, 16.7 mmol) and Zn dust (1.09 g, 16.7 mmol) in THF (8 ml), MeOH (4 ml) and water (4 ml) yielding 0.59 g N1-((S)-1-(((S)-2,3-dihydrobenzo[b][1,4]dioxin-2-yl)methyl)piperidin-3-yl)-3,5-difluorobenzene-1,2-diamine.

LC-MS, m/z=376.8 (M+1)⁺.

### Step 3: 1-((S)-1-(((S)-2,3-Dihydrobenzo[b][1,4]dioxin-2-yl)methyl)piperidin-3-yl)-4,6-difluoro-2-methyl-1H-benzo[d]imidazole

A mixture of N1-((S)-1-(((S)-2,3-dihydrobenzo[b][1,4]dioxin-2-yl)methyl)piperidin-3-yl)-3,5-difluorobenzene-1,2-diamine (0.28 g, 0.75 mmol), trimethylorthoacetate (0.29 ml, 0.27 g, 2.24 mmol) and a crystal of p-TsOH in methanol (3 ml) was heated for 11 h at 100° C. in a sealed tube. After evaporation to dryness, the crude product was purified by flash chromatography yielding 0.19 g 1-((S)-1-(((S)-2,3-Dihydrobenzo[b][1,4]dioxin-2-yl)methyl)piperidin-3-yl)-4,6-difluoro-2-methyl-1H-benzo[d]imidazole.

¹H NMR (400 MHz, CDCl₃) δ ppm 1.73-2.03 (3H, m), 2.07-2.21 (1H, m), 2.23-2.35 (1H, m), 2.64 (3H, s), 2.67-2.87 (3H, m), 2.99-3.09 (1H, m), 3.09-3.17 (1H, m), 3.96-4.05 (1H, m), 4.25-4.47 (3H, m), 6.68-6.77 (1H, m), 6.80-6.90 (4H, m), 7.01-7.07 (1H, m).

### EXAMPLE 28: 1-((S)-1-(((S)-2,3-Dihydrobenzo[b][1,4]dioxin-2-yl)methyl)piperidin-3-yl)-5,6-difluoro-2-methyl-1H-benzo[d]imidazole

### Step 1: (S)—N-(4,5-Difluoro-2-nitrophenyl)-1-(((S)-2,3-dihydrobenzo[b][1,4]dioxin-2-yl)methyl)piperidin-3-amine

To a mixture of(S)-1-(((S)-2,3-dihydrobenzo[b][1,4]dioxin-2-yl)methyl)piperidin-3-amine (0.50 g, 2.01 mmol) and K₂CO₃ (0.28 g, 2.01 mmol) in DMF (5 ml) at 0° C. was slowly added a solution of 1,2,4-trifluoro-5-nitrobenzene (0.36 g, 2.01 mmol) in DMF (3 ml). The resulting mixture was stirred overnight at RT. The mixture was diluted with EtOAc and washed with water and brine, dried and evaporated to dryness. The residue was stirred with Et₂O (10 ml) and the solid was filtered off. The filtrate was evaporated to dryness and the residue was purified by flash chromatography yielding 67 mg (S)—N-(4,5-difluoro-2-nitrophenyl)-1-(((S)-2,3-dihydrobenzo[b][1,4]dioxin-2-yl)methyl)piperidin-3-amine.

LC-MS, m/z=406.8 (M+1)⁺.

### Step 2: N1-((S)-1-(((S)-2,3-Dihydrobenzo[b][1,4]dioxin-2-yl)methyl)piperidin-3-yl)-4,5-difluorobenzene-1,2-diamine

Prepared using general procedure F from (S)—N-(4,5-difluoro-2-nitrophenyl)-1-(((S)-2,3-dihydrobenzo[b][1,4]dioxin-2-yl)methyl)piperidin-3-amine (67 mg, 0.17 mmol), NH₄Cl (88 mg, 1.65 mmol) and Zn dust (0.11 g, 1.65 mmol) in THF (2 ml), MeOH (1 ml) and water (1 ml) yielding 61 mg N1-((S)-1-(((S)-2,3-dihydrobenzo[b][1,4]-dioxin-2-yl)methyl)piperidin-3-yl)-4,5-difluorobenzene-1,2-diamine.

LC-MS, m/z=376.7 (M+1)⁺.

### Step 3: 1-((S)-1-(((S)-2,3-Dihydrobenzo[b][1,4]dioxin-2-yl)methyl)piperidin-3-yl)-5,6-difluoro-2-methyl-1H-benzo[d]imidazole

Prepared as described in example 27, step 3 from N1-((S)-1-(((S)-2,3-dihydrobenzo[b][1,4]dioxin-2-yl)methyl)piperidin-3-yl)-4,5-difluorobenzene-1,2-diamine (61 mg, 0.16 mmol), trimethyl orthoacetate (62 μl, 59 mg, 0.49 mmol) and a crystal of p-TsOH in MeOH (1 ml) yielding 33 mg 1-((S)-1-(((S)-2,3-dihydrobenzo[b][1,4]dioxin-2-yl)methyl)piperidin-3-yl)-5,6-difluoro-2-methyl-1H-benzo[d]imidazole.

¹H NMR (400 MHz, CDCl₃) δ ppm 1.73-1.88 (1H, m), 1.89-2.04 (2H, m), 2.05-2.19 (1H, m), 2.24-2.36 (1H, m), 2.63 (3H, s), 2.66-2.85 (3H, m), 3.00-3.16 (2H, m), 3.96-4.06 (1H, m), 4.24-4.47 (3H, m), 6.79-6.91 (4H, m), 7.28-7.36 (1H, m), 7.39-7.47 (1H, m).

### EXAMPLE 29: 1-((S)-1-(((S)-2,3-Dihydrobenzo[b][1,4]dioxin-2-yl)methyl)piperidin-3-yl)pyrrolidin-2-one

Prepared using general procedure A from (S)-1-(piperidin-3-yl)pyrrolidin-2-one hydrochloride (0.10 g, 0.49 mmol), (R)-2-(bromomethyl)-2,3-dihydrobenzo[b][1,4]dioxine (0.11 g, 0.49 mmol) and DIPEA (0.30 ml, 0.22 g, 1.71 mmol) in ACN (3 ml) yielding 40 mg 1-((S)-1-(((S)-2,3-dihydrobenzo[b][1,4]dioxin-2-yl)methyl)piperidin-3-yl)pyrrolidin-2-one.

¹H NMR (400 MHz, CDCl₃) δ ppm 1.35-1.49 (1H, m), 1.63-1.80 (3H, m), 1.93-2.05 (2H, m), 2.07-2.20 (2H, m), 2.37 (2H, t), 2.60-2.67 (2H, m), 2.76-2.85 (1H, m), 2.86-2.94 (1H, m), 3.30-3.46 (2H, m), 3.96-4.05 (1H, m), 4.07-4.19 (1H, m), 4.22-4.33 (2H, m), 6.78-6.89 (4H, m).

### EXAMPLE 30: 1-((S)-1-(((S)-2,3-Dihydrobenzo[b][1,4]dioxin-2-yl)methyl)piperidin-3-yl)-5-methylpyrrolidin-2-one

### Step 1: (3S)-tert-Butyl 3-(2-methyl-5-oxopyrrolidin-1-yl)piperidine-1-carboxylate

To a stirred suspension of (S)-tert-butyl 3-aminopiperidine-1-carboxylate (1.50 g, 7.48 mmol) in dry DCM (20 ml) was added methyl 4-oxopentanoate (0.97 g, 7.48 mmol), followed by NaBH(OAc)₃ (1.90 g, 8.98 mmol) at 0° C. The resulting suspension was stirred at RT for 6 h. The mixture was diluted with water and extracted with DCM. The combined organic layers were washed with brine, dried and evaporated to dryness. The residue was purified by flash chromatography yielding 0.80 g (3S)-tert-butyl 3-(2-methyl-5-oxopyrrolidin-1-yl)piperidine-1-carboxylate.

LC-MS, m/z=283.2 (M+1)⁺.

### Step 2: 5-Methyl-1-((S)-piperidin-3-yl)pyrrolidin-2-one hydrochloride

(3S)-tert-butyl 3-(2-methyl-5-oxopyrrolidin-1-yl)piperidine-1-carboxylate (0.60 g, 2.13 mmol) was treated with 1 M HCl in Et₂O (5 ml, 5 mmol) at 0° C. and stirred for 1 h at the same temperature. The solvent was evaporated off and the residue was triturated with Et₂O yielding 0.29 g 5-Methyl-1-((S)-piperidin-3-yl)pyrrolidin-2-one hydrochloride.

LC-MS, m/z=183.1 (M+1)⁺.

### Step 3: 1-((S)-1-(((S)-2,3-Dihydrobenzo[b][1,4]dioxin-2-yl)methyl)piperidin-3-yl)-5-methylpyrrolidin-2-one

Prepared using general procedure A from 5-methyl-1-((S)-piperidin-3-yl)pyrrolidin-2-one hydrochloride (0.26 g, 1.19 mmol), (R)-2-(bromomethyl)-2,3-dihydrobenzo[b][1,4]dioxine (0.33 g, 1.43 mmol) and K₂CO₃ (0.30 g, 2.14 mmol) in ACN (4 ml) yielding 0.15 g 1-((S)-1-(((S)-2,3-dihydrobenzo[b][1,4]dioxin-2-yl)methyl)piperidin-3-yl)-5-methylpyrrolidin-2-one.

¹H NMR (400 MHz, CDCl₃) δ ppm 1.21-1.29 (3H, m), 1.54-1.87 (5H, m), 2.08-2.74 (7H, m), 2.79-3.00 (2H, m), 3.67-3.85 (2H, m), 3.95-4.04 (1H, m), 4.23-4.33 (2H, m), 6.78-6.89 (4H, m).

### EXAMPLE 31: 1-((S)-1-(((S)-2,3-Dihydrobenzo[b][1,4]dioxin-2-yl)methyl)piperidin-3-yl)-3-phenylpyrrolidin-2-one

To a solution of 4-chloro-2-phenylbutanoic acid (0.22 g, 1.11 mmol, Journal of Pharmaceutical Sciences, 79(8), 758-62; 1990) and (S)-1-(((S)-2,3-dihydrobenzo[b][1,4]-dioxin-2-yl)methyl)piperidin-3-amine (0.25 g, 1.01 mmol) in EtOAc (2 ml) at 0° C. was added TEA (0.42 ml, 0.31 g, 3.02 mmol) and 50% solution of T3P in EtOAc (0.77 ml, 1.31 mmol). The resulting mixture was stirred overnight at RT, quenched with water and extracted with EtOAc. The organic phase was washed with water, dried and evaporated to dryness. The residue was dissolved in THF (5 ml) and treated with KOtBu (0.17 g, 1.51 mmol). After stirring overnight at RT, the mixture was diluted with water and extracted with EtOAc. The organic phases were washed with water and brine, dried and evaporated. The residue was purified by flash chromatography yielding 0.11 g 1-((S)-1-(((S)-2,3-dihydrobenzo[b][1,4]dioxin-2-yl)methyl)piperidin-3-yl)-3-phenylpyrrolidin-2-one.

¹H NMR (400 MHz, CDCl₃) δ ppm 1.41-1.59 (1H, m), 1.62-1.86 (3H, m), 2.04-2.27 (3H, m), 2.43-2.72 (3H, m), 2.77-2.88 (1H, m), 2.91-3.02 (1H, m), 3.33-3.58 (2H, m), 3.65 (1H, t), 3.97-4.06 (1H, m), 4.15-4.33 (3H, m), 6.78-6.90 (4H, m), 7.19-7.36 (5H, m).

### EXAMPLE 32: 1-((S)-1-(((S)-2,3-Dihydrobenzo[b][1,4]dioxin-2-yl)methyl)piperidin-3-yl)-4-phenylpyrrolidin-2-one

### Step 1: 4-Bromo-N—((S)-1-(((S)-2,3-dihydrobenzo[b][1,4]dioxin-2-yl)methyl)piperidin-3-yl)-3-phenylbutanamide

To a solution of(S)-1-(((S)-2,3-dihydrobenzo[b][1,4]dioxin-2-yl)methyl)piperidin-3-amine (0.12 g, 0.50 mmol) and 4-bromo-3-phenylbutanoyl chloride (0.13 g, 0.50 mmol, Farmaco, Edizione Scientifica (1968), 23(4), 321-43) in toluene (10 ml) was added TEA (0.21 ml, 0.15 g, 1.49 mmol) and the resulting mixture was stirred for 3 h at RT and refluxed for 6.5 h. After filtering through a Celite pad and evaporating to dryness, the residue was purified by flash chromatography yielding 48 mg 4-bromo-N—((S)-1-(((S)-2,3-dihydrobenzo[b][1,4]-dioxin-2-yl)methyl)piperidin-3-yl)-3-phenylbutanamide.

LC-MS, m/z=475.2 (M+1)⁺.

### Step 2: 1-((S)-1-(((S)-2,3-Dihydrobenzo[b][1,4]dioxin-2-yl)methyl)piperidin-3-yl)-4-phenylpyrrolidin-2-one

To a solution of 4-bromo-N—((S)-1-(((S)-2,3-dihydrobenzo[b][1,4]dioxin-2-yl)methyl)piperidin-3-yl)-3-phenylbutanamide (48 mg, 0.10 mmol) in THF (2 ml) was added KOtBu (17 mg, 0.15 mmol) and the resulting mixture was stirred for 3 d at RT. Another portion of KOtBu (17 mg, 0.15 mmol) was added and the stirring was continued for 4 h. The reaction mixture was quenched with MeOH and evaporated to dryness. The residue was dissolved in EtOAc, washed with water and brine, dried and evaporated. The residue was purified by flash chromatography yielding 31 mg 1-((S)-1-(((S)-2,3-dihydrobenzo[b][1,4]dioxin-2-yl)methyl)piperidin-3-yl)-4-phenylpyrrolidin-2-one.

¹H NMR (400 MHz, CDCl₃) δ ppm 1.15-1.86 (5H, m), 2.07-2.25 (2H, m), 2.51-2.70 (3H, m), 2.75-2.88 (1H, m), 2.90-3.00 (1H, m), 3.30-3.45 (1H, m), 3.46-3.59 (1H, m), 3.73-3.88 (1H, m), 3.95-4.06 (1H, m), 4.16-4.34 (3H, m), 6.78-6.89 (4H, m), 7.19-7.38 (5H, m).

### EXAMPLE 33: 1-((S)-1-(((S)-2,3-Dihydrobenzo[b][1,4]dioxin-2-yl)methyl)piperidin-3-yl)pyrrolidine-2,5-dione

A solution of (S)-1-(((S)-2,3-dihydrobenzo[b][1,4]dioxin-2-yl)methyl)piperidin-3-amine (0.30 g, 1.21 mmol) and succinic anhydride (0.13 g, 1.33 mmol) in xylenes (10 ml) was microwave heated at 150° C. for 3 h and evaporated to dryness. The residue was dissolved in Ac₂O (5 ml) and NaOAc (0.13 g, 1.57 mmol) was added. The resulting mixture was refluxed for 4 h, poured to water, basified with NaOH solution end extracted with EtOAc. The combined organic phases were washed with water and brine, dried and evaporated. The residue was triturated with 1:1 Et₂O-heptane yielding 0.21 g 1-((S)-1-(((S)-2,3-dihydrobenzo[b][1,4]dioxin-2-yl)methyl)piperidin-3-yl)pyrrolidine-2,5-dione.

¹H NMR (400 MHz, CDCl₃) δ ppm 1.57-1.81 (3H, m), 2.09-2.27 (2H, m), 2.59-2.89 (9H, m), 3.94-4.04 (1H, m), 4.17-4.34 (3H, m), 6.78-6.90 (4H, m).

### EXAMPLE 34: 2-((S)-1-(((S)-2,3-Dihydrobenzo[b][1,4]dioxin-2-yl)methyl)piperidin-3-yl)-3a,4,7,7a-tetrahydro-1H-4,7-methanoisoindole-1,3(2H)-dione

A mixture of (S)-1-(((S)-2,3-dihydrobenzo[b][1,4]dioxin-2-yl)methyl)piperidin-3-amine (0.17 g, 0.70 mmol) and 5-norbomene-endo-2,3-dicarboxylic anhydride (0.11 g, 0.70 mmol) in toluene (2 ml) was heated for 3 h at 120° C. in a sealed tube. The solvent was evaporated off and the residue was purified by flash chromatography yielding 0.16 g 2-((S)-1-(((S)-2,3-dihydrobenzo[b][1,4]dioxin-2-yl)methyl)piperidin-3-yl)-3a,4,7,7a-tetrahydro-1H-4,7-methanoisoindole-1,3(2H)-dione.

¹H NMR (400 MHz, CDCl₃) δ ppm 1.45-1.76 (5H, m), 1.98-2.21 (2H, m), 2.53-2.85 (5H, m), 3.13-3.24 (2H, m), 3.33-3.43 (2H, m), 3.92-4.11 (2H, m), 4.19-4.33 (2H, m), 6.08-6.13 (2H, m), 6.79-6.89 (4H, m).

### EXAMPLE 35: 1-((S)-1-(((S)-2,3-Dihydrobenzo[b][1,4]dioxin-2-yl)methyl)piperidin-3-yl)-3-methyl-3-phenylpyrrolidine-2,5-dione

A mixture of (S)-1-(((S)-2,3-dihydrobenzo[b][1,4]dioxin-2-yl)methyl)piperidin-3-amine (0.20 g, 0.81 mmol) and 3-methyl-3-phenyldihydrofuran-2,5-dione (0.15 g, 0.81 mmol) in xylenes (5 ml) was refluxed for 2 h and evaporated to dryness. The residue was filtered through a silica plug (DCM-MeOH) yielding 0.13 g 1-((S)-1-(((S)-2,3-dihydrobenzo-[b][1,4]dioxin-2-yl)methyl)piperidin-3-yl)-3-methyl-3-phenylpyrrolidine-2,5-dione.

¹H NMR (400 MHz, CDCl₃) δ ppm 1.57-1.84 (3H, m), 1.70 (3H, s), 2.12-2.27 (2H, m), 2.60-2.76 (2H, m), 2.77-2.93 (4H, m), 3.01-3.11 (1H, m), 3.95-4.05 (1H, m), 4.21-4.35 (3H, m), 6.79-6.90 (4H, m), 7.27-7.41 (5H, m).

### EXAMPLE 36: (R)-1-((S)-1-(((S)-2,3-Dihydrobenzo[b][1,4]dioxin-2-yl)methyl)piperidin-3-yl)-3-methyl-3-phenylpyrrolidine-2,5-dione and (S)-1-((S)-1-(((S)-2,3-dihydrobenzo[b][1,4]dioxin-2-yl)methyl)piperidin-3-yl)-3-methyl-3-phenylpyrrolidine-2,5-dione

The diastereomers of 1-((S)-1-(((S)-2,3-dihydrobenzo[b][1,4]dioxin-2-yl)methyl)piperidin-3-yl)-3-methyl-3-phenylpyrrolidine-2,5-dione were separated using preparative HPLC (Chiralpak IF, 95:5 MTBE+0.2% DEA:IPA+0.2% DEA, 20 ml/min, run time 10 min) yielding 35 mg diastereomer 1 (retention time 4.6 min),

¹H NMR (400 MHz, CDCl₃) δ ppm 1.58-1.80 (3H, m), 1.69 (3H, s), 2.11-2.27 (2H, m), 2.58-2.76 (2H, m), 2.76-2.93 (4H, m), 3.01-3.11 (1H, m), 3.94-4.04 (1H, m), 4.21-4.35 (3H, m), 6.78-6.89 (4H, m), 7.27-7.40 (5H, m);

and 42 mg diastereomer 2 (retention time 6.2 min),

¹H NMR (400 MHz, CDCl₃) δ ppm 1.60-1.80 (3H, m), 1.70 (3H, s), 2.12-2.27 (2H, m), 2.59-2.76 (2H, m), 2.76-2.92 (4H, m), 3.01-3.11 (1H, m), 3.95-4.04 (1H, m), 4.21-4.35 (3H, m), 6.79-6.90 (4H, m), 7.27-7.40 (5H, m).

### EXAMPLE 37: 3-((S)-1-(((S)-2,3-Dihydrobenzo[b][1,4]dioxin-2-yl)methyl)piperidin-3-yl)-1-phenyl-3-azabicyclo[3.1.0]hexane-2,4-dione

A mixture of (S)-1-(((S)-2,3-dihydrobenzo[b][1,4]dioxin-2-yl)methyl)piperidin-3-amine (0.33 g, 1.33 mmol) and 1-phenyl-3-oxabicyclo[3.1.0]hexane-2,4-dione (0.25 g, 1.33 mmol, Bulletin de la Societe Chimique de France (1964), (10), 2462-71) in toluene (8 ml) was refluxed for 3 h and evaporated to dryness. The residue was purified by flash chromatography yielding 0.20 g 3-((S)-1-(((S)-2,3-dihydrobenzo[b][1,4]dioxin-2-yl)methyl)piperidin-3-yl)-1-phenyl-3-azabicyclo[3.1.0]hexane-2,4-dione.

¹H NMR (400 MHz, CDCl₃) δ ppm 1.57-1.70 (2H, m), 1.70-1.79 (2H, m), 1.79-1.86 (1H, m), 2.02-2.24 (6H, m), 3.94-4.03 (1H, m), 4.03-4.15 (1H, m), 4.21-4.33 (2H, m), 6.78-6.90 (4H, m), 7.31-7.43 (5H, m).

### EXAMPLE 38: 1-((S)-1-(((S)-2,3-Dihydrobenzo[b][1,4]dioxin-2-yl)methyl)piperidin-3-yl)-4-phenylpyrrolidine-2,3-dione

### Step 1: Methyl 3-((S)-1-(((S)-2,3-dihydrobenzo[b][1,4]dioxin-2-yl)methyl)piperidin-3-ylamino)-2-phenylpropanoate

A mixture of (S)-1-(((S)-2,3-dihydrobenzo[b][1,4]dioxin-2-yl)methyl)piperidin-3-amine (0.42 g, 1.70 mmol) and methyl 2-phenylacrylate (0.28 g, 1.70 mmol) in THF (5 ml) was stirred at 40° C. for 5 h and at RT 3 d. The solvent was evaporated off and the residue was purified by flash chromatography yielding 0.21 g methyl 3-((S)-1-(((S)-2,3-dihydrobenzo[b][1,4]dioxin-2-yl)methyl)piperidin-3-ylamino)-2-phenylpropanoate.

LC-MS, m/z=411.5 (M+1)⁺.

### Step 2: 1-((S)-1-(((S)-2,3-Dihydrobenzo[b][1,4]dioxin-2-yl)methyl)piperidin-3-yl)-4-phenylpyrrolidine-2,3-dione

A solution of methyl 3-((S)-1-(((S)-2,3-dihydrobenzo[b][1,4]dioxin-2-yl)methyl)piperidin-3-ylamino)-2-phenylpropanoate (0.20 g, 0.48 mmol) in DCM (3 ml) was treated with TEA (99 μl, 72 mg, 0.71 mmol) and methyl 2-chloro-2-oxoacetate (48 μl, 64 mg, 0.52 mmol) and stirred overnight at RT. The reaction mixture was diluted with DCM, washed with water and brine, dried and evaporated. The residue was dissolved in THF (3 ml) and treated with 60% NaH dispersion (29 mg, 0.71 mmol) for 2 h at RT. NH₄Cl solution and EtOAc were added and the aqueous phase was extracted with EtOAc. The combined organic phases were washed with water and brine, dried and evaporated. The residue was purified by flash chromatography yielding 40 mg 1-((S)-1-(((S)-2,3-dihydrobenzo[b][1,4]dioxin-2-yl)methyl)piperidin-3-yl)-4-phenylpyrrolidine-2,3-dione.

¹H NMR (400 MHz, CDCl₃) δ ppm 1.54-1.95 (5H, m), 2.21-2.31 (1H, m), 2.31-2.40 (1H, m), 2.62-2.75 (2H, m), 2.79-2.88 (1H, m), 2.99-3.07 (1H, m), 3.99-4.07 (1H, m), 4.19-4.35 (5H, m), 6.78-6.90 (4H, m), 7.24-7.30 (1H, m), 7.35-7.42 (2H, m), 7.64-7.71 (2H, m).

### EXAMPLE 39: 1-((S)-1-(((S)-2,3-Dihydrobenzo[b][1,4]dioxin-2-yl)methyl)piperidin-3-yl)-4-phenyl-1H-pyrrol-2(5H)-one

A mixture of (S)-1-(((S)-2,3-dihydrobenzo[b][1,4]dioxin-2-yl)methyl)piperidin-3-amine (0.21 g, 0.84 mmol), (Z)-methyl 4-bromo-3-phenylbut-2-enoate (0.22 g, 0.84 mmol, Journal of Organic Chemistry, 75(23), 8319-8321; 2010) and TEA (0.18 ml, 0.13 g, 1.26 mmol) in toluene (5 ml) was refluxed for 3 h. The precipitate was filtered off and the filtrate evaporated to dryness. The residue was purified by flash chromatography yielding 0.11 g 1-((S)-1-(((S)-2,3-dihydrobenzo[b][1,4]dioxin-2-yl)methyl)piperidin-3-yl)-4-phenyl-1H-pyrrol-2(5H)-one.

¹H NMR (400 MHz, CDCl₃) δ ppm 1.50-1.64 (1H, m), 1.71-1.84 (2H, m), 1.84-1.95 (1H, m), 2.22-2.37 (2H, m), 2.62-2.74 (2H, m), 2.77-2.86 (1H, m), 2.97-3.06 (1H, m), 4.00-4.08 (1H, m), 4.24-4.36 (3H, m), 4.38-4.45 (2H, m), 6.44 (1H, s), 6.77-6.91 (4H, m), 7.37-7.45 (3H, m), 7.46-7.53 (2H, m).

### EXAMPLE 40: 1-((S)-1-(((S)-2,3-Dihydrobenzo[b][1,4]dioxin-2-yl)methyl)piperidin-3-yl)-4-methyl-1H-pyrrol-2(5H)-one

A mixture of (S)-1-(((S)-2,3-dihydrobenzo[b][1,4]dioxin-2-yl)methyl)piperidin-3-amine (0.28 g, 1.13 mmol), (Z)-methyl 4-bromo-3-methylbut-2-enoate (0.26 g, 1.35 mmol, Journal of the American Chemical Society, 135(25), 9358-9361; 2013) and TEA (0.24 ml, 0.17 g, 1.69 mmol) in toluene (5 ml) was refluxed for 3 h. The precipitate was filtered off and the filtrate evaporated to dryness. The residue was purified by flash chromatography yielding 0.11 g 1-((S)-1-(((S)-2,3-dihydrobenzo[b][1,4]dioxin-2-yl)methyl)piperidin-3-yl)-4-methyl-1H-pyrrol-2(5H)-one.

¹H NMR (400 MHz, CDCl₃) δ ppm 1.38-1.52 (1H, m), 1.62-1.88 (3H, m), 2.03-2.07 (3H, m), 2.13-2.25 (2H, m), 2.59-2.71 (2H, m), 2.75-2.84 (1H, m), 2.91-2.99 (1H, m), 3.82-3.92 (2H, m), 3.97-4.06 (1H, m), 4.16-4.33 (3H, m), 5.81-5.87 (1H, m), 6.78-6.91 (4H, m).

### EXAMPLE 41: 1-((S)-1-(((S)-2,3-Dihydrobenzo[b][1,4]dioxin-2-yl)methyl)piperidin-3-yl)-4-(4-fluorophenyl)-1H-pyrrol-2(5H)-one

A mixture of (S)-1-(((S)-2,3-dihydrobenzo[b][1,4]dioxin-2-yl)methyl)piperidin-3-amine (0.32 g, 1.29 mmol), (Z)-methyl 4-bromo-3-(4-fluorophenyl)but-2-enoate (0.35 g, 1.29 mmol, Journal of Organic Chemistry, 75(23), 8319-8321; 2010) and TEA (0.27 ml, 0.20 g, 1.93 mmol) in toluene (10 ml) was refluxed for 2 h. The precipitate was filtered off and the filtrate evaporated to dryness. The residue was purified by flash chromatography yielding 93 mg 1-((S)-1-(((S)-2,3-dihydrobenzo[b][1,4]dioxin-2-yl)methyl)piperidin-3-yl)-4-(4-fluorophenyl)-1H-pyrrol-2(5H)-one.

¹H NMR (400 MHz, CDCl₃) δ ppm 1.50-1.64 (1H, m), 1.66-1.93 (3H, m), 2.24-2.38 (2H, m), 2.59-2.75 (2H, m), 2.76-2.85 (1H, m), 2.96-3.05 (1H, m), 4.00-4.07 (1H, m), 4.22-4.35 (3H, m), 4.36-4.42 (2H, m), 6.35-6.40 (1H, m), 6.78-6.88 (4H, m), 7.07-7.14 (2H, m), 7.44-7.51 (2H, m).

### EXAMPLE 42: 1-((S)-1-(((S)-2,3-Dihydrobenzo[b][1,4]dioxin-2-yl)methyl)piperidin-3-yl)-4-methyl-3-phenyl-1H-pyrrol-2(5H)-one

A mixture of (S)-1-(((S)-2,3-dihydrobenzo[b][1,4]dioxin-2-yl)methyl)piperidin-3-amine (0.23 g, 0.93 mmol), (Z)-methyl 4-bromo-3-methyl-2-phenylbut-2-enoate (0.25 g, 0.93 mmol, U.S. Pat. No. 3,622,569) and TEA (0.20 ml, 0.14 g, 1.40 mmol) in toluene (5 ml) was refluxed until reaction was completed. The precipitate was filtered off and the filtrate evaporated to dryness. The residue was purified by preparative RP-HPLC yielding 22 mg 1-((S)-1-(((S)-2,3-dihydrobenzo[b][1,4]dioxin-2-yl)methyl)piperidin-3-yl)-4-methyl-3-phenyl-1H-pyrrol-2(5H)-one.

¹H NMR (400 MHz, CDCl₃) δ ppm 1.49-1.65 (1H, m), 1.70-1.83 (2H, m), 1.84-1.95 (1H, m), 2.15 (3H, s), 2.20-2.40 (2H, m), 2.63-2.77 (2H, m), 2.80-2.91 (1H, m), 3.00-3.15 (1H, m), 3.96 (2H, d), 3.99-4.07 (1H, m), 4.24-4.40 (3H, m), 6.78-6.90 (4H, m), 7.28-7.35 (1H, m), 7.37-7.44 (2H, m), 7.44-7.50 (2H, m).

### EXAMPLE 43: 1-((S)-1-(((S)-2,3-Dihydrobenzo[b][1,4]dioxin-2-yl)methyl)piperidin-3-yl)-4-(2-methoxyphenyl)-1H-pyrrol-2(5H)-one formate

A mixture of (S)-1-(((S)-2,3-dihydrobenzo[b][1,4]dioxin-2-yl)methyl)piperidin-3-amine (0.13 g, 0.52 mmol), (Z)-methyl 4-bromo-3-(2-methoxyphenyl)but-2-enoate (0.15 g, 0.52 mmol, Journal of Organic Chemistry, 75(23), 8319-8321; 2010) and TEA (0.11 ml, 79 mg, 0.78 mmol) in toluene (4 ml) was refluxed for 3.5 h. The precipitate was filtered off and the filtrate evaporated to dryness. The residue was purified by preparative RP-HPLC yielding 33 mg 1-((S)-1-(((S)-2,3-dihydrobenzo[b][1,4]dioxin-2-yl)methyl)piperidin-3-yl)-4-(2-methoxyphenyl)-1H-pyrrol-2(5H)-one formate.

¹H NMR (400 MHz, CDCl₃) δ ppm 1.78-1.99 (4H, m), 2.48-2.60 (1H, m), 2.79-2.95 (2H, m), 2.97-3.06 (1H, m), 3.08-3.18 (1H, m), 3.33-3.42 (1H, m), 3.92 (3H, s), 4.00-4.10 (1H, m), 4.19-4.33 (2H, m), 4.42-4.58 (3H, m), 6.63-6.69 (1H, m), 6.80-6.91 (4H, m), 6.95-7.03 (2H, m), 7.34-7.44 (2H, m), 8.18 (2H, br s).

### EXAMPLE 44: 1-((S)-1-(((S)-2,3-Dihydrobenzo[b][1,4]dioxin-2-yl)methyl)piperidin-3-yl)imidazolidin-2-one

To a solution of(S)-1-(((S)-2,3-dihydrobenzo[b][1,4]dioxin-2-yl)methyl)piperidin-3-amine (0.43 g, 1.73 mmol) in THF (15 ml) was added 2-chloroethyl isocyanate (0.15 ml, 0.18 g, 1.73 mmol). After 1 h at RT, 1M LiHMDS in THF (2.08 ml, 2.08 mmol) was added. After stirring overnight, 0.87 ml 1M LiHMDS in THF was added and the stirring was continued for 2 h. The reaction was quenched with MeOH (5 ml) and concentrated. EtOAc was added the solution was washed with water and brine, dried and evaporated. The residue was purified by flash chromatography yielding 0.20 g 1-((S)-1-(((S)-2,3-dihydrobenzo[b][1,4]-dioxin-2-yl)methyl)piperidin-3-yl)imidazolidin-2-one.

¹H NMR (400 MHz, CDCl₃) δ ppm 1.33-1.46 (1H, m), 1.63-1.82 (3H, m), 2.07-2.19 (2H, m), 2.59-2.69 (2H, m), 2.75-2.84 (1H, m), 2.91-3.00 (1H, m), 3.35-3.53 (4H, m), 3.84-3.95 (1H, m), 3.97-4.05 (1H, m), 4.24-4.32 (2H, m), 4.38 (1H, br s), 6.78-6.89 (4H, m).

### EXAMPLE 45: 1-((S)-1-(((S)-2,3-Dihydrobenzo[b][1,4]dioxin-2-yl)methyl)piperidin-3-yl)-4,4-dimethylimidazolidin-2-one

### Step 1: (S)-Benzyl 3-((2-((tert-butoxycarbonyl)amino)-2-methylpropyl)amino)-piperidine-1-carboxylate

To a solution of (S)-benzyl 3-aminopiperidine-1-carboxylate (4.00 g, 17.1 mmol) and tert-butyl (2-methyl-1-oxopropan-2-yl)carbamate (3.19 g, 17.1 mmol) in DCE (60 ml) was added NaBH(OAc)₃ (7.20 g, 34.1 mmol) at 0° C. The resulting mixture was stirred at RT for 16 h. The reaction was quenched with water and extracted with DCM. The combined organic layers were dried and evaporated to dryness. The residue was purified by flash chromatography yielding 4.10 g (S)-benzyl 3-((2-((tert-butoxycarbonyl)amino)-2-methylpropyl)amino)piperidine-1-carboxylate.

LC-MS, m/z=406.5 (M+1)⁺.

### Step 2: (S)-Benzyl 3-((2-amino-2-methylpropyl)amino)piperidine-1-carboxylate dihydrochloride

To a solution of (S)-benzyl 3-((2-((tert-butoxycarbonyl)amino)-2-methylpropyl)amino)-piperidine-1-carboxylate (4.00 g, 9.87 mmol) in MeOH (15 ml) was added HCl in MeOH (25 ml) at 0° C. and the resulting mixture was stirred at RT for 4 h. The solvent was evaporated off and the residue was triturated with pentane and Et₂O yielding 2.60 g (S)-benzyl 3-((2-amino-2-methylpropyl)amino)piperidine-1-carboxylate dihydrochloride.

LC-MS, m/z=306.2 (M+1)⁺.

### Step 3: (S)-Benzyl 3-(4,4-dimethyl-2-oxoimidazolidin-1-yl)piperidine-1-carboxylate

To a solution of (S)-benzyl 3-((2-amino-2-methylpropyl)amino)piperidine-1-carboxylate dihydrochloride (1.50 g, 4.39 mmol) in DMF (36 ml) was added TEA (1.30 ml, 0.94 g, 9.67 mmol) at 0° C., followed by CDI (1.07 g, 6.59 mmol) and the resulting mixture was stirred at RT for 16 h. The solvent was evaporated off and the residue was dissolved in EtOAc, washed with water, dried and evaporated to dryness. The residue was purified by flash chromatography yielding 0.50 g (S)-benzyl 3-(4,4-dimethyl-2-oxoimidazolidin-1-yl)piperidine-1-carboxylate.

LC-MS, m/z=332.4 (M+1)⁺.

### Step 4: (S)-4,4-Dimethyl-1-(piperidin-3-yl)imidazolidin-2-one

To solution of (S)-benzyl 3-(4,4-dimethyl-2-oxoimidazolidin-1-yl)piperidine-1-carboxylate (0.50 g, 1.51 mmol) in EtOAc (30 ml) was added 10% Pd/C (0.20 g) at RT and the reaction mass was hydrogenated under hydrogen gas balloon pressure at same temperature for 4 h. The reaction mixture was filtered and the filtrate was evaporated to dryness. The residue was triturated with pentane yielding 0.24 g (S)-4,4-dimethyl-1-(piperidin-3-yl)imidazolidin-2-one.

LC-MS, m/z=198.3 (M+1)⁺.

### Step 5: 1-((S)-1-(((S)-2,3-Dihydrobenzo[b][1,4]dioxin-2-yl)methyl)piperidin-3-yl)-4,4-dimethylimidazolidin-2-one

Prepared using General procedure A from (R)-2-(bromomethyl)-2,3-dihydrobenzo[b][1,4]dioxine (0.13 g, 0.56 mmol), (S)-4,4-dimethyl-1-(piperidin-3-yl)imidazolidin-2-one (0.10 g, 0.51 mmol) and K₂CO₃ (0.11 g, 0.76 mmol) in ACN (2 ml) yielding 0.11 g 1-((S)-1-(((S)-2,3-dihydrobenzo[b][1,4]dioxin-2-yl)methyl)piperidin-3-yl)-4,4-dimethylimidazolidin-2-one.

¹H NMR (400 MHz, CDCl₃) δ ppm 1.28 (6H, d), 1.31-1.43 (1H, m), 1.63-1.82 (3H, m), 2.06-2.16 (2H, m), 2.59-2.68 (2H, m), 2.74-2.83 (1H, m), 2.90-2.99 (1H, m), 3.11-3.24 (2H, m), 3.85-3.96 (1H, m), 3.97-4.05 (1H, m), 4.19 (1H, br s), 4.24-4.33 (2H, m), 6.78-6.89 (4H, m).

### EXAMPLE 46: (R)-1-((S)-1-(((S)-2,3-Dihydrobenzo[b][1,4]dioxin-2-yl)methyl)-piperidin-3-yl)-4-methylimidazolidin-2-one

### Step 1: (S)-Benzyl 3-(((R)-2-((tert-butoxycarbonyl)amino)propyl)amino)piperidine-1-carboxylate

To a solution of (S)-benzyl 3-aminopiperidine-1-carboxylate (6.76 g, 28.9 mmol) and (R)-tert-butyl (1-oxopropan-2-yl)carbamate (5.00 g, 28.9 mmol) in DCE (100 ml) was added NaBH(OAc)₃ (9.19 g, 43.4 mmol) at 0° C. The resulting mixture was stirred at RT for 36 h. The reaction was quenched with water and extracted with DCE. The combined organic layers were dried and evaporated to dryness. The residue was purified by flash chromatography yielding 2.60 g (S)-benzyl 3-(((R)-2-((tert-butoxycarbonyl)amino)propyl)amino)piperidine-1-carboxylate.

LC-MS, m/z=392.3 (M+1)⁺.

### Step 2: (S)-Benzyl 3-(((R)-2-aminopropyl)amino)piperidine-1-carboxylate dihydrochloride

To a solution of (S)-benzyl 3-(((R)-2-((tert-butoxycarbonyl)amino)propyl)amino)piperidine-1-carboxylate (2.10 g, 5.36 mmol) in Et₂O (20 mil) was added 1M HCl in Et₂O (20 ml, 20 mmol) at 0° C. and the resulting mixture was stirred at RT for 18 h. The solvent was evaporated off and the residue was triturated with pentane and Et₂O yielding 1.90 g (S)-benzyl 3-(((R)-2-aminopropyl)amino)piperidine-1-carboxylate dihydrochloride.

LC-MS, m/z=292.3 (M+1)⁺.

### Step 3: (S)-Benzyl 3-((R)-4-methyl-2-oxoimidazolidin-1-yl)piperidine-1-carboxylate

To a solution of (S)-benzyl 3-(((R)-2-aminopropyl)amino)piperidine-1-carboxylate dihydrochloride (2.00 g, 6.84 mmol) DMF (20 ml) were added TEA (2.10 ml, 1.52 g, 15.1 mmol) and CDI (1.11 g, 6.84 mmol) at 0° C. and the resulting mixture was stirred at RT for 18 h. The solvent was evaporated off and the residue was dissolved in EtOAc, washed with water, dried and evaporated to dryness. The residue was purified by flash chromatography yielding 0.80 g (S)-benzyl 3-((R)-4-methyl-2-oxoimidazolidin-1-yl)piperidine-1-carboxylate.

LC-MS, m/z=318.1 (M+1)⁺.

### Step 4: (R)-4-Methyl-1-((S)-piperidin-3-yl)imidazolidin-2-one

To solution of (S)-benzyl 3-((R)-4-methyl-2-oxoimidazolidin-1-yl)piperidine-1-carboxylate (0.80 g, 2.52 mmol) in EtOAc (20 ml) was added 10% Pd/C (0.25 g) at RT and the reaction mass was hydrogenated under hydrogen gas balloon pressure at same temperature for 8 h. The mixture was filtered and the filtrate was evaporated to dryness. The residue was triturated with pentane and Et₂O yielding 0.28 g (R)-4-methyl-1-((S)-piperidin-3-yl)imidazolidin-2-one.

LC-MS, m/z=184.1 (M+1)⁺.

### Step 5: (R)-1-((S)-1-(((S)-2,3-Dihydrobenzo[b][1,4]dioxin-2-yl)methyl)piperidin-3-yl)-4-methylimidazolidin-2-one

Prepared using General procedure A from (R)-2-(bromomethyl)-2,3-dihydrobenzo[b][1,4]-dioxine (0.14 g, 0.60 mmol), (R)-4-methyl-1-((S)-piperidin-3-yl)imidazolidin-2-one (0.10 g, 0.55 mmol) and K₂CO₃ (0.11 g, 0.81 mmol) in ACN (2 ml) yielding 80 mg (R)-1-((S)-1-(((S)-2,3-dihydrobenzo[b][1,4]dioxin-2-yl)methyl)piperidin-3-yl)-4-methylimidazolidin-2-one.

¹H NMR (400 MHz, CDCl₃) δ ppm 1.23 (3H, d), 1.31-1.43 (1H, m), 1.64-1.82 (3H, m), 2.06-2.16 (2H, m), 2.58-2.68 (2H, m), 2.75-2.84 (1H, m), 2.90-2.98 (1H, m), 2.98-3.06 (1H, m), 3.51 (1H, t), 3.70-3.81 (1H, m), 3.84-3.95 (1H, m), 3.97-4.04 (1H, m), 4.24-4.32 (2H, m), 4.37 (1H, br s), 6.79-6.90 (4H, m).

### EXAMPLE 47: 1-((S)-1-(((S)-2,3-Dihydrobenzo[b][1,4]dioxin-2-yl)methyl)piperidin-3-yl)-5-methylimidazolidin-2-one

### Step 1: (3S)-Benzyl 3-((1-((tert-butoxycarbonyl)amino)propan-2-yl)amino)piperidine-1-carboxylate

To a solution of (S)-benzyl 3-aminopiperidine-1-carboxylate (4.50 g, 19.2 mmol) and tert-butyl (2-oxopropyl)carbamate (3.99 g, 23.2 mmol) in DCE (80 ml) was added NaBH(OAc)₃ (8.10 g, 38.5 mmol) at 0° C. The resulting mixture was stirred at RT for 2 h. The reaction was quenched with water and extracted with EtOAc. The combined organic layers were dried and evaporated to dryness. The residue was purified by flash chromatography yielding 3.00 g (3S)-benzyl 3-((1-((tert-butoxycarbonyl)amino)propan-2-yl)amino)piperidine-1-carboxylate.

LC-MS, m/z=392.1 (M+1)⁺.

### Step 2: (3S)-Benzyl 3-((1-aminopropan-2-yl)amino)piperidine-1-carboxylate dihydrochloride

To a solution of (3S)-benzyl 3-((1-((tert-butoxycarbonyl)amino)propan-2-yl)amino)piperidine-1-carboxylate (0.10 g 0.26 mmol) in Et₂O (5 ml) was added 1M HCl in Et₂O (5 ml, 5 mmol) at 0° C. and the resulting mixture was stirred at RT for 8 h. The solvent was evaporated off and the residue was triturated with 1:5 Et₂O/pentane yielding 30 mg (3S)-benzyl 3-((1-aminopropan-2-yl)amino)piperidine-1-carboxylate dihydrochloride.

LC-MS, m/z=292.2 (M+1)⁺.

### Step 3: (3S)-Benzyl 3-(5-methyl-2-oxoimidazolidin-1-yl)piperidine-1-carboxylate

To an ice cold stirred solution of (3S)-benzyl 3-((1-aminopropan-2-yl)amino)piperidine-1-carboxylate dihydrochloride (1.00 g, 3.05 mmol) in DMF (10 ml) was added DIPEA (1.20 ml, 0.89 g, 6.71 mmol) and CDI (0.49 g, 3.05 mmol) and the resulting mixture was stirred at RT for 18 h. The reaction was quenched with water and extracted with EtOAc. The combined organic layers were washed with water and brine, dried and evaporated to dryness. The residue was purified by flash chromatography yielding 0.70 g (3S)-benzyl 3-(5-methyl-2-oxoimidazolidin-1-yl)piperidine-1-carboxylate.

LC-MS, m/z=318.0 (M+1)⁺.

### Step 4: 5-Methyl-1-((S)-piperidin-3-yl)imidazolidin-2-one

To solution of (3S)-benzyl 3-(5-methyl-2-oxoimidazolidin-1-yl)piperidine-1-carboxylate (1.20 g, 2.52 mmol) in EtOAc (20 ml) was added 10% Pd/C (0.50 g) and the reaction mixture was hydrogenated under hydrogen gas balloon pressure for 8 h. The mixture was filtered through a Celite pad and the solvent was evaporated off. The residue was triturated with pentane yielding 0.50 g 5-methyl-1-((S)-piperidin-3-yl)imidazolidin-2-one.

LC-MS, m/z=183.8 (M+1)⁺.

### Step 5: 1-((S)-1-(((S)-2,3-Dihydrobenzo[b][1,4]dioxin-2-yl)methyl)piperidin-3-yl)-5-methylimidazolidin-2-one

Prepared using General procedure A from (R)-2-(bromomethyl)-2,3-dihydrobenzo[b][1,4]dioxine (0.17 g, 0.76 mmol), 5-methyl-1-((S)-piperidin-3-yl)imidazolidin-2-one (0.13 g, 0.69 mmol) and K₂CO₃ (0.14 g, 1.03 mmol) in ACN (2 ml) yielding 0.11 g 1-((S)-1-(((S)-2,3-dihydrobenzo[b][1,4]dioxin-2-yl)methyl)piperidin-3-yl)-5-methylimidazolidin-2-one.

¹H NMR (400 MHz, CDCl₃) δ ppm 1.29 (3H, t), 1.54-1.89 (4H, m), 2.06-2.18 (1H, m), 2.51-2.75 (3H, m), 2.79-2.88 (1H, m), 2.89-3.03 (2H, m), 3.46-3.55 (2H, m), 3.75-3.87 (1H, m), 3.95-4.05 (1H, m), 4.24-4.36 (3H, m), 6.78-6.89 (4H, m).

### EXAMPLE 48: 1-((S)-1-(((S)-2,3-Dihydrobenzo[b][1,4]dioxin-2-yl)methyl)piperidin-3-yl)-3-phenylimidazolidin-2-one

A mixture of 1-((S)-1-(((S)-2,3-dihydrobenzo[b][1,4]dioxin-2-yl)methyl)piperidin-3-yl)imidazolidin-2-one (0.13 g, 0.40 mmol), iodobenzene (45 μl, 82 mg, 0.40 mmol), K₂CO₃ (0.11 g, 0.81 mmol), CuI (9.2 mg, 0.048 mmol) and N,N′-dimethylethylenediamine (8.6 μl, 7.1 mg, 0.081 mmol) in toluene (4 ml) was microwave heated at 140° C. for 7 h. 23 μl iodobenzene was added and the heating was continued for 4 h. The reaction mixture was filtered through a Celite pad and the filtrate was evaporated to dryness. The residue was purified by flash chromatography yielding 67 mg 1-((S)-1-(((S)-2,3-dihydrobenzo[b][1,4]-dioxin-2-yl)methyl)piperidin-3-yl)-3-phenylimidazolidin-2-one.

¹H NMR (400 MHz, DMSO-d₆) δ ppm 1.38-1.63 (1H, m), 1.64-1.79 (2H, m), 2.00-2.12 (1H, m), 2.13-2.26 (1H, m), 2.53-2.66 (3H, m), 2.74-2.85 (1H, m), 2.85-2.96 (1H, m), 3.39-3.59 (2H, m), 3.67-3.87 (3H, m), 3.90-4.03 (1H, m), 4.23-4.43 (2H, m), 6.74-6.92 (4H, m), 6.93-7.05 (1H, m), 7.24-7.38 (2H, m), 7.50-7.62 (2H, m).

### EXAMPLE 49: 1-((S)-1-(((S)-2,3-Dihydrobenzo[b][1,4]dioxin-2-yl)methyl)piperidin-3-yl)-3,4-dimethylimidazolidin-2-one

### Step 1: (3S)-Benzyl 3-(4-methyl-2-oxoimidazolidin-1-yl)piperidine-1-carboxylate

Prepared as described in example 46, steps 1-3 using racemic tert-butyl (1-oxopropan-2-yl)carbamate instead of (R)-tert-butyl (1-oxopropan-2-yl)carbamate. The crude product was used in the next step as such.

### Step 2: (3S)-Benzyl 3-(3,4-dimethyl-2-oxoimidazolidin-1-yl)piperidine-1-carboxylate

To an ice cold stirred solution of crude (3S)-benzyl 3-(4-methyl-2-oxoimidazolidin-1-yl)piperidine-1-carboxylate (2.20 g, 10.1 mmol) in DMF (40 ml) was added 60% NaH dispersion (2.43 g, 101.3 mmol) and the resulting mixture was stirred for 15 min. CH₃I (0.69 ml, 1.57 g, 11.2 mmol) was added to the above cold mixture and stirring was continued at RT for 2 h. The solvent was evaporated off and the residue was dissolved in EtOAc, washed with water, dried and evaporated to dryness. The residue was purified by flash chromatography yielding 2.00 g (3S)-benzyl 3-(3,4-dimethyl-2-oxoimidazolidin-1-yl)piperidine-1-carboxylate.

LC-MS, m/z=332.1 (M+1)⁺.

### Step 3: 3,4-Dimethyl-1-((S)-piperidin-3-yl)imidazolidin-2-one

To a solution of (3S)-benzyl 3-(3,4-dimethyl-2-oxoimidazolidin-1-yl)piperidine-1-carboxylate (2.00 g, 6.04 mmol) in EtOAc (20 ml) was added 10% Pd/C (0.50 g) and the reaction mixture was hydrogenated under hydrogen gas balloon pressure for 3 h. The mixture was filtered through a Celite pad and evaporated to dryness. The residue was triturated with 1:5 Et₂O/pentane yielding 0.70 g 3,4-dimethyl-1-((S)-piperidin-3-yl)imidazolidin-2-one.

LC-MS, m/z=198.1 (M+1)⁺.

### Step 4: 1-((S)-1-(((S)-2,3-Dihydrobenzo[b][1,4]dioxin-2-yl)methyl)piperidin-3-yl)-3,4-dimethylimidazolidin-2-one

Prepared using General procedure A from (R)-2-(bromomethyl)-2,3-dihydrobenzo[b][1,4]dioxine (0.26 g, 1.15 mmol), 3,4-dimethyl-1-((S)-piperidin-3-yl)imidazolidin-2-one (0.23 g, 1.15 mmol) and DIPEA (0.40 ml, 0.30 g, 2.30 mmol) in ACN (4 ml) yielding 0.29 g 1-((S)-1-(((S)-2,3-dihydrobenzo[b][1,4]dioxin-2-yl)methyl)piperidin-3-yl)-3,4-dimethylimidazolidin-2-one.

¹H NMR (400 MHz, CDCl₃) δ ppm 1.18-1.27 (3H, m), 1.31-1.51 (1H, m), 1.55-1.81 (3H, m), 2.05-2.19 (2H, m), 2.60-2.68 (2H, m), 2.72 (3H, s), 2.76-3.00 (3H, m), 3.35-3.52 (2H, m), 3.83-3.96 (1H, m), 3.97-4.05 (1H, m), 4.23-4.33 (2H, m), 6.78-6.90 (4H, m).

### EXAMPLE 50: 1-Benzyl-3-((S)-1-(((S)-2,3-dihydrobenzo[b][1,4]dioxin-2-yl)methyl)piperidin-3-yl)imidazolidin-2-one

A mixture of 1-((S)-1-(((S)-2,3-dihydrobenzo[b][1,4]dioxin-2-yl)methyl)piperidin-3-yl)imidazolidin-2-one (0.26 g, 0.82 mmol), benzyl chloride (0.10 ml, 0.11 g, 0.90 mmol) and KOtBu (0.10 g, 0.90 mmol) in DMF (2 ml) was stirred at 50° C. for 4 h. The reaction mixture was diluted with water and extracted with DCM. The combined organic phases were washed with water and brine, dried and evaporated to dryness. The residue was purified by flash chromatography yielding 0.10 g 1-benzyl-3-((S)-1-(((S)-2,3-dihydrobenzo[b][1,4]dioxin-2-yl)methyl)piperidin-3-yl)imidazolidin-2-one.

¹H NMR (400 MHz, CDCl₃) δ ppm 1.33-1.47 (1H, m), 1.63-1.85 (3H, m), 2.06-2.21 (2H, m), 2.58-2.71 (2H, m), 2.76-2.85 (1H, m), 2.93-3.02 (1H, m), 3.10-3.19 (2H, m), 3.24-3.39 (2H, m), 3.89-4.06 (2H, m), 4.25-4.33 (2H, m), 4.36 (2H, s), 6.78-6.90 (4H, m), 7.23-7.37 (5H, m).

### EXAMPLE 51: 1-((S)-1-(((S)-2,3-Dihydrobenzo[b][1,4]dioxin-2-yl)methyl)piperidin-3-yl)-3,4,4-trimethylimidazolidin-2-one

### Step 1: (S)-Benzyl 3-(3,4,4-trimethyl-2-oxoimidazolidin-1-yl)piperidine-1-carboxylate

To an ice cold stirred solution of (S)-benzyl 3-(4,4-dimethyl-2-oxoimidazolidin-1-yl)piperidine-1-carboxylate (1.70 g, 5.13 mmol) in DMF (40 ml) was added 60% NaH dispersion (1.23 g, 51.4 mmol) and the resulting mixture was stirred for 15 min. CH₃I (0.32 ml, 0.73 g, 5.13 mmol) was added and the mixture was stirred at RT for 1 h. The reaction was quenched with water and evaporated to dryness. The residue was dissolved in EtOAc, washed with water, dried and evaporated. The residue was purified by flash chromatography yielding 1.50 g (S)-benzyl 3-(3,4,4-trimethyl-2-oxoimidazolidin-1-yl)piperidine-1-carboxylate.

LC-MS, m/z=346.4 (M+1)⁺.

### Step 2: (S)-3,4,4-Trimethyl-1-(piperidin-3-yl)imidazolidin-2-one

To a solution of (S)-benzyl 3-(3,4,4-trimethyl-2-oxoimidazolidin-1-yl)piperidine-1-carboxylate (1.50 g, 4.34 mmol) in EtOAc (40 ml) was added 10% Pd/C (0.70 g) at RT and the reaction mixture was hydrogenated under balloon pressure for 4 h. The mixture was filtered through a Celite pad and evaporated to dryness. The residue was triturated with pentane and Et₂O yielding 0.50 g (S)-3,4,4-trimethyl-1-(piperidin-3-yl)imidazolidin-2-one.

LC-MS, m/z=212.1 (M+1)⁺.

### Step 3: 1-((S)-1-(((S)-2,3-Dihydrobenzo[b][1,4]dioxin-2-yl)methyl)piperidin-3-yl)-3,4,4-trimethylimidazolidin-2-one hydrochloride

Prepared using General procedure A from (R)-2-(bromomethyl)-2,3-dihydrobenzo[b][1,4]dioxine (0.13 g, 0.57 mmol), (S)-3,4,4-trimethyl-1-(piperidin-3-yl)imidazolidin-2-one (0.10 g, 0.47 mmol) and K₂CO₃ (0.13 g, 0.95 mmol) in ACN (2 ml) yielding, after conversion to HCl salt in DCM-dioxane, 67 mg 1-((S)-1-(((S)-2,3-dihydrobenzo[b][1,4]dioxin-2-yl)methyl)piperidin-3-yl)-3,4,4-trimethylimidazolidin-2-one hydrochloride.

¹H NMR (400 MHz, CDCl₃) δ ppm 1.18-1.29 (6H, m), 1.55-1.81 (1H, m), 1.89-2.21 (3H, m), 2.25-2.45 (1H, m), 2.65 (3H, s), 2.80-2.97 (1H, m), 3.01-3.24 (3H, m), 3.26-3.48 (2H, m), 3.64-3.92 (3H, m), 4.07-4.17 (1H, m), 4.24-4.32 (1H, m), 6.79-6.96 (4H, m), 12.63 (1H, br s).

### EXAMPLE 52: 1-((S)-1-(((S)-2,3-Dihydrobenzo[b][1,4]dioxin-2-yl)methyl)piperidin-3-yl)-3-(1-phenylethyl)imidazolidin-2-one hydrochloride

### Step 1: (S)-tert-Butyl-3-(2-(((benzyloxy)carbonyl)amino)acetamido)piperidine-1-carboxylate

To a solution of(S)-tert-butyl 3-aminopiperidine-1-carboxylate (10.0 g, 50.0 mmol) in DCM (500 ml) was added 2-(((benzyloxy)carbonyl)amino)acetic acid (11.5 g, 55.0 mmol), EDC.HCl (14.37 g, 75.0 mmol), HOBt (10.1 g, 75.0 mmol) and DMAP (12.2 g, 100.0 mmol) at 0° C. and the reaction mixture was stirred at RT for 16 h. The mixture was diluted with DCM, washed with water, dried and evaporated to dryness. The residue was purified by flash chromatography yielding 12.0 g (S)-tert-butyl 3-(2-(((benzyloxy)carbonyl)-amino)acetamido)piperidine-1-carboxylate.

LC-MS, m/z=392.1 (M+1)⁺.

### Step 2: (S)-tert-Butyl-3-((2-(((benzyloxy)carbonyl)amino)ethyl)amino)piperidine-1-carboxylate

To a solution of (S)-tert-butyl 3-(2-(((benzyloxy)carbonyl)amino)acetamido)piperidine-1-carboxylate (6.00 g, 15.3 mmol) in THF (200 ml) was added 1M BH₃.THF (23.0 ml, 23 mmol) at RT. The reaction mixture was refluxed for 3 h. After cooling to RT, the mixture was quenched with MeOH. The solvent was evaporated off and the residue was purified by flash chromatography yielding 1.50 g (S)-tert-butyl 3-((2-(((benzyloxy)carbonyl)amino)-ethyl)amino)piperidine-1-carboxylate.

LC-MS, m/z=378.3 (M+1)⁺.

### Step 3: (S)-tert-Butyl-3-((2-aminoethyl)amino)piperidine-1-carboxylate

A mixture of 10% Pd/C (0.50 g) and (S)-tert-butyl 3-((2-(((benzyloxy)carbonyl)amino)-ethyl)amino)piperidine-1-carboxylate (1.50 g, 3.97 mmol) in EtOAc (50 ml) was stirred at RT for 16 h under hydrogen balloon pressure. The mixture was filtered and the filtrate was evaporated to dryness yielding 0.60 g (S)-tert-butyl 3-((2-aminoethyl)amino)piperidine-1-carboxylate.

LC-MS, m/z=244.3 (M+1)⁺.

### Step 4: (S)-tert-Butyl-3-(2-oxoimidazolidin-1-yl)piperidine-1-carboxylate

To a stirred solution of (S)-tert-butyl 3-((2-aminoethyl)amino)piperidine-1-carboxylate (1.00 g, 4.11 mmol) in DMF (30 ml) was added TEA (1.50 ml, 1.09 g, 10.76 mmol) and CDI (1.66 g, 10.24 mmol) at RT and the reaction mixture was heated at 90° C. for 16 h in a sealed tube. The reaction mixture was diluted with water and extracted with EtOAc. The combined extracts were washed with water and brine, dried and evaporated. The residue was purified by flash chromatography yielding 0.42 g (S)-tert-butyl 3-(2-oxoimidazolidin-1-yl)piperidine-1-carboxylate.

LC-MS, m/z=270.3 (M+1)⁺.

### Step 5: (3S)-tert-Butyl-3-(2-oxo-3-(1-phenylethyl)imidazolidin-1-yl)piperidine-1-carboxylate

To a suspension of 60% NaH dispersion (0.15 g, 3.71 mmol) in THF (10 ml) was added (S)-tert-butyl 3-(2-oxoimidazolidin-1-yl)piperidine-1-carboxylate (0.50 g, 1.86 mmol) in THF (15 ml) at 0° C. and the resulting mixture was stirred for 20 min at 0° C. (1-Bromoethyl)-benzene (0.52 ml, 0.71 g, 3.81 mmol) and the reaction mixture was stirred at 50° C. for 4 h. The mixture was diluted with water and extracted with EtOAc, washed with water and brine, dried and evaporated. The residue was purified by flash chromatography yielding 0.40 g (3S)-tert-butyl 3-(2-oxo-3-(1-phenylethyl)imidazolidin-1-yl)piperidine-1-carboxylate.

LC-MS, m/z=374.3 (M+1)⁺.

### Step 6: 1-(1-Phenylethyl)-3-((S)-piperidin-3-yl)imidazolidin-2-one hydrochloride

(3S)-tert-Butyl 3-(2-oxo-3-(1-phenylethyl)imidazolidin-1-yl)piperidine-1-carboxylate (0.50 g, 1.34 mmol) was added to ice cold 4 M HCl in dioxane (20 ml, 80 mmol). The resulting mixture was stirred at RT for 3 h. The solvent was evaporated off and the residue was triturated with pentane yielding 0.44 g 1-(1-phenylethyl)-3-((S)-piperidin-3-yl)imidazolidin-2-one hydrochloride.

LC-MS, m/z=274.3 (M+1)⁺.

### Step 7: 1-((S)-1-(((S)-2,3-Dihydrobenzo[b][1,4]dioxin-2-yl)methyl)piperidin-3-yl)-3-(1-phenylethyl)imidazolidin-2-one hydrochloride

Prepared using general procedure A from (R)-2-(bromomethyl)-2,3-dihydrobenzo[b][1,4]-dioxine (0.12 g, 0.53 mmol), 1-(1-phenylethyl)-3-((S)-piperidin-3-yl)imidazolidin-2-one hydrochloride (0.15 g, 0.48 mmol) and K₂CO₃ (0.13 g, 0.97 mmol) in ACN (3 ml) yielding 86 mg 1-((S)-1-(((S)-2,3-dihydrobenzo[b][1,4]dioxin-2-yl)methyl)piperidin-3-yl)-3-(1-phenylethyl)imidazolidin-2-one hydrochloride after conversion of free base to HCl salt in Et₂O.

¹H NMR (400 MHz, DMSO-d₆) δ ppm 1.45 (3H, d), 1.54-2.02 (4H, m), 2.81-3.04 (2H, m), 3.07-3.71 (8H, m), 3.98-4.17 (2H, m), 4.26-4.38 (1H, m), 4.80-4.95 (1H, m), 4.98-5.10 (1H, m), 6.81-7.00 (4H, m), 7.22-7.42 (5H, m), 10.52 (1H, br s).

### EXAMPLE 53: 1-((S)-1-(((S)-2,3-Dihydrobenzo[b][1,4]dioxin-2-yl)methyl)piperidin-3-yl)tetrahydropyrimidin-2(1H)-one

### Step 1: (S)-Benzyl-3-((3-((tert-butoxycarbonyl)amino)propyl)amino)piperidine-1-carboxylate

To an ice cold stirred solution of 3-((tert-butoxycarbonyl)amino)propyl 4-methylbenzenesulfonate (15.0 g, 45.57 mmol) in dry DMF (150 ml) were added (S)-benzyl 3-aminopiperidine-1-carboxylate (10.67 g, 45.57 mmol) and K₂CO₃ (12.6 g, 91.18 mmol) and the mixture was stirred at 60° C. for 4 h. The reaction mixture was carefully quenched with ice water (100 ml) and extracted with EtOAc. The combined organic extracts were dried and concentrated under reduced pressure. The crude compound was purified by flash chromatography yielding 3.00 g (S)-benzyl 3-((3-((tert-butoxycarbonyl)amino)propyl)-amino)piperidine-1-carboxylate.

LC-MS, m/z=392.1 (M+1)⁺.

### Step 2: (S)-Benzyl-3-((3-aminopropyl)amino)piperidine-1-carboxylate dihydrochloride

To an ice cold stirred solution of (S)-benzyl 3-((3-((tert-butoxycarbonyl)amino)propyl)-amino)piperidine-1-carboxylate (3.00 g, 7.67 mmol) in MeOH (50 ml) was added 5 M HCl in MeOH (50 ml, 250 mmol) and the resulting mixture was stirred at RT for 1 h. The solvent was evaporated off and the residue was triturated with Et₂O yielding 2.40 g (S)-benzyl 3-((3-aminopropyl)amino)piperidine-1-carboxylate dihydrochloride.

LC-MS, m/z=292.1 (M+1)⁺.

### Step 3: (S)-Benzyl-3-(2-oxotetrahydropyrimidin-1(2H)-yl)piperidine-1-carboxylate

To an ice cold stirred solution of (S)-Benzyl 3-((3-aminopropyl)amino)piperidine-1-carboxylate dihydrochloride (2.40 g, 7.32 mmol) in DMF (60 ml) were added Et₃N (2.25 ml, 1.63 g, 16.1 mmol) and CDI (1.78 g, 10.98 mmol). Then the reaction mixture was stirred at RT for 16 h. The solvent was evaporated off and the residue was dissolved in EtOAc, washed with water, dried and evaporated. The residue was purified by flash chromatography yielding 1.00 g (S)-benzyl 3-(2-oxotetrahydropyrimidin-1 (2H)-yl)piperidine-1-carboxylate.

LC-MS, m/z=318.0 (M+1)⁺.

### Step 4: (S)-1-(Piperidin-3-yl)tetrahydropyrimidin-2(1H)-one

To solution of (S)-benzyl 3-(2-oxotetrahydropyrimidin-1 (2H)-yl)piperidine-1-carboxylate (0.70 g, 2.21 mmol) in EtOAc (20 ml) was added 10% Pd/C (0.40 g) at RT, and the reaction mixture was hydrogenated under hydrogen gas balloon pressure at same temperature for 4 h. The mixture was filtered and the filtrate was evaporated to dryness. The residue was triturated with pentane yielding 0.35 g (S)-1-(piperidin-3-yl)tetrahydropyrimidin-2(1H)-one.

LC-MS, m/z=184.2 (M+1)⁺.

### Step 5: 1-((S)-1-(((S)-2,3-Dihydrobenzo[b][1,4]dioxin-2-yl)methyl)piperidin-3-yl)tetrahydropyrimidin-2(1H)-one

Prepared using general procedure A from (R)-2-(bromomethyl)-2,3-dihydrobenzo[b][1,4]-dioxine (0.13 g, 0.55 mmol), (S)-1-(piperidin-3-yl)tetrahydropyrimidin-2(1H)-one (0.10 g, 0.55 mmol) and DIPEA (0.19 ml, 0.14 g, 1.09 mmol) in ACN (3 ml) yielding 63 mg 1-((S)-1-(((S)-2,3-dihydrobenzo[b][1,4]dioxin-2-yl)methyl)piperidin-3-yl)tetrahydropyrimidin-2(1H)-one.

¹H NMR (400 MHz, CDCl₃) δ ppm 1.36-1.51 (1H, m), 1.64-1.79 (3H, m), 1.84-1.94 (2H, m), 1.99-2.19 (2H, m), 2.58-2.70 (2H, m), 2.76-2.87 (1H, m), 2.89-2.99 (1H, m), 3.15-3.31 (4H, m), 3.97-4.06 (1H, m), 4.22-4.33 (2H, m), 4.36-4.48 (1H, m), 4.64 (1H, br s), 6.78-6.89 (4H, m).

### EXAMPLE 54: 1-((S)-1-(((S)-2,3-Dihydrobenzo[b][1,4]dioxin-2-yl)methyl)piperidin-3-yl)-3-methyltetrahydropyrimidin-2(1H)-one

### Step 1: (S)-Benzyl-3-(3-methyl-2-oxotetrahydropyrimidin-1(2H)-yl)piperidine-1-carboxylate

To an ice cold stirred solution of (S)-benzyl 3-(2-oxotetrahydropyrimidin-1(2H)-yl)piperidine-1-carboxylate (1.00 g, 3.15 mmol) in DMF (20 ml) was added 60% NaH dispersion (0.76 g, 31.5 mmol) and the resulting mixture was stirred for 15 min. CH₃I (0.20 ml, 0.46 g, 3.15 mmol) was added and the resulting mixture was stirred at RT for 1 h. The solvent was evaporated off and the residue was dissolved in EtOAc, washed with water, dried and evaporated. The residue was purified by flash chromatography yielding 0.80 g (S)-benzyl 3-(3-methyl-2-oxotetrahydropyrimidin-1 (2H)-yl)piperidine-1-carboxylate.

LC-MS, m/z=332.5 (M+1)⁺.

### Step 2: (S)-1-Methyl-3-(piperidin-3-yl)tetrahydropyrimidin-2(1H)-one

To a solution of (S)-benzyl 3-(3-methyl-2-oxotetrahydropyrimidin-1 (2H)-yl)piperidine-1-carboxylate (0.80 g, 2.42 mmol) in EtOAc (20 ml) was added 10% Pd/C (0.40 mg) at RT and the reaction mixture was hydrogenated under hydrogen gas balloon pressure at same temperature for 4 h. The mixture was filtered and the filtrate was evaporated to dryness yielding 0.45 g (S)-1-methyl-3-(piperidin-3-yl)tetrahydropyrimidin-2(1H)-one.

LC-MS, m/z=198.2 (M+1)⁺.

### Step 3: 1-((S)-1-(((S)-2,3-Dihydrobenzo[b][1,4]dioxin-2-yl)methyl)piperidin-3-yl)-3-methyltetrahydropyrimidin-2(1H)-one

Prepared using general procedure A from (R)-2-(bromomethyl)-2,3-dihydrobenzo[b][1,4]-dioxine (0.20 g, 0.85 mmol), (S)-1-methyl-3-(piperidin-3-yl)tetrahydropyrimidin-2(1H)-one (0.17 g, 0.85 mmol) and DIPEA (0.30 ml, 0.22 g, 1.70 mmol) in ACN (4 ml) yielding 0.13 g 1-((S)-1-(((S)-2,3-dihydrobenzo[b][1,4]dioxin-2-yl)methyl)piperidin-3-yl)-3-methyltetrahydropyrimidin-2(1H)-one.

¹H NMR (400 MHz, CDCl₃) δ ppm 1.34-1.49 (1H, m), 1.57-1.77 (3H, m), 1.86-1.95 (2H, m), 1.99-2.14 (2H, m), 2-59-2.67 (2H, m), 2.77-2.86 (1H, m), 2.87-2.96 (1H, m), 2.93 (3H, s), 3.13-3.29 (4H, m), 3.97-4.06 (1H, m), 4.23-4.34 (2H, m), 4.38-4.50 (1H, m), 6.78-6.89 (4H, m).

### EXAMPLE 55: 2-((S)-1-(((S)-2,3-Dihydrobenzo[b][1,4]dioxin-2-yl)methyl)piperidin-3-yl)-4-methoxyisoindolin-1-one

A mixture of (S)-1-(((S)-2,3-dihydrobenzo[b][1,4]dioxin-2-yl)methyl)piperidin-3-amine (0.20 g, 0.81 mmol), methyl 2-(bromomethyl)-3-methoxybenzoate (0.21 g, 0.81 mmol) and TEA (0.17 ml, 0.12 g, 1.21 mmol) in toluene (5 ml) was refluxed for 4 h. The precipitate was filtered off and the filtrate evaporated to dryness. The residue was purified by flash chromatography yielding 0.28 g 2-((S)-1-(((S)-2,3-dihydrobenzo[b][1,4]dioxin-2-yl)methyl)piperidin-3-yl)-4-methoxyisoindolin-1-one.

¹H NMR (400 MHz, CDCl₃) δ ppm 1.51-1.69 (1H, m), 1.71-1.85 (2H, m), 1.86-1.96 (1H, m), 2.20-2.39 (2H, m), 2.61-2.73 (2H, m), 2.80-2.89 (1H, m), 2.99-3.08 (1H, m), 3.91 (3H, s), 3.99-4.07 (1H, m), 4.26-4.50 (5H, m), 6.78-6.89 (4H, m), 6.97-7.02 (1H, m), 7.39-7.48 (2H, m).

### EXAMPLE 56: 2-((S)-1-(((S)-2,3-Dihydrobenzo[b][1,4]dioxin-2-yl)methyl)piperidin-3-yl)-4,5-difluoroisoindolin-1-one hydrochloride

A mixture of (S)-1-(((S)-2,3-dihydrobenzo[b][1,4]dioxin-2-yl)methyl)piperidin-3-amine (0.25 g, 1.01 mmol), methyl 2-(bromomethyl)-3,4-difluorobenzoate (0.27 g, 1.01 mmol, WO95/31446 A1) and TEA (0.21 ml, 0.15 g, 1.51 mmol) in toluene (7 ml) was refluxed for 1.5 h. The precipitate was filtered off and the filtrate evaporated to dryness. The residue was purified by flash chromatography yielding 0.27 g 2-((S)-1-(((S)-2,3-dihydrobenzo[b][1,4]-dioxin-2-yl)methyl)piperidin-3-yl)-4,5-difluoroisoindolin-1-one hydrochloride after conversion of free base to HCl salt in Et₂O.

¹H NMR (400 MHz, DMSO-d₆) δ ppm 1.80-2.11 (4H, m), 2.96-3.16 (1H, m), 3.34-3.47 (5H, m), 3.99-4.10 (1H, m), 4.30-4.40 (1H, m), 4.49-4.74 (3H, m), 4.83-4.97 (1H, m), 6.83-6.99 (4H, m), 7.57-7.66 (2H, m), 10.99 (1H, br s).

### EXAMPLE 57: 2-((S)-1-(((S)-2,3-Dihydrobenzo[b][1,4]dioxin-2-yl)methyl)piperidin-3-yl)-4-fluoroisoindolin-1-one hydrochloride

A mixture of (S)-1-(((S)-2,3-dihydrobenzo[b][1,4]dioxin-2-yl)methyl)piperidin-3-amine (0.25 g, 1.01 mmol), methyl 2-(bromomethyl)-3,4-difluorobenzoate (0.25 g, 1.01 mmol) and TEA (0.21 ml, 0.15 g, 1.51 mmol) in toluene (7 ml) was refluxed for 1.5 h. The precipitate was filtered off and the filtrate evaporated to dryness. The residue was purified by flash chromatography yielding 0.30 g 2-((S)-1-(((S)-2,3-dihydrobenzo[b][1,4]dioxin-2-yl)methyl)piperidin-3-yl)-4-fluoroisoindolin-1-one hydrochloride after conversion of free base to HCl salt in Et₂O.

¹H NMR (400 MHz, DMSO-d₆) δ ppm 1.82-2.09 (4H, m), 2.98-3.16 (1H, m), 3.32-3.66 (5H, m), 3.99-4.11 (1H, m), 4.31-4.40 (1H, m), 4.55-4.70 (3H, m), 4.85-4.98 (1H, m), 6.82-7.00 (4H, m), 7.45-7.54 (1H, m), 7.55-7.65 (2H, m), 11.06 (1H, br s).

### EXAMPLE 58: 2-((S)-1-(((S)-2,3-Dihydrobenzo[b][1,4]dioxin-2-yl)methyl)piperidin-3-yl)-5-fluoroisoindolin-1-one hydrochloride

A mixture of (S)-1-(((S)-2,3-dihydrobenzo[b][1,4]dioxin-2-yl)methyl)piperidin-3-amine (0.25 g, 1.01 mmol), methyl 2-(bromomethyl)-4-fluorobenzoate (0.25 g, 1.01 mmol) and TEA (0.21 ml, 0.15 g, 1.51 mmol) in toluene (7 ml) was refluxed for 2 h. The precipitate was filtered off and the filtrate evaporated to dryness. The residue was purified by flash chromatography yielding 0.23 g 2-((S)-1-(((S)-2,3-dihydrobenzo[b][1,4]dioxin-2-yl)methyl)piperidin-3-yl)-4-fluoroisoindolin-1-one hydrochloride after conversion of free base to HCl salt in EtOAc.

¹H NMR (400 MHz, DMSO-d₆) δ ppm 1.77-2.11 (4H, m), 2.99-3.18 (1H, m), 3.32-3.68 (5H, m), 4.00-4.10 (1H, m), 4.31-4.40 (1H, m), 4.48-4.69 (3H, m), 4.86-5.00 (1H, m), 6.83-6.97 (4H, m), 7.31-7.39 (1H, m), 7.51-7.58 (1H, m), 7.73-7.81 (1H, m), 11.05 (1H, br s).

### EXAMPLE 59: 2-((S)-1-(((S)-2,3-Dihydrobenzo[b][1,4]dioxin-2-yl)methyl)piperidin-3-yl)-5-methylisoindolin-1-one formate

A mixture of (S)-1-(((S)-2,3-dihydrobenzo[b][1,4]dioxin-2-yl)methyl)piperidin-3-amine (0.28 g, 1.13 mmol), methyl 2-(bromomethyl)-4-methylbenzoate (0.27 g, 1.13 mmol, Bioorganic & Medicinal Chemistry Letters, 16(6), 1532-1536; 2006) and TEA (0.24 ml, 0.17 g, 1.69 mmol) in toluene (7 ml) was refluxed for 1.5 h. The precipitate was filtered off and the filtrate evaporated to dryness. The residue was purified by preparative RP-HPLC yielding 58 mg 2-((S)-1-(((S)-2,3-dihydrobenzo[b][1,4]dioxin-2-yl)methyl)piperidin-3-yl)-5-methylisoindolin-1-one formate.

¹H NMR (400 MHz, CDCl₃) δ ppm 1.77-2.00 (4H, m), 2.46 (3H, s), 2.47-2.55 (1H, m), 2.75-2.93 (2H, m), 2.05-3.02 (1H, m), 3.08-3.18 (1H, m), 3.34-3.42 (1H, m), 4.00-4.08 (1H, m), 4.25-4.31 (1H, m), 4.31-4.44 (3H, m), 4.46-4.55 (1H, m), 6.78-6.90 (4H, m), 7.21-7.30 (2H, m), 7.60 (1H, br s), 7.70-7.75 (1H, m), 8.25 (1H, br s).

### EXAMPLE 60: 5-Chloro-2-((S)-1-(((S)-2,3-dihydrobenzo[b][1,4]dioxin-2-yl)methyl)piperidin-3-yl)isoindolin-1-one

A mixture of (S)-1-(((S)-2,3-dihydrobenzo[b][1,4]dioxin-2-yl)methyl)piperidin-3-amine (0.15 g, 0.60 mmol), methyl 2-(bromomethyl)-4-chlorobenzoate (0.16 g, 0.60 mmol) and TEA (0.13 ml, 92 mg, 0.91 mmol) in toluene (5 ml) was refluxed for 3.5 h. The precipitate was filtered off and the filtrate evaporated to dryness. The residue was purified by flash chromatography yielding 77 mg 5-chloro-2-((S)-1-(((S)-2,3-dihydrobenzo[b][1,4]dioxin-2-yl)methyl)piperidin-3-yl)isoindolin-1-one.

¹H NMR (400 MHz, CDCl₃) δ ppm 1.53-1.67 (1H, m), 1.71-1.85 (2H, m), 1.86-1.96 (1H, m), 2.22-2.41 (2H, m), 2.62-2.75 (2H, m), 2.80-2.90 (1H, m), 3.00-3.09 (1H, m), 3.99-4.07 (1H, m), 4.26-4.35 (2H, m), 4.37-4.48 (3H, m), 6.78-6.90 (4H, m), 7.40-7.47 (2H, m), 7.74-7.80 (1H, m).

### EXAMPLE 61: 2-((S)-1-(((S)-2,3-Dihydrobenzo[b][1,4]dioxin-2-yl)methyl)piperidin-3-yl)isoindoline-1,3-dione

Prepared using general procedure A from (R)-2-(bromomethyl)-2,3-dihydrobenzo[b][1,4]-dioxine (0.17 g, 0.75 mmol), (S)-2-(piperidin-3-yl)isoindoline-1,3-dione hydrochloride (0.20 g, 0.75 mmol, Faming Zhuanli Shenqing, 102516225, 27 Jun. 2012) and DIPEA (0.39 ml, 0.29 g, 2.25 mmol) in ACN (4 ml) yielding 84 mg 2-((S)-1-(((S)-2,3-Dihydrobenzo[b][1,4]dioxin-2-yl)methyl)piperidin-3-yl)isoindoline-1,3-dione.

¹H NMR (400 MHz, CDCl₃) δ ppm 1.62-1.86 (3H, m), 2.14-2.32 (2H, m), 2.60-2.80 (2H, m), 2.84.2.96 (3H, m), 3.96-4.07 (1H, m), 4.23-4.44 (3H, m), 6.78-6.90 (4H, m), 7.67-7.74 (2H, m), 7.80-7.85 (2H, m).

### EXAMPLE 62: 2-((S)-1-(((S)-2,3-Dihydrobenzo[b][1,4]dioxin-2-yl)methyl)piperidin-3-yl)-5-fluoroisoindoline-1,3-dione

A mixture of (S)-1-(((S)-2,3-dihydrobenzo[b][1,4]dioxin-2-yl)methyl)piperidin-3-amine (0.12 g, 0.48 mmol) and 5-fluoroisobenzofuran-1,3-dione (96 mg, 0.58 mmol) in AcOH (2 ml) was refluxed for 2 h. The solvent was evaporated off and the residue was dissolved in DCM. The solution was washed with 1 M NaOH, water and brine, dried and evaporated. The residue was purified by flash chromatography yielding 0.13 g 2-((S)-1-(((S)-2,3-dihydrobenzo[b][1,4]dioxin-2-yl)methyl)piperidin-3-yl)-5-fluoroisoindoline-1,3-dione.

¹H NMR (400 MHz, DMSO-d₆) δ ppm 1.48-1.64 (1H, m), 1.69-1.81 (2H, m), 2.01-2.15 (2H, m), 2.59-2.71 (3H, m), 2.84-3.01 (2H, m), 3.91-4.00 (1H, m), 4.08-4.20 (1H, m), 4.25-4.36 (2H, m), 6.77-6.88 (4H, m), 7.61-7.69 (1H, m), 7.71-7.75 (1H, m), 7.89-7.95 (1H, m).

### EXAMPLE 63: 2-((S)-1-(((S)-2,3-Dihydrobenzo[b][1,4]dioxin-2-yl)methyl)piperidin-3-yl)-4-fluoroisoindoline-1,3-dione hydrochloride

A mixture of (S)-1-(((S)-2,3-dihydrobenzo[b][1,4]dioxin-2-yl)methyl)piperidin-3-amine (0.10 g, 0.40 mmol) and 4-fluoroisobenzofuran-1,3-dione (81 mg, 0.49 mmol) in AcOH (2 ml) was refluxed for 3 h. The solvent was evaporated off and the residue was dissolved in DCM. The solution was washed with 1 M NaOH, water and brine, dried and evaporated. The residue was purified by flash chromatography yielding 97 mg 2-((S)-1-(((S)-2,3-dihydrobenzo[b][1,4]dioxin-2-yl)methyl)piperidin-3-yl)-4-fluoroisoindoline-1,3-dione hydrochloride after conversion of free base to HCl salt in DCM-dioxane.

¹H NMR (400 MHz, CDCl₃) δ ppm 1.95-2.09 (2H, m), 2.18-2.32 (1H, m), 2.44-2.63 (1H, m), 2.79-2.94 (1H, m), 3.10-3.24 (1H, m), 3.33-3.57 (2H, m), 3.64-3.79 (1H, m), 3.85-4.00 (1H, m), 4.12-4.21 (1H, m), 4.23-4.34 (1H, m), 4.95-5.10 (1H, m), 5.20-5.32 (1H, m), 6.80-6.93 (4H, m), 7.39-7.47 (1H, m), 7.67-7.82 (2H, m), 13.65 (1H, br s).

### EXAMPLE 64: 4-Chloro-2-((S)-1-(((S)-2,3-dihydrobenzo[b][1,4]dioxin-2-yl)methyl)piperidin-3-yl)isoindoline-1,3-dione hydrochloride

A mixture of (S)-1-(((S)-2,3-dihydrobenzo[b][1,4]dioxin-2-yl)methyl)piperidin-3-amine (0.10 g, 0.40 mmol) and 4-chloroisobenzofuran-1,3-dione (74 mg, 0.40 mmol) in AcOH (1 ml) was refluxed for 4 h. The solvent was evaporated off and the residue was dissolved in DCM. The solution was washed with 1 M NaOH, water and brine, dried and evaporated. The residue was purified by flash chromatography yielding 28 mg 4-Chloro-2-((S)-1-(((S)-2,3-dihydrobenzo[b][1,4]dioxin-2-yl)methyl)piperidin-3-yl)isoindoline-1,3-dione hydrochloride after conversion of free base to HCl salt in Et₂O-dioxane.

¹H NMR (400 MHz, CDCl₃) δ ppm 1.91-2.07 (2H, m), 2.19-2.60 (2H, m), 2.70-2.93 (1H, m), 3.03-3.21 (1H, m), 3.24-3.54 (2H, m), 3.56-3.95 (2H, m), 4.08-4.21 (1H, m), 4.24-4.34 (1H, m), 4.85-5.31 (2H, m), 6.80-6.94 (4H, m), 7.65-7.73 (2H, m), 7.76-7.84 (1H, m), 13.59 (1H, br s).

### EXAMPLE 65: 2-((S)-1-(((S)-2,3-Dihydrobenzo[b][1,4]dioxin-2-yl)methyl)piperidin-3-yl)-4-methoxyisoindoline-1,3-dione

A mixture of (S)-1-(((S)-2,3-dihydrobenzo[b][1,4]dioxin-2-yl)methyl)piperidin-3-amine (0.11 g, 0.45 mmol) and 4-methoxyisobenzofuran-1,3-dione (80 mg, 0.45 mmol) in xylenes (3 ml) was refluxed for 5 h. The solvent was evaporated off and the residue was purified by flash chromatography yielding 13 mg 2-((S)-1-(((S)-2,3-dihydrobenzo[b][1,4]dioxin-2-yl)methyl)piperidin-3-yl)-4-methoxyisoindoline-1,3-dione.

¹H NMR (400 MHz, CDCl₃) δ ppm 1.64-1.84 (3H, m), 2.18-2.30 (2H, m), 2.61-2.76 (2H, m), 2.85-2.93 (3H, m), 3.97-4.05 (1H, m), 4.02 (3H, s), 4.23-4.40 (3H, m), 6.78-6.89 (4H, m), 7.17-7.22 (1H, m), 7.40-7.44 (1H, m), 7.62-7.68 (1H, m).

### EXAMPLE 66: 2-((S)-1-(((S)-2,3-Dihydrobenzo[b][1,4]dioxin-2-yl)methyl)piperidin-3-yl)-4,5-dimethoxyisoindoline-1,3-dione hydrochloride

A mixture of (S)-1-(((S)-2,3-dihydrobenzo[b][1,4]dioxin-2-yl)methyl)piperidin-3-amine (0.21 g, 0.83 mmol) and 4,5-dimethoxyisobenzofuran-1,3-dione 0.17 g, 0.83 mmol) in xylenes (5 ml) was refluxed for 3 h. The solvent was evaporated off and the residue was purified by flash chromatography yielding 57 mg 2-((S)-1-(((S)-2,3-dihydrobenzo[b][1,4]-dioxin-2-yl)methyl)piperidin-3-yl)-4,5-dimethoxyisoindoline-1,3-dione hydrochloride after conversion of free base to HCl salt in Et₂O.

¹H NMR (400 MHz, DMSO-d₆) δ ppm 1.82-2.27 (4H, m), 3.00-3.21 (1H, m), 3.44-3.84 (5H, m), 3.92 (3H, s), 3.97 (3H, s), 4.00-4.08 (1H, m), 4.32-4.39 (1H, m), 4.57-4.71 (1H, m), 4.85-4.95 (1H, m), 6.81-6.98 (4H, m), 7.41 (1H, d), 7.59 (1H, d), 11.17 (1H, br s).

### EXAMPLE 67: 2-((S)-1-(((S)-2,3-Dihydrobenzo[b][1,4]dioxin-2-yl)methyl)piperidin-3-yl)-1-oxoisoindoline-5-carbonitrile

A flask was charged with methyl 2-(bromomethyl)-4-cyanobenzoate (0.5 g, 0.984 mmol, U.S. Pat. No. 5,591,752 A1), (S)-1-(((S)-2,3-dihydrobenzo[b][1,4]dioxin-2-yl)methyl)piperidin-3-amine (0.244 g, 0.984 mmol), toluene (5 mL) and triethylamine (0.206 mL, 1.476 mmol). Reaction is refluxed for 2 h, then cooled to room temperature and filtered. Solvents were evaporated and residue was purified with silica gel chromatography using EtOAc/heptanes to give 0.25 g of 2-((S)-1-(((S)-2,3-dihydrobenzo[b]-[1,4]dioxin-2-yl)methyl)piperidin-3-yl)-1-oxoisoindoline-5-carbonitrile as solids.

¹H NMR (400 MHz, CDCl₃) δ ppm 1.61-1.69 (m, 1H), 1.70-1.86 (m, 2H), 1.87-1.99 (m, 1H), 2.26-2.34 (m, 1H), 2.35-2.44 (m, 1H), 2.62-2.75 (m, 2H), 2.79-2.89 (m, 1H), 3.04 (dd, 1H), 4.03 (dd, 1H), 4.25-4.34 (m, 2H), 4.41-4.55 (m, 3H), 6.79-6.90 (m, 4H), 7.72-7.79 (m, 2H), 7.95 (dd, 1H).

### EXAMPLE 68: 2-((S)-1-(((S)-2,3-Dihydrobenzo[b][1,4]dioxin-2-yl)methyl)piperidin-3-yl)-5,6-dimethoxyisoindolin-1-one

A flask was charged with methyl 2-(bromomethyl)-4,5-dimethoxybenzoate (0.15 g, 0.519 mmol, J. Heterocycl. Chem. 1987, 24, 725-731), (S)-1-(((S)-2,3-dihydrobenzo[b][1,4]-dioxin-2-yl)methyl)piperidin-3-amine (0.129 g, 0.519 mmol), toluene (2 mL) and triethylamine (0.108 mL, 0.778 mmol). Reaction is refluxed for 2 h, then cooled to room temperature and filtered. Solvents were evaporated and residue was purified with silica gel chromatography using CH₂Cl₂/EtOAc/Et₃N (20:10:1) to give 0.172 g of 2-((S)-1-(((S)-2,3-dihydrobenzo[b][1,4]dioxin-2-yl)methyl)piperidin-3-yl)-5,6-dimethoxyiso-indolin-1-one as white solids.

¹H NMR (400 MHz, CDCl₃) δ ppm 1.51-1.63 (m, 1H), 1.69-1.83 (m, 2H), 1.85-1.95 (m, 1H), 2.20-2.36 (m, 2H), 2.63-2.72 (m, 2H), 2.79-2.88 (m, 1H), 2.99-3.07 (m, 1H), 3.94 (s, 3H), 3.94 (s, 3H), 4.04 (dd, 1H), 4.26-4.36 (m, 4H), 4.36-4.46 (m, 1H), 6.78-6.88 (m, 4H), 6.90 (s, 1H), 7.31 (s, 1H).

### EXAMPLE 69: 2-((S)-1-(((S)-2,3-Dihydrobenzo[b][1,4]dioxin-2-yl)methyl)piperidin-3-yl)-5-methoxyisoindolin-1-one

A flask was charged with methyl 2-(bromomethyl)-4-methoxybenzoate (0.5 g, 1.930 mmol, WO2011/85261 A1), (S)-1-(((S)-2,3-dihydrobenzo[b][1,4]dioxin-2-yl)methyl)piperidin-3-amine (0.527 g, 2.123 mmol), toluene (20 mL) and triethylamine (0.296 mL, 2.123 mmol). Reaction is refluxed for 4 h, then cooled to room temperature and filtered. Solvents were evaporated and residue was triturated with diethyl ether. Solids were filtered and dried in vacuum to give 0.238 g of 2-((S)-1-(((S)-2,3-dihydrobenzo[b]-[1,4]dioxin-2-yl)methyl)piperidin-3-yl)-5-methoxyisoindolin-1-one.

¹H NMR (400 MHz, CDCl₃) δ ppm 1.51-1.64 (m, 1H), 1.68-1.84 (m, 2H), 1.85-1.95 (m, 1H), 2.19-2.35 (m, 2H), 2.61-2.74 (m, 2H), 2.79-2.89 (m, 1H), 2.99-3.07 (m, 1H), 3.87 (s, 3H), 4.04 (dd, 1H), 4.26-4.35 (m, 2H), 4.35-4.48 (m, 3H), 6.78-6.89 (m, 4H), 6.90-6.93 (m, 1H), 6.98 (dd, 1H), 7.76 (d, 1H).

### EXAMPLE 70: 2-((S)-1-(((S)-2,3-Dihydrobenzo[b][1,4]dioxin-2-yl)methyl)piperidin-3-yl)-6-methoxyisoindolin-1-one

A flask was charged with methyl 2-(bromomethyl)-5-methoxybenzoate (0.209 g, 0.805 mmol, WO2007/84451 A1), (S)-1-(((S)-2,3-dihydrobenzo[b][1,4]dioxin-2-yl)methyl)piperidin-3-amine (0.200 g, 0.805 mmol), toluene (5 mL) and triethylamine (0.112 mL, 0.805 mmol). Reaction is refluxed for 3 h, then cooled to room temperature and filtered. Solvents were evaporated and residue was purified with silica gel chromatography using EtOAc/heptanes to give 0.120 g of 2-((S)-1-(((S)-2,3-dihydrobenzo[b][1,4]-dioxin-2-yl)methyl)piperidin-3-yl)-6-methoxyisoindolin-1-one.

¹H NMR (400 MHz, CDCl₃) δ ppm 1.51-1.65 (m, 1H), 1.69-1.84 (m, 2H), 1.86-1.96 (m, 1H), 2.20-2.36 (m, 2H), 2.62-2.74 (m, 2H), 2.81-2.90 (m, 1H), 3.01-3.08 (m, 1H), 3.86 (s, 3H), 4.03 (dd, 1H), 4.26-4.34 (m, 2H), 4.33-4.49 (m, 3H), 6.78-6.90 (m, 4H), 7.09 (dd, 1H), 7.32 (dd, 1H), 7.34 (d, 1H).

### EXAMPLE 71: N-((2-((S)-1-(((S)-2,3-Dihydrobenzo[b][1,4]dioxin-2-yl)methyl)piperidin-3-yl)-1-oxoisoindolin-5-yl)methyl)acetamide

### Step 1: Methyl 4-bromo-2-(bromomethyl)benzoate

To a cold stirred solution of methyl 4-bromo-2-methylbenzoate (8.9 g, 38.86 mmol) in tetrachloromethane (80 mL) was added N-bromosuccinimide (6.91 g, 38.86 mmol) and azobisisobutyronitrile (150 mg, 091 mmol) at room temperature. The resulting solution was heated to 80° C. for 16 h. The reaction mixture was cooled to room temperature, filtered and concentrated to afford 8.4 g of crude methyl 4-bromo-2-(bromomethyl)benzoate which was used as such in the next step.

¹H NMR (400 MHz, CDCl₃) δ ppm 2.58 (s, 3H), 3.88 (s, 3H), 7.35-7.42 (m, 2H), 7.77-7.78 (m, 1H).

### Step 2: (S)-tert-Butyl 3-(5-bromo-1-oxoisoindolin-2-yl)piperidine-1-carboxylate

To a stirred solution of methyl 4-bromo-2-(bromomethyl)benzoate (8.4 g, 27.27 mmol) and (S)-tert-butyl 3-aminopiperidine-1-carboxylate (5.45 g, 27.27 mmol) in acetonitrile (80 mL) was added K₂CO₃ (11.3 g, 81.83 mmol) at room temperature. The resulting solution was heated to 80° C. for 16 h Reaction mixture was concentrated under reduced pressure and the residue was dissolved in a mixture of EtOAc (200 mL) and water (200 mL). The organic layer was separated, dried over anhydrous Na₂SO₄ and concentrated. Crude product was purified with silica gel chromatography using 30% EtOAc/petroleum ether as eluent to afford 8.6 g of (S)-tert-butyl 3-(5-bromo-1-oxoisoindolin-2-yl)piperidine-1-carboxylate as sticky solids.

LC-MS (ES+) [M+1]: 395.1.

### Step 3: (S)-tert-Butyl 3-(5-cyano-1-oxoisoindolin-2-yl)piperidine-1-carboxylate

In a seal tube, a solution of (S)-tert-butyl 3-(5-bromo-1-oxoisoindolin-2-yl)piperidine-1-carboxylate (9.6 g, 24.30 mmol) in DMF (100 mL) was degassed with argon for 15 min, then dppf (0.538 g, 0.97 mmol), Pd₂(dba)₃ (0.667 g, 0.73 mmol) and ZnCN₂ (2.91 g, 24.79 mmol) were added to the reaction mixture. The resulting reaction mixture was heated to 120° C. for 20 h. The reaction mixture was filtered through celite pad and washed with EtOAc (100 mL). Filtrate was washed with water, organic layer was separated and aqueous layer was extracted with EtOAc (3×100 mL). The combined organic extracts were washed with water (2×100 mL), brine (100 mL). The organic layer was dried over anhydrous Na₂SO₄, filtered and concentrated. Crude product was purified with silica gel chromatography using 30% EtOAc/petroleum ether as eluent to afford 4.4 g of (S)-tert-butyl 3-(5-cyano-1-oxoisoindolin-2-yl)piperidine-1-carboxylate as yellow solid.

¹H NMR (400 MHz, DMSO-d₆): 1.25-1.57 (m, 15H), 1.72-1.81 (m, 3H), 1.84-1.93 (m, 2H), 2.62-2.80 (m, 1H), 2.82-3.03 (br, 1H), 3.83-4.02 (m, 3H), 4.50 (s, 2H), 7.61-7.72 (m, 2H), 7.84 (m, 1H).

### Step 4: (S)-tert-Butyl 3-(5-(aminomethyl)-1-oxoisoindolin-2-yl)piperidine-1-carboxylate

A suspension of (S)-tert-butyl 3-(5-cyano-1-oxoisoindolin-2-yl)piperidine-1-carboxylate (2.0 g, 5.86 mmol) and Raney nickel (1.5 g) in MeOH (30 mL) was subjected to hydrogenation (60 psi) at room temperature for 16 h. The reaction mixture was filtered through celite pad and washed with methanol. The filtrate was concentrated under reduced pressure. Crude product was purified with silica gel chromatography using 8% MeOH in CH₂Cl₂ as eluent to obtain 1.0 g of (S)-tert-butyl 3-(5-(aminomethyl)-1-oxoisoindolin-2-yl)piperidine-1-carboxylate as yellow solids.

LC-MS (ES+) [M+1]: 346.3.

### Step 5: (S)-tert-Butyl 3-(5-(acetamidomethyl)-1-oxoisoindolin-2-yl)piperidine-1-carboxylate

A stirred solution of (S)-tert-butyl 3-(5-(aminomethyl)-1-oxoisoindolin-2-yl)piperidine-1-carboxylate (200 mg, 0.57 mmol) in acetonitrile (10 mL) were added NaHCO₃ (146 mg, 1.73 mmol) and acetic anhydride (0.05 mL, 0.58 mmol) at room temperature and stirred for 16 h. Reaction mixture was concentrated under reduced pressure and the residue was dissolved in a mixture of EtOAc (50 mL) and water (50 mL). Organic layer was separated, dried over anhydrous Na₂SO₄ and concentrated. Crude product was purified with silica gel chromatography using 30% EtOAc/petroleum ether as eluent to afford 0.14 g of (S)-tert-butyl 3-(5-(acetamidomethyl)-1-oxoisoindolin-2-yl)piperidine-1-carboxylate as sticky solid.

LC-MS (ES+) [M+1]: 388.2.

### Step 6: (S)—N-((1-Oxo-2-(piperidin-3-yl)isoindolin-5-yl)methyl)acetamide

To a stirred solution of (S)-tert-butyl 3-(5-(acetamidomethyl)-1-oxoisoindolin-2-yl)piperidine-1-carboxylate (280 mg, 0.723 mmol) in CH₂Cl₂ (10 mL) was added trifluoroacetic acid (0.55 mL, 7.33 mmol) at 0° C. and allowed to stir at room temperature for 6 h. After completion of starting material, the reaction mixture was concentrated under reduced pressure. The crude product was purified by triturating with diethyl ether to give 0.28 g of (S)—N-((1-oxo-2-(piperidin-3-yl)isoindolin-5-yl)methyl)acetamide, trifluoroacetate as sticky solids.

LC-MS (ES+) [M+1]: 288.2.

### Step 7: N-((2-((S)-1-(((S)-2,3-Dihydrobenzo[b][1,4]dioxin-2-yl)methyl)piperidin-3-yl)-1-oxoisoindolin-5-yl)methyl)acetamide

A sealed tube was charged with (S)—N-((1-oxo-2-(piperidin-3-yl)isoindolin-5-yl)methyl)-acetamide, trifluoroacetate (0.120 g, 0.299 mmol), (R)-(2,3-dihydrobenzo[b][1,4]dioxin-2-yl)methyl 4-methylbenzenesulfonate (0.115 g, 0.359 mmol), potassium carbonate (0.124 g, 0.897 mmol) and acetonitrile (3 mL). Tube was sealed and reaction was heated with microwave irradiation to 120° C. for 5 hours. Mixture was cooled, filtered and washed with acetonitrile. Solvents were evaporated to dryness. Residue was purified with silica gel chromatography using EtOAc/heptanes and MeOH/CH₂Cl₂ to give 0.069 g of N-((2-((S)-1-(((S)-2,3-dihydrobenzo[b][1,4]dioxin-2-yl)methyl)piperidin-3-yl)-1-oxoisoindolin-5-yl)methyl)acetamide.

¹H NMR (400 MHz, CDCl₃) δ ppm 1.51-1.64 (m, 1H), 1.69-1.84 (m, 2H), 1.84-1.94 (m, 1H), 2.06 (s, 3H), 2.20-2.37 (m, 2H), 2.60-2.75 (m, 2H), 2.79-2.90 (m, 1H), 2.99-3.08 (m, 1H), 4.03 (dd, 1H), 4.25-4.33 (m, 2H), 4.38-4.47 (m, 3H), 4.52 (d, 2H), 5.83-6.04 (m, 1H), 6.77-6.91 (m, 4H), 7.31-7.41 (m, 2H), 7.77 (d, 1H).

### EXAMPLE 72: 3-((S)-1-(((S)-2,3-Dihydrobenzo[b][1,4]dioxin-2-yl)methyl)-piperidin-3-yl)quinazoline-2,4(1H,3H)-dione

### Step 1: 1-((S)-1-(((S)-2,3-Dihydrobenzo[b][1,4]dioxin-2-yl)methyl)piperidin-3-yl)urea

A flask was charged with (S)-1-(((S)-2,3-dihydrobenzo[b][1,4]dioxin-2-yl)methyl)piperidin-3-amine (1.0 g, 4.03 mmol) and 1 M HCl solution (8 mL, 8.00 mmol). To this was added potassium cyanate (0.98 g, 12.08 mmol) and reaction was mixed for 4 hours. Then another batch of potassium cyanate (0.98 g, 12.08 mmol) was added followed by pH adjustment to ˜3 with 6 M HCl solution. Mixture is stirred for 2 hours. Reaction was acidified by addition of 6 M HCl solution to pH 1-2. When gas evolution had ended, the mixture was basified by addition of solid NaHCO₃ until pH 9-10. Mixture was extracted three times with EtOAc. Combined organic extracts were washed with brine, dried with anhydrous Na₂SO₄ and evaporated to dryness. Crude product was purified with silica gel chromatography using 1-10% MeOH/CH₂Cl₂ to give 0.77 g of 1-((S)-1-(((S)-2,3-dihydrobenzo[b][1,4]dioxin-2-yl)methyl)piperidin-3-yl)urea as white solids.

LC-MS (ES+) [M+1]: 292.5.

### Step 2: 3-((S)-1-(((S)-2,3-Dihydrobenzo[b][1,4]-dioxin-2-yl)methyl)piperidin-3-yl)quinazoline-2,4(1H,3H)-dione

A sealed tube was charged with cesium carbonate (0.224 g, 0.686 mmol), 9-dimethyl-4,5-bis(diphenyl-phosphino)xanthene (9.93 mg, 0.017 mmol), bis(dibenzylideneacetonato)-palladium (4.9 mg, 8.6 μmol), methyl-2-bromobenzoate (0.048 mL, 0.343 mmol) and 1-((S)-1-(((S)-2,3-dihydrobenzo[b][1,4]dioxin-2-yl)methyl)piperidin-3-yl)urea (0.100 g, 0.343 mmol). Then air atmosphere was removed with nitrogen flow and dioxane (2 mL) was added. Tube was sealed and heated at 100° C. for 4 hours. Mixture was cooled to room temperature, diluted with EtOAc (5 mL) and saturated NaHCO₃ solution (5 mL). Phases were separated and aqueous phase was extracted with EtOAc (5 mL). Combined organic extracts were washed with brine (5 mL), dried with anhydrous Na₂SO₄ and evaporated to dryness. Crude product was purified with silica gel chromatography using 10-100% EtOAc/heptanes to give 70 mg of 3-((S)-1-(((S)-2,3-dihydrobenzo[b][1,4]-dioxin-2-yl)methyl)-piperidin-3-yl)quinazoline-2,4(1H,3H)-dione as off-white solids.

¹H NMR (400 MHz, CDCl₃) δ ppm 1.67-1.95 (m, 3H), 2.18-2.37 (m, 1H), 2.44-2.63 (m, 1H), 2.63-2.83 (m, 2H), 2.84-3.05 (m, 2H), 3.15-3.31 (m, 1H), 3.92-4.12 (m, 1H), 4.20-4.43 (m, 2H), 5.05-5.25 (m, 1H), 6.71-6.94 (m, 4H), 7.10 (d, 1H), 7.15-7.33 (m, 1H), 7.60 (t, 1H), 8.11 (d, 1H), 10.25 (br s, 1H).

### EXAMPLE 73: 3-((S)-1-(((S)-2,3-Dihydrobenzo[b][1,4]dioxin-2-yl)methyl)-piperidin-3-yl)-5-ethyl-1-methyl-5-phenylimidazolidine-2,4-dione

### Step 1: 2-(Ethoxycarbonylamino)-2-phenylbutanoic acid

A flask was charged with 2-amino-2-phenylbutanoic acid (2.0 g, 11.16 mmol) and 1 M sodium hydroxide solution (27.9 mL, 27.9 mmol). Mixture was cooled with ice bath and ethyl chloroformate (1.6 mL, 16.74 mmol) was added. Reaction was allowed to warm to room temperature and mixed over weekend. Mixture was cooled with ice bath and another portion of ethyl chloroformate (1.6 mL, 16.74 mmol) was added. After 4 hours, the reaction was basified by addition of 50% NaOH solution and stirred overnight. Reaction mixture was acidified to pH 1-2 by addition of 4 M HCl solution and then extracted with 20% IPA/EtOAc (4×). Combined organic extracts were dried with anhydrous Na₂SO₄ and evaporated to dryness to give 2.48 g of 2-(ethoxycarbonylamino)-2-phenylbutanoic acid as glassy solids.

LC-MS (ES−) [M−1]: 250.2.

### Step 2: Ethyl-1-((S)-1-(((S)-2,3-dihydrobenzo[b][1,4]dioxin-2-yl)methyl)piperidin-3-ylamino)-1-oxo-2-phenylbutan-2-ylcarbamate

A flask was charged with 2-(ethoxycarbonylamino)-2-phenylbutanoic acid (0.215 g, 0.856 mmol), (S)-1-(((S)-2,3-dihydrobenzo[b][1,4]dioxin-2-yl)methyl)piperidin-3-amine, p-toluenesulfonate (0.3 g, 0.713 mmol), trimethylamine (0.30 mL, 2.14 mmol) and dry CH₂Cl₂ (5 mL). Then HBTU (0.352 g, 0.927 mmol) was added and mixture was stirred at room temperature for 4.5 hours. To this was added saturated NaHCO₃ solution (5 mL) and phases were separated. Aqueous phase was extracted with CH₂Cl₂ (5 mL). Combined organic extracts were washed with brine (5 mL), dried with anhydrous Na₂SO₄ and evaporated to dryness. Crude product was purified with silica gel chromatography using EtOAc/heptanes to give 0.279 g of ethyl 1-((S)-1-(((S)-2,3-dihydrobenzo[b][1,4]-dioxin-2-yl)methyl)piperidin-3-ylamino)-1-oxo-2-phenylbutan-2-ylcarbamate as glassy solids.

LC-MS (ES+) [M+1]: 482.5.

### Step 3: 3-((S)-1-(((S)-2,3-Dihydrobenzo[b][1,4]dioxin-2-yl)methyl)piperidin-3-yl)-5-ethyl-5-phenylimidazolidine-2,4-dione

A flask was charged with ethyl 1-((S)-1-(((S)-2,3-dihydrobenzo[b][1,4]dioxin-2-yl)methyl)piperidin-3-ylamino)-1-oxo-2-phenylbutan-2-ylcarbamate (0.274 g, 0.569 mmol), potassium tert-butoxide (0.064 g, 0.569 mmol) and dry THF (4 mL) under nitrogen atmosphere. Mixture was stirred overnight at room temperature. Reaction was quenched by addition of saturated NH₄Cl solution (5 mL). Reaction mixture was extracted with EtOAc (2×5 mL). Combined organic extracts were washed with brine (5 mL), dried with anhydrous Na₂SO₄ and evaporated to dryness. Crude product was purified with silica gel chromatography using 10-100% EtOAc/heptanes to give 0.179 g of 3-((S)-1-(((S)-2,3-dihydrobenzo[b][1,4]dioxin-2-yl)methyl)piperidin-3-yl)-5-ethyl-5-phenylimidazolidine-2,4-dione as white foam.

LC-MS (ES+) [M+1]: 436.6.

### Step 4: 3-((S)-1-(((S)-2,3-Dihydrobenzo[b][1,4]dioxin-2-yl)methyl)piperidin-3-yl)-5-ethyl-1-methyl-5-phenylimidazolidine-2,4-dione

A flask was charged with 3-((S)-1-(((S)-2,3-dihydrobenzo[b][1,4]dioxin-2-yl)methyl)-piperidin-3-yl)-5-ethyl-5-phenylimidazolidine-2,4-dione (80 mg, 0.184 mmol) and dry DMF (2 mL) under nitrogen. To this was added 60% sodium hydride in mineral oil (9.6 mg, 0.239 mmol). Reaction was stirred for 15 min and iodomethane (14 μL, 0.220 mmol) was added. When reaction was completed, the mixture was evaporated to dry onto silica gel and purified with silica gel chromatography using 0-100% EtOAc/heptanes to give 74 mg of 3-((S)-1-(((S)-2,3-dihydrobenzo[b][1,4]dioxin-2-yl)methyl)piperidin-3-yl)-5-ethyl-1-methyl-5-phenylimidazolidine-2,4-dione as glassy solids.

¹H NMR (400 MHz, CDCl₃) δ ppm 0.83-0.91 (m, 3H), 1.64-1.79 (m, 3H), 1.94-2.06 (m, 1H), 2.07-2.26 (m, 2H), 2.51-2.74 (m, 3H), 2.79 (d, 3H), 2.79-2.96 (m, 3H), 3.94-4.04 (m, 1H), 4.13-4.34 (m, 3H), 6.77-6.89 (m, 4H), 7.22-7.29 (m, 2H), 7.31-7.44 (m, 3H).

### EXAMPLE 74: 3-((S)-1-(((S)-2,3-Dihydrobenzo[b][1,4]dioxin-2-yl)methyl)piperidin-3-yl)-5-methyl-5-phenylimidazolidine-2,4-dione

### Step 1: 2-(Ethoxycarbonylamino)-2-phenylpropanoic acid

A flask was charged with 2-amino-2-phenylpropanoic acid (1.15 g, 6.96 mmol) and 1 M sodium hydroxide solution (27.8 mL, 27.8 mmol). Ethyl chloroformate (1.9 mL, 20.89 mmol) was added in dropwise manner. Reaction was mixed overnight. Mixture was cooled with ice bath and acidified to pH 1-2 by addition of 4 M HCl solution. Resulting white solids were filtered and washed with 1 M HCl solution. Product was dried in 40° C. vacuum oven to give 0.814 g of 2-(ethoxycarbonylamino)-2-phenylpropanoic acid as white solids.

LC-MS (ES−) [M−1]: 236.2.

### Step 2: Ethyl-1-((S)-1-(((S)-2,3-dihydrobenzo[b][1,4]dioxin-2-yl)methyl)-piperidin-3-ylamino)-1-oxo-2-phenylpropan-2-ylcarbamate

A flask was charged with 2-(ethoxycarbonylamino)-2-phenylpropanoic acid (0.248 g, 1.046 mmol), (S)-1-(((S)-2,3-dihydrobenzo[b][1,4]dioxin-2-yl)methyl)piperidin-3-amine, p-toluenesulfonate (0.4 g, 0.951 mmol), trimethylamine (0.40 mL, 2.85 mmol) and dry CH₂Cl₂ (5 mL). Then HBTU (0.433 g, 1.141 mmol) was added and mixture was stirred overnight at room temperature. To this was added saturated NaHCO₃ solution (5 mL) and phases were separated. Aqueous phase was extracted with CH₂Cl₂ (5 mL). Combined organic extracts were washed with brine (5 mL), dried with anhydrous Na₂SO₄ and evaporated to dryness. Crude product was purified with silica gel chromatography using 10-100% EtOAc/heptanes to give 0.266 g of ethyl 1-((S)-1-(((S)-2,3-dihydrobenzo[b][1,4]dioxin-2-yl)methyl)-piperidin-3-ylamino)-1-oxo-2-phenylpropan-2-ylcarbamate as sticky solids.

LC-MS (ES+) [M+1]: 468.7.

### Step 3: 3-((S)-1-(((S)-2,3-Dihydrobenzo[b][1,4]dioxin-2-yl)methyl)piperidin-3-yl)-5-methyl-5-phenylimidazolidine-2,4-dione

A flask was charged with ethyl 1-((S)-1-(((S)-2,3-dihydrobenzo[b][1,4]dioxin-2-yl)methyl)-piperidin-3-ylamino)-1-oxo-2-phenylpropan-2-ylcarbamate (0.256 g, 0.548 mmol), potassium tert-butoxide (0.068 g, 0.602 mmol) and dry THF (4 mL) under nitrogen atmosphere. Mixture was stirred for 1.5 h at room temperature. Reaction was quenched by addition of saturated NH₄Cl solution (5 mL). Reaction mixture was extracted with EtOAc (2×5 mL). Combined organic extracts were washed with brine (5 mL), dried with anhydrous Na₂SO₄ and evaporated to dryness to give 0.226 g of 3-((S)-1-(((S)-2,3-dihydrobenzo[b][1,4]dioxin-2-yl)methyl)piperidin-3-yl)-5-methyl-5-phenylimidazolidine-2,4-dione as semisolids.

¹H NMR (400 MHz, CDCl₃) δ ppm 1.53-1.78 (m, 3H), 1.81 (s, 3H), 2.03-2.24 (m, 2H), 2.56-2.90 (m, 5H), 3.93-4.03 (m, 1H), 4.06-4.19 (m, 1H), 4.19-4.34 (m, 2H), 5.96 (s, 1H), 6.76-6.90 (m, 4H), 7.30-7.43 (m, 3H), 7.43-7.51 (m, 2H).

### EXAMPLE 75: 6-((S)-1-(((S)-2,3-Dihydrobenzo[b][1,4]dioxin-2-yl)methyl)piperidin-3-yl)-5H-pyrrolo[3,4-b]pyridine-5,7(6H)-dione

A sealed tube was charged with (S)-1-(((S)-2,3-dihydrobenzo[b][1,4]dioxin-2-yl)methyl)piperidin-3-amine (0.15 g, 0.604 mmol), 2,3-pyridinedicarboxylic anhydride (0.090 g, 0.604 mmol) and toluene (3 mL). Vial was sealed and heated with microwave irradiation to 120° C. for 12 h. Solvents were evaporated. Residue partitioned between CH₂Cl₂ ad 1 M NaOH solution. Organic phase was washed with brine, dried with anhydrous Na₂SO₄ and evaporated to dryness. Crude product was purified with silica gel chromatography using 2% MeOH/CH₂Cl₂ to give 56 mg of 6-((S)-1-(((S)-2,3-dihydrobenzo[b][1,4]dioxin-2-yl)methyl)piperidin-3-yl)-5H-pyrrolo[3,4-b]pyridine-5,7(6H)-dione.

¹H NMR (400 MHz, CDCl₃) δ ppm 1.64-1.89 (m, 3H), 2.15-2.35 (m, 2H), 2.61-2.83 (m, 2H), 2.86-3.00 (m, 3H), 4.02 (dd, 1H), 4.23-4.36 (m, 2H), 4.37-4.52 (m, 1H), 6.76-6.92 (m, 4H), 7.61 (dd, 1H), 8.14 (dd, 1H), 8.97 (dd, 1H).

### EXAMPLE 76:2-((S)-1-(((S)-2,3-Dihydrobenzo[b][1,4]dioxin-2-yl)methyl)piperidin-3-yl)-2,3-dihydro-1H-pyrrolo[3,4-c]pyridin-1-one

### Step 1: Methyl-3-(chloromethyl)isonicotinate

To a stirred solution of methyl 3-methylisonicotinate (9.0 g, 59.60 mmol, Tetrahedron 2013, 69, 6799-6803) in tetrachloromethane (200 mL) was added azobisisobutyronitrile (0.098 g, 0.59 mmol), N-chlorosuccinimide (12.06 g, 89.40 mmol) and acetic acid (3.6 mL) at 0° C. The resulting mixture was stirred at room temperature in presence of 200 W Tungsten lamp for 16 h. Reaction mixture was neutralized with saturated NaHCO₃ solution and extracted with CH₂Cl₂ (2×100 mL). Organic layer was dried with anhydrous Na₂SO₄, filtered and concentrated under reduced pressure to afford 5.0 g crude methyl 3-(chloromethyl)isonicotinate as brown gummy liquid which was used as such in the next step.

LC-MS (ES+) [M+1]: 186.1.

### Step 2: (S)-tert-Butyl 3-(1-oxo-1H-pyrrolo[3,4-c]pyridin-2(3H)-yl)piperidine-1-carboxylate

To a stirred solution of(S)-tert-butyl 3-aminopiperidine-1-carboxylate (5.0 g, 25.0 mmol) and crude methyl 3-(chloromethyl)isonicotinate (4.62 g, 25.0 mmol) in THF (50.0 mL) was added 50% NaH in mineral oil (1.8 g, 37.5 mmol) at 0° C. and the reaction mixture was allowed to stir at room temperature for 16 h. Reaction mixture was poured into ice cold water and extracted with EtOAc (2×50 mL). Combined organic extracts were dried with anhydrous Na₂SO₄, filtered and concentrated under reduced pressure. The crude compound was purified by reverse phase chromatography to afford 0.3 g of (S)-tert-butyl 3-(1-oxo-1H-pyrrolo[3,4-c]pyridin-2(3H)-yl)piperidine-1-carboxylate as brown liquid.

LC-MS (ES+) [M+1]: 318.2.

### Step 3: (S)-2-(Piperidin-3-yl)-2,3-dihydro-1H-pyrrolo[3,4-c]pyridin-1-one, hydrochloride

To a solution of (S)-tert-butyl 3-(1-oxo-1H-pyrrolo[3,4-c]pyridin-2(3H)-yl)piperidine-1-carboxylate (0.3 g, 0.95 mmol) in 1,4-dioxane (5 mL) was added 4 M HCl in 1,4-dioxane (15 mL) at 0° C. and reaction was stirred at room temperature for 4 h. After completion of starting material, the reaction mixture was concentrated under reduced pressure. Crude product was triturated with n-pentane and diethyl ether to afford (S)-2-(piperidin-3-yl)-2,3-dihydro-1H-pyrrolo[3,4-c]pyridin-1-one, hydrochloride as off white solid. LC-MS (ES+) [M+1]: 218.1.

### Step 4: 2-((S)-1-(((S)-2,3-Dihydrobenzo[b][1,4]dioxin-2-yl)methyl)piperidin-3-yl)-2,3-dihydro-1H-pyrrolo[3,4-c]pyridin-1-one, hydrochloride

A sealed tube was charged with (S)-2-(piperidin-3-yl)-2,3-dihydro-1H-pyrrolo[3,4-c]pyridin-1-one, hydrochloride (0.120 g, 0.509 mmol), (R)-(2,3-dihydrobenzo[b][1,4]-dioxin-2-yl)methyl 4-methylbenzenesulfonate (0.212 g, 0.663 mmol), potassium carbonate (0.191 g, 1.381 mmol) and acetonitrile (4 mL). Tube was sealed and reaction was heated with microwave irradiation to 120° C. for 4 hours. Mixture was cooled, filtered and washed with acetonitrile. Solvents were evaporated to dryness. Residue was purified with silica gel chromatography using 2% MeOH/CH₂Cl₂ and MeOH/EtOAc to give 0.129 g of product. This material was transferred to its corresponding HCl salt by dissolving in CH₂Cl₂ (3 mL) and addition of 0.4 M HCl in dioxane (1 mL). Formed solids were filtered and washed with CH₂Cl₂. Product was dried in vacuum oven at 40° C. to give 0.078 g of 2-((S)-1-(((S)-2,3-dihydrobenzo[b][1,4]dioxin-2-yl)methyl)piperidin-3-yl)-2,3-dihydro-1H-pyrrolo[3,4-c]pyridin-1-one, hydrochloride.

¹H NMR (400 MHz, DMSO-d₆) δ ppm 1.78-2.17 (m, 4H), 3.12 (br s, 1H), 3.22-3.87 (m, 5H), 3.96-4.16 (m, 1H), 4.37 (d, 1H), 4.49-4.83 (m, 3H), 4.95 (br s, 1H), 6.72-7.06 (m, 4H), 7.74 (br s, 1H), 8.76 (d, 1H), 8.97 (s, 1H), 11.48 (br s, 1H).

### EXAMPLE 77: 6-((S)-1-(((S)-2,3-Dihydrobenzo[b][1,4]dioxin-2-yl)methyl)piperidin-3-yl)-5H-pyrrolo[3,4-b]pyridin-7(6H)-one

### Step 1: (S)-tert-Butyl 3-(((2-chloropyridin-3-yl)methyl)amino)piperidine-1-carboxylate

A mixture of (S)-tert-butyl 3-aminopiperidine-1-carboxylate (3.0 g, 14.97 mmol) and 3-chloropicolin-aldehyde (2.5 g, 17.97 mmol) in methanol (50.0 mL) was stirred at room temperature for 27 h. The reaction mixture was cooled to 0° C., NaBH₄ (1.7 g, 44.91 mmol) was added portion wise and then reaction was stirred at room temperature for 3 h. Solvent was evaporated under reduced pressure and the residue was diluted with EtOAc (150 mL). The organic layer was washed with water (2×60 mL), dried over anhydrous Na₂SO₄ and concentrated. Crude product was purified with silica gel chromatography using 3% MeOH/CH₂Cl₂ as eluent to afford 1.5 g of (S)-tert-butyl 3-(((2-chloropyridin-3-yl)methyl)amino)piperidine-1-carboxylate.

LC-MS (ES+) [M+1]: 326.2.

### Step 2: (S)-tert-Butyl-3-(7-oxo-5H-pyrrolo[3,4-b]pyridin-6(7H)-yl)piperidine-1-carboxylate

In a steel bomb was taken (S)-tert-butyl 3-(((2-chloropyridin-3-yl)methyl)amino)piperidine-1-carboxylate (500 mg, 1.53 mmol), PdCl₂(dppf) (125 mg, 0.15 mmol), Et₃N (0.43 mL, 3.07 mmol) in ethanol (15 mL). The mixture was heated at 120° C. in the presence of CO gas at 600 psi for 24 h. The reaction mixture was cooled to room temperature, filtered through a pad of celite and washed with EtOAc (20 mL). The filtrate was concentrated under reduced pressure. Crude product was purified with silica gel chromatography using 3-4% MeOH/CH₂Cl₂ to afford 0.165 g of(S)-tert-butyl 3-(7-oxo-5H-pyrrolo[3,4-b]pyridin-6(7H)-yl)piperidine-1-carboxylate.

LC-MS (ES+) [M+1]: 318.2.

### Step 3: (S)-6-(Piperidin-3-yl)-5H-pyrrolo[3,4-b]pyridin-7(6H)-one hydrochloride

To an ice cold stirred solution of (S)-tert-butyl 3-(7-oxo-5H-pyrrolo[3,4-b]pyridin-6(7H)-yl)piperidine-1-carboxylate (600 mg, 3.45 mmol) in 1,4-dioxane (12 mL) was added 4.0 M HCl in dioxane (8 mL) and stirred at room temperature for 6 h. Solvent was evaporated under reduced pressure. Crude product was triturated with Et₂O to give 0.272 g of (S)-6-(piperidin-3-yl)-5H-pyrrolo[3,4-b]pyridin-7(6H)-one hydrochloride as white solids.

LC-MS (ES+) [M+1]: 218.2.

### Step 4: 6-((S)-1-(((S)-2,3-Dihydrobenzo[b][1,4]dioxin-2-yl)methyl)piperidin-3-yl)-5H-pyrrolo[3,4-b]pyridin-7(6H)-one, hydrochloride

A sealed tube was charged with (S)-6-(piperidin-3-yl)-5H-pyrrolo[3,4-b]pyridin-7(6H)-one (0.120 g, 0.473 mmol), (R)-(2,3-dihydrobenzo[b][1,4]dioxin-2-yl)methyl 4-methylbenzenesulfonate (0.212 g, 0.663 mmol), potassium carbonate (0.191 g, 1.381 mmol) and acetonitrile (4 mL). Tube was sealed and reaction was heated with microwave irradiation to 120° C. for 4 hours. Mixture was cooled, filtered and washed with acetonitrile. Solvents were evaporated to dryness. Residue was purified with silica gel chromatography using 2% MeOH/CH₂Cl₂ to give 0.13 g of product as oil. This material was transferred to its corresponding HCl salt by dissolving in CH₂Cl₂ (3 mL) and addition of 0.4 M HCl in dioxane (1 mL). Formed solids were filtered and washed with CH₂Cl₂. Product was dried in vacuum oven at 40° C. to give 0.071 g of 6-((S)-1-(((S)-2,3-dihydrobenzo[b][1,4]-dioxin-2-yl)methyl)piperidin-3-yl)-5H-pyrrolo[3,4-b]pyridin-7(6H)-one, hydrochloride.

¹H NMR (400 MHz, CDCl₃) δ ppm 1.95-2.16 (m, 2H), 2.30-2.68 (m, 2H), 2.93-3.13 (m, 1H), 3.18-3.34 (m, 1H), 3.35-3.59 (m, 2H), 4.02 (br s, 1H), 4.11-4.21 (m, 1H), 4.24-4.32 (m, 1H), 4.32-4.46 (m, 1H), 4.46-4.69 (m, 2H), 5.23 (br s, 1H), 6.79-6.94 (m, 4H), 7.49 (dd, 1H), 7.87 (dd, 1H), 8.80 (dd, 1H), 13.00 (br s, 1H).

### EXAMPLE 78: 6-((S)-1-(((S)-2,3-Dihydrobenzo[b][1,4]dioxin-2-yl)methyl)piperidin-3-yl)-5H-pyrrolo[3,4-d]pyrimidin-7(6H)-one

### Step 1: (4-Chloro-2-(methylthio)pyrimidin-5-yl)methanol

To a stirred solution of ethyl 4-chloro-2-(methylthio)pyrimidine-5-carboxylate (20.0 g, 86.20 mmol) in CH₂Cl₂ (400 mL) was added 1 M diisobutylaluminum hydride in toluene (172.0 mL, 172.41 mmol) at −78° C. slowly and the resulting reaction mixture was allowed to stir at 0° C. for 2 h. The reaction mixture was quenched with 20% aqueous sodium potassium tartrate solution (800 mL) and then EtOAc (800 mL) was added. The organic layer was separated and the aqueous layer was extracted with EtOAc (2×200 mL). The combined organic extracts were dried over Na₂SO₄, filtered and concentrated under reduced pressure. Crude product was purified with silica gel chromatography using 20% EtOAc/petroleum ether as eluent afforded 4.5 g of (4-chloro-2-(methylthio)pyrimidin-5-yl)methanol as gummy liquid.

LC-MS (ES+) [M+1]: 191.0.

### Step 2: (S)-tert-Butyl-3-(((4-chloro-2-(methylthio)pyrimidin-5-yl)methyl)amino)-piperidine-1-carboxylate

To a stirred solution (4-chloro-2-(methylthio)pyrimidin-5-yl)methanol (4.3 g, 22.63 mmol) in CH₂Cl₂ (45 mL) were added triethylamine (7.95 mL, 56.57 mmol) and methanesulfonyl chloride (2.1 mL, 27.15 mmol) at 0° C. and stirred at room temperature for 2 h. The reaction mixture was diluted with CH₂Cl₂ (50 mL) and washed with water (50 mL), brine (50 mL). The organic layer was dried over anhydrous Na₂SO₄, filtered and concentrated to give 3.9 g of crude product as oil. This material was used as such in the next step.

### Step 3: (S)-tert-Butyl-3-(((4-chloro-2-(methylthio)pyrimidin-5-yl)methyl)amino)-piperidine-1-carboxylate

To a stirred solution of crude product from step 2 (3.9 g) and (S)-tert-butyl 3-aminopiperidine-1-carboxylate (2.92 g, 14.51 mmol) in CH₃CN (40 mL) was added K₂CO₃ (5.03 g, 36.28 mmol) at room temperature and reaction was stirred for 16 h. The reaction was concentrated under reduced pressure and the residue was dissolved in mixture of EtOAc (100 mL) and water (100 mL). The organic layer was separated and dried over anhydrous Na₂SO₄ and concentrated. Crude product was purified with silica gel chromatography using 30% EtOAc/petroleum ether as eluent afforded 2.7 g of (S)-tert-butyl 3-(((4-chloro-2-(methylthio)pyrimidin-5-yl)methyl)amino)piperidine-1-carboxylate as sticky mass.

¹H NMR (400 MHz, DMSO-d₆) δ ppm 1.38 (s, 9H), 2.63-2.69 (m, 1H), 2.82-2.91 (m, 1H), 3.55-3.68 (br s, 1H), 3.74-3.80 (m, 2H), 8.63 (s, 1H).

### Step 4: (S)-tert-Butyl-3-(2-(methylthio)-7-oxo-5H-pyrrolo[3,4-d]pyrimidin-6(7H)-yl)piperidine-1-carboxylate

Into a steel bomb was added (S)-tert-butyl 3-(((4-chloro-2-(methylthio)pyrimidin-5-yl)methyl)amino)-piperidine-1-carboxylate (2.7 g, 7.26 mmol), sodium acetate (1.19 g, 14.52 mmol) and PdCl₂(dppf)*CH₂Cl₂ (296 mg, 0.362 mmol) in ethanol (80 mL). Reaction was heated to 140° C. under CO gas (500 psi) for 16 h. The reaction mixture was concentrated under reduced pressure and the residue was dissolved in EtOAc (100 mL). Organic phase was washed with water (2×100 mL), brine (100 mL), dried over anhydrous Na₂SO₄, filtered and concentrated. Crude product was purified with silica gel chromatography using 70% EtOAc/petroleum ether as eluent to afford 1.6 g of (S)-tert-butyl 3-(2-(methylthio)-7-oxo-5H-pyrrolo[3,4-d]pyrimidin-6(7H)-yl)piperidine-1-carboxylate as yellow solid.

¹H NMR (400 MHz, DMSO-d₆) δ ppm 1.4 (s, 9H), 1.72-1.83 (m, 2H), 1.86-1.95 (m, 1H), 2.6 (s, 3H), 2.68-2.82 (m, 1H), 2.85-3.11 (m, 1H), 3.83-3.94 (m, 1H), 3.95-4.09 (m, 2H), 4.50 (s, 2H), 8.99 (s, 1H).

### Step 5: (S)-tert-Butyl-3-(7-oxo-5H-pyrrolo[3,4-d]pyrimidin-6(7H)-yl)piperidine-1-carboxylate

To a suspension of (S)-tert-butyl 3-(2-(methylthio)-7-oxo-5H-pyrrolo[3,4-d]pyrimidin-6(7H)-yl)piperidine-1-carboxylate (400 mg, 1.09 mmol) and Pd/C (150 mg) in THF was added triethyl silane (0.52 mL, 3.29 mmol) at 0° C. The reaction mixture was stirred at room temperature for 16 h. The reaction mixture was filtered through celite pad and the filtrate was concentrated under reduced pressure. Crude product was purified with silica gel chromatography using 90% EtOAc/petroleum ether as eluent to afford 0.140 g of (S)-tert-butyl 3-(7-oxo-5H-pyrrolo[3,4-d]pyrimidin-6(7H)-yl)piperidine-1-carboxylate as off white solid.

LC-MS (ES+) [M-Boc+1]: 219.2.

### Step 6: (S)-6-(Piperidin-3-yl)-5H-pyrrolo[3,4-d]pyrimidin-7(6H)-one, hydrochloride

A solution of (S)-tert-butyl 3-(7-oxo-5H-pyrrolo[3,4-d]pyrimidin-6(7H)-yl)piperidine-1-carboxylate (140 mg, 0.408 mmol) in 4 M HCl dioxane (5.0 mL) was allowed to stir at room temperature for 3 h. After completion of starting material, the reaction mixture was concentrated under reduced pressure. The crude product was purified by triturating with n-pentane to give 84 mg of (S)-6-(piperidin-3-yl)-5H-pyrrolo[3,4-d]pyrimidin-7(6H)-one, hydrochloride as off white solid.

LC-MS (ES+) [M+1]: 219.2.

### Step 7: 6-((S)-1-(((S)-2,3-Dihydrobenzo[b][1,4]dioxin-2-yl)methyl)piperidin-3-yl)-5H-pyrrolo[3,4-d]pyrimidin-7(6H)-one

A sealed tube was charged with (S)-6-(piperidin-3-yl)-5H-pyrrolo[3,4-d]pyrimidin-7(6H)-one, hydrochloride (0.084 g, 0.330 mmol), (R)-(2,3-dihydrobenzo[b][1,4]dioxin-2-yl)methyl 4-methylbenzenesulfonate (0.148 g, 0.462 mmol), potassium carbonate (0.133 g, 0.962 mmol) and acetonitrile (3 mL). Tube was sealed and reaction was heated with microwave irradiation to 120° C. for 4 hours. Mixture was cooled, filtered and washed with acetonitrile. Solvents were evaporated to dryness. Residue was purified with silica gel chromatography using 5% MeOH/CH₂Cl₂ to give 0.019 g of product as solid. This material was triturated with cold ether and filtered. Product was dried in vacuum oven at 40° C. to give 0.017 g of 6-((S)-1-(((S)-2,3-dihydrobenzo[b][1,4]dioxin-2-yl)methyl)piperidin-3-yl)-5H-pyrrolo[3,4-d]pyrimidin-7(6H)-one.

¹H NMR (400 MHz, CDCl₃) δ ppm 1.63-1.87 (m, 3H), 1.88-1.98 (m, 1H), 2.31-2.41 (m, 1H), 2.41-2.51 (m, 1H), 2.62-2.75 (m, 2H), 2.76-2.86 (m, 1H), 2.99-3.07 (m, 1H), 4.03 (dd, 1H), 4.26-4.34 (m, 2H), 4.52-4.64 (m, 3H), 6.79-6.91 (m, 4H), 8.99 (s, 1H), 9.44 (s, 1H).

### EXAMPLE 79: 5-((S)-1-(((S)-2,3-Dihydrobenzo[b][1,4]dioxin-2-yl)methyl)piperidin-3-yl)-4H-pyrrolo[3,4-d]thiazol-6(5H)-one

### Step 1: Ethyl-2-bromo-4-(bromomethyl)thiazole-5-carboxylate

To a stirred solution of ethyl 2-bromo-4-methylthiazole-5-carboxylate (5.67 g, 22.67 mmol) in tetrachloromethane (50 mL) was added N-bromosuccinimide (4.43 g, 24.93 mmol), benzoyl peroxide (275 mg, 1.13 mmol) at room temperature and then reaction was refluxed for 16 h. The reaction mixture was concentrated under reduced pressure to obtain 6.5 g of crude ethyl 2-bromo-4-(bromomethyl)thiazole-5-carboxylate as light brown solid, which was pure enough to proceed further.

LC-MS (ES+) [M+1]: 327.9.

### Step 2: (S)-Ethyl-4-(((1-((benzyloxy)carbonyl)-piperidin-3-yl)amino)methyl)-2-bromothiazole-5-carboxylate

A flask was charged with ethyl 2-bromo-4-(bromomethyl)-thiazole-5-carboxylate (2.4 g, 7.29 mmol) and dry THF (15.0 mL). Mixture was cooled to 0° C., then 60% NaH in mineral oil (436 mg, 10.93 mmol) was added and reaction was stirred for 30 minutes. To this was added (S)-benzyl 3-aminopiperidine-1-carboxylate (1.7 g, 7.29 mmol) portion wise and reaction was stirred at the same temperature for 3 h. Reaction mixture was diluted with EtOAc (10 mL) and then quenched with ice water. The layers were separated and the aqueous layer was back extracted with EtOAc (30 mL). The combined organic extracts were dried over anhydrous Na₂SO₄ and concentrated under reduced pressure. Crude product was purified with silica gel chromatography using 50% EtOAc/petroleum ether to afford 0.842 g of (S)-ethyl 4-(((1-((benzyloxy)carbonyl)-piperidin-3-yl)amino)methyl)-2-bromothiazole-5-carboxylate as yellow liquid.

LC-MS (ES+) [M+1]: 482.1.

### Step 3: (S)-4-(((1-((Benzyloxy)carbonyl)piperidin-3-yl)amino)methyl)-2-bromothiazole-5-carboxylic acid

To a stirred solution of (S)-ethyl 4-(((1-((benzyloxy)carbonyl)-piperidin-3-yl)amino)-methyl)-2-bromothiazole-5-carboxylate (4.0 g, 8.29 mmol) in THF: H₂O (90 mL, 1:1) was added lithium hydroxide (797 mg, 33.19 mmol) and reaction was stirred at room temperature for 16 h. The reaction mixture was concentrated under reduced pressure; the residue was diluted with water (10 mL) and acidified with 2 N aqueous HCl (pH ˜5). The aqueous layer was extracted with EtOAc (2×40 mL). Combined organic extracts were dried over anhydrous Na₂SO₄ and concentrated under reduced pressure to give 3.3 g of (S)-4-(((1-((benzyloxy)carbonyl)piperidin-3-yl)amino)methyl)-2-bromothiazole-5-carboxylic acid as yellow solid.

LC-MS (ES+) [M+1]: 454.1.

### Step 4: (S)-Benzyl-3-(2-bromo-6-oxo-4H-pyrrolo[3,4-d]thiazol-5(6H)-yl)piperidine-1-carboxylate

To a stirred solution of (S)-4-(((1-((benzyloxy)carbonyl)piperidin-3-yl)amino)methyl)-2-bromothiazole-5-carboxylic acid (7.0 g, 15.41 mmol) in CH₂Cl₂ (100 mL) was added Et₃N (4.3 mL, 30.82 mmol) and benzotriazol-1-yl-oxytripyrrolidinophosphonium hexafluorophosphate (8.0 g, 15.41 mmol) at room temperature and then reaction was heated to 50° C. for 36 h. Reaction mixture was diluted with water (50 mL) and extracted with CH₂Cl₂ (2×60 mL). The combined organic layers were dried over anhydrous Na₂SO₄ and concentrated to obtain the crude compound. Crude product was purified with silica gel chromatography using 65% EtOAc/petroleum ether to afford 3.9 g of (S)-benzyl 3-(2-bromo-6-oxo-4H-pyrrolo[3,4-d]thiazol-5(6H)-yl)piperidine-1-carboxylate as off white solids.

LC-MS (ES+) [M+1]: 436.1.

### Step 5: (S)-Benzyl-3-(6-oxo-4H-pyrrolo[3,4-d]thiazol-5(6H)-yl)piperidine-1-carboxylate

A mixture of (S)-benzyl 3-(2-bromo-6-oxo-4H-pyrrolo[3,4-d]thiazol-5(6H)-yl)piperidine-1-carboxylate (2.8 g, 6.41 mmol), 10% Pd/C (1.5 g) and Na₂CO₃ (1.7 g, 16.04 mmol) in MeOH (110 mL) was hydrogenated under 50 psi at room temperature for 36 h. The reaction mixture was filtered through a pad of celite and washed with 10% methanol in EtOAc; filtrate was concentrated under reduced pressure to obtain the crude compound. Crude product was purified with silica gel chromatography using 70% EtOAc/petroleum ether to afford 0.750 g of (S)-benzyl 3-(6-oxo-4H-pyrrolo[3,4-d]thiazol-5(6H)-yl)piperidine-1-carboxylate as brown solid.

LC-MS (ES+) [M+1]: 358.1.

### Step 6: (S)-5-(Piperidin-3-yl)-4H-pyrrolo[3,4-d]thiazol-6(5H)-one, trifluoroacetate

A mixture of (S)-benzyl 3-(6-oxo-4H-pyrrolo[3,4-d]thiazol-5 (6H)-yl)piperidine-1-carboxylate (500 mg, 1.39 mmol) and TFA (2.1 mL, 27.8 mmol) was heated to 80° C. for 6 h. Solvent was evaporated under reduced pressure to obtain the crude compound. Crude products was purified by triturating with Et₂O and petroleum ether (1:1) to give 0.325 g of (S)-5-(piperidin-3-yl)-4H-pyrrolo[3,4-d]thiazol-6(5H)-one, trifluoroacetate as off white solids.

LC-MS (ES+) [M+1]: 224.2.

### Step 7: 5-((S)-1-(((S)-2,3-Dihydrobenzo[b][1,4]dioxin-2-yl)methyl)piperidin-3-yl)-4H-pyrrolo[3,4-d]thiazol-6(5H)-one

A sealed tube was charged with (S)-5-(piperidin-3-yl)-4H-pyrrolo[3,4-d]thiazol-6(5H)-one, trifluoroacetate (0.10 g, 0.297 mmol), (R)-(2,3-dihydrobenzo[b][1,4]dioxin-2-yl)methyl 4-methylbenzenesulfonate (0.172 g, 0.537 mmol), potassium carbonate (0.155 g, 1.120 mmol) and acetonitrile (3 mL). Tube was sealed and reaction was heated with microwave irradiation to 120° C. for 5 hours. Mixture was cooled, filtered and washed with acetonitrile. Solvents were evaporated to dryness. Residue was purified with silica gel chromatography using EtOAc/heptanes to give 0.070 g of 5-((S)-1-(((S)-2,3-dihydrobenzo[b][1,4]dioxin-2-yl)methyl)piperidin-3-yl)-4H-pyrrolo[3,4-d]thiazol-6(5H)-one.

¹H NMR (400 MHz, CDCl₃) δ ppm 1.50-1.65 (m, 1H), 1.67-1.86 (m, 2H), 1.88-1.99 (m, 1H), 2.21-2.38 (m, 2H), 2.60-2.75 (m, 2H), 2.79-2.90 (m, 1H), 3.01-3.12 (m, 1H), 4.02 (dd, 1H), 4.26-4.35 (m, 2H), 4.36-4.46 (m, 1H), 4.50 (d, 2H), 6.77-6.92 (m, 4H), 9.40 (s, 1H).

### EXAMPLE 80: 5-((S)-1-(((S)-2,3-Dihydrobenzo[b][1,4]dioxin-2-yl)methyl)piperidin-3-yl)-1-methyl-5,6-dihydropyrrolo[3,4-c]pyrazol-4(1H)-one

### Step 1: Methyl-5-formyl-1-methyl-1H-pyrazole-4-carboxylate

To a stirred solution of LDA (2.0 M in THF) (10.7 mL, 21.42 mmol) in THF (10.0 mL) was added methyl 1-methyl-1H-pyrazole-4-carboxylate (1.0 g, 7.14 mmol) in THF (10.0 mL) at −78° C. slowly and stirred for 2 h. Dimethylformamide (2.5 mL, 32.84 mmol) was added to the reaction mixture at −78° C. and then allowed to stir at 0° C. for 2 h. The reaction mixture was quenched with aqueous 1 M HCl solution (10 mL) and extracted with EtOAc (3×10 mL). The combined organic extracts were dried over anhydrous Na₂SO₄, filtered and concentrated under reduced pressure. Crude product was purified with silica gel chromatography using 20% EtOAc/petroleum ether to afford 0.200 g of methyl 5-formyl-1-methyl-1H-pyrazole-4-carboxylate as pale yellow solid.

¹H NMR (400 MHz, CDCl₃) δ ppm 3.91 (s, 3H), 4.20 (s, 3H), 7.91 (s, 1H), 10.50 (s, 1H).

### Step 2: (S)-Benzyl-3-(((4-(methoxycarbonyl)-1-methyl-1H-pyrazol-5-yl)methyl)amino)-piperidine-1-carboxylate

To a cold stirred solution of methyl 5-formyl-1-methyl-1H-pyrazole-4-carboxylate (600 mg, 3.57 mmol) and (S)-benzyl 3-aminopiperidine-1-carboxylate (835 mg, 3.57 mmol) in dichloroethane (20 mL) was added NaHB(OAc)₃ (1.89 g, 8.93 mmol) at 0° C. The resulting reaction mixture was stirred at room temperature for 16 h. The reaction mixture was diluted with CH₂Cl₂ (50 mL), washed with water (50 mL), brine (50 mL), dried over anhydrous Na₂SO₄, filtered and concentrated to afford 1.3 g of crude (S)-benzyl 3-(((4-(methoxycarbonyl)-1-methyl-1H-pyrazol-5-yl)methyl)amino)piperidine-1-carboxylate as sticky mass.

LC-MS (ES+) [M+1]: 387.2.

### Step 3: (S)-5-(((1-((Benzyloxy)carbonyl)piperidin-3-yl)amino)methyl)-1-methyl-1H-pyrazole-4-carboxylic acid

To a stirred solution of (S)-benzyl 3-(((4-(methoxycarbonyl)-1-methyl-1H-pyrazol-5-yl)methyl)amino)-piperidine-1-carboxylate (1.3 g, 3.36 mmol) in THF (13 mL) and water (13 mL) was added LiOH.H₂O (706 mg, 16.83 mmol) at room temperature and stirred for 16 h. The reaction was concentrated under reduced pressure and the residue was dissolved in water (100 mL). The aqueous layer was extracted with EtOAc (20 mL) and the organic layer was discarded. The pH of the aqueous layer was adjusted to 7 and extracted into 15% MeOH in CH₂Cl₂. Organic extracts were dried over anhydrous Na₂SO₄ and concentrated to afford 1.0 g of (S)-5-(((1-((benzyloxy)carbonyl)piperidin-3-yl)amino)methyl)-1-methyl-1H-pyrazole-4-carboxylic acid as off white solids.

LC-MS (ES+) [M+1]: 373.2.

### Step 4: (S)-Benzyl-3-(1-methyl-4-oxopyrrolo[3,4-c]pyrazol-5(1H,4H,6H)-yl)piperidine-1-carboxylate

To a solution of (S)-5-(((1-((benzyloxy)carbonyl)piperidin-3-yl)amino)methyl)-1-methyl-1H-pyrazole-4-carboxylic acid (1.0 g, 2.688 mmol) in CHCl₃ (20 mL) was added NEt₃ (0.75 mL, 5.376 mmol) and benzotriazol-1-yl-oxytripyrrolidinophosphonium hexafluorophosphate (2.09 g, 4.03 mmol) at room temperature The reaction mixture was heated to 70° C. for 16 h. The reaction mixture cooled and washed with water (2×20 mL) and brine (20 mL). The organic layer was dried over anhydrous Na₂SO₄, filtered and concentrated. Crude product was purified with silica gel chromatography using MeOH in CH₂Cl₂ as eluent to give 0.800 g of (S)-benzyl 3-(1-methyl-4-oxopyrrolo[3,4-c]pyrazol-5 (1H,4H,6H)-yl)piperidine-1-carboxylate as sticky solids.

LC-MS (ES+) [M+1]: 355.2.

### Step 5: (S)-1-Methyl-5-(piperidin-3-yl)-5,6-dihydropyrrolo[3,4-c]pyrazol-4(1H)-one

To a solution of (S)-benzyl 3-(1-methyl-4-oxopyrrolo[3,4-c]pyrazol-5(1H,4H,6H)-yl)piperidine-1-carboxylate (700 mg, 1.977 mmol) in EtOAc (20 mL) was added 10% Pd/C and subjected to hydrogenation at 15 Psi (H₂ gas) for 16 h. The reaction mixture was filtered through celite pad and cake was washed with EtOAc. The combined filtrate was concentrated under reduced pressure to obtain crude product. Trituration with diethyl ether yielded 0.250 g of (S)-1-methyl-5-(piperidin-3-yl)-5,6-dihydropyrrolo[3,4-c]pyrazol-4(1H)-one as off white solids.

LC-MS (ES+) [M+1]: 221.2.

### Step 6: 5-((S)-1-(((S)-2,3-Dihydrobenzo[b][1,4]dioxin-2-yl)methyl)piperidin-3-yl)-1-methyl-5,6-dihydropyrrolo[3,4-c]pyrazol-4(1H)-one

A sealed tube was charged with (S)-1-methyl-5-(piperidin-3-yl)-5,6-dihydropyrrolo[3,4-c]pyrazol-4(1H)-one (0.10 g, 0.454 mmol), (R)-(2,3-dihydrobenzo[b][1,4]dioxin-2-yl)methyl 4-methylbenzenesulfonate (0.175 g, 0.545 mmol), potassium carbonate (0.157 g, 1.135 mmol) and acetonitrile (3 mL). Tube was sealed and reaction was heated with microwave irradiation to 120° C. for 7 hours. Mixture was cooled, filtered and washed with acetonitrile. Solvents were evaporated to dryness. Residue was purified with silica gel chromatography using EtOAc/heptanes and MeOH/EtOAc to give 0.120 g of material. This material was evaporated dry with ether to solidify product. Product was dried in vacuum to give 0.090 g of 5-((S)-1-(((S)-2,3-dihydrobenzo[b][1,4]dioxin-2-yl)methyl)piperidin-3-yl)-1-methyl-5,6-dihydropyrrolo[3,4-c]pyrazol-4(1H)-one.

¹H NMR (400 MHz, CDCl₃) δ ppm 1.46-1.59 (m, 1H), 1.66-1.82 (m, 2H), 1.83-1.93 (m, 1H), 2.20-2.35 (m, 2H), 2.60-2.72 (m, 2H), 2.75-2.85 (m, 1H), 2.95-3.04 (m, 1H), 3.86 (s, 3H), 4.03 (dd, 1H), 4.24-4.40 (m, 5H), 6.79-6.90 (m, 4H), 7.63 (s, 1H).

### EXAMPLE 81: 2-((S)-1-(((S)-2,3-Dihydrobenzo[b][1,4]dioxin-2-yl)methyl)piperidin-3-yl)-2,3-dihydrobenzo[d]isothiazole 1,1-dioxide

### Step 1: (S)-tert-Butyl-3-(methylamino)piperidine-1-carboxylate

To a stirred solution of aqueous formaldehyde (1.85 ml, 25 mmol, 37%) and molecular sieves in methanol was added (S)-tert-butyl 3-aminopiperidine-1-carboxylate (5.0 g, 25 mmol) and reaction was stirred at room temperature for 24 h. Sodium borohydride (1.52 g, 40 mmol) was added to the above mixture at room temperature and mixture was stirred for 16 h. The reaction mixture was quenched with addition of ice water (30 mL) and then extracted with EtOAc (3×100 mL). Combined organic extracts were dried over anhydrous Na₂SO₄ and concentrated to obtain 3.0 g of crude (S)-tert-butyl 3-(methylamino)piperidine-1-carboxylate as pale yellow liquid. This material was used as such in the next step.

¹H NMR (400 MHz, CDCl₃) δ ppm 1.38 (s, 9H), 1.5-1.7 (m, 4H), 1.71-1.90 (m, 2H), 2.18-2.27 (m, 3H), 2.75-2.90 (m, 1H), 3.50-3.92 (m, 2H).

### Step 2: (S)-tert-Butyl-3-(2-bromo-N-methylphenyl-sulfonamido)piperidine-1-carboxylate

To an ice cold stirred solution of crude (S)-tert-butyl 3-(methylamino)piperidine-1-carboxylate (3.0 g, 14 mmol) in CH₂Cl₂ (100 mL) was added Et₃N (2.1 mL, 15.4 mmol), DMAP (342 mg, 2.8 mmol) and 2-bromobenzene-1-sulfonyl chloride (3.9 g, 15.4 mmol) and stirred at rt for 16 h. The reaction mixture was diluted with CH₂Cl₂ (100 mL) and washed with water. The organic layer was dried over anhydrous Na₂SO₄ and concentrated under reduced pressure. Crude product was purified with silica gel chromatography using 10% EtOAc/petroleum ether to afford 1.2 g of(S)-tert-butyl 3-(2-bromo-N-methylphenyl-sulfonamido)piperidine-1-carboxylate as pale yellow thick mass.

LC-MS (ES+) [M+1]: 433.5.

### Step 3: (S)-tert-Butyl-3-(1,1-dioxidobenzo[d]isothiazol-2(3H)-yl)piperidine-1-carboxylate

To a solution of (S)-tert-butyl 3-(2-bromo-N-methylphenyl-sulfonamido)piperidine-1-carboxylate (1.2 g, 2.7 mmol) in mesitylene (80 mL) was added Cs₂CO₃ (1.35 g, 4.1 mmol) and degassed with argon for 20 minutes. Pd(OAc)₂ (31 mg, 0.13 mmol), PCy₃.HBF₄ (101 mg, 0.27 mmol) and pivalic acid (84 mg, 0.8 mmol) were added to the above mixture then further degassed for 20 minutes. The above mixture was heated at 150° C. in sealed tube for 16 h. The reaction mixture was cooled to room temperature and diluted with EtOAc (80 mL) and washed with water. The organic layer was dried over anhydrous Na₂SO₄ and concentrated under reduced pressure. Crude product was purified with silica gel chromatography using 20% EtOAc/petroleum ether to afford 0.400 g of (S)-tert-butyl 3-(1,1-dioxidobenzo[d]isothiazol-2(3H)-yl)piperidine-1-carboxylate as pale yellow thick mass.

LC-MS (ES+) [M+1]: 353.2.

### Step 4: (S)-2-(Piperidin-3-yl)-2,3-dihydrobenzo[d]isothiazole 1,1-dioxide, hydrochloride

A mixture of (S)-tert-butyl 3-(1,1-dioxidobenzo[d]isothiazol-2(3H)-yl)piperidine-1-carboxylate (700 mg, 1.98 mmol) and 3 M HCl in dioxane (15 mL) was stirred at room temperature for 1 h. Solvent was evaporated under reduced pressure to obtain the crude compound. Crude products was purified by triturating with n-pentane of afford 0.500 g of (S)-2-(piperidin-3-yl)-2,3-dihydrobenzo[d]isothiazole 1,1-dioxide, hydrochloride as off white solids.

LC-MS (ES+) [M+1]: 253.0.

### Step 5: 2-((S)-1-(((S)-2,3-Dihydrobenzo[b][1,4]dioxin-2-yl)methyl)piperidin-3-yl)-2,3-dihydrobenzo[d]isothiazole 1,1-dioxide

A sealed tube was charged with (S)-2-(piperidin-3-yl)-2,3-dihydrobenzo[d]isothiazole 1,1-dioxide, hydrochloride (0.127 g, 0.441 mmol), (2R)-2-(bromomethyl)-2,3-dihydro-1,4-benzodioxin (0.138 g, 0.604 mmol), potassium carbonate (0.139 g, 1.007 mmol) and acetonitrile (3 mL). Tube was sealed and reaction was heated with microwave irradiation to 120° C. for 4 hours. Mixture was cooled, filtered and washed with acetonitrile. Solvents were evaporated to dryness. Residue was purified with silica gel chromatography using EtOAc/heptanes to give 0.128 g of 2-((S)-1-(((S)-2,3-dihydrobenzo[b][1,4]dioxin-2-yl)methyl)piperidin-3-yl)-2,3-dihydrobenzo[d]isothiazole-1,1-dioxide.

¹H NMR (400 MHz, DMSO-d₆) δ ppm 1.52-1.71 (m, 2H), 1.71-1.81 (m, 1H), 1.90-1.97 (m, 1H), 2.13-2.25 (m, 1H), 2.42 (dd, 1H), 2.64 (d, 2H), 2.74-2.83 (m, 1H), 3.07-3.15 (m, 1H), 3.57-3.69 (m, 1H), 3.96 (dd, 1H), 4.24-4.41 (m, 2H), 4.49-4.64 (m, 2H), 6.75-6.91 (m, 4H), 7.53-7.64 (m, 2H), 7.71 (ddd, 1H), 7.84 (d, 1H).

### EXAMPLE 82: 1-((S)-1-(((S)-2,3-Dihydrobenzo[b][1,4]dioxin-2-yl)methyl)piperidin-3-yl)-6-fluoro-1H-benzo[d]imidazole

### Step 1: (S)-1-(((S)-2,3-Dihydrobenzo[b][1,4]-dioxin-2-yl)methyl)-N-(5-fluoro-2-nitrophenyl)piperidin-3-amine

A flask was charged with (S)-1-(((S)-2,3-dihydrobenzo[b][1,4]dioxin-2-yl)methyl)piperidin-3-amine (0.40 g, 1.611 mmol), 2,4-difluoronitrobenzene (0.282 g, 1.772 mmol), potassium carbonate (0.267 g, 1.933 mmol) and dry DMF (2 mL). Reaction was heated to 60° C. for 4 hours. Mixture was cooled to room temperature and partitioned between water (10 mL) and EtOAc (10 mL). Aqueous phase was extracted with EtOAc (5 mL). Combined organic extracts were washed with brine, dried with anhydrous Na₂SO₄ and evaporated to dryness. Crude product was purified with silica gel chromatography using 5-100% EtOAc/heptanes to give 0.537 g of (S)-1-(((S)-2,3-dihydrobenzo[b][1,4]-dioxin-2-yl)methyl)-N-(5-fluoro-2-nitrophenyl)piperidin-3-amine as yellow oil.

LC-MS (ES+) [M+1]: 388.5.

### Step 2: N1-((S)-1-(((S)-2,3-Dihydrobenzo[b][1,4]dioxin-2-yl)methyl)piperidin-3-yl)-5-fluoro-benzene-1,2-diamine

A flask was charged with (S)-1-(((S)-2,3-dihydrobenzo[b][1,4]dioxin-2-yl)methyl)-N-(5-fluoro-2-nitrophenyl)piperidin-3-amine (0.533 g, 1.376 mmol), ammonium chloride (0.736 g, 13.76 mmol), THF (4 mL), MeOH (2 mL) and water (2 mL). To this was added zinc powder (0.900 g, 13.76 mmol) and reaction was stirred at room temperature for 1 h. Mixture was filtered through celite and washed with EtOAc (10 mL) and water (5 mL). Phases were separated and aqueous phase was extracted with EtOAc (10 mL). Combined organic extracts were washed with brine, dried with anhydrous Na₂SO₄ and evaporated to dryness to give 0.487 g of N1-((S)-1-(((S)-2,3-dihydrobenzo[b][1,4]dioxin-2-yl)methyl)piperidin-3-yl)-5-fluoro-benzene-1,2-diamine as red oil.

LC-MS (ES+) [M+1]: 358.1.

### Step 3: 1-((S)-1-(((S)-2,3-Dihydrobenzo[b][1,4]dioxin-2-yl)methyl)piperidin-3-yl)-6-fluoro-1H-benzo[d]imidazole

A flask was charged with N1-((S)-1-(((S)-2,3-dihydrobenzo[b][1,4]dioxin-2-yl)methyl)piperidin-3-yl)-5-fluoro-benzene-1,2-diamine (0.164 g, 0.459 mmol), formic acid (2 mL) and then heated to 70-80° C. for 2 h. Reaction was cooled to room temperature and solvent was evaporated. Residue was partitioned between EtOAc (10 mL) and 2 M NaOH solution (10 mL). Aqueous phase was extracted with EtOAc (10 mL). Combined organic extracts were washed with brine, dried with anhydrous Na₂SO₄ and evaporated to dryness. Crude product was purified with reverse phase chromatography using 10-100% MeCN/0.1% NH₄OH buffer to give 0.120 g of 1-((S)-1-(((S)-2,3-dihydrobenzo[b][1,4]-dioxin-2-yl)methyl)piperidin-3-yl)-6-fluoro-1H-benzo[d]imidazole as glassy solids.

¹H NMR (400 MHz, CDCl₃) δ ppm 1.69-2.01 (m, 3H), 2.05-2.16 (m, 1H), 2.52-2.62 (m, 1H), 2.67 (dd, 1H), 2.73-2.87 (m, 3H), 3.12-3.21 (m, 1H), 4.02 (dd, 1H), 4.30 (dd, 1H), 4.33-4.50 (m, 2H), 6.78-6.96 (m, 4H), 7.02 (ddd, 1H), 7.10 (dd, 1H), 7.73 (dd, 1H), 8.32 (br s, 1H).

### EXAMPLE 83: 1-((S)-1-(((S)-2,3-Dihydrobenzo[b][1,4]dioxin-2-yl)methyl)piperidin-3-yl)-6-fluoro-2-methyl-1H-benzo[d]imidazole

A flask was charged with N1-((S)-1-(((S)-2,3-dihydrobenzo[b][1,4]dioxin-2-yl)methyl)-piperidin-3-yl)-5-fluoro-benzene-1,2-diamine (0.164 g, 0.459 mmol), acetic anhydride (0.048 mL, 0.505 mmol), acetic acid (2 mL) and then heated to reflux for 7.5 h. Reaction was cooled to room temperature and solvent was evaporated. Residue was partitioned between EtOAc (10 mL) and 2 M NaOH solution (10 mL). Aqueous phase was extracted with EtOAc (10 mL). Combined organic extracts were washed with brine, dried with anhydrous Na₂SO₄ and evaporated to dryness. Crude product was purified with reverse phase chromatography using 10-100% MeCN/0.1% NH₄OH buffer to give 0.127 g of 1-((S)-1-(((S)-2,3-dihydrobenzo[b][1,4]dioxin-2-yl)methyl)piperidin-3-yl)-6-fluoro-2-methyl-1H-benzo[d]imidazole as solid.

¹H NMR (400 MHz, CDCl₃) δ ppm 1.74-1.88 (m, 1H), 1.88-2.04 (m, 2H), 2.16 (ddd, 1H), 2.30 (dt, 1H), 2.62 (s, 3H), 2.66-2.81 (m, 2H), 2.81-2.89 (t, 1H), 3.00-3.08 (m, 1H), 3.08-3.18 (m, 1H), 3.96-4.06 (m, 1H), 4.25-4.35 (m, 2H), 4.35-4.48 (m, 1H), 6.79-6.90 (m, 4H), 6.95 (ddd, 1H), 7.22 (dd, 1H), 7.58 (dd, 1H).

### EXAMPLE 84: 1-((S)-1-(((S)-2,3-Dihydrobenzo[b][1,4]dioxin-2-yl)methyl)piperidin-3-yl)-2-ethyl-6-fluoro-1H-benzo[d]imidazole

A flask was charged with N1-((S)-1-(((S)-2,3-dihydrobenzo[b][1,4]dioxin-2-yl)methyl)piperidin-3-yl)-5-fluoro-benzene-1,2-diamine (0.15 g, 0.420 mmol), propionic anhydride (0.057 mL, 0.441 mmol), propionic acid (1 mL) and then heated to reflux for 4 h. Reaction was cooled to room temperature and solvent was evaporated. Residue was partitioned between EtOAc (10 mL), saturated Na₂CO₃ solution (10 mL) and some water. Aqueous phase was extracted with EtOAc (10 mL). Combined organic extracts were washed with brine, dried with anhydrous Na₂SO₄ and evaporated to dryness. Crude product was purified with reverse phase chromatography using 10-100% MeCN/0.5% HCO₂H buffer followed by silica gel chromatography with 0-10% MeOH/CH₂Cl₂ to give 0.097 g of 1-((S)-1-(((S)-2,3-dihydrobenzo[b][1,4]dioxin-2-yl)methyl)piperidin-3-yl)-2-ethyl-6-fluoro-1H-benzo[d]imidazole as solid.

¹H NMR (400 MHz, CDCl₃) δ ppm 1.43 (t, 3H), 1.84 (s, 1H), 1.89-2.05 (m, 2H), 2.13-2.27 (m, 1H), 2.27-2.37 (m, 1H), 2.63-2.85 (m, 2H), 2.85-3.00 (m, 3H), 3.01-3.10 (m, 1H), 3.10-3.20 (m, 1H), 4.00 (dd, 1H), 4.25-4.37 (m, 2H), 4.39-4.50 (m, 1H), 6.79-6.90 (m, 4H), 6.97 (ddd, 1H), 7.23 (dd, 1H), 7.65 (dd, 1H).

### EXAMPLE 85: 1-((S)-1-(((S)-2,3-Dihydrobenzo[b][1,4]dioxin-2-yl)methyl)piperidin-3-yl)-6-fluoro-2-isopropyl-1H-benzo[d]imidazole

A flask was charged with N1-((S)-1-(((S)-2,3-dihydrobenzo[b][1,4]dioxin-2-yl)methyl)-piperidin-3-yl)-5-fluoro-benzene-1,2-diamine (0.153 g, 0.428 mmol), isobutyric anhydride (0.075 mL, 0.449 mmol), chlorobenzene (2 mL) and then heated to reflux for 46 h. Reaction was cooled to room temperature. Mixture was partitioned between CH₂Cl₂ (10 mL) and saturated Na₂CO₃ solution (10 mL). Aqueous phase was extracted with CH₂Cl₂ (10 mL). Combined organic extracts were washed with brine, dried with anhydrous Na₂SO₄ and evaporated to dryness. Crude product was purified with silica gel chromatography with 0-10% MeOH/CH₂Cl₂ followed by silica gel chromatography using 30-100% EtOAc/heptanes to give 0.064 g of 1-((S)-1-(((S)-2,3-dihydrobenzo[b][1,4]dioxin-2-yl)methyl)piperidin-3-yl)-6-fluoro-2-isopropyl-1H-benzo[d]imidazole as white solid.

¹H NMR (400 MHz, CDCl₃) δ ppm 1.43 (d, 3H), 1.46 (d, 3H), 1.74-1.88 (m, 1H), 1.89-2.02 (m, 2H), 2.16-2.27 (m, 1H), 2.27-2.40 (m, 1H), 2.66-2.83 (m, 2H), 2.91 (t, 1H), 2.99-3.07 (m, 1H), 3.08-3.15 (m, 1H), 3.17-3.29 (m, 1H), 4.00 (dd, 1H), 4.23-4.35 (m, 2H), 4.41-4.53 (m, 1H), 6.79-6.89 (m, 4H), 6.95 (ddd, 1H), 7.23 (dd, 1H), 7.65 (dd, 1H).

### EXAMPLE 86: 1-((S)-1-(((S)-2,3-Dihydrobenzo[b][1,4]dioxin-2-yl)methyl)piperidin-3-yl)-6-fluoro-1H-benzo[d]imidazol-2(3H)-one

A flask was charged with N1-((S)-1-(((S)-2,3-dihydrobenzo[b][1,4]dioxin-2-yl)methyl)-piperidin-3-yl)-5-fluoro-benzene-1,2-diamine (0.164 g, 0.459 mmol), 1,1′-carbonyldi-imidazole (0.082 g, 0.505 mmol) and CH₂Cl₂ (2 mL). Reaction was stirred at room temperature for 1 h. Solvent was evaporated, replaced with MeCN (2 mL) and reaction was heated to 60° C. for 2 h. Another batch of 1,1′-carbonyldi-imidazole (0.082 g, 0.505 mmol) was added and reaction was heated at 60° C. for 2 h. Mixture was cooled to room temperature and partitioned between EtOAc (10 mL), water (10 mL) and some saturated NH₄Cl solution. Aqueous phase was extracted with EtOAc (10 mL). Combined organic extracts were washed with brine, dried with anhydrous Na₂SO₄ and evaporated to dryness. Crude product was purified with reverse phase chromatography using 10-100% MeCN/0.1% NH₄OH buffer to give 0.119 g of 1-((S)-1-(((S)-2,3-dihydrobenzo[b][1,4]dioxin-2-yl)methyl)piperidin-3-yl)-6-fluoro-1H-benzo[d]imidazol-2(3H)-one as pink solids.

¹H NMR (400 MHz, CDCl₃) δ ppm 1.71-1.99 (m, 3H), 2.18 (ddd, 1H), 2.28 (dt, 1H), 2.66-2.80 (m, 2H), 2.85 (t, 1H), 2.93-3.02 (m, 1H), 3.02-3.11 (m, 1H), 3.97-4.09 (m, 1H), 4.25-4.36 (m, 2H), 4.36-4.94 (m, 1H), 6.68-6.90 (m, 5H), 6.94 (dd, 1H), 7.01 (dd, 1H), 9.80 (br s, 1H).

### EXAMPLE 87: 1-((S)-1-(((S)-2,3-Dihydrobenzo[b][1,4]dioxin-2-yl)methyl)piperidin-3-yl)-5-fluoro-1H-benzo[d]imidazole

### Step 1: (S)-1-(((S)-2,3-Dihydrobenzo[b][1,4]dioxin-2-yl)methyl)-N-(4-fluoro-2-nitrophenyl)piperidin-3-amine

A flask was charged with (S)-1-(((S)-2,3-dihydrobenzo[b][1,4]dioxin-2-yl)methyl)piperidin-3-amine (0.40 g, 1.611 mmol), 2,5-difluoronitrobenzene (0.282 g, 1.772 mmol), potassium carbonate (0.267 g, 1.933 mmol) and dry DMF (2 mL). Reaction was heated to 60° C. for 3 hours. Mixture was cooled to room temperature and partitioned between water (10 mL) and EtOAc (10 mL). Aqueous phase was extracted with EtOAc (5 mL). Combined organic extracts were washed with brine, dried with anhydrous Na₂SO₄ and evaporated to dryness. Crude product was purified with silica gel chromatography using 5-100% EtOAc/heptanes to give 0.619 g of(S)-1-(((S)-2,3-dihydrobenzo[b][1,4]-dioxin-2-yl)methyl)-N-(4-fluoro-2-nitrophenyl)piperidin-3-amine as orange oil.

LC-MS (ES+) [M+1]: 388.4.

### Step 2: N1-((S)-1-(((S)-2,3-Dihydrobenzo[b][1,4]dioxin-2-yl)methyl)piperidin-3-yl)-4-fluorobenzene-1,2-diamine

A flask was charged with (S)-1-(((S)-2,3-dihydrobenzo[b][1,4]dioxin-2-yl)methyl)-N-(4-fluoro-2-nitrophenyl)piperidin-3-amine (0.589 g, 1.520 mmol), ammonium chloride (0.813 g, 15.20 mmol), THF (4 mL), MeOH (2 mL) and water (2 mL). To this was added zinc powder (0.994 g, 15.20 mmol) and reaction was stirred at room temperature for 1.5 h. Mixture was filtered through celite and washed with EtOAc (10 mL) and water (10 mL). Phases were separated and aqueous phase was extracted with EtOAc (10 mL). Combined organic extracts were washed with brine, dried with anhydrous Na₂SO₄ and evaporated to dryness to give 0.525 g of N1-((S)-1-(((S)-2,3-dihydrobenzo[b][1,4]dioxin-2-yl)methyl)piperidin-3-yl)-4-fluorobenzene-1,2-diamine as red oil.

LC-MS (ES+) [M+1]: 358.6.

### Step 3: 1-((S)-1-(((S)-2,3-Dihydrobenzo[b][1,4]dioxin-2-yl)methyl)piperidin-3-yl)-5-fluoro-1H-benzo[d]imidazole

A flask was charged with N1-((S)-1-(((S)-2,3-dihydrobenzo[b][1,4]dioxin-2-yl)methyl)piperidin-3-yl)-4-fluorobenzene-1,2-diamine (0.181 g, 0.506 mmol), formic acid (2 mL) and then heated to 80° C. for 1.5 h. Reaction was cooled to room temperature and solvent was evaporated. Residue was partitioned between EtOAc (10 mL) and 2 M NaOH solution (10 mL). Aqueous phase was extracted with EtOAc (10 mL). Combined organic extracts were washed with brine, dried with anhydrous Na₂SO₄ and evaporated to dryness. Crude product was purified with reverse phase chromatography using 10-100% MeCN/0.1% NH₄OH buffer to give 0.110 g of 1-((S)-1-(((S)-2,3-dihydrobenzo[b][1,4]-dioxin-2-yl)methyl)piperidin-3-yl)-5-fluoro-1H-benzo[d]imidazole as semisolids.

¹H NMR (400 MHz, CDCl₃) δ ppm 1.68-1.99 (m, 3H), 2.05-2.17 (m, 1H), 2.51-2.62 (m, 1H), 2.66 (dd, 1H), 2.71-2.86 (m, 3H), 3.19 (dd, 1H), 4.02 (dd, 1H), 4.30 (dd, 1H), 4.32-4.39 (m, 1H), 4.42-4.54 (m, 1H), 6.79-6.96 (m, 4H), 7.06 (dt, 1H), 7.33 (dd, 1H), 7.48 (dd, 1H), 8.34 (br s, 1H).

### EXAMPLE 88: 1-((S)-1-(((S)-2,3-Dihydrobenzo[b][1,4]dioxin-2-yl)methyl)piperidin-3-yl)-5-fluoro-1H-benzo[d]imidazol-2(3H)-one

A flask was charged with N1-((S)-1-(((S)-2,3-dihydrobenzo[b][1,4]dioxin-2-yl)methyl)piperidin-3-yl)-4-fluorobenzene-1,2-diamine (0.181 g, 0.506 mmol), 1,1′-carbonyldiimidazole (0.115 g, 0.708 mmol) and MeCN (2 mL). Reaction was stirred sealed at room temperature over weekend. Mixture was cooled to room temperature and partitioned between EtOAc (10 mL), water (10 mL) and some saturated NH₄Cl solution. Aqueous phase was extracted with EtOAc (10 mL). Combined organic extracts were washed with brine, dried with anhydrous Na₂SO₄ and evaporated to dryness. Crude product was purified with reverse phase chromatography using 10-100% MeCN/0.1% NH₄OH buffer to give 0.130 g of 1-((S)-1-(((S)-2,3-dihydrobenzo[b][1,4]-dioxin-2-yl)methyl)piperidin-3-yl)-5-fluoro-1H-benzo[d]imidazol-2(3H)-one as pink solids.

¹H NMR (400 MHz, CDCl₃) δ ppm 1.68-1.99 (m, 3H), 2.12-2.36 (m, 2H), 2.59-2.81 (m, 2H), 2.87 (t, 1H), 2.93-3.01 (m, 1H), 3.02-3.12 (m, 1H), 3.94-4.10 (m, 1H), 4.24-4.37 (m, 2H), 4.37-4.49 (m, 1H), 6.72-6.96 (m, 6H), 7.07 (dd, 1H), 10.39 (s, 1H).

### EXAMPLE 89: 6-Chloro-1-((S)-1-(((S)-2,3-dihydrobenzo[b][1,4]dioxin-2-yl)methyl)piperidin-3-yl)-1H-benzo[d]imidazole

### Step 1: (S)—N-(5-Chloro-2-nitrophenyl)-1-(((S)-2,3-dihydrobenzo[b][1,4]dioxin-2-yl)methyl)piperidin-3-amine

A flask was charged with (S)-1-(((S)-2,3-dihydrobenzo[b][1,4]dioxin-2-yl)methyl)piperidin-3-amine (0.50 g, 2.014 mmol), 2,4-dichloronitrobenzene (0.387 g, 2.014 mmol), potassium carbonate (0.334 g, 2.416 mmol) and dry DMF (2 mL). Reaction was heated to 120° C. for 6 hours. Mixture was cooled to room temperature and partitioned between water (10 mL) and EtOAc (10 mL). Aqueous phase was extracted with EtOAc (10 mL). Combined organic extracts were washed with brine, dried with anhydrous Na₂SO₄ and evaporated to dryness. Crude product was purified with silica gel chromatography using 5-100% EtOAc/heptanes to give 0.461 g of (S)—N-(5-chloro-2-nitrophenyl)-1-(((S)-2,3-dihydrobenzo[b][1,4]dioxin-2-yl)methyl)piperidin-3-amine as yellow oil.

LC-MS (ES+) [M+1]: 404.1.

### Step 2: 5-Chloro-N1-((S)-1-(((S)-2,3-dihydrobenzo[b][1,4]dioxin-2-yl)methyl)piperidin-3-yl)benzene-1,2-diamine

A flask was charged with (S)—N-(5-chloro-2-nitrophenyl)-1-(((S)-2,3-dihydrobenzo[b][1,4]-dioxin-2-yl)methyl)piperidin-3-amine (0.450 g, 1.114 mmol), ammonium chloride (0.596 g, 11.14 mmol), THF (4 mL), MeOH (2 mL) and water (2 mL). To this was added zinc powder (0.729 g, 11.14 mmol) and reaction was stirred at room temperature for 1 h. Mixture was filtered through celite and washed with EtOAc (10 mL) and water (5 mL). Phases were separated and aqueous phase was extracted with EtOAc (10 mL). Combined organic extracts were washed with brine, dried with anhydrous Na₂SO₄ and evaporated to dryness to give 0.454 g of 5-chloro-N1-((S)-1-(((S)-2,3-dihydrobenzo[b][1,4]dioxin-2-yl)methyl)piperidin-3-yl)benzene-1,2-diamine as clear oil.

LC-MS (ES+) [M+1]: 374.1.

### Step 3: 6-Chloro-1-((S)-1-(((S)-2,3-dihydrobenzo[b][1,4]dioxin-2-yl)methyl)piperidin-3-yl)-1H-benzo[d]imidazole

A flask was charged with 5-chloro-N1-((S)-1-(((S)-2,3-dihydrobenzo[b][1,4]dioxin-2-yl)methyl)piperidin-3-yl)benzene-1,2-diamine (0.120 g, 0.321 mmol), formic acid (2 mL) and then heated to 80° C. for 2 h. Reaction was cooled to room temperature and solvent was evaporated. Residue was partitioned between EtOAc (10 mL) and 2 M NaOH solution (10 mL). Aqueous phase was extracted with EtOAc (10 mL). Combined organic extracts were washed with brine, dried with anhydrous Na₂SO₄ and evaporated to dryness. Crude product was purified with silica gel chromatography using 0-10% MeOH/CH₂Cl₂ to give 0.090 g of 6-chloro-1-((S)-1-(((S)-2,3-dihydrobenzo[b][1,4]dioxin-2-yl)methyl)piperidin-3-yl)-1H-benzo[d]imidazole as semisolids.

¹H NMR (400 MHz, CDCl₃) δ ppm 1.69-2.00 (m, 3H), 2.04-2.16 (m, 1H), 2.53-2.63 (m, 1H), 2.63-2.72 (m, 1H), 2.74-2.86 (m, 3H), 3.11-3.20 (m, 1H), 4.02 (dd, 1H), 4.25-4.33 (m, 1H), 4.33-4.40 (m, 1H), 4.41-4.49 (m, 1H), 6.80-6.94 (m, 4H), 7.23-7.27 (m, 1H), 7.42 (d, 1H), 7.72 (dd, 1H), 8.35 (br s, 1H).

### EXAMPLE 90: 6-Chloro-1-((S)-1-(((S)-2,3-dihydrobenzo[b][1,4]dioxin-2-yl)methyl)piperidin-3-yl)-2-methyl-1H-benzo[d]imidazole

A flask was charged with 5-chloro-N1-((S)-1-(((S)-2,3-dihydrobenzo[b][1,4]dioxin-2-yl)methyl)piperidin-3-yl)benzene-1,2-diamine (0.138 g, 0.369 mmol), acetic anhydride (0.037 mL, 0.388 mmol), acetic acid (1 mL) and then heated to reflux for 7 h. Reaction was cooled to room temperature and solvent was evaporated. Residue was partitioned between EtOAc (10 mL) and saturated Na₂CO₃ solution (10 mL). Aqueous phase was extracted with EtOAc (10 mL). Combined organic extracts were washed with brine, dried with anhydrous Na₂SO₄ and evaporated to dryness. Crude product was purified with silica gel chromatography using 0-10% MeOH/CH₂Cl₂ to give 0.077 g of 6-chloro-1-((S)-1-(((S)-2,3-dihydrobenzo[b][1,4]dioxin-2-yl)methyl)piperidin-3-yl)-2-methyl-1H-benzo[d]imidazole as semisolids.

¹H NMR (400 MHz, CDCl₃) δ ppm 1.74-1.89 (m, 1H), 1.89-2.03 (m, 2H), 2.11-2.25 (m, 1H), 2.32 (dt, 1H), 2.61-2.66 (m, 3H), 2.68-2.82 (m, 2H), 2.85 (t, 1H), 3.00-3.08 (m, 1H), 3.08-3.20 (m, 1H), 3.96-4.06 (m, 1H), 4.26-4.36 (m, 2H), 4.35-4.47 (m, 1H), 6.79-6.91 (m, 4H), 7.17 (dd, 1H), 7.50 (d, 1H), 7.58 (d, 1H).

### EXAMPLE 91: 5-Chloro-1-((S)-1-(((S)-2,3-dihydrobenzo[b][1,4]dioxin-2-yl)methyl)piperidin-3-yl)-1H-benzo[d]imidazole

### Step 1: (S)—N-(4-Chloro-2-nitrophenyl)-1-(((S)-2,3-dihydrobenzo[b][1,4]dioxin-2-yl)methyl)piperidin-3-amine

A flask was charged with (S)-1-(((S)-2,3-dihydrobenzo[b][1,4]dioxin-2-yl)methyl)piperidin-3-amine (0.40 g, 1.611 mmol), 2,5-dichloronitrobenzene (0.309 g, 1.611 mmol), potassium carbonate (0.267 g, 1.933 mmol) and dry DMF (2 mL). Reaction was heated to 120° C. for 5 hours. Mixture was cooled to room temperature and partitioned between water (10 mL) and EtOAc (10 mL). Aqueous phase was extracted with EtOAc (10 mL). Combined organic extracts were washed with brine, dried with anhydrous Na₂SO₄ and evaporated to dryness. Crude product was purified with silica gel chromatography using 5-100% EtOAc/heptanes to give 0.235 g of (S)—N-(4-chloro-2-nitrophenyl)-1-(((S)-2,3-dihydrobenzo[b][1,4]dioxin-2-yl)methyl)piperidin-3-amine as orange oil.

LC-MS (ES+) [M+1]: 404.3.

### Step 2: 4-Chloro-N1-((S)-1-(((S)-2,3-dihydrobenzo[b][1,4]dioxin-2-yl)methyl)piperidin-3-yl)benzene-1,2-diamine

A flask was charged with (S)—N-(4-chloro-2-nitrophenyl)-1-(((S)-2,3-dihydrobenzo[b][1,4]-dioxin-2-yl)methyl)piperidin-3-amine (0.230 g, 0.570 mmol), ammonium chloride (0.305 g, 5.70 mmol), THF (2 mL), MeOH (1 mL) and water (1 mL). To this was added zinc powder (0.373 g, 5.70 mmol) and reaction was stirred at room temperature for 0.5 h. Mixture was filtered through celite and washed with EtOAc (10 mL) and water (5 mL). Phases were separated and aqueous phase was extracted with EtOAc (10 mL). Combined organic extracts were washed with brine, dried with anhydrous Na₂SO₄ and evaporated to dryness to give 0.209 g of 4-chloro-N1-((S)-1-(((S)-2,3-dihydrobenzo[b][1,4]dioxin-2-yl)methyl)piperidin-3-yl)benzene-1,2-diamine as clear oil.

LC-MS (ES+) [M+1]: 374.5.

### Step 3: 5-Chloro-1-((S)-1-(((S)-2,3-dihydrobenzo[b][1,4]dioxin-2-yl)methyl)piperidin-3-yl)-1H-benzo[d]imidazole

A flask was charged with 4-chloro-N1-((S)-1-(((S)-2,3-dihydrobenzo[b][1,4]dioxin-2-yl)methyl)piperidin-3-yl)benzene-1,2-diamine (0.208 g, 0.556 mmol), formic acid (2 mL) and then heated to 70° C. for 2 h. Reaction was cooled to room temperature and solvent was evaporated. Residue was partitioned between EtOAc (10 mL) and 1 M NaOH solution (10 mL). Aqueous phase basified by addition of Na₂CO₃ and then extracted with EtOAc (10 mL). Combined organic extracts were washed with brine, dried with anhydrous Na₂SO₄ and evaporated to dryness. Crude product was purified with reverse phase chromatography using 10-100% MeCN/0.1% NH₄OH buffer to give 0.160 g of 5-chloro-1-((S)-1-(((S)-2,3-dihydrobenzo[b][1,4]dioxin-2-yl)methyl)piperidin-3-yl)-1H-benzo[d]imidazole as solids.

¹H NMR (400 MHz, CDCl₃) δ ppm 1.69-1.99 (m, 3H), 2.04-2.16 (m, 1H), 2.52-2.62 (m, 1H), 2.66 (dd, 1H), 2.72-2.86 (m, 3H), 3.18 (dd, 1H), 4.02 (dd, 1H), 4.30 (dd, 1H), 4.32-4.40 (tm, 1H), 4.42-4.52 (m, 1H), 6.80-6.93 (m, 4H), 7.24-7.29 (m, 1H), 7.33 (d, 1H), 7.79 (dd, 1H), 8.35 (br s, 1H).

### EXAMPLE 92: 2-((S)-1-(((S)-2,3-Dihydrobenzo[b][1,4]dioxin-2-yl)methyl)piperidin-3-yl)tetrahydrocyclopenta[c]pyrrole-1,3(2H,3aH)-dione hydrochloride

To a solution of (S)-1-(((S)-2,3-dihydrobenzo[b][1,4]dioxin-2-yl)methyl)piperidin-3-amine (0.30 g, 1.21 mmol) in xylenes (6 ml) was added cyclopentane-1,2-dicarboxylic acid anhydride (0.25 g, 1.8 mmol) and the solution was heated to reflux. After 4 hours the reaction mixture was cooled to rt and 1M HCl (10 ml) was added. Filtration of the precipitate that formed upon addition of HCl afforded 0.33 g of 2-((S)-1-(((S)-2,3-dihydrobenzo[b][1,4]dioxin-2-yl)methyl)piperidin-3-yl)tetrahydrocyclopenta[c]pyrrole-1,3(2H,3aH)-dione hydrochloride as white solid.

¹H NMR (400 MHz, DMSO-d₆) δ ppm 1.15-1.30 (1H, m), 1.63-1.74 (2H, m), 1.75-2.01 (6H, m), 2.03-2.16 (1H, m), 2.92-3.12 (1H, m), 3.13-3.21 (2H, m), 3.38-3.65 (5H, m), 4.04 (1H, dd), 4.36 (1H, dd), 4.44-4.56 (1H, m), 4.86-4.97 (1H, m), 6.85-6.94 (4H, m), 11.35 (1H, br s).

### EXAMPLE 93: (3aR,7aS)-2-((S)-1-(((S)-2,3-Dihydrobenzo[b][1,4]dioxin-2-yl)methyl)piperidin-3-yl)hexahydro-1H-isoindole-1,3(2H)-dione hydrochloride

Prepared as in example 92 from (S)-1-(((S)-2,3-dihydrobenzo[b][1,4]dioxin-2-yl)methyl)piperidin-3-amine (0.30 g, 1.21 mmol) and cis-1,2-cyclohexanedicarboxylic anhydride (0.37 g, 2.42 mmol). Product (0.30 g) was obtained as while solid after trituration of the crude product with EtOAc.

¹H NMR (400 MHz, DMSO-d₆) δ ppm 1.21-1.47 (4H, m), 1.51-1.66 (2H, m), 1.67-1.82 (3H, m), 1.86-2.01 (2H, m), 2.00-2.18 (1H, m), 2.90-2.99 (2H, m), 3.00-3.14 (1H, m), 3.40-3.70 (5H, m), 4.05 (1H, dd), 4.35 (1H, dd), 4.43-4.58 (1H, m), 4.86-4.98 (1H, m), 6.84-6.95 (4H, m), 11.30 (1H, br s).

### EXAMPLE 94: 2-((S)-1-(((S)-2,3-Dihydrobenzo[b][1,4]dioxin-2-yl)methyl)-piperidin-3-yl)-4,5,6,7-tetrahydro-1H-isoindole-1,3(2H)-dione hydrochloride

Prepared as in example 92 from (S)-1-(((S)-2,3-dihydrobenzo[b][1,4]dioxin-2-yl)methyl)piperidin-3-amine (0.30 g, 1.21 mmol) and 3,4,5,6-tetrahydrophthalic anhydride (0.28 g, 1.80 mmol). Product (0.23 g) was obtained as while solid without further purification.

¹H NMR (400 MHz, DMSO-d₆) δ ppm 1.63-1.85 (5H, m), 1.87-2.13 (3H, m), 2.18-2.30 (4H, m), 3.00-3.15 (1H, m), 3.39-3.74 (5H, m), 4.03 (1H, dd), 4.31-4.39 (1H, m), 4.43-4.56 (1H, m), 4.85-4.97 (1H, m), 6.82-6.97 (4H, m), 11.29 (1H, br s).

### EXAMPLE 95: 3-((S)-1-(((S)-2,3-Dihydrobenzo[b][1,4]dioxin-2-yl)methyl)piperidin-3-yl)-3-azabicyclo[3.1.0]hexane-2,4-dione

To a solution of (S)-1-(((S)-2,3-dihydrobenzo[b][1,4]dioxin-2-yl)methyl)piperidin-3-amine (0.20 g, 0.81 mmol) in xylenes (4 ml) was added 3-oxabicyclo(3.1.0)-hexane-2,4-dione (0.18 g, 1.61 mmol) and the solution was refluxed. After 5 hours the reaction mixture was cooled to rt and 1M HCl (7 ml) was added. Water layed was washed with EtOAc, made basic (pH 10) with Na₂CO₃ and extracted with EtOAc. The combined organic layers were washed with brine, dried (Na₂SO₄) and concentrated to afford 0.19 g of 3-((S)-1-(((S)-2,3-dihydrobenzo[b][1,4]dioxin-2-yl)methyl)piperidin-3-yl)-3-azabicyclo[3.1.0]hexane-2,4-dione as white solid.

¹H NMR (400 MHz, CDCl₃) δ ppm 1.27-1.31 (1H, m), 1.46-1.53 (1H, m), 1.53-1.67 (2 H, m), 1.67-1.75 (1H, m), 1.99-2.11 (1H, m), 2.12-2.21 (1H, m), 2.39-2.46 (2H, m), 2.58-2.77 (4H, m), 2.79-2.86 (1H, m), 3.93-4.05 (2H, m), 4.20-4.32 (2H, m), 6.79-6.88 (4H, m).

### EXAMPLE 96: 1-((S)-1-(((S)-2,3-Dihydrobenzo[b][1,4]dioxin-2-yl)methyl)-piperidin-3-yl)-3-ethylpyrimidine-2,4,6(1H,3H,5H)-trione

To a solution of (S)-1-(((S)-2,3-dihydrobenzo[b][1,4]dioxin-2-yl)methyl)piperidin-3-amine (0.12 g, 0.50 mmol) in CH₂Cl₂ (5 ml) was added ethyl isocyanate (0.040 ml, 0.50 mmol). After 5.5 hours reaction mixture was diluted with CH₂Cl₂ (20 ml) and malonyl chloride (0.053 ml, 0.55 mmol) was added dropwise. After 16 hours brine (20 ml) was added and the organic layer was separated and concentrated. Purification of the evaporation residue by column chromatography (silica gel, MeOH—CH₂Cl₂) afforded 0.042 g of 1-((S)-1-(((S)-2,3-dihydrobenzo[b][1,4]dioxin-2-yl)methyl)piperidin-3-yl)-3-ethylpyrimidine-2,4,6(1H,3H,5H)-trione as yellow solid.

¹H NMR (400 MHz, CDCl₃) δ ppm 1.21 (3H, t), 1.67-1.86 (3H, m), 2.22-2.38 (2H, m), 2.75-2.85 (2H, m), 2.92-3.05 (2H, m), 3.05-3.15 (1H, m), 3.65 (2H, br s), 3.93 (2H, q), 4.01 (1H, dd), 4.29 (1H, dd), 4.32-4.41 (1H, m), 4.85-4.98 (1H, m), 6.80-6.90 (4H, m).

### EXAMPLE 97: 1-((S)-1-(((S)-2,3-Dihydrobenzo[b][1,4]dioxin-2-yl)methyl)-piperidin-3-yl)-3-methylpyrrolidin-2-one

### Step 1-2: 3-Methyl-1-((S)-piperidin-3-yl)pyrrolidin-2-one hydrochloride

To a solution of (S)-tert-butyl 3-aminopiperidine-1-carboxylate (3.81 g, 19.1 mmol) and ethyl 2-methyl-4-oxobutanoate (2.50 g, 17.3 mmo) (Organic Letters 2012, pp. 3268-3271) in dichloroethane (100 ml) at 0° C. was added NaBH(OAc)₃ and mixture was then stirred at rt. After 6 h cool water was slowly added and the mixture was extracted with CH₂Cl₂. The organic layer was dried (Na₂SO₄) and concentrated under reduced pressure. Purification of the evaporation residue by column chromatography (silica gel, EtOAc in pet ether) afforded 1.5 g of (3S)-tert-butyl 3-(3-methyl-2-oxopyrrolidin-1-yl)piperidine-1-carboxylate as as brown liquid.

(3S)-Tert-butyl 3-(3-methyl-2-oxopyrrolidin-1-yl)piperidine-1-carboxylate (0.50 mg, 5.3 mmol) was taken in a solution of HCl in diethylether at 0° C. and stirred at rt. After 1 h the reaction mixture was concentrated under reduced pressure to obtain 0.56 g of 3-methyl-1-((S)-piperidin-3-yl)pyrrolidin-2-one hydrochloride as off white solid.

LC-MS (ES+) [M+1]: 183.32.

### Step 3: 1-((S)-1-(((S)-2,3-Dihydrobenzo[b][1,4]dioxin-2-yl)methyl)piperidin-3-yl)-3-methylpyrrolidin-2-one

A mixture of 3-methyl-1-((S)-piperidin-3-yl)pyrrolidin-2-one hydrochloride (0.10 g, 0.46 mmol), (R)-2-(bromomethyl)-2,3-dihydrobenzo[b][1,4]dioxine (0.126 g, 0.55 mmol) and K₂CO₃ (0.114 g, 0.82 mmol) in MeCN (1.6 ml) was heated to 120° C. in microwave reactor. After 4 hours, the reaction mixture was cooled to rt and solvents were evaporated. Evaporation residue was taken into a mixture of water (10 ml) and EtOAc (10 ml) and layers were separated. Aqueous phase was extracted with EtOAc. Combined organic layers were washed with water and brine, dried (Na₂SO₄) and concentrated. Purification of the evaporation residue by reverse phase column chromatography (C18, 0.1% aq. HCOOH/MeCN) afforded 0.080 g of 1-((S)-1-(((S)-2,3-dihydrobenzo[b][1,4]dioxin-2-yl)methyl)piperidin-3-yl)-3-methylpyrrolidin-2-one as colorless oil.

¹H NMR (400 MHz, CDCl₃) δ ppm 1.17-1.21 (3H, m, CH₃ from both diastereomers), 1.34-1.49 (1H, m), 1.52-1.63 (1H, m), 1.64-1.78 (3H, m), 2.08-2.26 (3H, m), 2.38-2.50 (1H, m), 2.60-2.66 (2H, m), 2.77-2.85 (1H, m), 2.85-2.94 (1H, m), 3.19-3.40 (2H, m), 3.97-4.05 (1H, m), 4.06-4.16 (1H, m), 4.23-4.32 (2H, m), 6.79-6.89 (4H, m).

### EXAMPLE 98: 1-((S)-1-(((S)-2,3-Dihydrobenzo[b][1,4]dioxin-2-yl)methyl)piperidin-3-yl)-3-methylpyrrolidin-2-one, diastereomer 1

The title compound was purified by preparative chiral HPLC-chromatography from the mixture of diasteromers obtained in example 97. Conditions: Chiralpak IC column, eluent A: MTBE+0.2% diethylamine, eluent B: THF+0.2% diethylamine, 5% B in A, flowrate 20 ml/min. Diastereomer 1 is the faster eluting peak (RT 12.6 min, prep column).

¹H NMR (400 MHz, CDCl₃) δ ppm 1.19 (3H, d), 1.33-1.47 (1H, m), 1.52-1.63 (1H, m), 1.63-1.78 (3H, m), 2.08-2.27 (3H, m), 2.37-2.50 (1H, m), 2.63-2.68 (2H, m), 2.78-2.86 (1H, m), 2.86-2.92 (1H, m), 3.25-3.34 (2H, m), 3.98-4.04 (1H, m), 4.07-4.17 (1H, m), 4.24-4.32 (2H, m), 6.79-6.91 (4H, m).

### EXAMPLE 99: 1-((S)-1-(((S)-2,3-Dihydrobenzo[b][1,4]dioxin-2-yl)methyl)piperidin-3-yl)-3-methylpyrrolidin-2-one, diastereomer 2

The title compound was purified by preparative chiral HPLC-chromatography from the mixture of diasteromers obtained in example 97. Conditions: Chiralpak IC column, eluent A: MTBE+0.2% diethylamine, eluent B: THF+0.2% diethylamine, 5% B in A, flowrate 20 ml/min. Diastereomer 2 is the slower eluting peak. (RT 15.2 min, prep column)

¹H NMR (400 MHz, CDCl₃) δ ppm 1.19 (3H, d), 1.37-1.51 (1H, m), 1.52-1.64 (1H, m), 1.64-1.77 (3H, m), 2.07-2.27 (3H, m), 2.39-2.51 (1H, m), 2.59-2.68 (2H, m), 2.78-2.85 (1H, m), 2.87-2.94 (1H, m), 3.19-3.27 (1H, m), 3.33-3.40 (1H, m), 3.97-4.05 (1H, m), 4.06-4.17 (1H, m), 4.23-4.32 (2H, m), 6.79-6.89 (4H, m).

### EXAMPLE 100: 1-((S)-1-(((S)-2,3-Dihydrobenzo[b][1,4]dioxin-2-yl)methyl)piperidin-3-yl)-3,3-dimethylpyrrolidin-2-one

### Step 1: (S)-tert-Butyl 3-(3,3-dimethyl-2-oxopyrrolidin-1-yl)piperidine-1-carboxylate

To a stirred suspension of (3S)-tert-butyl 3-(3-methyl-2-oxopyrrolidin-1-yl)piperidine-1-carboxylate (0.50 g, 1.77 mmol) in THF (8 ml) at −78° C. was added lithium diisopropylamine (0.85 ml, 2.12 mmol) over a period of 5 min. After 30 min a solution of methyl iodide (0.16 ml, 2.66 mmol) in THF (2 ml) was added drop wise via syringe over 10 min. The reaction was warmed up to rt. After 16 hours sat. NH₄Cl solution wad added. The layers were separated and the aqueous phase was extracted with EtOAc. The combined organic layers were washed with brine, dried (Na₂SO₄) and concentrated under reduced pressure. Purification of the evaporation residue by column chromatography (silica gel, EtOAc in pet ether) afforded 0.20 g of (S)-tert-butyl 3-(3,3-dimethyl-2-oxopyrrolidin-1-yl)piperidine-1-carboxylate as pale yellow thick liquid.

LC-MS (ES+) [M+1]: 297.01.

### Step 2: (S)-3,3-Dimethyl-1-(piperidin-3-yl)pyrrolidin-2-one hydrochloride

A solution of (S)-tert-butyl 3-(3,3-dimethyl-2-oxopyrrolidin-1-yl)piperidine-1-carboxylate (0.20 g, 0.67 mmol) in HCl/dioxane (3 ml) was stirred at rt. After 1 h most of the solvent was distilled off and evaporation residue was co-distilled with toluene (2×). Purification of the final evaporation residue by trituration with diethyl ether afforded 0.100 g of (S)-3,3-dimethyl-1-(piperidin-3-yl)pyrrolidin-2-one hydrochloride as pale yellow solid.

LC-MS (ES+) [M+1]: 197.1.

### Step 3: 1-((S)-1-(((S)-2,3-Dihydrobenzo[b][1,4]dioxin-2-yl)methyl)piperidin-3-yl)-3,3-dimethylpyrrolidin-2-one

A mixture of (S)-3,3-dimethyl-1-(piperidin-3-yl)pyrrolidin-2-one hydrochloride (0.10 g, 0.43 mol), (R)-2-(bromomethyl)-2,3-dihydrobenzo[b][1,4]dioxine (0.116 g, 0.51 mmol) and K₂CO₃ (0.105 g, 0.76 mmol) in MeCN (1.5 ml) was heated to 120° C. in microwave reactor. After 4 hours, the reaction mixture was cooled to rt and solvents were evaporated. Evaporation residue was taken into a mixture of water (10 ml) and EtOAc (10 ml) and layers were separated. Aqueous phase was extracted with EtOAc. Combined organic layers were washed with water and brine, dried (Na₂SO₄) and concentrated. Purification of the evaporation residue by column chromatography (silica gel, EtOAc-heptane) afforded 0.10 g of 1-((S)-1-(((S)-2,3-dihydrobenzo[b][1,4]dioxin-2-yl)methyl)piperidin-3-yl)-3,3-dimethylpyrrolidin-2-one as colorless oil.

¹H NMR (400 MHz, CDCl₃) δ ppm 1.13 (6H, d), 1.36-1.48 (1H, m), 1.63-1.78 (3H, m), 1.82 (2H, t), 2.08-2.19 (2H, m), 2.61-2.67 (2H, m), 2.78-2.85 (1H, m), 2.86-2.92 (1H, m), 3.19-3.34 (2H, m), 3.97-4.04 (1H, m), 4.04-4.13 (1H, m), 4.24-4.34 (2H, m), 6.79-6.89 (4H, m)

### EXAMPLE 101: 1-((S)-1-(((S)-2,3-Dihydrobenzo[b][1,4]dioxin-2-yl)methyl)piperidin-3-yl)-3-methylimidazolidin-2-one

To a solution of 1-((S)-1-(((S)-2,3-dihydrobenzo[b][1,4]dioxin-2-yl)methyl)piperidin-3-yl)imidazolidin-2-one (0.125 g, 0.39 mmol) in DMF (1.5 ml) at 0° C. was added NaH (0.024 g, 0.59 mmol, 60% dispersion in mineral oil). After 20 minutes MeI (27 μl, 0.43 mmol) was added. After 3 hours water (5 ml) was added and the mixture was extracted with EtOAc (3×). Combined organic layers were washed with brine, dried (Na₂SO₄) and concentrated. The evaporation residue was dissolved in heptane and concentrated to dryness. Purification of the evaporation residue by column chromatography (silica gel, MeOH—CH₂Cl₂) afforded 0.060 g of 1-((S)-1-(((S)-2,3-dihydrobenzo[b][1,4]-dioxin-2-yl)methyl)piperidin-3-yl)-3-methylimidazolidin-2-one as colorless oil.

¹H NMR (400 MHz, CDCl₃) δ ppm 1.34-1.45 (1H, m), 1.63-1.80 (3H, m), 2.07-2.17 (2H, m), 2.61-2.66 (2H, m), 2.77 (3H, s), 2.78-2.83 (1H, m), 2.90-2.98 (1H, m), 3.20-3.39 (4H, m), 3.83-3.93 (1H, m), 3.96-4.05 (1H, m), 4.23-4.32 (2H, m), 6.79-6.88 (4H, m)

### EXAMPLE 102: 3-((S)-1-(((S)-2,3-Dihydrobenzo[b][1,4]dioxin-2-yl)methyl)piperidin-3-yl)imidazolidine-2,4-dione

### Step 1: tert-Butyl-2-((S)-1-(((S)-2,3-dihydrobenzo[b][1,4]dioxin-2-yl)methyl)piperidin-3-ylamino)-2-oxoethylcarbamate

To a suspension of (S)-1-(((S)-2,3-dihydrobenzo[b][1,4]dioxin-2-yl)methyl)piperidin-3-amine p-toluenesulfonate (2.0 g, 4.8 mmol) and EtOAc (4 ml) were added pyridine (1.53 ml, 19.0 mmol) and N-tert-butoxycarbnonylglycine (0.96 g, 5.5 mmol) and the mixture was cooled to −10° C. To the mixture was added 1-propanephosphonic acid cyclic anhydride (5.4 ml, 9.0 mmol, 50% solution in EtOAc) and the reaction temperature was let to rise spontaneously towards rt. After 20 hours EtOAc (60 ml) was added and the solution was washed with saturated NaHCO₃ (2×30 ml), dried (Na₂SO₄) and concentrated. Toluene was added and thorough evaporation of solvents afforded 1.97 g of tert-butyl 2-((S)-1-(((S)-2,3-dihydrobenzo[b][1,4]dioxin-2-yl)methyl)piperidin-3-ylamino)-2-oxoethylcarbamate as yellow oil.

LC-MS (ES+) [M+1]: 406.33

### Step 2: 2-Amino-N—((S)-1-(((S)-2,3-dihydrobenzo[b][1,4]dioxin-2-yl)methyl)piperidin-3-yl)acetamide dihydrochloride

To a solution of tert-butyl 2-((S)-1-(((S)-2,3-dihydrobenzo[b][1,4]dioxin-2-yl)methyl)piperidin-3-ylamino)-2-oxoethylcarbamate (2.0 g, 4.9 mmol) in methanol (16 ml) was added a solution of HCl in dioxane (7.3 ml, 29 mmol, 4 M). After 5 hours solvents were evaporated. Keeping the evaporation residue in high vacuum afforded 1.94 g of 2-amino-N—((S)-1-(((S)-2,3-dihydrobenzo[b][1,4]dioxin-2-yl)methyl)piperidin-3-yl)acetamide dihydrochloride as white solid.

LC-MS (ES+) [M+1]: 306.19

### Step 3: 3-((S)-1-(((S)-2,3-Dihydrobenzo[b][1,4]dioxin-2-yl)methyl)piperidin-3-yl)imidazolidine-2,4-dione

To a mixture of 2-amino-N—((S)-1-(((S)-2,3-dihydrobenzo[b][1,4]dioxin-2-yl)methyl)-piperidin-3-yl)acetamide dihydrochloride (1.94 g, 5.1 mmol) and triethylamine (2.14 ml, 15.4 mmol) in MeCN (50 ml) was added N,N′-carbonyldiimidazole (1.0 g, 6.2 mmol). After 4 hours the reaction mixture was heated on 80° C. bath. After 2 hours the solution was cooled to rt and solvents were evaporated. The evaporation residue was taken into CH₂Cl₂ and the solution was washed with saturated NaHCO₃ and water. Solvents were evaporated. Purification of the evaporation residue with column chromatography (silica gel, EtOAc-heptane) afforded 1.29 g of 3-((S)-1-(((S)-2,3-dihydrobenzo[b][1,4]dioxin-2-yl)methyl)piperidin-3-yl)imidazolidine-2,4-dione as colorless oil.

¹H NMR (400 MHz, CDCl₃) δ ppm 1.63-1.81 (3H, m), 2.09-2.25 (2H, m), 2.58-2.78 (2H, m), 2.78-2.90 (3H, m), 3.92 (2H, d), 3.96-4.03 (1H, m), 4.11-4.22 (1H, m), 4.22-4.33 (2H, m), 5.54 (1H, s), 6.79-6.89 (4H, m).

### EXAMPLE 103: 3-((S)-1-(((S)-2,3-Dihydrobenzo[b][1,4]dioxin-2-yl)methyl)piperidin-3-yl)-1-methylimidazolidine-2,4-dione

To a solution of 3-((S)-1-(((S)-2,3-dihydrobenzo[b][1,4]dioxin-2-yl)methyl)piperidin-3-yl)imidazolidine-2,4-dione (0.070 g, 0.21 mmol) in DMF (1 ml) at 0° C. was added NaH (0.011 g, 0.28 mmol, 60% dispersion in mineral oil). After 20 minutes MeI (17 μl, 0.28 mmol) was added. After 1 hour water (3 ml) was added and the mixture was extracted with CH₂Cl₂ (3×). Combined organic layers were washed with brine, dried (Na₂SO₄) and concentrated. Purification of the evaporation residue by reverse phase column chromatography (C18, 0.1% NH₄OH in MeCN) afforded 0.026 g of 3-((S)-1-(((S)-2,3-dihydrobenzo[b][1,4]dioxin-2-yl)methyl)piperidin-3-yl)-1-methylimidazolidine-2,4-dione as white solid.

¹H NMR (400 MHz, CDCl₃) δ ppm 1.58-1.78 (3H, m), 2.09-2.24 (2H, m), 2.60-2.74 (2H, m), 2.76-2.88 (3H, m), 2.97 (3H, s), 3.80 (2H, s), 3.99 (1H, dd), 4.10-4.20 (1H, m), 4.22-4.32 (2H, m), 6.79-6.88 (4H, m).

### EXAMPLE 104: 3-((S)-1-(((S)-2,3-Dihydrobenzo[b][1,4]dioxin-2-yl)methyl)piperidin-3-yl)-1-isopropylimidazolidine-2,4-dione

To a solution of 3-((S)-1-(((S)-2,3-dihydrobenzo[b][1,4]dioxin-2-yl)methyl)piperidin-3-yl)imidazolidine-2,4-dione (0.20 g, 0.60 mmol) in DMF (2 ml) at 0° C. was added NaH (0.031 g, 0.79 mmol, 60% dispersion in mineral oil). After 20 minutes 2-iodopropane (42 μl, 0.42 mmol) was added and the reaction was stirred at rt. After 4.5 hours more NaH (0.010 g, 0.26 mmol) and 2-iodopropane (21 μl, 0.21 mmol) were added. After 19 hours sat. NH₄Cl (3 ml) and water (3 ml) were added and the mixture was extracted with EtOAc (3×). Combined organic layers were washed with brine, dried (Na₂SO₄) and concentrated to dryness. Purification of the evaporation residue by reverse phase column chromatography (C18, 0.1% NH₄OH/MeCN) afforded 0.041 g of 3-((S)-1-(((S)-2,3-dihydrobenzo[b][1,4]-dioxin-2-yl)methyl)piperidin-3-yl)-1-isopropylimidazolidine-2,4-dione as white solid.

¹H NMR (400 MHz, CDCl₃) δ ppm 1.19 (6H, d), 1.60-1.80 (3H, m), 2.09-2.24 (2H, m), 2.59-2-67 (1H, m) 2.67-2.74 (1H, m), 2.78-2.89 (3H, m), 3.73 (2H, s), 3.99 (1H, dd), 4.09-4.19 (1H, m), 4.22-4.40 (3H, m), 6.79-6.89 (4H, m).

### EXAMPLE 105: 3-((S)-1-(((S)-2,3-Dihydrobenzo[b][1,4]dioxin-2-yl)methyl)-piperidin-3-yl)-1-ethylimidazolidine-2,4-dione

To a solution of 3-((S)-1-(((S)-2,3-dihydrobenzo[b][1,4]dioxin-2-yl)methyl)piperidin-3-yl)imidazolidine-2,4-dione (0.20 g, 0.60 mmol) in DMF (2 ml) at 0° C. was added NaH (0.031 g, 0.79 mmol, 60% dispersion in mineral oil). After 20 minutes iodoethane (0.10 ml, 1.20 mmol) was added and the reaction was stirred at rt. After 3 hours sat. NH₄Cl (3 ml) and water (3 ml) were added and the mixture was extracted with EtOAc (3×). Combined organic layers were washed with brine, dried (Na₂SO₄) and concentrated. Purification of the evaporation residue by reverse phase column chromatography (C18, 0.1% NH₄OH/MeCN) afforded 0.098 g of 3-((S)-1-(((S)-2,3-dihydrobenzo[b][1,4]dioxin-2-yl)methyl)piperidin-3-yl)-1-ethylimidazolidine-2,4-dione as white solid.

¹H NMR (400 MHz, CDCl₃) δ ppm 1.18 (3H, t), 1.58-1.79 (3H, m), 2.09-2.25 (2H, m), 2.60-2.67 (1H, m) 2.67-2.74 (1H, m), 2.78-2.89 (3H, m), 3.43 (2H, q), 3.79 (2H, s), 3.99 (1H, dd), 4.10-4.20 (1H, m), 4.22-4.34 (2H, m), 6.79-6.89 (4H, m).

### EXAMPLE 106: 1-Cyclopentyl-3-((S)-1-(((S)-2,3-dihydrobenzo[b][1,4]dioxin-2-yl)methyl)piperidin-3-yl)imidazolidine-2,4-dione

To a solution of 3-((S)-1-(((S)-2,3-dihydrobenzo[b][1,4]dioxin-2-yl)methyl)piperidin-3-yl)imidazolidine-2,4-dione (0.20 g, 0.60 mmol) in DMF (2 ml) at 0° C. was added NaH (0.031 g, 0.79 mmol, 60% dispersion in mineral oil). After 20 minutes cyclopentylbromide (0.130 ml, 1.12 mmol) was added and the reaction was stirred at rt. After 7 hours more NaH (0.016 g, 0.40 mmol) and cyclopentylbromide (0.065 ml, 0.56 mmol) were added. After 17 hours sat. NH₄Cl (3 ml) and water (3 ml) were added and the mixture was extracted with EtOAc (3×). Combined organic layers were washed with brine, dried (Na₂SO₄) and concentrated. Purification of the evaporation residue by reverse phase column chromatography (C18, 0.1% NH₄OH/MeCN) afforded 0.069 g of 1-cyclopentyl-3-((S)-1-(((S)-2,3-dihydrobenzo[b][1,4]dioxin-2-yl)methyl)piperidin-3-yl)imidazolidine-2,4-dione as white solid.

¹H NMR (400 MHz, CDCl₃) δ ppm 1.44-1.55 (2H, m), 1.56-1.79 (7H, m), 1.86-1.97 (2H, m), 2.09-2.25 (2H, m), 2.60-2.74 (2H, m), 2.77-2.89 (3H, m), 3.75 (2H, s), 3.99 (1H, dd), 4.09-4.20 (1H, m), 4.21-4.33 (2H, m), 4.43 (1H, quin), 6.79-6.89 (4H, m).

### EXAMPLE 107: 3-((S)-1-(((S)-2,3-Dihydrobenzo[b][1,4]dioxin-2-yl)methyl)piperidin-3-yl)-1-isobutylimidazolidine-2,4-dione

To a solution of 3-((S)-1-(((S)-2,3-dihydrobenzo[b][1,4]dioxin-2-yl)methyl)piperidin-3-yl)imidazolidine-2,4-dione (0.20 g, 0.60 mmol) in DMF (2 ml) at 0° C. was added NaH (0.031 g, 0.79 mmol, 60% dispersion in mineral oil). After 20 minutes 1-iodo-2-methylpropane (0.140 ml, 1.21 mmol) was added and the reaction was stirred at rt. After 6 hours more NaH (0.016 g, 0.40 mmol) and 1-iodo-2-methylpropane (0.070 ml, 0.61 mmol) were added. After 17 hours sat. NH₄Cl (3 ml) and water (3 ml) were added and the mixture was extracted with EtOAc (3×). Combined organic layers were washed with brine, dried (Na₂SO₄) and concentrated. Purification of the evaporation residue by reverse phase column chromatography (C18, 0.1% NH₄OH/MeCN) afforded 0.077 g of 3-((S)-1-(((S)-2,3-dihydrobenzo[b][1,4]dioxin-2-yl)methyl)piperidin-3-yl)-1-isobutylimidazolidine-2,4-dione as white solid.

¹H NMR (400 MHz, CDCl₃) δ ppm 0.93 (6H, d), 1.59-1.78 (3H, m), 1.83-1.95 (1H, m), 2.09-2.24 (2H, m), 2.60-2.75 (2H, m), 2.77-2.89 (3H, m), 3.16 (2H, d), 3.79 (2H, s), 4.00 (1H, dd), 4.10-4.20 (1H, m), 4.22-4.33 (2H, m), 6.79-6.89 (4H, m).

### EXAMPLE 108: 1-(Cyclopropylmethyl)-3-((S)-1-(((S)-2,3-dihydrobenzo[b][1,4]dioxin-2-yl)methyl)piperidin-3-yl)imidazolidine-2,4-dione

To a solution of 3-((S)-1-(((S)-2,3-dihydrobenzo[b][1,4]dioxin-2-yl)methyl)piperidin-3-yl)imidazolidine-2,4-dione (0.20 g, 0.60 mmol) in DMF (2 ml) at 0° C. was added NaH (0.031 g, 0.79 mmol, 60% dispersion in mineral oil). After 20 minutes (bromomethyl)cyclopropane (0.140 ml, 1.21 mmol) was added and the reaction was stirred at rt. After 17 hours sat. NH₄Cl (3 ml) and water (3 ml) were added and the mixture was extracted with EtOAc (3×). Combined organic layers were washed with brine, dried (Na₂SO₄) and concentrated. Purification of the evaporation residue by reverse phase column chromatography (C18, 0.1% NH₄OH/MeCN) afforded 0.091 g of 1-(cyclopropylmethyl)-3-((S)-1-(((S)-2,3-dihydrobenzo[b][1,4]dioxin-2-yl)methyl)piperidin-3-yl)imidazolidine-2,4-dione as oil.

¹H NMR (400 MHz, CDCl₃) δ ppm 0.21-0.27 (2H, m), 0.55-0.61 (2H, m), 0.87-0.99 (1H, m), 1.59-1.79 (3H, m), 2.09-2.25 (2H, m), 2.60-2.75 (2H, m), 2.78-2.89 (3H, m), 3.23 (2H, d), 3.90 (2H, s), 3.99 (1H, dd), 4.10-4.21 (1H, m), 4.22-4.33 (2H, m), 6.79-6.89 (4H, m).

### EXAMPLE 109: 2-(3-((S)-1-(((S)-2,3-Dihydrobenzo[b][1,4]dioxin-2-yl)methyl)piperidin-3-yl)-2,4-dioxoimidazolidin-1-yl)-N,N-dimethylacetamide

To a solution of 3-((S)-1-(((S)-2,3-dihydrobenzo[b][1,4]dioxin-2-yl)methyl)piperidin-3-yl)imidazolidine-2,4-dione (0.090 g, 0.27 mmol) in DMF (1 ml) at 0° C. was added NaH (0.014 g, 0.35 mmol, 60% dispersion in mineral oil). After 20 minutes 2-chloro-N,N-dimethylacetamide (0.028 ml, 0.27 mmol) was added and the reaction mixture was stirred at rt. After 2 hours sat. NH₄Cl (3 ml) was added and the mixture was extracted with EtOAc (3×). Combined organic layers were washed with brine, dried (Na₂SO₄) and concentrated. Purification of the evaporation residue by reverse phase column chromatography (C18, aq. HCOOH/MeCN) afforded 0.013 g of 2-(3-((S)-1-(((S)-2,3-dihydrobenzo[b][1,4]dioxin-2-yl)methyl)piperidin-3-yl)-2,4-dioxoimidazolidin-1-yl)-N,N-dimethylacetamide as colorless oil.

¹H NMR (400 MHz, CDCl₃) δ ppm 1.62-1.80 (3H, m), 2.09-2.24 (2H, m), 2.60-2.74 (2H, m), 2.78-2.91 (3H, m), 2.96 (3H, s), 3.01 (3H, s), 3.96-4.04 (3H, m), 4.11-4.22 (3H, m), 4.22-4.32 (2H, m), 6.79-6.89 (4H, m).

### EXAMPLE 110: 3-((S)-1-(((S)-2,3-Dihydrobenzo[b][1,4]dioxin-2-yl)methyl)piperidin-3-yl)-5,5-dimethylimidazolidine-2,4-dione

### Step 1: Methyl-(1-(((S)-1-(((S)-2,3-dihydrobenzo[b][1,4]dioxin-2-yl)methyl)piperidin-3-yl)amino)-2-methyl-1-oxopropan-2-yl)carbamate

To a suspension of of 2-(methoxycarbonylamino)-2-methylpropanoic acid (WO2011/004276A1) (1.1 g, 6.6 mmol) in CH₂Cl₂ (22 ml) was added diisopropylethylamine (1.9 ml, 10.9 mmol), O-(benzotriazol-1-yl)-N,N,N′,N′-tetramethyluronium hexafluorophosphate (2.75 g, 7.25 mmol), (S)-1-(((S)-2,3-dihydrobenzo[b][1,4]dioxin-2-yl)methyl)piperidin-3-amine (1.5 g, 6.0 mmol) and finally N-methyl pyrrolidone (7 ml). After 66 hours saturated solution of oxalic acid (20 ml) and water (10 ml) were added, the mixture was shaken and the layers were separated. The organic layer was washed with 5% oxalic acid solution (2×), 1M NaOH solution (2×) and water. All aqueous layers were combined and solution was made basic (pH 12) by adding Na₂CO₃ and 1 M NaOH. Basic solution was extracted with EtOAc (3×). All organic layers were combined, washed with brine, dried (Na₂SO₄). Evaporation of the solvents and keeping the residue at high vacuum overnight gave 3.16 g of crude product as a yellow oil.

LC-MS (ES+) [M+1]: 392.85

### Step 2: 3-((S)-1-(((S)-2,3-Dihydrobenzo[b][1,4]dioxin-2-yl)methyl)piperidin-3-yl)-5,5-dimethylimidazolidine-2,4-dione

The crude product from step 1 was dried azeotropically with toluene and dissolved in THF (60 ml). To the solution was added potassium tert-butylate (0.68 g, 6.03 mmol). After 2 h saturated NH₄Cl (60 ml) was added, followed by aqueous 25% NH₃ (3 ml). Phases were separated and the water phase was extracted with EtOAc. Combined organic layers were washed with water and brine, dried (Na₂SO₄) and concentrated to dryness. Yield 2.0 g. Purification of 300 mg portion of the crude product by reverse phase column chromatography (C18, 0.1% NH₄OH/MeCN) afforded 0.153 g of 3-((S)-1-(((S)-2,3-dihydrobenzo[b][1,4]dioxin-2-yl)methyl)piperidin-3-yl)-5,5-dimethylimidazolidine-2,4-dione as white solid.

¹H NMR (400 MHz, CDCl₃) δ ppm 1.41 (6H, s), 1.56-1.79 (3H, m), 2.09-2.24 (2H, m), 2.64 (1H, dd), 2.72, (1H, dd), 2.76-2.89 (3H, m), 4.00 (1H, dd), 4.07-4.17 (1H, m), 4.23-4.34 (2H, m), 5.61 (1H, br s), 6.79-6.89 (4H, m).

### EXAMPLE 111: 3-((S)-1-(((S)-2,3-Dihydrobenzo[b][1,4]dioxin-2-yl)methyl)-piperidin-3-yl)-1,5,5-trimethylimidazolidine-2,4-dione

To a solution of 3-((S)-1-(((S)-2,3-dihydrobenzo[b][1,4]dioxin-2-yl)methyl)piperidin-3-yl)-5,5-dimethylimidazolidine-2,4-dione (0.116 g, 0.32 mmol) in DMF (1 ml) at 0° C. was added NaH (0.026 g, 0.65 mmol, 60% dispersion in mineral oil). After 20 minutes MeI (28 μl, 0.45 mmol) was added. After 1 hour saturated NH₄Cl (3 ml) was added and the mixture was extracted with EtOAc (3×). Combined organic layers were washed with brine, dried (Na₂SO₄) and concentrated to dryness. Purification of the evaporation residue by reverse phase column chromatography (C18, 0.1% NH₄OH in MeCN) afforded 0.085 g of 3-((S)-1-(((S)-2,3-dihydrobenzo[b][1,4]dioxin-2-yl)methyl)piperidin-3-yl)-1,5,5-trimethylimidazolidine-2,4-dione as white solid.

¹H NMR (400 MHz, CDCl₃) δ ppm 1.35 (6H, s), 1.58-1.79 (3H, m), 2.06-2.25 (2H, m), 2.59-2.74 (2H, m), 2.75-2.90 (6H, m), 3.99 (1H, dd), 4.09-4.20 (1H, m), 4.22-4.34 (2H, m), 6.79-6.88 (4H, m).

### EXAMPLE 112: (R)-3-((S)-1-(((S)-2,3-Dihydrobenzo[b][1,4]dioxin-2-yl)methyl)-piperidin-3-yl)-5-methylimidazolidine-2,4-dione

### Step 1: tert-Butyl-(R)-1-((S)-1-(((S)-2,3-dihydrobenzo[b][1,4]dioxin-2-yl)methyl)-piperidin-3-ylamino)-1-oxopropan-2-ylcarbamate

To a solution of Boc-D-alanine (0.15 g, 0.79 mmol) in CH₂Cl₂ (3 ml) was added diisopropylethylamine (0.25 ml, 1.42 mmol), O-(benzotriazol-1-yl)-N,N,N′,N′-tetramethyluronium hexafluorophosphate (0.36 g, 0.95 mmol) and finally a solution of(S)-1-(((S)-2,3-dihydrobenzo[b][1,4]dioxin-2-yl)methyl)piperidin-3-amine (0.22 g, 0.87 mmol) in N-methylpyrrolidone (1 ml). After 3 days EtOAc (35 ml) was added and the solution was washed with water and brine. Combined aqueous phases were back-extracted with EtOAc. Combined organic layers were dried (Na₂SO₄) and evaporated to dryness. Purification of the oily evaporation residue by reverse phase column chromatography (C18, 0.1% NH₄OH in MeCN) afforded 0.28 g of tert-butyl-(R)-1-((S)-1-(((S)-2,3-dihydrobenzo[b][1,4]dioxin-2-yl)methyl)piperidin-3-ylamino)-1-oxopropan-2-ylcarbamate as yellow oil.

LC-MS (ES+) [M+1]: 420.18.

### Step 2: (R)-2-Amino-N—((S)-1-(((S)-2,3-dihydrobenzo[b][1,4]dioxin-2-yl)methyl)piperidin-3-yl)propanamide (bis)trifluoroacetate

tert-Butyl (R)-1-((S)-1-(((S)-2,3-dihydrobenzo[b][1,4]dioxin-2-yl)methyl)piperidin-3-ylamino)-1-oxopropan-2-ylcarbamate (0.28 g, 0.67 mmol) was mixed with trifluoroacetic acid (5 ml). After 1.5 hours trifluoroacetic acid was evaporated. The evaporation residue was taken into mixture of CH₂Cl₂ and toluene and evaporated. The residue containing (R)-2-amino-N—((S)-1-(((S)-2,3-dihydrobenzo[b][1,4]dioxin-2-yl)methyl)piperidin-3-yl)propanamide (bis)trifluoroacetate (0.36 g)—yellow, partly crystalline oil was used as such in the next step.

LC-MS (ES+) [M+1]: 320.11.

### Step 3: (R)-3-((S)-1-(((S)-2,3-Dihydrobenzo[b][1,4]dioxin-2-yl)methyl)piperidin-3-yl)-5-methylimidazolidine-2,4-dione

To a solution of (R)-2-amino-N—((S)-1-(((S)-2,3-dihydrobenzo[b][1,4]dioxin-2-yl)methyl)piperidin-3-yl)propanamide (bis)trifluoroacetate (0.36 g, 0.66 mmol) in MeCN (5 ml) was added triethylamine (0.28 ml, 1.97 mmol). After 5 minutes N,N-carbonyldiimidazole (0.12 g, 0.72 mmol) was added. After 2.5 hours the reaction mixture was heated to 80° C. After further 3.5 hours the reaction mixture was cooled to rt and concentrated to dryness. Dichloromethane (20 ml) was added and the solution was washed with water and sat. NaHCO₃, dried (Na₂SO₄) and concentrated to dryness. Purification of the evaporation residue by reverse phase column chromatography (C18, 0.1% NH₄OH in MeCN) afforded 0.16 g of (R)-3-((S)-1-(((S)-2,3-dihydrobenzo[b][1,4]dioxin-2-yl)methyl)piperidin-3-yl)-5-methylimidazolidine-2,4-dione as white solid.

¹H NMR (400 MHz, CDCl₃) δ ppm 1.43 (3H, d), 1.57-1.79 (3H, m), 2.08-2.25 (2H, m), 2.59-2.75 (2H, m), 2.77-2.90 (3H, m), 3.95-4.05 (2H, m), 4.08-4.18 (1H, m), 4.22-4.33 (2H, m), 6.06 (1H, br s), 6.79-6.89 (4H, m).

### EXAMPLE 113: (S)-3-((S)-1-(((S)-2,3-Dihydrobenzo[b][1,4]dioxin-2-yl)methyl)-piperidin-3-yl)-5-methylimidazolidine-2,4-dione

### Step 1: tert-Butyl-(S)-1-((S)-1-(((S)-2,3-dihydrobenzo[b][1,4]dioxin-2-yl)methyl)-piperidin-3-ylamino)-1-oxopropan-2-ylcarbamate

To a suspension of (S)-1-(((S)-2,3-dihydrobenzo[b][1,4]dioxin-2-yl)methyl)piperidin-3-amine (0.240 g, 0.97 mmol) in EtOAc (0.6 ml) were added pyridine (1.53 ml, 19.0 mmol) and (S)-2-((tert-butoxycarbonyl)amino)propanoic acid (0.21 g, 1.1 mmol) and the mixture was cooled to −10° C. To the mixture was added 1-propanephosphonic acid cyclic anhydride (1.1 ml, 1.84 mmol, 50% solution in EtOAc) and the reaction temperature was let to rise spontaneously towards rt. After 1 day, EtOAc (20 ml) was added and the solution was washed with saturated NaHCO₃ (2×15 ml), dried (Na₂SO₄) and concentrated to dryness. Purification of the evaporation residue by reverse phase column chromatography (C18, 0.5% HCOOH/MeCN) afforded 0.050 g of tert-butyl (S)-1-((S)-1-(((S)-2,3-dihydrobenzo[b][1,4]dioxin-2-yl)methyl)piperidin-3-ylamino)-1-oxopropan-2-ylcarbamate as white solid.

LC-MS (ES+) [M+1]: 420.83.

### Step 2: (S)-2-Amino-N—((S)-1-(((S)-2,3-dihydrobenzo[b][1,4]dioxin-2-yl)methyl)-piperidin-3-yl)propanamide dihydrochloride

To a solution of tert-butyl (S)-1-((S)-1-(((S)-2,3-dihydrobenzo[b][1,4]dioxin-2-yl)methyl)piperidin-3-ylamino)-1-oxopropan-2-ylcarbamate in MeOH (1 ml) was added solution of HCl in dioxane (0.18 ml, 0.72 mmol, 6M solution). After 1 day, evaporation of solvents afforded 0.047 g of (S)-2-amino-N—((S)-1-(((S)-2,3-dihydrobenzo[b][1,4]dioxin-2-yl)methyl)piperidin-3-yl)propanamide dihydrochloride as white solid.

LC-MS (ES+) [M+1]: 320.19.

### Step 3: (S)-3-((S)-1-(((S)-2,3-Dihydrobenzo[b][1,4]dioxin-2-yl)methyl)-piperidin-3-yl)-5-methylimidazolidine-2,4-dione

To a suspension of (S)-2-amino-N—((S)-1-(((S)-2,3-dihydrobenzo[b][1,4]dioxin-2-yl)methyl)piperidin-3-yl)propanamide dihydrochloride (0.047 g, 0.12 mmol) in MeCN (0.8 ml) was added triethylamine (0.050 ml, 0.36 mmol) and N,N-carbonyldiimidazole (0.023 g, 0.14 mmol) was added. After 4.5 hours the reaction mixture was heated to 80° C. After further 1.5 hours the reaction mixture was cooled to room temperature and then stirred at rt overnight. Solvents were evaporated and the residue was taken in dichloromethane (10 ml). The solution was washed with water, dried and concentrated to dryness. Purification of the evaporation residue by reverse phase column chromatography (C18, 0.1% NH₄OH/MeCN) afforded 0.022 g of (S)-3-((S)-1-(((S)-2,3-dihydrobenzo[b][1,4]dioxin-2-yl)methyl)-piperidin-3-yl)-5-methylimidazolidine-2,4-dione as white solid.

¹H NMR (400 MHz, CDCl₃) δ ppm 1.43 (3H, d), 1.55-1.79 (3H, m), 2.10-2.25 (2H, m), 2.60-2.75 (2H, m), 2.76-2.89 (3H, m), 3.96-4.05 (2H, m), 4.09-4.19 (1H, m), 4.23-4.33 (2H, m), 5.58 (1H, br s), 6.79-6.89 (4H, m).

### EXAMPLE 114: 3-((S)-1-(((S)-2,3-Dihydrobenzo[b][1,4]dioxin-2-yl)methyl)piperidin-3-yl)-5-phenylimidazolidine-2,4-dione

### Step 1: 3-((S)-1-(((S)-2,3-Dihydrobenzo[b][1,4]dioxin-2-yl)methyl)piperidin-3-yl)-5-phenylimidazolidine-2,4-dione, mixture of diastereomers

To a solution of Boc-L-phenylglycine (0.33 g, 1.33 mmol) in CH₂Cl₂ (4.5 ml) was added diisopropylethylamine (0.38 ml, 2.18 mmol), O-(benzotriazol-1-yl)-N,N,N′,N′-tetramethyluronium hexafluorophosphate (0.55 g, 1.45 mmol) and finally a solution of(S)-1-(((S)-2,3-dihydrobenzo[b][1,4]dioxin-2-yl)methyl)piperidin-3-amine (0.30 g, 1.21 mmol) in N-methylpyrrolidone (1.5 ml). After 1 day EtOAc (35 ml) was added and the solution was washed with water and brine. Combined aqueous phases were back-extracted with EtOAc. Combined organic layers were dried (Na₂SO₄) and evaporated to dryness. Purification of the oily evaporation residue by reverse phase column chromatography (C18, 0.1% NH₄OH/MeCN) afforded 0.52 g of 3-((S)-1-(((S)-2,3-dihydrobenzo[b][1,4]dioxin-2-yl)methyl)piperidin-3-yl)-5-phenylimidazolidine-2,4-dione as a mixture of diastereomers.

LC-MS (ES+) [M+1]: 482.17.

### Step 2: 2-Amino-N—((S)-1-(((S)-2,3-dihydrobenzo[b][1,4]dioxin-2-yl)methyl)piperidin-3-yl)-2-phenylacetamide (bis)trifluoroacetate

3-((S)-1-(((S)-2,3-dihydrobenzo[b][1,4]dioxin-2-yl)methyl)piperidin-3-yl)-5-phenylimidazolidine-2,4-dione (0.52 g, 1.08 mmol) was mixed with trifluoroacetic acid (8 mil). After 1.5 hours trifluoroacetic acid was evaporated. The evaporation residue was taken into mixture of CH₂Cl₂ and toluene and evaporated. The residual 2-amino-N—((S)-1-(((S)-2,3-dihydrobenzo[b][1,4]dioxin-2-yl)methyl)piperidin-3-yl)-2-phenylacetamide (bis)trifluoroacetate (0.7 g)—yellow oil was used as such in the next step.

LC-MS (ES+) [M+1]: 382.18.

### Step 3: 3-((S)-1-(((S)-2,3-Dihydrobenzo[b][1,4]dioxin-2-yl)methyl)piperidin-3-yl)-5-phenylimidazolidine-2,4-dione

To a solution of 2-amino-N—((S)-1-(((S)-2,3-dihydrobenzo[b][1,4]dioxin-2-yl)methyl)-piperidin-3-yl)-2-phenylacetamide (bis)trifluoroacetate (0.41 g, 0.68 mmol) in MeCN (5 ml) was added triethylamine (0.28 ml, 2.03 mmol). After 5 minutes N,N-carbonyldiimidazole (0.12 g, 0.74 mmol) was added. After 3.5 hours the reaction mixture was heated to 45° C. (inside temp.). After further 3 hours the reaction mixture was cooled to room temperature and then stirred at rt overnight. Solvents were evaporated and the residue was taken in CH₂Cl₂ (20 mil). The solution was washed with water and saturated NaHCO₃, dried (Na₂SO₄) and concentrated to dryness. Purification of the evaporation residue by reverse phase column chromatography (C18, 0.1% NH₄OH/MeCN) afforded 0.11 g of 3-((S)-1-(((S)-2,3-dihydrobenzo[b][1,4]dioxin-2-yl)methyl)piperidin-3-yl)-5-phenylimidazolidine-2,4-dione as light brown solid.

¹H NMR (400 MHz, CDCl₃) δ ppm 1.60-1.80 (3H, m), 2.05-2.25 (2H, m), 2.56-2.92 (5H, m), 3.94-4.03 (1H, m), 4.12-4.33 (3H, m), 4.98 (1H, s), 5.75 (1H, br d), 6.78-6.89 (4H, m), 7.31-7.45 (5H, m).

### EXAMPLE 115: 3-((S)-1-(((S)-2,3-Dihydrobenzo[b][1,4]dioxin-2-yl)methyl)-piperidin-3-yl)-1,5-dimethylimidazolidine-2,4-dione

Title compound was obtained as a side product from experiment conducted as in example 103 when using 3-((S)-1-(((S)-2,3-dihydrobenzo[b][1,4]dioxin-2-yl)methyl)piperidin-3-yl)imidazolidine-2,4-dione (0.37 g, 1.05 mmol), NaH (0.067 g 1.68 mmol) and iodomethane (0.085 ml, 1.4 mmol). Purification of the crude product by reverse phase column chromatography (C18, 0.1% NH₄OH/MeCN) afforded 0.044 g of 3-((S)-1-(((S)-2,3-dihydrobenzo[b][1,4]dioxin-2-yl)methyl)piperidin-3-yl)-1,5-dimethylimidazolidine-2,4-dione as colorless oil.

¹H NMR (400 MHz, CDCl₃) δ ppm 1.41 (3H, d), 1.55-1.78 (3H, m), 2.06-2.24 (2H, m), 2.59-2.74 (2H, m), 2.74-2.88 (3H, m), 2.92 (3H, s), 3.75-3.82 (1H, m), 3.99 (1H, dd), 4.09-4.19 (1H, m), 4.22-4.34 (2H, m), 6.79-6.89 (4H, m).

### EXAMPLE 116: 3-((S)-1-(((S)-2,3-Dihydrobenzo[b][1,4]dioxin-2-yl)methyl)-piperidin-3-yl)-1-isopropyl-5,5-dimethylimidazolidine-2,4-dione

### Step 1: (S)-tert-Butyl-3-(2-(methoxycarbonylamino)-2-methylpropanamido)piperidine-1-carboxylate

To a suspension of 2-(methoxycarbonylamino)-2-methylpropanoic acid (WO2011/004276A1) (0.96 g, 6.0 mmol) in CH₂Cl₂ (20 ml) was added diisopropylethylamine (1.7 ml, 9.8 mmol), O-(benzotriazol-1-yl)-N,N,N′,N′-tetramethyluronium hexafluorophosphate (2.48 g, 6.53 mmol) and finally a solution of(S)-3-Amino-1-N-Boc-piperidine (1.09 g, 5.4 mmol) in N-methyl pyrrolidone (7 ml). After 3 days EtOAc (75 ml) was added and the mixture was washed with brine, dried and concentrated to dryness. Purification of the evaporation residue by column chromatography (EtOAc-hept) afforded 2.0 g of (S)-tert-butyl 3-(2-(methoxycarbonylamino)-2-methylpropanamido)piperidine-1-carboxylate containing some residual tetramethylurea from the coupling reagent.

LC-MS (ES−) [M−1]: 342.27

### Step 2: (S)-tert-Butyl-3-(4,4-dimethyl-2,5-dioxoimidazolidin-1-yl)piperidine-1-carboxylate

To a solution of (S)-tert-butyl 3-(2-(methoxycarbonylamino)-2-methylpropanamido)-piperidine-1-carboxylate (1.15 g, 3.35 mmol) in THF (30 ml) was added potassium tert-butoxide (0.38 g, 2.25 mmol). After 2 hours saturated NH₄Cl (40 ml) was added, followed by small amount of water to dissolve precipitate. Phases were separated and the water phase was extracted with EtOAc. Combined organic phases were washed with brine, dried (Na₂SO₄) and concentrated to dryness. Evaporation residue containing 0.98 g of (S)-tert-butyl 3-(4,4-dimethyl-2,5-dioxoimidazolidin-1-yl)piperidine-1-carboxylate was used as such in the next step.

LC-MS (ES+) [M+1]: 312.16

### Step 3: (S)-tert-Butyl-3-(3-isopropyl-4,4-dimethyl-2,5-dioxoimidazolidin-1-yl)piperidine-1-carboxylate

To a solution of (S)-tert-butyl 3-(4,4-dimethyl-2,5-dioxoimidazolidin-1-yl)piperidine-1-carboxylate (0.10 g, 0.32 mmol) in DMF (1 ml) at 0° C. was added NaH (0.022 g, 0.55 mmol, 60% dispersion in mineral oil). After 20 minutes 2-iodopropane (42 μl, 0.42 mmol) was added and the reaction was stirred at rt. More NaH (0.022 g, 0.55 mmol) was added after 4 hours and more 2-iodopropane (40 μl, 0.55 mmol), (20 μl, 0.28 mmol) after 4 hours and 25 hours. After 28 hours water (5 ml) was added and the mixture was extracted with EtOAc (3×). Combined organic layers were washed with brine, dried (Na₂SO₄) and concentrated to dryness. Evaporation residue (0.1 g) containing a mixture of (S)-tert-butyl 3-(3-isopropyl-4,4-dimethyl-2,5-dioxoimidazolidin-1-yl)piperidine-1-carboxylate and starting material was used as such in the next step.

LC-MS (ES+) [M+1]: 354.15.

### Step 4: (R)-2-Amino-N—((S)-1-(((S)-2,3-dihydrobenzo[b][1,4]dioxin-2-yl)methyl)-piperidin-3-yl)propanamide (bis)trifluoroacetate

(S)-tert-Butyl 3-(3-isopropyl-4,4-dimethyl-2,5-dioxoimidazolidin-1-yl)piperidine-1-carboxylate (0.1 g, 0.28 mmol) was mixed with trifluoroacetic acid (1.5 ml). After 2 hours trifluoroacetic acid was evaporated. The evaporation residue was taken into mixture of CH₂Cl₂ and toluene and solvents were evaporated. The residual mixture containing (S)-1-isopropyl-5,5-dimethyl-3-(piperidin-3-yl)imidazolidine-2,4-dione (bis)trifluoroacetate (0.11 g) was used as such in the next step.

(ES+) [M+1]: 354.11.

### Step 5: 3-((S)-1-(((S)-2,3-Dihydrobenzo[b][1,4]dioxin-2-yl)methyl)piperidin-3-yl)-1-isopropyl-5,5-dimethylimidazolidine-2,4-dione

A mixture of of (S)-1-isopropyl-5,5-dimethyl-3-(piperidin-3-yl)imidazolidine-2,4-dione (bis)trifluoroacetate (0.11 g, 0.30 mol), (R)-2-(bromomethyl)-2,3-dihydrobenzo[b][1,4]dioxine (0.075 g, 0.33 mmol) and K₂CO₃ (0.083 g, 0.60 mmol) and diisopropylethylamine (52 μl, 0.30 mmol) in MeCN (1 ml) was heated to 120° C. in microwave reactor. After 4 hours, the reaction mixture was cooled to rt and solvents were evaporated. Evaporation residue was taken into CH₂Cl₂ (20 ml) and the mixture was washed with sat. NaHCO₃, dried (Na₂SO₄) and concentrated to dryness. Purification of the evaporation residue by reverse phase column chromatography (C18, 0.1% aq. HCOOH/MeCN) afforded 0.046 g of 3-((S)-1-(((S)-2,3-dihydrobenzo[b][1,4]dioxin-2-yl)methyl)piperidin-3-yl)-1-isopropyl-5,5-dimethyl-imidazolidine-2,4-dione, as colorless oil.

¹H NMR (400 MHz, CDCl₃) δ ppm 1.34 (6H, s), 1.43 (6H, d), 1.56-1.77 (3H, m), 2.07-2.23 (2H, m), 2.60-2.67 (1H, m), 2.67-2.74 (1H, m), 2.77-2.88 (3H, m), 3.43 (1H, spt), 4.00 (1H, dd), 4.06-4.16 (1H, m), 4.23-4.34 (2H, m), 6.79-6.88 (4H, m).

### EXAMPLE 117: 1-tert-Butyl-3-((S)-1-(((S)-2,3-dihydrobenzo[b][1,4]dioxin-2-yl)methyl)piperidin-3-yl)imidazolidine-2,4-dione

To a solution of (S)-1-(((S)-2,3-dihydrobenzo[b][1,4]dioxin-2-yl)methyl)piperidin-3-amine (0.30 g, 1.21 mmol) in DMF (3 ml) was added tert-butylisocyanate (0.14 ml, 1.21 mmol). After 2.5 hours the reaction mixture was cooled to 0° C. and NaH (0.058 g, 1.45 mmol, 60%-dispersion in mineral oil) was added. After 45 minutes chloroacetylchloride (0.1 ml, 1.2 mmol) was added and the mixture was stirred for 3 h after which it was brought to rt. After further 2 h 1,8-diazabicyclo(5,4,0)undec-7-ene (0.36 ml, 2.42 mmol) and chloroacetyl chloride (0.1 ml, 1.2 mmol) were added and the solution was stirred at rt for 20 h. Saturated K₂CO₃ was added and the reaction mixture was stirred for 30 min. Mixture was extracted with EtOAc (3×). Combined organic layers were washed with brine, dried (Na₂SO₄) and solvents were evaporated. Purification of the evaporation residue by column chromatography (silica gel, EtOAc-heptane) afforded 0.067 g of 1-tert-butyl-3-((S)-1-(((S)-2,3-dihydrobenzo[b][1,4]dioxin-2-yl)methyl)piperidin-3-yl)imidazolidine-2,4-dione as colorless oil.

¹H NMR (400 MHz, CDCl₃) δ ppm 1.42 (9H, s), 1.56-1.78 (3H, m), 2.08-2.24 (2H, m), 2.60-2.67 (1H, m), 2.67-2.74 (1H, m), 2.77-2.88 (3H, m), 3.82 (2H, s), 3.99 (1H, dd), 4.06-4.16 (1H, m), 4.22-4.34 (2H, m), 6.79-6.89 (4H, m).

### EXAMPLE 118: 1-Benzyl-3-((S)-1-(((S)-2,3-dihydrobenzo[b][1,4]dioxin-2-yl)methyl)-piperidin-3-yl)imidazolidine-2,4-dione

### Step 1: 2-(Benzylamino)-N—((S)-1-(((S)-2,3-dihydrobenzo[b][1,4]dioxin-2-yl)methyl)piperidin-3-yl)acetamide

To a suspension of (S)-1-(((S)-2,3-dihydrobenzo[b][1,4]dioxin-2-yl)methyl)piperidin-3-amine p-toluenesulfonate (0.4 g, 0.95 mmol) and Na₂CO₃ (0.40 g, 2.8 mmol) in MeCN (4 mil) at 0° C. was added chloroacetyl chloride (76 μl, 0.95 mmol) and the mixture was stirred at rt. After 2 hours diisopropylethylamine (0.17 ml, 0.95 mmol) and benzylamine (0.15 ml, 1.30 mmol) were added and the mixture was stirred on 80° C. bath. After 9 hour of heating solvents were evaporated and the evaporation residue was taken into a mixture of EtOAc (20 mil) and 1M NaOH (20 ml). The mixture was shaken, phases were separated and the aqueous phase was extracted with EtOAc. Combined organic layers were washes with brine, dried (Na₂SO₄) and solvents were evaporated. Purification of the evaporation residue by reverse phase column chromatography (C18, 0.1% NH₄OH/MeCN) afforded 0.21 g of 2-(benzylamino)-N—((S)-1-(((S)-2,3-dihydrobenzo[b][1,4]dioxin-2-yl)methyl)piperidin-3-yl)acetamide as colorless oil.

LC-MS (ES+) [M+1]: 396.33.

### Step 2: 1-Benzyl-3-((S)-1-(((S)-2,3-dihydrobenzo[b][1,4]dioxin-2-yl)methyl)piperidin-3-yl)imidazolidine-2,4-dione

To a solution of 2-(benzylamino)-N—((S)-1-(((S)-2,3-dihydrobenzo[b][1,4]dioxin-2-yl)methyl)piperidin-3-yl)acetamide (0.20 g, 0.51 mmol) in MeCN (5 ml) was added triethylamine (70 μl, 0.51 mmol) and N,N-carbonyldiimidazole (0.098 g, 0.61 mmol). After 4 hours the reaction mixture was heated to 80° C. (heating block). After 2.5 hours more N,N-carbonyldiimidazole (0.082 g, 0.51 mmol) was added and the mixture was stirred at rt overnight and then at 80° C. for 3 hours. Solvents were evaporated and to the residue was added 1M HCl (15 ml). Solution pH was made acidic by addition of Na₂CO₃ and it was then extracted with EtOAc. Combined organic layers were washed with brine, dried (Na₂SO₄) and solvents were evaporated. Purification of the evaporation residue by column chromatography (silica gel, EtOAc-heptane) afforded 0.12 g of 1-benzyl-3-((S)-1-(((S)-2,3-dihydrobenzo[b][1,4]dioxin-2-yl)methyl)piperidin-3-yl)imidazolidine-2,4-dione as colorless oil.

¹H NMR (400 MHz, CDCl₃) δ ppm 1.58-1.81 (3H, m), 2.10-2.26 (2H, m), 2.62-2.69 (1H, m), 2.69-2.76 (1H, m), 2.80-2.92 (3H, m), 3.68 (2H, s), 3.97-4.03 (1H, m), 4.14-4.24 (1H, m), 4.24-4.34 (2H, m), 4.53 (2H, s), 6.80-6.89 (4H, m), 7.22-7.26 (2H, m), 7.30-7.40 (3H, m).

### EXAMPLE 119: 1-Cyclopropyl-3-((S)-1-(((S)-2,3-dihydrobenzo[b][1,4]dioxin-2-yl)methyl)piperidin-3-yl)imidazolidine-2,4-dione

### Step 1: 2-(Cyclopropylamino)-N—((S)-1-(((S)-2,3-dihydrobenzo[b][1,4]dioxin-2-yl)methyl)piperidin-3-yl)acetamide

To a suspension of (S)-1-(((S)-2,3-dihydrobenzo[b][1,4]dioxin-2-yl)methyl)piperidin-3-amine (0.20 g, 0.81 mmol) and K₂CO₃ (0.33 g, 2.4 mmol) in MeCN (4 ml) at 0° C. was added chloroacetyl chloride (64 μl, 0.81 mmol) and the mixture was stirred at rt. After 1.5 hours cyclopropylamine (67 μl, 0.97 mmol) was added and the mixture was heated in a closed vessel at 120° C. After 4 hours the mixture was cooled and stirred at rt overnight. Mixture was filtered and filtrate was concentrated. Purification of the evaporation residue by reverse phase column chromatography (C18, 0.1% NH₄OH/MeCN) afforded 0.074 g of 2-(cyclopropylamino)-N—((S)-1-(((S)-2,3-dihydrobenzo[b][1,4]dioxin-2-yl)methyl)piperidin-3-yl)acetamide as colorless oil.

LC-MS (ES+) [M+1]: 346.16.

### Step 2: 1-Cyclopropyl-3-((S)-1-(((S)-2,3-dihydrobenzo[b][1,4]dioxin-2-yl)methyl)-piperidin-3-yl)imidazolidine-2,4-dione

To a solution of 2-(cyclopropylamino)-N—((S)-1-(((S)-2,3-dihydrobenzo[b][1,4]dioxin-2-yl)methyl)piperidin-3-yl)acetamide (0.074 g, 0.21 mmol) in MeCN (2 ml) was added triethylamine (30 μl, 0.21 mmol) and N,N-carbonyldiimidazole (0.042 g, 0.26 mmol). After 7 hours more N,N-carbonyldiimidazole (0.030 g, 0.19 mmol) was added and the reaction was stirred overnight. The reaction mixture was then stirred at 80° C. (heating block) for 6 hours and then at rt for 4 days. Solvents were evaporated, the residue was taken in CH₂Cl₂ and washed with sat. NaHCO₃. Organic layer was dried (Na₂SO₄) and concentrated to dryness. Purification of the evaporation residue by column chromatography (silica gel, EtOAc-heptane) afforded 0.042 g of 1-cyclopropyl-3-((S)-1-(((S)-2,3-dihydrobenzo[b][1,4]dioxin-2-yl)methyl)piperidin-3-yl)imidazolidine-2,4-dione as white solid.

¹H NMR (400 MHz, CDCl₃) δ ppm 0.71-0.87 (4H, m), 1.56-1.80 (3H, m), 2.06-2.24 (2H, m), 2.57-2.74 (3H, m), 2.75-2.88 (3H, m), 3.76 (2H, s), 3.96-4.04 (1H, m), 4.08-4.19 (1H, m), 4.21-4.34 (2H, m), 6.78-6.90 (4H, m).

### EXAMPLE 120: 3-((S)-1-(((S)-2,3-Dihydrobenzo[b][1,4]dioxin-2-yl)methyl)-piperidin-3-yl)-1-(oxetan-3-yl)imidazolidine-2,4-dione

### Step 1: N—((S)-1-(((S)-2,3-Dihydrobenzo[b][1,4]dioxin-2-yl)methyl)piperidin-3-yl)-2-(oxetan-3-ylamino)acetamide

To a suspension of (S)-1-(((S)-2,3-dihydrobenzo[b][1,4]dioxin-2-yl)methyl)piperidin-3-amine (0.4 g, 1.61 mmol) and K₂CO₃ (0.67 g, 4.8 mmol) in MeCN (6 ml) at 0° C. was added chloroacetyl chloride (76 μl, 0.95 mmol) and the mixture was stirred at rt. After 2 hours diisopropylethylamine (0.28 ml, 1.61 mmol) and 3-oxetanamine hydrochloride (0.14 g, 1.29 mmol) were added and the mixture was stirred in closed vessel at 120° C. After 6 hours mixture was cooled and stirred at rt overnight. Heating was continued for 2.5 hours after which more 3-oxetanamine hydrochloride (0.10 g, 0.91 mmol) and K₂CO₃ (0.22 g, 1.59 mmol) were added. Mixture was stirred at 120° C. for further 3.5 hours and then at room temperature overnight. Reaction mixture was filtered and filtrate was concentrated. Purification of the evaporation residue by reverse phase column chromatography (C18, 0.1% NH₄OH/MeCN) afforded 0.27 g of N—((S)-1-(((S)-2,3-dihydrobenzo[b][1,4]dioxin-2-yl)methyl)piperidin-3-yl)-2-(oxetan-3-ylamino)acetamide as colorless oil.

LC-MS (ES+) [M+1]: 362.17.

### Step 2: 3-((S)-1-(((S)-2,3-Dihydrobenzo[b][1,4]dioxin-2-yl)methyl)piperidin-3-yl)-1-(oxetan-3-yl)imidazolidine-2,4-dione

To a solution of N—((S)-1-(((S)-2,3-dihydrobenzo[b][1,4]dioxin-2-yl)methyl)piperidin-3-yl)-2-(oxetan-3-ylamino)acetamide (0.27 g, 0.75 mmol) in MeCN (7.5 ml) was added triethylamine (0.10 ml, 0.75 mmol) and N,N-carbonyldiimidazole (0.18 g, 1.12 mmol). After 5 hours the reaction mixture was heated to 80° C. (heating block). After 2.5 hours more N,N-carbonyldiimidazole (0.060 g, 0.37 mmol) was added and the mixture was stirred at rt overnight. More N,N-carbonyldiimidazole (0.060 g, 0.37 mmol) was added and the mixture was heated to 80° C. After 4 hours solvents were evaporated and the residue was dissolved in EtOAc. Solution was washed with saturated NaHCO₃, dried (Na₂SO₄) and concentrated. Purification of the evaporation residue by reverse phase column chromatography (C18, 0.1% NH₄OH-MeCN) afforded 0.175 g of 3-((S)-1-(((S)-2,3-dihydrobenzo[b][1,4]dioxin-2-yl)methyl)piperidin-3-yl)-1-(oxetan-3-yl)imidazolidine-2,4-dione as white solid.

¹H NMR (400 MHz, CDCl₃) δ ppm 1.57-1.79 (3H, m), 2.07-2.24 (2H, m), 2.60-2.67 (1H, m), 2.67-2.74 (1H, m), 2.75-2.88 (3H, m), 3.98 (1H, dd), 4.08 (2H, s), 4.11-4.20 (1H, m), 4.21-4.32 (2H, m), 4.71-4.77 (2H, m), 4.84-4.90 (2H, m), 5.29-5.38 (1H, m), 6.79-6.88 (4H, m).

### EXAMPLE 121: 1-(3,3-Difluorocyclobutyl)-3-((S)-1-(((S)-2,3-dihydrobenzo[b][1,4]dioxin-2-yl)methyl)piperidin-3-yl)imidazolidine-2,4-dione

### Step 1: 2-(3,3-Difluorocyclobutylamino)-N—((S)-1-(((S)-2,3-dihydrobenzo[b][1,4]dioxin-2-yl)methyl)piperidin-3-yl)acetamide

To a suspension of (S)-1-(((S)-2,3-dihydrobenzo[b][1,4]dioxin-2-yl)methyl)piperidin-3-amine (0.20 g, 0.81 mmol) and K₂CO₃ (0.56 g, 4.0 mmol) in MeCN (3 ml) at 0° C. was added chloroacetyl chloride (64 μl, 0.81 mmol) and the mixture was stirred at rt. After 2 hours diisopropylethylamine (0.14 ml, 0.81 mmol) and 3,3-difluoro-cyclobutanamine hydrochloride (0.23 g, 1.61 mmol) were added and the mixture was stirred in closed vessel at 120° C. After 4 hours mixture was cooled and filtered and the filtrate was concentrated. Evaporation residue that contained 2-(3,3-difluorocyclobutylamino)-N—((S)-1-(((S)-2,3-dihydrobenzo[b][1,4]dioxin-2-yl)methyl)piperidin-3-yl)acetamide was used as such in the next step

LC-MS (ES+) [M+1]: 396.26.

### Step 2: 1-(3,3-Difluorocyclobutyl)-3-((S)-1-(((S)-2,3-dihydrobenzo[b][1,4]dioxin-2-yl)methyl)piperidin-3-yl)imidazolidine-2,4-dione

To a solution of 2-(3,3-difluorocyclobutylamino)-N—((S)-1-(((S)-2,3-dihydrobenzo[b][1,4]dioxin-2-yl)methyl)piperidin-3-yl)acetamide (0.28 g, 0.71 mmol) in MeCN (6.5 ml) was added triethylamine (0.10 ml, 0.71 mmol) and N,N-carbonyldiimidazole (0.172 g, 1.06 mmol). After 3 hours more N,N-carbonyldiimidazole (0.115 g, 0.71 mmol) was added and the mixture was heated 2 h at 80° C. (heating block) and then at rt for 3 days. Solvents were evaporated and the residue was dissolved in CH₂Cl₂. Solution was washed with saturated NaHCO₃, dried (Na₂SO₄) and concentrated. Purification of the evaporation residue by reverse phase column chromatography (C18, 0.1% HCOOH-MeCN) afforded 0.080 g of 1-(3,3-difluorocyclobutyl)-3-((S)-1-(((S)-2,3-dihydrobenzo[b][1,4]dioxin-2-yl)methyl)piperidin-3-yl)imidazolidine-2,4-dione as white solid.

¹H NMR (400 MHz, CDCl₃) δ ppm 1.56-1.79 (3H, m), 2.07-2.24 (2H, m), 2.60-2.66 (1H, m), 2.67-3.01 (8H, m), 3.85 (2H, s), 3.99 (1H, dd), 4.10-4.20 (1H, m), 4.21-4.31 (2H, m), 4.45-4.56 (1H, m), 6.79-6.88 (4H, m).

### EXAMPLE 122: 6-((S)-1-(((S)-2,3-Dihydrobenzo[b][1,4]dioxin-2-yl)methyl)-piperidin-3-yl)-4,6-diazaspiro[2.4]heptane-5,7-dione

### Step 1: tert-Butyl-1-((S)-1-(((S)-2,3-dihydrobenzo[b][1,4]dioxin-2-yl)methyl)piperidin-3-ylcarbamoyl)cyclopropylcarbamate

To a suspension of 1-(tert-butoxycarbonylamino)cyclopropanecarboxylic acid (0.26 g, 1.31 mmol) (U.S. Pat. No. 7,202,279 B1) in CH₂Cl₂ (4.5 mil) was added diisopropylethylamine (0.58 ml, 3.33 mmol), O-(benzotriazol-1-yl)-N,N,N′,N′-tetramethyluronium hexafluorophosphate (0.54 g, 1.43 mmol), (S)-1-(((S)-2,3-dihydrobenzo[b][1,4]dioxin-2-yl)methyl)piperidin-3-amine p-toluenesulfonate (0.50 g, 1.19 mmol) and N-methyl pyrrolidone (1.5 ml). After 20 hours 1M NaOH (20 ml) was added the phases were separated. Aqueous layer was extracted with EtOAc and the combined organic phases were was washed with brine, dried and concentrated to dryness. The yellow oily crude product (1.0 g) that contained tert-butyl 1-((S)-1-(((S)-2,3-dihydrobenzo[b][1,4]dioxin-2-yl)methyl)piperidin-3-ylcarbamoyl)cyclopropylcarbamate along with some residual tetramethylurea from the coupling reagent was used as such in the next step.

LC-MS (ES+) [M+1]: 432.42

### Step 2: 6-((S)-1-(((S)-2,3-Dihydrobenzo[b][1,4]dioxin-2-yl)methyl)piperidin-3-yl)-4,6-diazaspiro[2.4]heptane-5,7-dione

The crude product of tert-butyl-1-((S)-1-(((S)-2,3-dihydrobenzo[b][1,4]dioxin-2-yl)methyl)piperidin-3-ylcarbamoyl)cyclopropylcarbamate (0.51 g, 1.18 mmol) was dried azeotropically with toluene and dissolved in THF (12 ml). To the solution was added potassium tert-butylate (0.40 g, 3.55 mmol). After 3 days saturated NH₄Cl (15 ml) was added. Phases were separated and the water phase was extracted with EtOAc. Combined organic layers were washed with brine, dried (Na₂SO₄) and concentrated to dryness. Purification of the crude product by reverse phase column chromatography (C18, 0.1% NH₄OH/MeCN) afforded 0.30 g of 6-((S)-1-(((S)-2,3-dihydrobenzo[b][1,4]dioxin-2-yl)methyl)piperidin-3-yl)-4,6-diazaspiro[2.4]heptane-5,7-dione as white solid.

¹H NMR (400 MHz, CDCl₃) δ ppm 1.19-1.32 (2H, m), 1.42-1.54 (2H, m), 1.63-1.80 (3H, m), 2.10-2.25 (2H, m), 2.60-2.68 (1H, m), 2.68-2.75 (1H, m), 2.79-2.92 (3H, m), 4.00 (1H, dd), 4.14-4.34 (3H, m), 5.96 (1H, br s), 6.79-6.89 (4H, m).

### EXAMPLE 123: 6-((S)-1-(((S)-2,3-Dihydrobenzo[b][1,4]dioxin-2-yl)methyl)-piperidin-3-yl)-4-methyl-4,6-diazaspiro[2.4]heptane-5,7-dione

To a solution of 6-((S)-1-(((S)-2,3-dihydrobenzo[b][1,4]dioxin-2-yl)methyl)piperidin-3-yl)-4,6-diazaspiro[2.4]heptane-5,7-dione (0.10 g, 0.28 mmol) in DMF (1 ml) at 0° C. was added NaH (0.022 g, 0.56 mmol, 60% dispersion in mineral oil). After 20 minutes MeI (24 μl, 0.45 mmol) was added. After 2 hours saturated NH₄Cl (4 ml) was added and the mixture was extracted with EtOAc (3×). Combined organic layers were washed with brine, dried (Na₂SO₄) and concentrated. Purification of the evaporation residue by trituration with 1:1 methyl-tert-butyl ether-heptane afforded 0.059 g of 6-((S)-1-(((S)-2,3-dihydrobenzo[b][1,4]dioxin-2-yl)methyl)piperidin-3-yl)-4-methyl-4,6-diazaspiro[2.4]-heptane-5,7-dione as white solid.

¹H NMR (400 MHz, CDCl₃) δ ppm 1.27-1.33 (2H, m), 1.33-1.39 (2H, m), 1.61-1.79 (3H, m), 2.11-2.25 (2H, m), 2.60-2.68 (1H, m), 2.68-2.74 (4H, m), 2.79-2.91 (3H, m), 4.00 (1H, dd), 4.17-4.32 (3H, m), 6.79-6.89 (4H, m).

### EXAMPLE 124: 2-(6-((S)-1-(((S)-2,3-Dihydrobenzo[b][1,4]dioxin-2-yl)methyl)-piperidin-3-yl)-5,7-dioxo-4,6-diazaspiro[2.4]heptan-4-yl)-N,N-dimethylacetamide

To a solution of 6-((S)-1-(((S)-2,3-dihydrobenzo[b][1,4]dioxin-2-yl)methyl)piperidin-3-yl)-4,6-diazaspiro[2.4]heptane-5,7-dione (0.10 g, 0.28 mmol) in DMF (1 ml) at 0° C. was added NaH (0.015 g, 0.36 mmol, 60% dispersion in mineral oil). After 20 minutes 2-chloro-N,N-dimethylacetamide (29 μl, 0.28 mmol) was added and the reaction mixture was stirred at rt. After 3 hours saturated NH₄Cl (4 ml) was added and the mixture was extracted with EtOAc (3×). Combined organic layers were washed with brine, dried (Na₂SO₄) and concentrated. Purification of the evaporation residue by reverse phase column chromatography (C18, 0.1% NH₄OH/MeCN) afforded 0.11 g of 2-(6-((S)-1-(((S)-2,3-dihydrobenzo[b][1,4]dioxin-2-yl)methyl)piperidin-3-yl)-5,7-dioxo-4,6-diazaspiro[2.4]-heptan-4-yl)-N,N-dimethylacetamide as white solid.

¹H NMR (400 MHz, CDCl₃) δ ppm 1.23-1.29 (2H, m), 1.35-1.42 (2H, m), 1.58-1.84 (3H, m), 2.11-2.25 (2H, m), 2.59-2.67 (1H, m), 2.68-2.75 (1H, m), 2.79-2.93 (3H, m), 2.95 (3H, s), 3.03 (3H, s), 3.88 (2H, s), 3.96-4.03 (1H, m), 4.19-4.32 (3H, m), 6.78-6.89 (4H, m).

### EXAMPLE 125: 1-((S)-1-(((S)-2,3-Dihydrobenzo[b][1,4]dioxin-2-yl)methyl)-piperidin-3-yl)-3-ethylimidazolidine-2,4,5-trione

To a suspension of (S)-1-(((S)-2,3-dihydrobenzo[b][1,4]dioxin-2-yl)methyl)piperidin-3-amine p-toluenesulfonate (0.3 g, 0.71 mmol) in acetonitrile (2.5 ml) were added triethylamine (0.10 ml, 0.72 mmol) and ethyl isocyanate (56 μl, 0.71 mmol). The mixture was stirred at rt for 3 hours after which more ethyl isocyanate (15 μl, 0.19 mmol) was added. After 2 hours solvents were evaporated from the reaction mixture and the residue was dissolved in EtOAc. Solution was washed with sat. NaHCO₃ and brine, dried and concentrated to dryness. To the oily evaporation residue was added THF (2.5 ml), the solution was cooled to 0° C. and oxalyl chloride (64 μl, 0.76 mmol) was added. After 2 hours solvents were evaporated, the residue was taken in a mixture of CH₂Cl₂ and water, sat. NaHCO₃ was added until the pH of the water phase pH was 8. Phases were separated and the water phase was extracted with CH₂Cl₂. Combined organic layers were dried (Na₂SO₄) and solvents were evaporated. Purification of the evaporation residue by filtration through silica (EtOAc-heptane) afforded 0.14 g of 1-((S)-1-(((S)-2,3-dihydrobenzo[b][1,4]dioxin-2-yl)methyl)piperidin-3-yl)-3-ethylimidazolidine-2,4,5-trione as colorless oil.

¹H NMR (400 MHz, CDCl₃) δ ppm 1.29 (3H, t), 1.60-1.74 (1H, m), 1.75-1.85 (2H, m), 2.04-2.17 (1H, m), 2.17-2.27 (1H, m), 2.62-2.81 (3H, m), 2.84-2.97 (2H, m), 3.70 (2H, q), 3.96-4.03 (1H, m), 4.22-4.34 (3H, m), 6.80-6.89 (4H, m).

### EXAMPLE 126: 1-Cyclohexyl-3-((S)-1-(((S)-2,3-dihydrobenzo[b][1,4]dioxin-2-yl)methyl)piperidin-3-yl)imidazolidine-2,4,5-trione

To a suspension of (S)-1-(((S)-2,3-dihydrobenzo[b][1,4]dioxin-2-yl)methyl)piperidin-3-amine p-toluenesulfonate (0.30 g, 0.71 mmol) in acetonitrile (2.5 ml) were added triethylamine (0.10 ml, 0.72 mmol) and ethyl cyclohexyl isocyanate (91 μl, 0.71 mmol). After 1 hour solvents were evaporated and the residue was taken into mixture of in EtOAc and sat. NaHCO₃. The resulting suspension was filtered. The precipitate was washed with water and dried in vacuo. The organic portion of filtrate was washed with brine, dried (Na₂SO₄) and concentrated. Combining the two residues gave 0.21 g of 1-cyclohexyl-3-((S)-1-(((S)-2,3-dihydrobenzo[b][1,4]dioxin-2-yl)methyl)piperidin-3-yl)urea as white solid. The combined residues were dissolved in THF (2.5 ml), the solution was cooled to 0° C. and oxalyl chloride (52 μl, 0.76 mmol) was added. After 4 hours solvents were evaporated, the residue was taken in a mixture of CH₂Cl₂ and water and sat. NaHCO₃ was added until the pH of the water phase pH was 8. Phases were separated and the water phase was extracted with CH₂Cl₂. Combined organic layers were dried (Na₂SO₄) and solvents were evaporated. Purification of the evaporation residue by filtration through silica (EtOAc-heptane) afforded 0.15 g of 1-cyclohexyl-3-((S)-1-(((S)-2,3-dihydrobenzo[b][1,4]dioxin-2-yl)methyl)piperidin-3-yl)imidazolidine-2,4,5-trione as white solid.

¹H NMR (400 MHz, CDCl₃) δ ppm 1.16-1.40 (4H, m), 1.60-1.91 (7H, m), 2.0-2.16 (3H, m), 2.17-2.26 (1H, m), 2.61-2.69 (1H, m), 2.69-2.80 (2H, m), 2.84-2.96 (2H, m), 3.95-4.07 (2H, m), 4.21-4.32 (3H, m), 6.80-6.89 (4H, m).

### EXAMPLE 127: 1-Cyclopentyl-3-((S)-1-(((S)-2,3-dihydrobenzo[b][1,4]dioxin-2-yl)methyl)piperidin-3-yl)imidazolidine-2,4,5-trione

To a suspension of (S)-1-(((S)-2,3-dihydrobenzo[b][1,4]dioxin-2-yl)methyl)piperidin-3-amine p-toluenesulfonate (0.3 g, 0.71 mmol) in acetonitrile (2.5 ml) were added triethylamine (0.10 ml, 0.72 mmol) and ethyl cyclopentyl isocyanate (80 μl, 0.71 mmol). The mixture was stirred at rt for 5 hours after which more cyclopentyl isocyanate (20 μl, 0.18 mmol) and triethylamine (20 μl, 0.14 mmol) were added. After 1 hour solvents were evaporated and the residue was taken into mixture of in EtOAc and sat. NaHCO₃. The resulting suspension was filtered. The precipitate was washed with water and dried in vacuo. The organic portion of filtrate was washed with brine, dried (Na₂SO₄) and concentrated. Combining the two residues gave 0.19 g of 1-cyclopentyl-3-((S)-1-(((S)-2,3-dihydrobenzo[b][1,4]dioxin-2-yl)methyl)piperidin-3-yl)urea as white solid. The combined residues were dissolved in THF (2.5 ml), the solution was cooled to 0° C. and oxalyl chloride (62 μl, 0.76 mmol) was added. After 4 hours solvents were evaporated, the residue was taken a mixture of CH₂Cl₂ and water, sat. NaHCO₃ was added until the pH of the water phase pH was 8. Phases were separated and the water phase was extracted with CH₂Cl₂. Combined organic layers were dried (Na₂SO₄) and solvents were evaporated. Purification of the evaporation residue by filtration through silica (EtOAc-heptane) afforded 0.18 g of 1-cyclopentyl-3-((S)-1-(((S)-2,3-dihydrobenzo[b][1,4]dioxin-2-yl)methyl)-piperidin-3-yl)imidazolidine-2,4,5-trione as white solid.

¹H NMR (400 MHz, CDCl₃) δ ppm 1.58-1.73 (3H, m), 1.74-1.85 (2H, m), 1.86-2.16 (7H, m), 2.17-2.27 (1H, m), 2.60-2.81 (3H, m), 2.83-2.98 (2H, m), 3.95-4.04 (1H, m), 4.20-4.34 (3H, m), 4.45-4.57 (1H, m), 6.79-6.90 (4H, m).

### EXAMPLE 128: 1-((S)-1-(((S)-2,3-Dihydrobenzo[b][1,4]dioxin-2-yl)methyl)-piperidin-3-yl)-3-((R)-1-phenylethyl)imidazolidine-2,4,5-trione

To a suspension of (S)-1-(((S)-2,3-dihydrobenzo[b][1,4]dioxin-2-yl)methyl)piperidin-3-amine p-toluenesulfonate (0.11 g, 0.26 mmol) in acetonitrile (2.5 ml) were added triethylamine (0.36 μl, 0.26 mmol) and R-(+)-alpha-methylbenzyl isocyanate (37 μl, 0.26 mmol). After 3 hours solvents were evaporated from the reaction mixture and the residue was dissolved in EtOAc. Solution was washed with sat. NaHCO₃ and brine, dried and concentrated to dryness. To the oily evaporation residue was added THF (1.3 ml), the solution was cooled to 0° C. and oxalyl chloride (21 μl, 0.25 mmol) was added. After 4 hours solvents were evaporated, the residue was taken in a mixture of CH₂Cl₂ and water, sat. NaHCO₃ was added until the pH of the water phase pH was 8. Phases were separated and the water phase was extracted with CH₂Cl₂. Combined organic layers were dried (Na₂SO₄) and solvents were evaporated. Purification of the evaporation residue by filtration through silica (EtOAc-heptane), followed by trituration with MTBE afforded 0.031 g of 1-((S)-1-(((S)-2,3-dihydrobenzo[b][1,4]dioxin-2-yl)methyl)piperidin-3-yl)-3-((R)-1-phenylethyl)imidazolidine-2,4,5-trione as white solid.

¹H NMR (400 MHz, CDCl₃) δ ppm 1.61-1.70 (1H, m), 1.72-1.82 (2H, m), 1.83-1.92 (3H, m), 1.99-2.14 (1H, m), 2.14-2.24 (1H, m), 2.58-2.77 (3H, m), 2.78-2.97 (2H, m), 3.93-4.02 (1H, m), 4.18-4.32 (3H, m), 5.36-5.48 (1H, m), 6.77-6.89 (4H, m), 7.28-7.51 (5H, m).

### EXAMPLE 129: 1-((S)-1-(((S)-2,3-Dihydrobenzo[b][1,4]dioxin-2-yl)methyl)-piperidin-3-yl)-3-phenylimidazolidine-2,4,5-trione

To a suspension of (S)-1-(((S)-2,3-dihydrobenzo[b][1,4]dioxin-2-yl)methyl)piperidin-3-amine (0.10 g, 0.41 mmol) in acetonitrile (1.3 ml) was added phenyl isocyanate (45 μl, 0.41 mmol). After 3 hours solvents were evaporated from the reaction mixture. Residue was dissolved in THF (1.5 ml), the solution was cooled to 0° C. and oxalyl chloride (35 μl, 0.42 mmol) was added. After 3 hours triethylamine (106 μl, 0.76 mmol) and THF (1 ml) were added. After further 3 hours solvents were evaporated, the residue was taken in a mixture of CH₂Cl₂ and water and sat. NaHCO₃ was added until the pH of the water phase pH was 8. Phases were separated and the water phase was extracted with CH₂Cl₂. Combined organic layers were dried (Na₂SO₄) and solvents were evaporated. Purification of the evaporation residue by reverse phase column chromatography (C18, 0.1% HCOOH/MeCN) afforded 0.007 g of 1-((S)-1-(((S)-2,3-dihydrobenzo[b][1,4]dioxin-2-yl)methyl)piperidin-3-yl)-3-phenylimidazolidine-2,4,5-trione as colorless glass.

¹H NMR (400 MHz, CDCl₃) δ ppm 1.63-1.77 (1H, m), 1.78-1.94 (2H, m), 2.11-2.29 (2H, m), 2.64-2.71 (1H, m), 2.71-2.78 (1H, m), 2.80-2.88 (1H, m), 2.88-2.95 (1H, m), 2.99-3.06 (1H, m), 3.97-4.04 (1H, m), 4.25-4.32 (2H, m), 4.34-4.45 (1H, m), 6.80-6.90 (4H, m), 7.38-7.47 (3H, m), 7.48-7.54 (2H, m).

### EXAMPLE 130: 1-((S)-1-(((S)-2,3-Dihydrobenzo[b][1,4]dioxin-2-yl)methyl)-piperidin-3-yl)-3-isopropylimidazolidine-2,4,5-trione

To a suspension of (S)-1-(((S)-2,3-dihydrobenzo[b][1,4]dioxin-2-yl)methyl)piperidin-3-amine (0.10 g, 0.40 mmol) in acetonitrile (1.3 ml) was added isopropyl isocyanate (40 μl, 0.40 mmol). After 3 hours triethylamine (112 μl, 0.80 mmol) and isopropyl isocyanate (79 μl, 0.81 mmol) were added. After further 2 hours solvents were evaporated. Evaporation residue was dissolved in THF (3 ml), the solution was cooled to 0° C. and oxalyl chloride (53 μl, 0.63 mmol) was added. Reaction mixture was stirred at rt for 5 hours. Solvents were evaporated, the residue was taken in a mixture of CH₂Cl₂ and water and sat. NaHCO₃ was added until the pH of the water phase pH was 8. Phases were separated and the water phase was extracted with CH₂Cl₂. Combined organic layers were dried (Na₂SO₄) and concentrated to dryness. Purification of the evaporation residue by filtration through silica (EtOAc-heptane, twice) afforded 0.090 g of 1-((S)-1-(((S)-2,3-dihydrobenzo[b][1,4]dioxin-2-yl)methyl)piperidin-3-yl)-3-isopropylimidazolidine-2,4,5-trione as white solid.

¹H NMR (400 MHz, CDCl₃) δ ppm 1.46 (6H, d), 1.59-1.74 (1H, m), 1.74-1.86 (2H, m), 2.03-2.16 (1H, m), 2.17-2.27 (1H, m), 2.61-2.80 (3H, m), 2.85-2.97 (2H, m), 3.95-4.04 (1H, m), 4.21-4.33 (3H, m), 4.44 (1H, spt), 6.80-6.89 (4H, m).

### EXAMPLE 131: 1-Benzyl-3-((S)-1-(((S)-2,3-dihydrobenzo[b][1,4]dioxin-2-yl)methyl)piperidin-3-yl)imidazolidine-2,4,5-trione

To a suspension of (S)-1-(((S)-2,3-dihydrobenzo[b][1,4]dioxin-2-yl)methyl)piperidin-3-amine (0.1 g, 0.40 mmol) in acetonitrile (1.3 ml) was added benzyl isocyanate (50 μl, 0.40 mmol). After 4 hours solvents were evaporated and the evaporation residue was dissolved in THF (2 ml), the solution was cooled to 0° C. and oxalyl chloride (37 μl, 0.44 mmol) was added. Reaction mixture was stirred at rt for 3 hours. Solvents were evaporated, the residue was taken in a mixture of CH₂Cl₂ and water and sat. NaHCO₃ was added until the pH of the water phase pH was 8. Phases were separated and the water phase was extracted with CH₂Cl₂. Combined organic layers were dried (Na₂SO₄) and concentrated to dryness. Purification of the evaporation residue by filtration through silica (EtOAc-heptane) afforded 0.10 g of 1-benzyl-3-((S)-1-(((S)-2,3-dihydrobenzo[b][1,4]dioxin-2-yl)methyl)piperidin-3-yl)imidazolidine-2,4,5-trione as white solid.

¹H NMR (400 MHz, CDCl₃) δ ppm 1.62-1.72 (1H, m), 1.73-1.84 (2H, m), 2.00-2.14 (1H, m), 2.15-2.26 (1H, m), 2.59-2.79 (3H, m), 2.83-2.95 (2H, m), 3.93-4.03 (1H, m), 4.20-4.33 (3H, m), 4.76 (2H, s), 6.78-6.89 (4H, m), 7.24-7.44 (5H, m).

### EXAMPLE 132: 1-((S)-1-(((S)-2,3-Dihydrobenzo[b][1,4]dioxin-2-yl)methyl)-piperidin-3-yl)-3-propylimidazolidine-2,4,5-trione

To a suspension of (S)-1-(((S)-2,3-dihydrobenzo[b][1,4]dioxin-2-yl)methyl)piperidin-3-amine (0.15 g, 0.60 mmol) in acetonitrile (2 ml) was added propyl isocyanate (59 μl, 0.63 mmol). After 2 hours solvents were evaporated. Evaporation residue was dissolved in THF (2 ml), the solution was cooled to 0° C. and oxalyl chloride (54 μl, 0.64 mmol) was added. Reaction mixture was stirred at rt for 2 hours. Solvents were evaporated, the residue was taken in a mixture of CH₂Cl₂ and water and sat. NaHCO₃ was added until the pH of the water phase pH was 8. Phases were separated and the water phase was extracted with CH₂Cl₂. Combined organic layers were dried (Na₂SO₄) and concentrated to dryness. Purification of the evaporation residue by filtration through silica (EtOAc-heptane) afforded 0.16 g of 1-((S)-1-(((S)-2,3-dihydrobenzo[b][1,4]dioxin-2-yl)methyl)piperidin-3-yl)-3-propylimidazolidine-2,4,5-trione as white solid.

¹H NMR (400 MHz, CDCl₃) δ ppm 0.94 (3H, t), 1.60-1.86 (5H, m), 2.02-2.16 (1H, m), 2.16-2.27 (1H, m), 2.60-2.82 (3H, m), 2.83-2.99 (2H, m), 3.60 (2H, t), 3.95-4.04 (1H, m), 4.21-4.34 (3H, m), 6.77-6.90 (4H, m).

### EXAMPLE 133: 1-((S)-1-(((S)-2,3-Dihydrobenzo[b][1,4]dioxin-2-yl)methyl)-piperidin-3-yl)-3-((S)-1-phenylethyl)imidazolidine-2,4,5-trione

### Step 1: 1-((S)-1-(((S)-2,3-Dihydrobenzo-[b][1,4]dioxin-2-yl)methyl)piperidin-3-yl)-3-((S)-1-phenylethyl)urea

To a solution of (S)-1-(((S)-2,3-dihydrobenzo[b][1,4]dioxin-2-yl)methyl)piperidin-3-amine (0.30 g, 1.21 mmol) in DMF (3 ml) was added S-(−)-alpha-methylbenzyl isocyanate (0.17 ml, 1.21 mmol). After 2 h water (10 ml) was added and the solution was extracted with EtOAc. Combined organic layers were washed with brine, dried and concentrated to dryness. Purification of the evaporation residue by reverse phase column chromatography (0.1% NH₄OH-MeCN) afforded 0.27 g of 1-((S)-1-(((S)-2,3-dihydrobenzo-[b][1,4]dioxin-2-yl)methyl)piperidin-3-yl)-3-((S)-1-phenylethyl)urea as white solid.

LC-MS (ES−) [M−1]: 394.27.

### Step 2: 1-((S)-1-(((S)-2,3-Dihydrobenzo[b][1,4]dioxin-2-yl)methyl)piperidin-3-yl)-3-((S)-1-phenylethyl)imidazolidine-2,4,5-trione

To solution of 1-((S)-1-(((S)-2,3-dihydrobenzo[b][1,4]dioxin-2-yl)methyl)piperidin-3-yl)-3-((S)-1-phenylethyl)urea (0.10 g, 0.25 mmol) in acetonitrile (1.5 ml) at 0° C. was added oxalyl chloride (21 μl, 0.25 mmol). Reaction mixture was refluxed for 3 hours after which more oxalyl chloride (21 μl, 0.25 mmol) and MeCN (1 ml) were added. After refluxing for additional 3 hours solvents were evaporated. The residue was taken in a mixture of CH₂Cl₂ and water and sat. NaHCO₃ was added until the pH of the water phase pH was 8. Phases were separated and the water phase was extracted with CH₂Cl₂. Combined organic layers were dried (Na₂SO₄) and concentrated to dryness. Purification of the evaporation residue by column chromatography (silica gel, EtOAc-heptane) afforded 0.036 g of 1-((S)-1-(((S)-2,3-dihydrobenzo[b][1,4]dioxin-2-yl)methyl)piperidin-3-yl)-3-((S)-1-phenylethyl)imidazolidine-2,4,5-trione white solid.

¹H NMR (400 MHz, CDCl₃) δ ppm 1.58-1.70 (1H, m), 1.73-1.82 (2H, m), 1.89 (3H, d), 1.99-2.13 (1H, m), 2.15-2.23 (1H, m), 2.60-2.67 (1H, m), 2.67-2.77 (2H, m), 2.79-2.97 (2H, m), 3.94-4.02 (1H, m), 4.19-4.30 (3H, m), 5.43 (1H, q), 6.79-6.88 (4H, m), 7.29-7.39 (3H, m), 7.44-7.49 (2H, m).

### EXAMPLE 134: 1-tert-Butyl-3-((S)-1-(((S)-2,3-dihydrobenzo[b][1,4]dioxin-2-yl)methyl)piperidin-3-yl)imidazolidine-2,4,5-trione

Starting material 1-tert-butyl-3-((S)-1-(((S)-2,3-dihydrobenzo[b][1,4]dioxin-2-yl)methyl)-piperidin-3-yl)urea was obtained as a by-product from Example 117.

LC-MS (ES−) [M−1]: 346.23

To a solution of 1-tert-butyl-3-((S)-1-(((S)-2,3-dihydrobenzo[b][1,4]dioxin-2-yl)methyl)piperidin-3-yl)urea (0.23 g, 0.66 mmol) at 0° C. was added oxalyl chloride (62 μl, 0.73 mmol). Reaction mixture was stirred at rt for 6 hours. Solvents were evaporated, the residue was taken in a mixture of CH₂Cl₂ and water and sat. NaHCO₃ was added until the pH of the water phase pH was 8. Phases were separated and the water phase was extracted with CH₂Cl₂. Combined organic layers were dried (Na₂SO₄) and concentrated to dryness. Purification of the evaporation residue by column chromatography (silica gel, EtOAc-heptane) afforded 0.16 g of 1-tert-butyl-3-((S)-1-(((S)-2,3-dihydrobenzo[b][1,4]dioxin-2-yl)methyl)piperidin-3-yl)imidazolidine-2,4,5-trione as white solid.

¹H NMR (400 MHz, CDCl₃) δ ppm 1.64 (s, 9H) 1.63-1.74 (1H, m), 1.74-1.83 (2H, m), 2.03-2.16 (1H, m), 2.17-2.26 (1H, m), 2.61-2.69 (1H, m), 2.69-2.81 (2H, m), 2.82-2.98 (2H, m), 3.95-4.03 (1H, m), 4.20-4.32 (3H, m), 6.80-6.89 (4H, m).

### EXAMPLE 135: 1-((S)-1-(((S)-2,3-Dihydrobenzo[b][1,4]dioxin-2-yl)methyl)-piperidin-3-yl)-3-(2-(dimethylamino)ethyl)imidazolidine-2,4,5-trione

### Step 1: 1-((S)-1-(((S)-2,3-Dihydrobenzo[b][1,4]dioxin-2-yl)methyl)piperidin-3-yl)-3-(2-(dimethylamino)ethyl)urea

To a suspension of (S)-1-(((S)-2,3-dihydrobenzo[b][1,4]dioxin-2-yl)methyl)piperidin-3-amine (0.10 g, 0.40 mmol) in acetonitrile (1.3 ml) was added 2-chloroethyl isocyanate (34 μl, 0.40 mmol). After 2 hours solvents were evaporated. The evaporation residue was dissolved in 33%-solution of dimethylamine in ethanol (0.54 ml, 3.14 mmol) and the solution was heated to 50° C. After 8 hours reaction mixture was brought to rt. After 3 days, reaction mixture was concentrated to dryness. Purification of the evaporation residue by reverse phase column chromatography (C18, 0.1% NH₄OH/MeCN) afforded 0.090 g of 1-((S)-1-(((S)-2,3-dihydrobenzo[b][1,4]dioxin-2-yl)methyl)piperidin-3-yl)-3-(2-(dimethylamino)ethyl)urea as yellowish oil.

LC-MS (ES−) [M−1]: 361.37.

### Step 2: 1-((S)-1-(((S)-2,3-Dihydrobenzo[b][1,4]-dioxin-2-yl)methyl)piperidin-3-yl)-3-(2-(dimethylamino)ethyl)imidazolidine-2,4,5-trione

To a solution of 1-((S)-1-(((S)-2,3-dihydrobenzo[b][1,4]dioxin-2-yl)methyl)piperidin-3-yl)-3-(2-(dimethylamino)ethyl)urea (0.090 g, 0.25 mmol) in THF (1.3 ml) at 0° C. was added oxalyl chloride (23 μl, 0.27 mmol). Reaction mixture was stirred at rt for 3 hours. Solvents were evaporated, the residue was taken in a mixture of CH₂Cl₂ and water and sat. NaHCO₃ was added until the pH of the water phase pH was 8. Phases were separated and the water phase was extracted with CH₂Cl₂. Combined organic layers were dried (Na₂SO₄) and concentrated to dryness. Purification of the evaporation residue by column chromatography (silica gel, MeOH—CH₂Cl₂) afforded 0.052 g of 1-((S)-1-(((S)-2,3-dihydrobenzo[b][1,4]-dioxin-2-yl)methyl)piperidin-3-yl)-3-(2-(dimethylamino)ethyl)imidazolidine-2,4,5-trione as white solid.

¹H NMR (400 MHz, CDCl₃) δ ppm 1.58-1.73 (1H, m), 1.74-1.87 (2H, m), 2.02-2.16 (1H, m), 2.17-2.23 (1H, m), 2.24 (6H, s), 2.56 (2H, t), 2.61-2.80 (3H, m), 2.85-2.99 (2H, m), 3.73 (2H, t), 3.95-4.04 (1H, m), 4.22-4.34 (3H, m), 6.79-6.89 (4H, m).

### EXAMPLE 136: 1-((S)-1-(((S)-2,3-Dihydrobenzo[b][1,4]dioxin-2-yl)methyl)piperidin-3-yl)-3-(tetrahydro-2H-pyran-4-yl)imidazolidine-2,4,5-trione

### Step 1: 1-((S)-1-(((S)-2,3-Dihydrobenzo[b][1,4]dioxin-2-yl)methyl)piperidin-3-yl)-3-(tetrahydro-2H-pyran-4-yl)urea

To a solution of tetrahydro-pyran-4-carboxylic acid (0.20 g, 1.54 mmol) in toluene (2 ml) at 0° C. was added triethylamine (0.21 ml, 1.54 mmol) and diphenylphosphoryl azide (0.33 ml, 1.54 mmol). Reaction mixture was stirred at 0° C. for 2 h, then at rt overnight and finally 2 h at 100° C. Reaction mixture was cooled to rt and diluted with toluene (5 ml). To the reaction mixture was added a solution (S)-1-(((S)-2,3-dihydrobenzo[b][1,4]dioxin-2-yl)methyl)-piperidin-3-amine (0.38 g, 1.54 mmol) in CH₂Cl₂ (2 ml). After 20 hours the reaction mixture was concentrated to dryness and the evaporation residue was taken in EtOAc. The suspension was washed with saturated NaHCO₃. Collection of the precipitate by filtration afforded 0.25 g of 1-((S)-1-(((S)-2,3-dihydrobenzo[b][1,4]dioxin-2-yl)methyl)piperidin-3-yl)-3-(tetrahydro-2H-pyran-4-yl)urea as white solid.

LC-MS (ES+) [M+1]: 376.60.

### Step 2: 1-((S)-1-(((S)-2,3-Dihydrobenzo[b][1,4]dioxin-2-yl)methyl)piperidin-3-yl)-3-(tetrahydro-2H-pyran-4-yl)imidazolidine-2,4,5-trione

To a solution of 1-((S)-1-(((S)-2,3-dihydrobenzo[b][1,4]dioxin-2-yl)methyl)piperidin-3-yl)-3-(tetrahydro-2H-pyran-4-yl)urea (0.25 g, 0.67 mmol) in THF (3.5 ml) at 0° C. was added oxalyl chloride (62 μl, 0.73 mmol). Reaction mixture was stirred at rt for 4 hours. Solvents were evaporated, the residue was taken in a mixture of CH₂Cl₂ and water and sat. NaHCO₃ was added until the pH of the water phase pH was 8. Phases were separated and the water phase was extracted with CH₂Cl₂. Combined organic layers were dried (Na₂SO₄) and concentrated to dryness. Purification of the evaporation residue by filtration through silica (EtOAc-heptane) afforded 0.20 g of 1-((S)-1-(((S)-2,3-dihydrobenzo[b][1,4]dioxin-2-yl)methyl)piperidin-3-yl)-3-(tetrahydro-2H-pyran-4-yl)imidazolidine-2,4,5-trione as white solid.

¹H NMR (400 MHz, CDCl₃) δ ppm 1.60-1.74 (3H, m), 1.75-1.85 (2H, m), 2.04-2.16 (1H, m), 2.18-2.27 (1H, m), 2.39-2.52 (2H, m), 2.62-2.80 (3H, m), 2.86-2.97 (2H, m), 3.39-3.48 (2H, m), 3.96-4.03 (1H, m), 4.05-4.11 (2H, m), 4.22-4.32 (4H, m), 6.80-6.89 (4H, m).

### EXAMPLE 137: 1-((S)-1-(((S)-2,3-Dihydrobenzo[b][1,4]dioxin-2-yl)methyl)piperidin-3-yl)-3-(piperidin-4-yl)imidazolidine-2,4,5-trione dihydrochloride

### Step 1: tert-Butyl-4-(3-((S)-1-(((S)-2,3-dihydrobenzo[b][1,4]dioxin-2-yl)methyl)-piperidin-3-yl)ureido)piperidine-1-carboxylate

To a solution of bis(trichloromethyl)carbonate (0.18 g, 0.61 mmol) in CH₂Cl₂ (3.5 ml) was slowly added a solution of tert-butyl 4-aminopiperidine-1-carboxylate (0.33 g, 2.70 mmol) and N,N-diisopropylethylamine (0.62 ml, 3.58 mmol) in CH₂Cl₂ (5 ml). After 2 h (S)-1-(((S)-2,3-dihydrobenzo[b][1,4]dioxin-2-yl)methyl)piperidin-3-amine (0.15 g, 0.61 mmol) was added. After 4 h saturated NaHCO₃ (10 mil) was added and the mixture was stirred for 15 minutes. Phases were separated and aqueous phase was extracted with CH₂Cl₂. Combined organic layers were dried (Na₂SO₄) and concentrated to dryness. Purification of the evaporation residue by reverse phase column chromatography (C18, 0.1% NH₄OH-MeCN) afforded 0.16 g of tert-butyl 4-(3-((S)-1-(((S)-2,3-dihydrobenzo[b][1,4]dioxin-2-yl)methyl)piperidin-3-yl)ureido)piperidine-1-carboxylate as colorless oil.

LC-MS (ES−) [M−1]: 373.45.

### Step 2: tert-Butyl-4-(3-((S)-1-(((S)-2,3-Dihydrobenzo[b][1,4]dioxin-2-yl)methyl)-piperidin-3-yl)-2,4,5-trioxoimidazolidin-1-yl)piperidine-1-carboxylate

To a solution of tert-butyl 4-(3-((S)-1-(((S)-2,3-dihydrobenzo[b][1,4]dioxin-2-yl)methyl)piperidin-3-yl)ureido)piperidine-1-carboxylate (0.16 g, 0.34 mmol) in THF (3 ml) at 0° C. was added oxalyl chloride (31 μl, 0.37 mmol). Reaction mixture was stirred at rt for 5 hours. Solvents were evaporated, the residue was taken in a mixture of CH₂Cl₂ and water and sat. NaHCO₃ was added until the pH of the water phase pH was 8. Phases were separated and the water phase was extracted with CH₂Cl₂. Combined organic layers were dried (Na₂SO₄) and concentrated to dryness. Purification of the evaporation residue by by filtration through silica (EtOAc-heptane) afforded 0.10 g of tert-butyl 4-(3-((S)-1-(((S)-2,3-dihydrobenzo[b][1,4]dioxin-2-yl)methyl)piperidin-3-yl)-2,4,5-trioxoimidazolidin-1-yl)piperidine-1-carboxylate as white solid.

(ES+) [M+1]: 529.90.

### Step 3: 1-((S)-1-(((S)-2,3-Dihydrobenzo[b][1,4]dioxin-2-yl)methyl)piperidin-3-yl)-3-(piperidin-4-yl)imidazolidine-2,4,5-trione dihydrochloride

To a solution of tert-butyl 4-(3-((S)-1-(((S)-2,3-dihydrobenzo[b][1,4]dioxin-2-yl)methyl)piperidin-3-yl)-2,4,5-trioxoimidazolidin-1-yl)piperidine-1-carboxylate (0.090 g, 0.17 mmol) in dioxane (1 ml) was added HCl-dioxane (0.34 ml, 1.36 mmol, 4M solution in dioxane). After 4 h HCl (0.34 ml, 1.36 mmol, 4M solution in dioxane) was added. After further 20 hours solvents were evaporated. Trituration of the evaporation residue with diethyl ether afforded 0.090 g of 1-((S)-1-(((S)-2,3-dihydrobenzo[b][1,4]dioxin-2-yl)methyl)piperidin-3-yl)-3-(piperidin-4-yl)imidazolidine-2,4,5-trione dihydrochloride as white solid.

¹H NMR (600 MHz, D₂O) δ ppm 1.79-1.90 (1H, m), 1.90-2.00 (3H, m), 2.01-2.24 (2H, m), 2.33-2.43 (2H, m), 2.99-3.07 (2H, m) 3.44-3.52 (4H, m), 3.53-3.75 (4H, m), 4.00 (1H, dd), 4.23-4.27 (1H, m), 4.30-4.38 (1H, m), 4.45-4.58 (1H, m), 4.74-4.80 (1H, m), 6.85-6.93 (4H, m).

### EXAMPLE 138: 1-((S)-1-(((S)-2,3-Dihydrobenzo[b][1,4]dioxin-2-yl)methyl)piperidin-3-yl)-3-methylimidazolidine-2,4,5-trione

### Step 1: 1-((S)-1-(((S)-2,3-Dihydrobenzo[b][1,4]dioxin-2-yl)methyl)piperidin-3-yl)imidazolidine-2,4,5-trione

To a solution of 1-((S)-1-(((S)-2,3-dihydrobenzo[b][1,4]dioxin-2-yl)methyl)piperidin-3-yl)urea (0.27 g, 0.93 mmol) in THF (5 ml) at 0° C. was added oxalyl chloride (98 μl, 1.16 mmol). Reaction mixture was stirred at rt for 1 hour. Solvents were evaporated, the residue was taken in a mixture of CH₂Cl₂ and water and sat. NaHCO₃ was added until the pH of the water phase pH was 7-8. Phases were separated and the water phase was extracted with EtOAc. Combined organic layers were washed with brine, dried (Na₂SO₄) and concentrated to dryness. Crude product containing 0.28 g of 1-((S)-1-(((S)-2,3-dihydrobenzo[b][1,4]-dioxin-2-yl)methyl)piperidin-3-yl)imidazolidine-2,4,5-trione as yellowish solid was used as such in the next step.

LC-MS (ES+) [M+1]: 346.56

### Step 2: 1-((S)-1-(((S)-2,3-Dihydrobenzo[b][1,4]dioxin-2-yl)methyl)piperidin-3-yl)-3-methylimidazolidine-2,4,5-trione

To a solution of 1-((S)-1-(((S)-2,3-dihydrobenzo[b][1,4]dioxin-2-yl)methyl)piperidin-3-yl)imidazolidine-2,4,5-trione (0.14 g, 0.41 mmol) in DMF (2 ml) at 0° C. was added NaH (0.019 g, 0.49 mmol, 60% dispersion in mineral oil). After 20 minutes MeI (35 μl, 0.57 mmol) was added. After 3 hours saturated NH₄Cl (8 ml) was added and the mixture was extracted with EtOAc (3×). Combined organic layers were washed with brine, dried (Na₂SO₄) and concentrated. Purification of the evaporation residue by by filtration through silica (EtOAc-heptane) afforded 0.030 g of 1-((S)-1-(((S)-2,3-dihydrobenzo[b][1,4]dioxin-2-yl)methyl)piperidin-3-yl)-3-methylimidazolidine-2,4,5-trione as white solid.

¹H NMR (400 MHz, CDCl₃) δ ppm 1.60-1.74 (1H, m), 1.75-1.85 (2H, m), 2.04-2.16 (1H, m), 2.17-2.27 (1H, m), 2.62-2.80 (3H, m), 2.82-2.98 (2H, m), 3.16 (3H, s), 3.95-4.03 (1H, m), 4.22-4.35 (3H, m), 6.80-6.89 (4H, m).

### EXAMPLE 139: 1-(1-Acetylpiperidin-4-yl)-3-((S)-1-(((S)-2,3-dihydrobenzo[b][1,4]-dioxin-2-yl)methyl)piperidin-3-yl)imidazolidine-2,4,5-trione

To a mixture of 1-((S)-1-(((S)-2,3-dihydrobenzo[b][1,4]dioxin-2-yl)methyl)piperidin-3-yl)-3-(piperidin-4-yl)imidazolidine-2,4,5-trione dihydrochloride (0.030 g, 0.060 mmol) and triethylamine (29 μl, 0.21 mmol) in CH₂Cl₂ (0.3 ml) at 0° C. was added acetyl chloride (6 μl, 0.084 mmol) and mixture was stirred at room temperature. After 2.5 h CH₂Cl₂ (5 ml) and water (5 ml) were added and pH of the water phase was adjusted to 8 with sat. NaHCO₃. Phases were separated and water phase was extracted with CH₂Cl₂. Combined organic layers were dried (Na₂SO₄) and concentrated to dryness. The evaporation residue contained 0.020 g of 1-(1-acetylpiperidin-4-yl)-3-((S)-1-(((S)-2,3-dihydrobenzo[b][1,4]dioxin-2-yl)methyl)piperidin-3-yl)imidazolidine-2,4,5-trione as colorless glass.

¹H NMR (400 MHz, CDCl₃) δ ppm 1.61-1.84 (5H, m), 2.02-2.12 (1H, m), 2.13 (3H, s), 2.15-2.39 (3H, m), 2.50-2.60 (1H, m), 2.62-2.80 (3H, m), 2.86-2.97 (2H, m), 3.06-3.17 (1H, m), 3.91-4.03 (2H, m), 4.19-4.32 (4H, m), 4.77-4.85 (1H, m), 6.80-6.89 (4H, m)

### EXAMPLE 140: 1-((S)-1-(((S)-2,3-Dihydrobenzo[b][1,4]dioxin-2-yl)methyl)piperidin-3-yl)-3-(pyridin-4-ylmethyl)imidazolidine-2,4,5-trione

To a solution of 1-((S)-1-(((S)-2,3-dihydrobenzo[b][1,4]dioxin-2-yl)methyl)piperidin-3-yl)imidazolidine-2,4,5-trione (0.15 g, 0.43 mmol) in DMF (1.5 ml) at 0° C. was added NaH (0.042 g, 1.04 mmol, 60% dispersion in mineral oil). After 20 minutes 4-(Bromomethyl)pyridine hydrobromide (0.110 g, 0.43 mmol) was added and the reaction mixture was stirred at 0° C. for 30 min and then at rt. After 3 hours saturated NH₄Cl (5 ml) was added and the mixture was extracted with EtOAc (3×). Combined organic layers were washed with brine, dried (Na₂SO₄) and concentrated. Purification of the evaporation residue by column chromatography (silica gel, EtOAc-heptane) and trituration by methyl tert-butylether-heptane afforded 0.082 g of 1-((S)-1-(((S)-2,3-dihydrobenzo[b][1,4]dioxin-2-yl)methyl)piperidin-3-yl)-3-(pyridin-4-ylmethyl)imidazolidine-2,4,5-trione as white solid.

¹H NMR (400 MHz, CDCl₃) δ ppm 1.59-1.73 (1H, m), 1.75-1.86 (2H, m), 2.02-2.14 (1H, m), 2.17-2.26 (1H, m), 2.61-2.79 (3H, m), 2.85-2.97 (2H, m), 3.95-4.03 (1H, m), 4.22-4.34 (3H, m), 4.76 (2H, s), 6.80-6.88 (4H, m), 7.26-7.29 (2H, m), 8.61-8.64 (2H, m).

### EXAMPLE 141: 1-((S)-1-(((S)-2,3-Dihydrobenzo[b][1,4]dioxin-2-yl)methyl)piperidin-3-yl)-3-isobutylimidazolidine-2,4,5-trione

### Step 1: 1-((S)-1-(((S)-2,3-Dihydrobenzo[b][1,4]dioxin-2-yl)methyl)piperidin-3-yl)-3-isobutylurea

To a solution of isovaleric acid (0.125 g, 1.22 mmol) in toluene (2 ml) at 0° C. was added triethylamine (0.17 ml, 1.22 mmol) and diphenylphosphoryl azide (0.27 ml, 1.26 mmol). Reaction mixture was refluxed for 1.5 h and cooled to rt. A solution (S)-1-(((S)-2,3-dihydrobenzo[b][1,4]dioxin-2-yl)methyl)piperidin-3-amine (0.304 g, 1.22 mmol) in CH₂Cl₂ (2 ml) was added. After 3 hours the reaction mixture was concentrated to dryness and the evaporation residue was dissolved in EtOAc. The solution was washed with saturated NaHCO₃ (3×) and brine, dried (Na₂SO₄) and concentrated to dryness. The residue (0.34 g) containing 1-((S)-1-(((S)-2,3-dihydrobenzo[b][1,4]dioxin-2-yl)methyl)piperidin-3-yl)-3-isobutylurea was used as such in the next step.

LC-MS (ES+) [M+1]: 348.84.

### Step 2: 1-((S)-1-(((S)-2,3-Dihydrobenzo[b][1,4]dioxin-2-yl)methyl)piperidin-3-yl)-3-isobutylimidazolidine-2,4,5-trione

To a solution of 1-((S)-1-(((S)-2,3-dihydrobenzo[b][1,4]dioxin-2-yl)methyl)piperidin-3-yl)-3-isobutylurea (0.34 g, 0.98 mmol) in in THF (4 ml) at 0° C. was added oxalyl chloride (91 μl, 0.73 mmol). Reaction mixture was stirred at rt for 3.5 hours. Solvents were evaporated, the residue was taken in a mixture of CH₂Cl₂ and water and sat. NaHCO₃ was added until the pH of the water phase pH was 8. Phases were separated and the water phase was extracted with CH₂Cl₂. Combined organic layers were dried (Na₂SO₄) and concentrated to dryness. Purification of the evaporation residue by filtration through silica (EtOAc-heptane) afforded 0.30 g of 1-((S)-1-(((S)-2,3-dihydrobenzo[b][1,4]dioxin-2-yl)methyl)piperidin-3-yl)-3-isobutylimidazolidine-2,4,5-trione as white solid.

¹H NMR (400 MHz, CDCl₃) δ ppm 0.93 (6H, d), 1.58-1.74 (1H, m), 1.75-1.87 (2H, m), 2.00-2.16 (2H, m), 2.16-2.28 (1H, m), 2.60-2.82 (3H, m), 2.84-2.98 (2H, m), 3.45 (2H, d), 3.94-4.04 (1H, m), 4.21-4.36 (3H, m), 6.78-6.89 (4H, m).

### EXAMPLE 142: 1-((S)-1-(((S)-2,3-Dihydrobenzo[b][1,4]dioxin-2-yl)methyl)piperidin-3-yl)-3-(pyridin-2-ylmethyl)imidazolidine-2,4,5-trione

To a solution of 1-((S)-1-(((S)-2,3-dihydrobenzo[b][1,4]dioxin-2-yl)methyl)piperidin-3-yl)imidazolidine-2,4,5-trione (0.15 g, 0.43 mmol) in DMF (1.5 ml) at 0° C. was added NaH (0.042 g, 1.04 mmol, 60% dispersion in mineral oil). After 20 minutes 2-(bromomethyl)-pyridine hydrobromide (0.132 g, 0.52 mmol) was added and the reaction mixture was stirred at 0° C. for 30 min and then at rt. After 4 hours saturated NH₄Cl (5 ml) was added and the mixture was extracted with EtOAc (3×). Combined organic layers were washed with brine, dried (Na₂SO₄) and concentrated. Purification of the evaporation residue by filtration through silica (EtOAc-heptane) afforded 0.10 g of 1-((S)-1-(((S)-2,3-dihydrobenzo[b][1,4]-dioxin-2-yl)methyl)piperidin-3-yl)-3-(pyridin-2-ylmethyl)imidazolidine-2,4,5-trione as white solid.

¹H NMR (400 MHz, CDCl₃) δ ppm 1.63-1.73 (1H, m), 1.75-1.89 (2H, m), 2.06-2.26 (2H, m), 2.62-2.83 (3H, m), 2.85-2.92 (1H, m), 2.94-3.00 (1H, m), 3.96-4.03 (1H, m), 4.23-4.37 (3H, m), 4.94 (2H, s), 6.80-6.89 (4H, m), 7.18-7.23 (1H, m), 7.26-7.30 (1H, m), 7.65-7.71 (1H, m), 8.49-8.52 (1H, m).

### EXAMPLE 143: 1-((S)-1-(((S)-2,3-Dihydrobenzo[b][1,4]dioxin-2-yl)methyl)piperidin-3-yl)-3-(pyridin-3-ylmethyl)imidazolidine-2,4,5-trione

To a solution of 1-((S)-1-(((S)-2,3-dihydrobenzo[b][1,4]dioxin-2-yl)methyl)piperidin-3-yl)imidazolidine-2,4,5-trione (0.15 g, 0.43 mmol) in DMF (1.5 ml) at 0° C. was added NaH (0.042 g, 1.04 mmol, 60% dispersion in mineral oil). After 20 minutes 3-(bromomethyl)-pyridine hydrobromide (0.132 g, 0.52 mmol) was added and the reaction mixture was stirred at 0° C. for 30 min and then at rt. After 6 hours saturated NH₄Cl (5 ml) was added and the mixture was extracted with EtOAc (3×). Combined organic layers were washed with brine, dried (Na₂SO₄) and concentrated. Purification of the evaporation residue by column chromatography (EtOAc-heptane) afforded 0.10 g of 1-((S)-1-(((S)-2,3-dihydrobenzo[b][1,4]dioxin-2-yl)methyl)piperidin-3-yl)-3-(pyridin-3-ylmethyl)-imidazolidine-2,4,5-trione as white solid.

¹H NMR (400 MHz, CDCl₃) δ ppm 1.56-1.73 (1H, m), 1.74-1.88 (2H, m), 1.99-2.14 (1H, m), 2.15-2.27 (1H, m), 2.59-2.79 (3H, m), 2.84-2.97 (2H, m), 3.93-4.04 (1H, m), 4.20-4.35 (3H, m), 4.79 (2H, s), 6.78-6.91 (4H, m), 7.27-7.33 (1H, m), 7.74 (1H, d), 8.59 (1H, d), 8.68 (1H, br s).

### EXAMPLE 144: 1-((S)-1-(((S)-2,3-Dihydrobenzo[b][1,4]dioxin-2-yl)methyl)piperidin-3-yl)-1H-benzo[d]imidazol-2(3H)-one

### Step 1: (S)-1-(((S)-2,3-Dihydrobenzo[b][1,4]dioxin-2-yl)methyl)-N-(2-nitrophenyl)-piperidin-3-amine

A mixture of (S)-1-(((S)-2,3-dihydrobenzo[b][1,4]dioxin-2-yl)methyl)piperidin-3-amine (0.80 g, 3.22 mmol), 2-fluoronitrobenzene (0.37 ml, 3.54 mmol) and K₂CO₃ (0.58 g, 4.19 mmol) in DMF (6 ml) was heated at 60° C. After 4 hours water (20 ml) was added and the mixture was extracted with CH₂Cl₂. Combined organic layers were washed with water, dried (Na₂SO₄) and concentrated to dryness. The residue (1.1 g) containing (S)-1-(((S)-2,3-dihydrobenzo[b][1,4]dioxin-2-yl)methyl)-N-(2-nitrophenyl)piperidin-3-amine was used as such in the next step.

LC-MS (ES+) [M+1]: 370.44.

### Step 2: N1-((S)-1-(((S)-2,3-Dihydrobenzo[b][1,4]-dioxin-2-yl)methyl)piperidin-3-yl)benzene-1,2-diamine

To a mixture of (S)-1-(((S)-2,3-dihydrobenzo[b][1,4]dioxin-2-yl)methyl)-N-(2-nitrophenyl)-piperidin-3-amine (0.30 g, 0.81 mmol) and NH₄Cl (0.43 g, 8.12 mmol) in THF (3 ml), MeOH (1.5 ml) and water (1.5 ml) was added zinc (0.53 g, 8.12 mmol). After 2 hours the reaction mixture was filtered through celite and celite cake was washed with EtOAc and water. Phases were separated from the filtrate and the water phase was extracted with EtOAc. Combined organic layers were washed with brine, dried (Na₂SO₄) and concentrated to dryness. The residue contained N1-((S)-1-(((S)-2,3-dihydrobenzo[b][1,4]-dioxin-2-yl)methyl)piperidin-3-yl)benzene-1,2-diamine that could be used as such in next steps.

LC-MS (ES+) [M+1]: 340.47.

### Step 3: 1-((S)-1-(((S)-2,3-Dihydrobenzo[b][1,4]dioxin-2-yl)methyl)piperidin-3-yl)-1H-benzo[d]imidazol-2(3H)-one

To a solution of N1-((S)-1-(((S)-2,3-dihydrobenzo[b][1,4]dioxin-2-yl)methyl)piperidin-3-yl)benzene-1,2-diamine (0.045 g, 0.13 mmol) in THF (1.3 ml) was added N,N-carbonyldiimidazole (0.021 g, 0.13 mmol). After 44 hours more N,N-carbonyldiimidazole (0.013 g, 0.080 mmol) was added. After one day EtOAc and sat. NaHCO₃ were added and phases were separated. Aqueous phase was extracted with EtOAc. Combined organic layers were washed with brine, dried (Na₂SO₄) and concentrated. Purification of the evaporation residue by column chromatography (silica gel, MeOH—CH₂Cl₂) afforded 0.025 g of 1-((S)-1-(((S)-2,3-dihydrobenzo[b][1,4]dioxin-2-yl)methyl)piperidin-3-yl)-1H-benzo[d]imidazol-2(3H)-one as colorless oil.

¹H NMR (400 MHz, CDCl₃) δ ppm 1.71-1.97 (3H, m), 2.18-2.33 (2H, m), 2.66-2.79 (2H, m), 2.86-2.94 (1H, m), 2.94-3.00 (1H, m), 3.04-3.10 (1H, m), 3.99-4.06 (1H, m), 4.26-4.36 (2H, m), 4.41-4.52 (1H, m), 6.78-6.89 (4H, m), 7.02-7.12 (3H, m), 7.15-7.22 (1H, m), 9.45 (1H, s).

### EXAMPLE 145: 1-((S)-1-(((S)-2,3-Dihydrobenzo[b][1,4]dioxin-2-yl)methyl)piperidin-3-yl)-1H-benzo[d][1,2,3]triazole

To a solution of N1-((S)-1-(((S)-2,3-dihydrobenzo[b][1,4]dioxin-2-yl)methyl)piperidin-3-yl)benzene-1,2-diamine (0.10 g, 0.30 mmol) in glacial acetic acid (1 ml) and water (0.5 ml) at 0° C. was added a solution of NaNO₂ (0.023 g, 0.34 mmol) in water (0.5 ml). Reaction mixture was heated to 80° C. After 1 h, reaction mixture was cooled to rt and concentrated to dryness. Sat. NaHCO₃ and CH₂Cl₂ were added and phases were separated. Aqueous phase was extracted with CH₂Cl₂. Combined organic layers were dried (Na₂SO₄) and concentrated to dryness. Evaporation residue contained 0.078 g of 1-((S)-1-(((S)-2,3-dihydrobenzo[b][1,4]dioxin-2-yl)methyl)piperidin-3-yl)-1H-benzo[d][1,2,3]triazole as brown oil.

¹H NMR (400 MHz, CDCl₃) δ ppm 1.79-2.02 (2H, m), 2.18-2.31 (2H, m), 2.33-2.42 (1H, m), 2.68-2.91 (2H, m), 2.87 (1H, t), 3.00-3.08 (1H, m), 3.32-3.39 (1H, m), 3.97-4.04 (1H, m), 4.27-4.36 (2H, m), 4.80-4.90 (1H, m), 6.79-6.89 (4H, m), 7.34-7.39 (1H, m), 7.46-7.51 (1H, m), 7.57-7.61 (1H, m), 8.07 (1H, dt).

### EXAMPLE 146: 1-((S)-1-(((S)-2,3-Dihydrobenzo[b][1,4]dioxin-2-yl)methyl)piperidin-3-yl)-1,3-dihydrobenzo[c][1,2,5]thiadiazole 2,2-dioxide

A solution of N1-((S)-1-(((S)-2,3-dihydrobenzo[b][1,4]dioxin-2-yl)methyl)piperidin-3-yl)benzene-1,2-diamine (0.050 g, 0.15 mmol) and sulfamide (0.020 g, 0.21 mmol) in pyridine (0.75 ml) was heated to 130° C. After 5 hours reaction mixture was cooled and concentrated to dryness. Purification of the evaporation residue by reverse phase column chromatography (C18, 0.1% NH₄OH-MeCN) afforded 0.020 g of 1-((S)-1-(((S)-2,3-dihydrobenzo[b][1,4]dioxin-2-yl)methyl)piperidin-3-yl)-1,3-dihydrobenzo[c][1,2,5]-thiadiazole 2,2-dioxide as white solid.

¹H NMR (400 MHz, CDCl₃) δ ppm 1.65-1.79 (1H, m), 1.83-1.92 (1H, m), 1.99-2.12 (1H, m), 2.19-2.32 (2H, m), 2.62-2.81 (3H, m), 2.91-2.98 (1H, m), 3.28-3.35 (1H, m), 4.00 (1H, dd), 4.03-4.13 (1H, m), 4.27-4.34 (2H, m), 6.80-6.95 (7H, m), 7.02-7.07 (1H, m).

### EXAMPLE 147: 1-((S)-1-(((S)-2,3-Dihydrobenzo[b][1,4]dioxin-2-yl)methyl)piperidin-3-yl)-3-methyl-1H-benzo[d]imidazol-2(3H)-one

To a solution of 1-((S)-1-(((S)-2,3-dihydrobenzo[b][1,4]dioxin-2-yl)methyl)piperidin-3-yl)-1H-benzo[d]imidazol-2(3H)-one (0.053 g, 0.145 mmol) in DMF (0.7 ml) at 0° C. was added NaH (0.09 g, 0.22 mmol, 60% dispersion in mineral oil). After 20 minutes MeI (11 μl, 0.18 mmol) was added. After 2 hours water (8 ml) was added and the mixture was extracted with CH₂Cl₂ (3×) and EtOAc (3×). Combined organic layers were washed with brine, dried (Na₂SO₄) and concentrated. Purification of the evaporation residue by column chromatography (silica gel, CH₂Cl₂-MeOH) afforded 0.027 g of 1-((S)-1-(((S)-2,3-dihydrobenzo[b][1,4]dioxin-2-yl)methyl)piperidin-3-yl)-3-methyl-1H-benzo[d]imidazol-2(3H)-one as colorless oil.

¹H NMR (400 MHz, CDCl₃) δ ppm 1.69-1.94 (3H, m), 2.17-2.32 (2H, m), 2.65-2.78 (2H, m), 2.85-2.99 (2H, m), 3.00-3.07 (1H, m), 3.40 (3H, s), 3.98-4.05 (1H, m), 4.24-4.33 (2H, m), 4.39-4.49 (1H, m), 6.77-6.88 (4H, m), 6.95-7.00 (1H, m), 7.04-7.12 (2H, m), 7.14-7.19 (1H, m).

### EXAMPLE 148: 1-((S)-1-(((S)-2,3-Dihydrobenzo[b][1,4]dioxin-2-yl)methyl)piperidin-3-yl)-1H-benzo[d]imidazole

A solution of N1-((S)-1-(((S)-2,3-dihydrobenzo[b][1,4]dioxin-2-yl)methyl)piperidin-3-yl)benzene-1,2-diamine (0.10 g, 0.30 mmol) in formic acid (2 ml) was heated to 70° C. After 4 hours reaction was cooled and solvents were evaporated. Evaporation residue was taken in a mixture of CH₂Cl₂ and sat. NaHCO₃, layers were separated and the aqueous layer was further extracted with CH₂Cl₂. Combined organic layers were dried (Na₂SO₄) and concentrated to dryness. Purification of the evaporation residue by reverse phase column chromatography (C18, 0.1% NH₄OH-MeCN) afforded 0.055 g of 1-((S)-1-(((S)-2,3-dihydrobenzo[b][1,4]dioxin-2-yl)methyl)piperidin-3-yl)-1H-benzo[d]imidazole as brown solid.

¹H NMR (400 MHz, CDCl₃) δ ppm 1.70-2.01 (3H, m), 2.09-2.17 (1H, m), 2.50-2.59 (1H, m), 2.63-2.71 (1H, m), 2.71-2.87 (3H, m), 3.18-3.25 (1H, m), 4.02 (1H, dd), 4.28-4.39 (2H, m), 4.46-4.55 (1H, m), 6.81-6.92 (4H, m), 7.26-7.33 (2H, m), 7.40-7.45 (1H, m), 7.79-7.85 (1H, m), 8.29 (1H, s).

### EXAMPLE 149: 1-((S)-1-(((S)-2,3-Dihydrobenzo[b][1,4]dioxin-2-yl)methyl)piperidin-3-yl)-3-phenyl-1H-benzo[d]imidazol-2(3H)-one

To mixture of 1-((S)-1-(((S)-2,3-dihydrobenzo[b][1,4]dioxin-2-yl)methyl)piperidin-3-yl)-1H-benzo[d]imidazol-2(3H)-one (0.073 g, 0.20 mmol), 4 Å molecular sieves (40 mg, powdered), phenylboronic acid (0.049 g, 0.40 mmol) and triethylamine (56 μl, 0.40 mmol) in CH₂Cl₂ (1 mil) were added CuSO₄ (0.0032 g, 0.020 mmol) and TEMPO (0.034 g, 0.22 mmol). Reaction mixture was stirred under air atmosphere. After 7 days, the reaction mixture was filtered through celite and the filter cake was washed with CH₂Cl₂. Filtrate was concentrated to dryness. Purification of the evaporation residue by reverse phase column chromatography (C18, 0.1% NH₄OH-MeCN) afforded 0.011 g of 1-((S)-1-(((S)-2,3-dihydrobenzo[b][1,4]dioxin-2-yl)methyl)piperidin-3-yl)-3-phenyl-1H-benzo[d]imidazol-2(3H)-one as white solid.

¹H NMR (400 MHz, CDCl₃) δ ppm 1.72-1.92 (2H, m), 1.94-2.02 (1H, m), 2.24-2.38 (2H, m), 2.69 (1H, dd), 2.77 (1H, dd), 2.94-3.06 (2H, m), 3.09-3.15 (1H, m), 4.03 (1H, dd), 4.27-4.36 (2H, m), 4.46-4.55 (1H, m), 6.79-6.89 (4H, m), 7.02-7.16 (3H, m), 7.22-7.26 (1H, m), 7.37-7.43 (1H, m), 7.50-7.54 (4H, m).

### EXAMPLE 150: 1-((S)-1-(((S)-2,3-Dihydrobenzo[b][1,4]dioxin-2-yl)methyl)piperidin-3-yl)-2-methyl-1H-benzo[d]imidazole

A solution of N1-((S)-1-(((S)-2,3-dihydrobenzo[b][1,4]dioxin-2-yl)methyl)piperidin-3-yl)benzene-1,2-diamine (0.12 g, 0.35 mmol) in acetic acid (1.8 ml) was heated to 130° C. After 5 hours reaction was cooled and solvents were evaporated. The evaporation residue was taken in a mixture of EtOAc and sat. Na₂CO₃, layers were separated and the aqueous layer was further extracted with EtOAc. Combined organic layers were washed with brine, dried (Na₂SO₄) and concentrated to dryness. Purification of the evaporation residue by reverse phase column chromatography (C18, 0.1% NH₄OH-MeCN) afforded 0.075 g of 1-((S)-1-(((S)-2,3-dihydrobenzo[b][1,4]dioxin-2-yl)methyl)piperidin-3-yl)-2-methyl-1H-benzo[d]imidazole as brownish solid.

¹H NMR (400 MHz, CDCl₃) δ ppm 1.74-2.04 (3H, m), 2.18-2.36 (2H, m), 2.65 (3H, s), 2.67-2.74 (1H, m), 2.74-2.81 (1H, m), 2.89-2.97 (1H, m), 3.01-3.08 (1H, m), 3.09-3.15 (1H, m), 3.98-4.04 (1H, m), 4.27-4.35 (2H, m), 4.40-4.50 (1H, m), 6.80-6.89 (4H, m), 7.16-7.23 (2H, m), 7.50-7.55 (1H, m), 7.66-7.71 (1H, m).

### EXAMPLE 151: 1-((S)-1-(((S)-2,3-Dihydrobenzo[b][1,4]dioxin-2-yl)methyl)piperidin-3-yl)-2-(methoxymethyl)-1H-benzo[d]imidazole

To a solution of 2-methoxyethanol (26 μl, 0.32 mmol) in a mixture of EtOAc (1.2 ml) and DMSO (0.6 ml) at 0° C. was added 1-propanephosphonic acid cyclic anhydride (0.12 ml, 0.59 mmol). The reaction mixture was stirred at rt. After 2 hours the reaction mixture was added to a flask containing N1-((S)-1-(((S)-2,3-dihydrobenzo[b][1,4]dioxin-2-yl)methyl)piperidin-3-yl)benzene-1,2-diamine (0.10 g, 0.30 mmol). After 20 hours water (7 ml) was added and the solution was made basic by addition of sat. NaHCO₃. Mixture was extracted with EtOAc and the combined organic layers were washer with brine, dried (Na₂SO₄) and concentrated to dryness. Purification of the evaporation residue by reverse phase column chromatography (C18, 0.1% HCOOH-MeCN) afforded 0.010 g of 1-((S)-1-(((S)-2,3-dihydrobenzo[b][1,4]dioxin-2-yl)methyl)piperidin-3-yl)-2-(methoxymethyl)-1H-benzo[d]imidazole as brown oil.

¹H NMR (400 MHz, CDCl₃) δ ppm 1.76-1.95 (2H, m), 1.98-2.07 (1H, m), 2.21-2.38 (2H, m), 2.68-2.80 (2H, m), 2.87-2.96 (1H, m), 2.98-3.05 (1H, m), 3.11-3.18 (1H, m), 3.39 (3H, s), 3.98-4.05 (1H, m), 4.26-4.34 (2H, m), 4.63-4.73 (1H, m), 4.73-4.81 (2H, m), 6.79-6.89 (4H, m), 7.22-7.29 (2H, m), 7.57-7.63 (1H, m), 7.73-7.79 (1H, m).

### EXAMPLE 152: 1-(6-Chloro-1-((S)-1-(((S)-2,3-dihydrobenzo[b][1,4]dioxin-2-yl)methyl)piperidin-3-yl)-1H-benzo[d]imidazol-2-yl)-N,N-dimethylmethanamine

### Step 1: (S)—N-(5-Chloro-2-nitrophenyl)-1-(((S)-2,3-dihydrobenzo[b][1,4]dioxin-2-yl)methyl)piperidin-3-amine

A mixture of (S)-1-(((S)-2,3-dihydrobenzo[b][1,4]dioxin-2-yl)methyl)piperidin-3-amine (1.50 g, 3.57 mmol), 2,4-dichloronitrobenzene (0.69 g, 3.57 mmol) and K₂CO₃ (1.09 g, 7.85 mmol) in DMF (12 ml) was heated at 120° C. After 7 hours water (40 ml) was added and pH was adjusted to 11-12 with 6M NaOH. The mixture was extracted with EtOAc. Combined organic layers were washed with 1M NaOH, water and brine, dried (Na₂SO₄) and concentrated to dryness. Purification of the evaporation residue by column chromatography (silica gel, EtOAc-heptane) afforded (S)—N-(5-chloro-2-nitrophenyl)-1-(((S)-2,3-dihydrobenzo[b][1,4]dioxin-2-yl)methyl)piperidin-3-amine as yellow oily solid.

LC-MS (ES+) [M+1]: 404.32.

### Step 2: 5-Chloro-N1-((S)-1-(((S)-2,3-dihydrobenzo[b][1,4]dioxin-2-yl)methyl)piperidin-3-yl)benzene-1,2-diamine

To a mixture of (S)—N-(5-chloro-2-nitrophenyl)-1-(((S)-2,3-dihydrobenzo[b][1,4]dioxin-2-yl)methyl)piperidin-3-amine (1.32 g, 3.27 mmol) and NH₄Cl (1.75 g, 32.7 mmol) in THF (13 mil), MeOH (7 mil) and water (7 mil) at 0° C. was added zinc (2.14 g, 32.7 mmol). Reaction mixture was stirred at 0° C. for 5 minutes and then at rt. After 2 hours the reaction mixture was filtered through celite and celite cake was washed with EtOAc. Filtrate was washed with brine, dried (Na₂SO₄) and concentrated to dryness. The residue contained 1.03 g 5-chloro-N1-((S)-1-(((S)-2,3-dihydrobenzo[b][1,4]dioxin-2-yl)methyl)piperidin-3-yl)benzene-1,2-diamine that could be used as such in next steps.

LC-MS (ES−) [M−1]: 372.33.

### Step 3: 6-Chloro-2-(chloromethyl)-1-((S)-1-(((S)-2,3-dihydrobenzo[b][1,4]dioxin-2-yl)methyl)piperidin-3-yl)-1H-benzo[d]imidazole

A solution of 5-chloro-N1-((S)-1-(((S)-2,3-dihydrobenzo[b][1,4]dioxin-2-yl)methyl)-piperidin-3-yl)benzene-1,2-diamine (0.68 g, 1.82 mmol) and chloroacetic acid (0.26 g, 2.73 mmol) in 5M aqueous HCl (3.4 ml) was heated in microwave reactor at 100° C. for 1 h. Heating was then continued traditionally in a closed vessel at 110° C. After 27 hours water (5 ml) was added and the pH of the mixture was adjusted to 8 with aqueous NaOH. Emerging precipitate was filtered and filtrate was extracted with EtOAc. Combined organic layers were washed with brine, dried (Na₂SO₄) and concentrated to dryness. Purification of the combined mixture of the precipitate and the evaporation residue by reverse phase column chromatography (C18, 0.1% NH₄OH-MeCN) afforded 0.068 g of 6-chloro-2-(chloromethyl)-1-((S)-1-(((S)-2,3-dihydrobenzo[b][1,4]dioxin-2-yl)methyl)piperidin-3-yl)-1H-benzo[d]imidazole as reddish solid.

LC-MS (ES+) [M+1]: 432.28

### Step 4: 1-(6-Chloro-1-((S)-1-(((S)-2,3-dihydrobenzo[b][1,4]dioxin-2-yl)methyl)piperidin-3-yl)-1H-benzo[d]imidazol-2-yl)-N,N-dimethylmethanamine

A solution of 6-chloro-2-(chloromethyl)-1-((S)-1-(((S)-2,3-dihydrobenzo[b][1,4]dioxin-2-yl)methyl)piperidin-3-yl)-1H-benzo[d]imidazole (0.068 g, 0.16 mmol) in 33-% dimethylamine-ethanol (1 ml, 5.9 mmol) was heated in microwave reactor to 100° C. After 1 h solvents were evaporated. Purification of the evaporation residue by reverse phase column chromatography (C18, 0.1% NH₄OH-MeCN) afforded 0.021 g of 1-(6-chloro-1-((S)-1-(((S)-2,3-dihydrobenzo[b][1,4]dioxin-2-yl)methyl)piperidin-3-yl)-1H-benzo[d]imidazol-2-yl)-N,N-dimethylmethanamine as reddish solid.

¹H NMR (400 MHz, CDCl₃) δ ppm 1.74-2.04 (3H, m), 2.15-2.27 (1H, m), 2.26 (6H, s), 2.32-2.40 (1H, m), 2.69-2.84 (3H, m), 2.95-3.03 (1H, m), 3.08-3.16 (1H, m), 3.64-3.77 (2H, m), 4.02 (1H, dd), 4.24-4.37 (2H, m), 4.70-4.81 (1H, m), 6.79-6.89 (4H, m), 7.19 (1H, dd), 7.56 (1H, d), 7.63 (1H, d).

### EXAMPLE 153: 1-(6-Chloro-1-((S)-1-(((S)-2,3-dihydrobenzo[b][1,4]dioxin-2-yl)methyl)piperidin-3-yl)-1H-benzo[d]imidazol-2-yl)-N-methylmethanamine

A mixture of 6-chloro-2-(chloromethyl)-1-((S)-1-(((S)-2,3-dihydrobenzo[b][1,4]dioxin-2-yl)methyl)piperidin-3-yl)-1H-benzo[d]imidazole (0.050 g, 0.12 mmol) and 2M methylamine in ethanol (0.50 ml, 1.0 mmol) was heated to 70° C. in microwave reactor. After 1 h solvents were evaporated. Purification of the evaporation residue by reverse phase column chromatography (C18, 0.1% NH₄OH-MeCN) afforded 0.018 g of 1-(6-chloro-1-((S)-1-(((S)-2,3-dihydrobenzo[b][1,4]dioxin-2-yl)methyl)piperidin-3-yl)-1H-benzo[d]imidazol-2-yl)-N-methylmethanamine as white solid.

¹H NMR (400 MHz, CDCl₃) δ ppm 1.74-1.96 (2H, m), 1.96-2.05 (1H, m), 2.13-2.26 (1H, m) 2.29-2.39 (1H, m), 2.50 (3H, s), 2.68-2.87 (3H, m), 2.98-3.06 (1H, m), 3.11-3.19 (1H, m), 3.98-4.05 (3H, m), 4.26-4.35 (2H, m), 4.61-4.72 (1H, m), 6.79-6.89 (4H, m), 7.19 (1H, dd), 7.55 (1H, d), 7.62 (1H, d).

### EXAMPLE 154: 1-((S)-1-(((S)-2,3-Dihydrobenzo[b][1,4]dioxin-2-yl)methyl)piperidin-3-yl)-4-fluoro-1H-benzo[d]imidazol-2(3H)-one

### Step 1: (S)-1-(((S)-2,3-Dihydrobenzo[b][1,4]dioxin-2-yl)methyl)-N-(3-fluoro-2-nitrophenyl)piperidin-3-amine

A mixture of (S)-1-(((S)-2,3-dihydrobenzo[b][1,4]dioxin-2-yl)methyl)piperidin-3-amine (0.20 g, 0.81 mmol), 2,6-difluoronitrobenzene (0.85 ml, 0.81 mmol) and K₂CO₃ (0.17 g, 1.21 mmol) in DMF (2.5 ml) was heated at 70° C. After 5 hours water (10 ml) was added and the mixture was extracted with CH₂Cl₂. Combined organic layers were washed with water, dried (Na₂SO₄) and concentrated to dryness. The residue (0.20 g) containing (S)-1-(((S)-2,3-dihydrobenzo[b][1,4]dioxin-2-yl)methyl)-N-(3-fluoro-2-nitrophenyl)piperidin-3-amine was used as such in the next step.

(ES+) [M+1]: 388.15.

### Step 2: N1-((S)-1-(((S)-2,3-Dihydrobenzo[b][1,4]dioxin-2-yl)methyl)piperidin-3-yl)-3-fluorobenzene-1,2-diamine

To a solution of(S)-1-(((S)-2,3-dihydrobenzo[b][1,4]dioxin-2-yl)methyl)-N-(3-fluoro-2-nitrophenyl)piperidin-3-amine (0.20 g, 0.52 mmol) in MeOH (2.5 ml) and formic acid (0.08 mil) was added ammonium formate (0.16 g, 2.6 mmol) and 10% Pd/C (0.055 g, 0.052 mmol). After 1 h reaction mixture was filtered through a pad of celite and the filter cake was washed with CH₂Cl₂ and MeOH. Filtrate was concentrated to dryness and the evaporation residue was dissolved in sat. NaHCO₃. Solution was extracted with CH₂Cl₂ and combined organic layers were dried (Na₂SO₄) and concentrated to dryness. Evaporation residue (0.13 g) contained N1-((S)-1-(((S)-2,3-dihydrobenzo[b][1,4]dioxin-2-yl)methyl)piperidin-3-yl)-3-fluorobenzene-1,2-diamine as red brown oil.

(ES+) [M+1]: 358.18.

### Step 3: 1-((S)-1-(((S)-2,3-Dihydrobenzo[b][1,4]dioxin-2-yl)methyl)piperidin-3-yl)-4-fluoro-1H-benzo[d]imidazol-2(3H)-one

To a solution of N1-((S)-1-(((S)-2,3-dihydrobenzo[b][1,4]dioxin-2-yl)methyl)piperidin-3-yl)-3-fluorobenzene-1,2-diamine (0.13 g, 0.36 mmol) in acetonitrile (6 ml) was added N,N-carbonyldiimidazole (0.077 g, 0.47 mmol). After 20 hours solvents were evaporated and the evaporation residue was dissolved in EtOAc. Solution was washed with water and water phase was back extracted with EtOAc. Combined organic layers were washed with brine, dried (Na₂SO₄) and concentrated to dryness. Purification of the evaporation residue by reverse phase column chromatography (C18, 0.1% NH₄OH-MeCN) afforded 0.080 g of 1-((S)-1-(((S)-2,3-dihydrobenzo[b][1,4]dioxin-2-yl)methyl)piperidin-3-yl)-4-fluoro-1H-benzo[d]imidazol-2(3H)-one as pale red solid.

¹H NMR (400 MHz, CDCl₃) δ ppm 1.70-1.96 (3H, m), 2.15-2.32 (2H, m), 2.66-2.79 (2H, m), 2.83-2.91 (1H, m), 2.92-2.99 (1H, m), 3.03-3.10 (1H, m), 3.99-4.05 (1H, m), 4.26-4.34 (2H, m), 4.37-4.48 (1H, m), 6.79-6.89 (5H, m), 6.94-7.04 (2H, m), 8.60 (1H, s).

### EXAMPLE 155: 3-((S)-1-(((S)-2,3-Dihydrobenzo[b][1,4]dioxin-2-yl)methyl)piperidin-3-yl)-5-methoxy-2-methyl-3H-imidazo[4,5-b]pyridine

### Step 1: N—((S)-1-(((S)-2,3-Dihydrobenzo[b][1,4]dioxin-2-yl)methyl)piperidin-3-yl)-6-methoxy-3-nitropyridin-2-amine

A mixture of (S)-1-(((S)-2,3-dihydrobenzo[b][1,4]dioxin-2-yl)methyl)piperidin-3-amine (0.11 g, 0.45 mmol), 2-chloro-6-methoxy-3-nitropyridine (0.093 g, 0.50 mmol) and K₂CO₃ (0.062 g, 0.45 mmol) in DMF (1.5 ml) was heated to 100° C. After 1.5 hours reaction mixture was cooled to rt, 1M HCl (12 ml) was added and the pH of the resulting mixture was adjusted to 10 with 1M NaOH. Mixture was extracted with EtOAc (3×). Combined organic layers were washed with brine, dried (Na₂SO₄) and concentrated to dryness. The residue (0.070 g) containing N—((S)-1-(((S)-2,3-dihydrobenzo[b][1,4]dioxin-2-yl)methyl)piperidin-3-yl)-6-methoxy-3-nitropyridin-2-amine was used as such in the next step.

LC-MS (ES+) [M+1]: 401.31.

### Step 2: N2-((S)-1-(((S)-2,3-Dihydrobenzo[b][1,4]dioxin-2-yl)methyl)piperidin-3-yl)-6-methoxypyridine-2,3-diamine

To a mixture of N—((S)-1-(((S)-2,3-dihydrobenzo[b][1,4]dioxin-2-yl)methyl)piperidin-3-yl)-6-methoxy-3-nitropyridin-2-amine (0.070 g, 0.18 mmol) and NH₄Cl (0.094 g, 1.75 mmol) in THF (0.8 ml), MeOH (0.4 ml) and water (0.4 ml) at 0° C. was added zinc (0.11 g, 1.75 mmol) and the reaction mixture was stirred at 0° C. for 5 min and then at rt. After 4 hours the reaction mixture was filtered through celite and celite cake was washed with EtOAc. The filtrate was washed with brine, dried (Na₂SO₄) and concentrated to dryness. The residue (0.040 g) contained N2-((S)-1-(((S)-2,3-dihydrobenzo[b][1,4]dioxin-2-yl)methyl)piperidin-3-yl)-6-methoxypyridine-2,3-diamine as purple oil that could be used as such in next step.

LC-MS (ES+) [M+1]: 371.30.

### Step 3: 3-((S)-1-(((S)-2,3-Dihydrobenzo[b][1,4]dioxin-2-yl)methyl)piperidin-3-yl)-5-methoxy-2-methyl-3H-imidazo[4,5-b]pyridine

A mixture of N2-((S)-1-(((S)-2,3-dihydrobenzo[b][1,4]dioxin-2-yl)methyl)piperidin-3-yl)-6-methoxypyridine-2,3-diamine (0.040 g, 0.11 mmol) and acetic anhydride (11 μl, 0.12 mmol) in acetic acid (0.7 ml) was heated to 130° C. in a closed vial. After 2.5 hours temperature was risen to 150° C. After further 14 hours acetic acid (0.7 ml) was added and the mixture was heated to 160° C. After 7 hours acetic acid was evaporated and the evaporation residue was taken in a mixture of EtOAc and sat. NaHCO₃. Phases were separated and the aqueous phase was extracted with EtOAc (2×). Combined organic layers were washed with brine, dried (Na₂SO₄) and concentrated to dryness. Purification of the evaporation residue by reverse phase column chromatography (C18, 0.1% NH₄OH-MeCN) afforded 0.009 g of 3-((S)-1-(((S)-2,3-dihydrobenzo[b][1,4]dioxin-2-yl)methyl)piperidin-3-yl)-5-methoxy-2-methyl-3H-imidazo[4,5-b]pyridine as reddish solid.

¹H NMR (400 MHz, CDCl₃) δ ppm 1.69-1.85 (1H, m), 1.85-1.96 (2H, m), 2.26-2.35 (1H, m), 2.61 (3H, s), 2.64-2.74 (2H, m), 2.75-2.82 (1H, m), 2.94-3.12 (2H, m), 3.32 (1H, t), 3.96 (3H, s), 4.01 (1H, dd), 4.28-4.36 (2H, m), 4.36-4.46 (1H, m), 6.61 (1H, d), 6.80-6.89 (4H, m), 7.78 (1H, d).

### EXAMPLE 156: 3-((S)-1-(((S)-2,3-Dihydrobenzo[b][1,4]dioxin-2-yl)methyl)-piperidin-3-yl)-2-methyl-3H-imidazo[4,5-b]pyridine

### Step 1: N—((S)-1-(((S)-2,3-Dihydrobenzo[b][1,4]dioxin-2-yl)methyl)piperidin-3-yl)-3-nitropyridin-2-amine

A mixture of (S)-1-(((S)-2,3-dihydrobenzo[b][1,4]dioxin-2-yl)methyl)piperidin-3-amine p-toluene sulfonate (0.25 g, 0.60 mmol), 2-chloro-3-nitropyridine (0.094 g, 0.60 mmol) and K₂CO₃ (0.123 g, 0.89 mmol) in DMF (4 ml) was heated to 120° C. in microwave reactor. After 2 hours more 2-chloro-3-nitropyridine (0.015 g, 0.095 mmol) and K₂CO₃ (0.10 g, 0.73 mmol) were added and the mixture was heated in microwave reactor at 120° C. for 1 h. Water (15 ml) was added and the mixture was extracted with EtOAc (3×). Combined organic layers were washed with 1M NaOH (2×) and brine, dried (Na₂SO₄) and concentrated to dryness. The residue (0.17 g) containing N—((S)-1-(((S)-2,3-dihydrobenzo[b][1,4]dioxin-2-yl)methyl)piperidin-3-yl)-3-nitropyridin-2-amine was used as such in the next step.

LC-MS (ES+) [M+1]: 371.30.

### Step 2: N2-((S)-1-(((S)-2,3-Dihydrobenzo[b][1,4]dioxin-2-yl)methyl)piperidin-3-yl)pyridine-2,3-diamine

To a mixture of N—((S)-1-(((S)-2,3-dihydrobenzo[b][1,4]dioxin-2-yl)methyl)piperidin-3-yl)-3-nitropyridin-2-amine (0.17 g, 0.46 mmol) and NH₄Cl (0.25 g, 4.60 mmol) in THF (2 ml), MeOH (1 ml) and water (1 ml) at 0° C. was added zinc (0.30 g, 4.60 mmol) and the reaction mixture was stirred at 0° C. for 5 min and then at rt. After 1.5 hours the reaction mixture was filtered through celite and celite cake was washed with EtOAc. The filtrate was washed with brine, dried (Na₂SO₄) and concentrated to dryness. The residue (0.18 g) contained N2-((S)-1-(((S)-2,3-dihydrobenzo[b][1,4]dioxin-2-yl)methyl)piperidin-3-yl)pyridine-2,3-diamine as brown oil that could be used as such in the next step.

LC-MS (ES−) [M−1]: 339.35.

### Step 3: 3-((S)-1-(((S)-2,3-Dihydrobenzo[b][1,4]dioxin-2-yl)methyl)piperidin-3-yl)-2-methyl-3H-imidazo[4,5-b]pyridine

A mixture of N2-((S)-1-(((S)-2,3-dihydrobenzo[b][1,4]dioxin-2-yl)methyl)piperidin-3-yl)pyridine-2,3-diamine (0.160 g, 0.47 mmol) and acetic anhydride (49 μl, 0.52 mmol) in acetic acid (2.4 ml) was heated to 130° C. in a closed vial. After 6 hours acetic acid was evaporated in vacuo and the evaporation residue was taken in a mixture of EtOAc and sat. NaHCO₃. Phases were separated and the aqueous phase was extracted with EtOAc (2×). Combined organic layers were washed with brine, dried (Na₂SO₄) and concentrated to dryness. Purification of the evaporation residue by reverse phase column chromatography (C18, 0.1% NH₄OH-MeCN) afforded an oily substance that turned into solid upon trituration with methyl-tert-butyl ether-heptane. Residue contained 0.034 g of 3-((S)-1-(((S)-2,3-dihydrobenzo[b][1,4]dioxin-2-yl)methyl)piperidin-3-yl)-2-methyl-3H-imidazo[4,5-b]pyridine as light brown solid.

¹H NMR (400 MHz, CDCl₃) δ ppm 1.72-1.86 (1H, m), 1.86-1.97 (2H, m), 2.36-2.45 (1H, m), 2.63-2.76 (5H, m), 2.77-2.85 (1H, m), 2.96-3.08 (2H, m), 3.30-3.38 (1H, m), 3.97-4.04 (1H, m), 4.28-4.35 (2H, m), 4.47-4.57 (1H, m), 6.79-6.89 (4H, m), 7.15 (1H, dd), 7.90 (1H, dd), 8.28 (1H, dd).

### EXAMPLE 157: 1-((S)-1-(((S)-2,3-Dihydrobenzo[b][1,4]dioxin-2-yl)methyl)-piperidin-3-yl)-2-methyl-1H-imidazo[4,5-b]pyridine

### Step 1: N—((S)-1-(((S)-2,3-Dihydrobenzo[b][1,4]dioxin-2-yl)methyl)piperidin-3-yl)-2-nitropyridin-3-amine

A mixture of (S)-1-(((S)-2,3-dihydrobenzo[b][1,4]dioxin-2-yl)methyl)piperidin-3-amine (0.25 g, 1.01 mmol), 2-nitro-3-fluoropyridine (0.14 g, 1.01 mmol) and K₂CO₃ (0.167 g, 1.21 mmol) in DMF (4 ml) was heated in sealed viel to 120° C. After 2 hours mixture was cooled, water (15 ml) was added and the mixture was extracted with EtOAc (3×). Combined organic layers were washed with 1M NaOH (2×) and brine, dried (Na₂SO₄) and concentrated to dryness. The residue (0.31 g) containing N—((S)-1-(((S)-2,3-dihydrobenzo[b][1,4]dioxin-2-yl)methyl)piperidin-3-yl)-2-nitropyridin-3-amine as yellow oil was used as such in the next step.

LC-MS (ES+) [M+1]: 371.27.

### Step 2: N3-((S)-1-(((S)-2,3-Dihydrobenzo[b][1,4]dioxin-2-yl)methyl)piperidin-3-yl)pyridine-2,3-diamine

To a mixture of N—((S)-1-(((S)-2,3-dihydrobenzo[b][1,4]dioxin-2-yl)methyl)piperidin-3-yl)-2-nitropyridin-3-amine (0.31 g, 0.84 mmol) and NH₄Cl (0.45 g, 8.37 mmol) in THF (4 ml), MeOH (2 ml) and water (2 ml) at 0° C. was added zinc (0.55 g, 8.37 mmol) and the reaction mixture was stirred at 0° C. for 5 min and then at rt. After 2 hours the reaction mixture was filtered through celite and celite cake was washed with EtOAc. The filtrate was washed with brine, dried (Na₂SO₄) and concentrated to dryness. The residue (0.32 g) contained N3-((S)-1-(((S)-2,3-dihydrobenzo[b][1,4]dioxin-2-yl)methyl)piperidin-3-yl)pyridine-2,3-diamine as brown oil that could be used as such in next step.

LC-MS (ES−) [M−1]: 339.27.

### Step 3: 1-((S)-1-(((S)-2,3-Dihydrobenzo[b][1,4]dioxin-2-yl)methyl)piperidin-3-yl)-2-methyl-1H-imidazo[4,5-b]pyridine

A mixture of N3-((S)-1-(((S)-2,3-dihydrobenzo[b][1,4]dioxin-2-yl)methyl)piperidin-3-yl)pyridine-2,3-diamine (0.30 g, 0.88 mmol) and acetic anhydride (0.10 ml, 0.97 mmol) in acetic acid (5.4 ml) was heated to 130° C. in a closed vial. After 5 hours acetic anhydride (50 μl, 0.49 mmol) was added and mixture was heated again 130° C. After further 2 hours acetic anhydride (50 μl, 0.49 mmol) was added and heating was continued at 130° C. After 4 hours solvents were evaporated in vacuo and the evaporation residue was taken in a mixture of EtOAc and sat. NaHCO₃. Phases were separated and the aqueous phase was extracted with EtOAc (2×). Combined organic layers were washed with brine, dried (Na₂SO₄) and concentrated to dryness. Purification of the evaporation residue by reverse phase column chromatography (C18, water-MeCN) afforded 0.17 g of 1-((S)-1-(((S)-2,3-dihydrobenzo[b][1,4]dioxin-2-yl)methyl)piperidin-3-yl)-2-methyl-1H-imidazo[4,5-b]pyridine as light brown solid.

¹H NMR (400 MHz, CDCl₃) δ ppm 1.78-1.88 (1H, m), 1.89-1.97 (1H, m), 1.98-2.07 (1H, m), 2.08-2.20 (1H, m), 2.26-2.35 (1H, m), 2.68-2.87 (6H, m), 3.02-3.09 (1H, m), 3.12-3.19 (1H, m), 4.01 (1H, dd), 4.26-4.36 (2H, m), 4.41-4.52 (1H, m), 6.80-6.90 (4H, m), 7.11 (1H, dd), 7.82 (1H, dd), 8.47 (1H, dd).

### EXAMPLE 158: 1-((S)-1-(((S)-2,3-Dihydrobenzo[b][1,4]dioxin-2-yl)methyl)piperidin-3-yl)-2-methyl-1H-imidazo[4,5-c]pyridine

### Step 1: N—((S)-1-(((S)-2,3-Dihydrobenzo[b][1,4]dioxin-2-yl)methyl)piperidin-3-yl)-3-nitropyridin-4-amine

A mixture of (S)-1-(((S)-2,3-dihydrobenzo[b][1,4]dioxin-2-yl)methyl)piperidin-3-amine (0.10 g, 0.40 mmol), 3-nitro-4-chloropyridine (0.064 g, 0.40 mmol) and K₂CO₃ (0.067 g, 0.48 mmol) in DMF (4 ml) was heated in sealed vial to 120° C. After 2 hours mixture was cooled, water (15 mil) was added and the mixture was extracted with EtOAc (3×). Combined organic layers were washed with 1M NaOH (2×) and brine, dried (Na₂SO₄) and concentrated to dryness. The residue (0.11 g) containing N—((S)-1-(((S)-2,3-dihydrobenzo[b][1,4]dioxin-2-yl)methyl)piperidin-3-yl)-3-nitropyridin-4-amine as orange oil was used as such in the next step.

LC-MS (ES−) [M−1]: 369.32.

### Step 2: N4-((S)-1-(((S)-2,3-Dihydrobenzo[b][1,4]dioxin-2-yl)methyl)piperidin-3-yl)pyridine-3,4-diamine

To a mixture of N—((S)-1-(((S)-2,3-dihydrobenzo[b][1,4]dioxin-2-yl)methyl)piperidin-3-yl)-3-nitropyridin-4-amine (0.11 g, 0.30 mmol) and NH₄Cl (0.16 g, 2.97 mmol) in THF (1.5 ml), MeOH (0.7 mil) and water (0.7 mil) at 0° C. was added zinc (0.19 g, 2.97 mmol) and the reaction mixture was stirred at 0° C. for 5 min and then at rt. After 2 hours the reaction mixture was filtered through celite and celite cake was washed with EtOAc. The filtrate was washed with brine, dried (Na₂SO₄) and concentrated to dryness. The residue (0.12 g) contained N4-((S)-1-(((S)-2,3-dihydrobenzo[b][1,4]dioxin-2-yl)methyl)piperidin-3-yl)pyridine-3,4-diamine as brown oil that could be used as such in next step.

LC-MS (ES+) [M+1]: 341.22.

### Step 3: 1-((S)-1-(((S)-2,3-Dihydrobenzo[b][1,4]dioxin-2-yl)methyl)piperidin-3-yl)-2-methyl-1H-imidazo[4,5-c]pyridine

A mixture of N4-((S)-1-(((S)-2,3-dihydrobenzo[b][1,4]dioxin-2-yl)methyl)piperidin-3-yl)pyridine-3,4-diamine (0.12 g, 0.35 mmol) and acetic anhydride (0.050 ml, 0.53 mmol) in acetic acid (2.4 mil) was heated to 130° C. in a closed vial. After 3 hours solvents were evaporated in vacuo and the evaporation residue was taken in a mixture of EtOAc and sat. NaHCO₃. Phases were separated and the aqueous phase was extracted with EtOAc (2×). Combined organic layers were washed with brine, dried (Na₂SO₄) and concentrated to dryness. Purification of the evaporation residue by reverse phase column chromatography (C18, water-MeCN) afforded 0.040 g of 1-((S)-1-(((S)-2,3-dihydrobenzo[b][1,4]dioxin-2-yl)methyl)piperidin-3-yl)-2-methyl-1H-imidazo[4,5-c]pyridine as light brown solid.

¹H NMR (400 MHz, CDCl₃) δ ppm 1.79-1.88 (1H, m), 1.90-1.98 (1H, m), 1.99-2.06 (1H, m), 2.11-2.23 (1H, m), 2.27-2.36 (1H, m), 2.67-2.89 (6H, m), 3.03-3.10 (1H, m), 3.11-3.18 (1H, m), 4.01 (1H, dd), 4.26-4.36 (2H, m), 4.41-4.50 (1H, m), 6.80-6.90 (4H, m), 7.45 (1H, dd), 8.35 (1H, d), 8.98 (1H, d)

### EXAMPLE 159: 3-((S)-1-(((S)-2,3-Dihydrobenzo[b][1,4]dioxin-2-yl)methyl)piperidin-3-yl)-2-ethyl-3H-imidazo[4,5-b]pyridine

A solution of N2-((S)-1-(((S)-2,3-dihydrobenzo[b][1,4]dioxin-2-yl)methyl)piperidin-3-yl)pyridine-2,3-diamine (0.200 g, 0.59 mmol) and propionic acid anhydride in propionic acid (4 ml) was heated to 145° C. in a sealed vial. After 4 hours solvents were evaporated in vacuo and the evaporation residue was taken in a mixture of EtOAc and sat. NaHCO₃. Phases were separated and the aqueous phase was extracted with EtOAc (2×). Combined organic layers were washed with brine, dried (Na₂SO₄) and concentrated to dryness. Purification of the evaporation residue by reverse phase column chromatography (C18, water-MeCN) afforded 0.040 g of 3-((S)-1-(((S)-2,3-dihydrobenzo[b][1,4]dioxin-2-yl)methyl)piperidin-3-yl)-2-ethyl-3H-imidazo[4,5-b]pyridine as brown oil.

¹H NMR (400 MHz, CDCl₃) δ ppm 1.47 (3H, t), 1.72-1.85 (1H, m), 1.87-1.95 (2H, m), 2.38-2.47 (1H, m), 2.67-2.84 (3H, m), 2.94-3.06 (4H, m), 3.40 (1H, t), 4.00 (1H, dd), 4.27-4.34 (2H, m), 4.44-4.53 (1H, m), 6.79-6.89 (4H, m), 7.15 (1H, dd), 7.94 (1H, dd), 8.27 (1H, dd).

### EXAMPLE 160: 9-((S)-1-(((S)-2,3-Dihydrobenzo[b][1,4]dioxin-2-yl)methyl)piperidin-3-yl)-8-methyl-9H-purine

### Step 1: 2-Chloro-N—((S)-1-(((S)-2,3-dihydrobenzo[b][1,4]dioxin-2-yl)methyl)piperidin-3-yl)-5-nitropyrimidin-4-amine

To a solution of 2,4-dichloro-5-nitropyrimidine (0.16 g, 0.81 mmol) in THF (5 ml) at −78° C. was added N,N-diisopropylethylamine (0.28 ml, 1.61 mmol) and a solution of (S)-1-(((S)-2,3-dihydrobenzo[b][1,4]dioxin-2-yl)methyl)piperidin-3-amine (0.20 g, 0.81 mmol) in THF (2 ml). Reaction mixture was stirred at −78° C. After 2 hours water was added and the mixture was extracted with EtOAc. Combined organic layers were washed with brine, dried (Na₂SO₄) and concentrated to dryness. The evaporation residue (0.32 g) contained 2-chloro-N—((S)-1-(((S)-2,3-dihydrobenzo[b][1,4]dioxin-2-yl)methyl)piperidin-3-yl)-5-nitropyrimidin-4-amine as yellow-orange solid that could be used as such in the next step.

LC-MS (ES−) [M−1]: 404.28.

### Step 2: N4-((S)-1-(((S)-2,3-Dihydrobenzo[b][1,4]dioxin-2-yl)methyl)piperidin-3-yl)pyrimidine-4,5-diamine

To a solution of 2-chloro-N—((S)-1-(((S)-2,3-dihydrobenzo[b][1,4]dioxin-2-yl)methyl)-piperidin-3-yl)-5-nitropyrimidin-4-amine (0.32 g, 0.79 mmol) in MeOH (5 ml) was added HCl (0.33 ml, 4.0 mmol, 37-% solution) and 10% Pd/C (0.11 g, 0.10 mmol). The mixture was subjected to H₂-atmosphere (50 psi). Pd/C (10%, 0.11 g, 0.10 mmol) was added several times to the reaction mixture during the hydrogenation process until the conversion was adequate (˜around 15 h). Reaction mixture was filtered through celite and the celite cake was washed with MeOH. Filtrate was concentrated and the evaporation residue was taken in sat. NaHCO₃. Mixture was extracted with EtOAc and the combined organic layers were washed with brine, dried (Na₂SO₄) and concentrated to dryness. Purification of the evaporation residue by reverse phase column chromatography (C18, 0.1% NH₄OH-MeCN) afforded 0.040 g of N4-((S)-1-(((S)-2,3-dihydrobenzo[b][1,4]dioxin-2-yl)methyl)piperidin-3-yl)pyrimidine-4,5-diamine as white solid.

LC-MS (ES+) [M+1]: 342.21.

### Step 3: 9-((S)-1-(((S)-2,3-Dihydrobenzo[b][1,4]-dioxin-2-yl)methyl)piperidin-3-yl)-8-methyl-9H-purine

A mixture of N4-((S)-1-(((S)-2,3-dihydrobenzo[b][1,4]dioxin-2-yl)methyl)piperidin-3-yl)pyrimidine-4,5-diamine (0.040 g, 0.12 mmol) and acetic anhydride (0.017 ml, 0.18 mmol) in acetic acid (1 ml) was heated to 130° C. in a closed vial. After 4 hours solvents were evaporated in vacuo and the evaporation residue was taken in a mixture of EtOAc and sat. NaHCO₃. Phases were separated and the aqueous phase was extracted with EtOAc (2×). Combined organic layers were washed with brine, dried (Na₂SO₄) and concentrated to dryness. Purification of the evaporation residue by reverse phase column chromatography (C18, 0.1% NH₄OH-MeCN) afforded 0.020 g of 9-((S)-1-(((S)-2,3-dihydrobenzo[b][1,4]-dioxin-2-yl)methyl)piperidin-3-yl)-8-methyl-9H-purine as white solid.

¹H NMR (400 MHz, CDCl₃) δ ppm 1.72-1.86 (1H, m), 1.88-1.98 (2H, m), 2.36-2.44 (1H, m), 2.60-2.75 (5H, m), 2.78-2.85 (1H, m), 2.98-3.09 (2H, m), 3.30 (1H, t), 4.00 (1H, dd), 4.27-4.35 (2H, m), 4.43-4.53 (1H, m), 6.80-6.89 (4H, m), 8.87 (1H, s), 8.97 (1H, s).

### EXAMPLE 161: 9-((S)-1-(((S)-2,3-Dihydrobenzo[b][1,4]dioxin-2-yl)methyl)-piperidin-3-yl)-9H-purine

A solution of N4-((S)-1-(((S)-2,3-dihydrobenzo[b][1,4]dioxin-2-yl)methyl)piperidin-3-yl)pyrimidine-4,5-diamine (0.060 g, 0.18 mmol) in formic acid (1.2 ml) was heated to 70° C. After 2 hours mixture was heated to 100° C. After further 9 hours, reaction mixture was cooled and solvents were evaporated in vacuo. The evaporation residue was taken in water and pH was adjusted to 9-10 with 5 M NaOH. Solution was extracted with EtOAc. Combined organic layers were washed with brine, dried (Na₂SO₄) and concentrated to dryness. Purification of the evaporation residue by reverse phase column chromatography (C18, 0.1% NH₄OH-MeCN) afforded 0.030 g of 9-((S)-1-(((S)-2,3-dihydrobenzo[b][1,4]dioxin-2-yl)methyl)piperidin-3-yl)-9H-purine as white solid.

¹H NMR (400 MHz, CDCl₃) δ ppm 1.68-1.87 (2H, m), 1.99-2.15 (2H, m), 2.63-2.86 (4H, m), 2.98-3.12 (2H, m), 4.02 (1H, dd), 4.30 (1H, dd), 4.34-4.41 (1H, m), 4.87-4.94 (1H, m), 6.81-6.95 (4H, m), 8.76 (1H, br s), 8.98 (1H, s), 9.15 (1H, s).

### EXAMPLE 162: 2-((S)-1-(((S)-2,3-Dihydrobenzo[b][1,4]dioxin-2-yl)methyl)piperidin-3-yl)-5,5-dimethylisothiazolidine 1,1-dioxide

### Step 1-2: (3S)-tert-Butyl 3-(5-methyl-1,1-dioxidoisothiazolidin-2-yl)piperidine-1-carboxylate

To a stirred solution of (S)-tert-butyl 3-aminopiperidine-1-carboxylate (2.0 g, 10.0 mmol) in CH₂Cl₂ (50 mL) was added Et₃N (2.1 mL, 15.0 mmol) followed by 4-chlorobutane-2-sulfonyl chloride (2.29 g, 12.0 mmol) (Justus Liebigs Annalen der Chemie, 1962, vol. 651, p. 17-29) at 0° C. and reaction mixture was stirred at rt. After 16 h the reaction mixture was diluted with CH₂Cl₂ and washed with water. The organic layer was dried (Na₂SO₄) and concentrated under reduced pressure to obtain crude compound. Purification of the crude product by column chromatography (20% ethyl acetate in pet ether) afforded 0.50 g of (3S)-tert-butyl 3-(3-chloro-1-methylpropylsulfonamido)piperidine-1-carboxylate as pale yellow liquid. The product (0.50 g, 1.41. mmol) was dissolved in ethanol (20 mL) and to the solution was added triethylamine (0.20 ml, 1.41 mmol) and NaOH (0.056 g, 1.41 mmol) at rt. The reaction mixture was heated to reflux. After 2 h the reaction mixture was cooled to rt and concentrated under reduced pressure. Water was added and the mixture was extracted with EtOAc. The organic layer was dried over anhydrous Na₂SO₄ and solvents were evaporated. Purification of the evaporation residue by column chromatography (silica gel, 30% ethyl acetate in pet ether) afforded 0.270 g of (3S)-tert-butyl 3-(5-methyl-1,1-dioxidoisothiazolidin-2-yl)piperidine-1-carboxylate as pale yellow liquid.

### Step 3: (S)-tert-Butyl 3-(5,5-dimethyl-1,1-dioxidoisothiazolidin-2-yl)piperidine-1-carboxylate

To as solution of (3S)-tert-butyl 3-(5-methyl-1,1-dioxidoisothiazolidin-2-yl)piperidine-1-carboxylate (2.1 g, 6.60) in THF (50 ml)) at 0° C. was added n-BuLi (8.25 ml, 13.2 mmol) followed by MeI (0.82 ml, 13.2 mmol) The mixture was then heated to 80° C. After 16 h the reaction mixture was cooled to rt and ammonium chloride was added. Mixture was extracted with EtOAc. The organic layer was dried (Na₂SO₄) and solvents were evaporate. Purification of the evaporation residue by mass directed prep HPLC afforded 0.10 g of (S)-tert-butyl 3-(5,5-dimethyl-1,1-dioxidoisothiazolidin-2-yl)piperidine-1-carboxylate as off white solid.

LC-MS (ES+) [M+1]: 333.2.

### Step 4: (S)-5,5-Dimethyl-2-(piperidin-3-yl)isothiazolidine 1,1-dioxide hydrochloride

To a solution of (S)-tert-butyl 3-(5,5-dimethyl-1,1-dioxidoisothiazolidin-2-yl)piperidine-1-carboxylate (0.30 g, 0.9 mmol) in dioxane (10 mL) was added solution of HCl in dioxane (10 mL) at 0° C. and reaction mixture was stirred at rt. After 1 h most of the solvent was distilled off. Evaporation residue was co-distilled twice with toluene. Trituration of the final evaporation residue with diethyl ether afforded 0.190 g of (S)-5,5-dimethyl-2-(piperidin-3-yl)isothiazolidine 1,1-dioxide hydrochloride as off white solid.

LC-MS (ES+) [M+1]: 233.0.

### Step 5: 2-((S)-1-(((S)-2,3-Dihydrobenzo[b][1,4]dioxin-2-yl)methyl)piperidin-3-yl)-5,5-dimethylisothiazolidine 1,1-dioxide

A mixture of of (S)-5,5-dimethyl-2-(piperidin-3-yl)isothiazolidine 1,1-dioxide hydrochloride (0.10 g, 0.37 mmol), (R)-2-(bromomethyl)-2,3-dihydrobenzo[b][1,4]dioxine (0.128 g, 0.56 mmol) and K₂CO₃ (0.129 g, 0.93 mmol) in MeCN (2 ml) was heated to 120° C. in microwave reactor. After 3 hours, the reaction mixture was cooled to rt, filtered and solvents were evaporated from the filtrate. Purification of the evaporation residue by column chromatography (silica gel, EtOAc-heptane) afforded 0.057 g of 2-((S)-1-(((S)-2,3-dihydrobenzo[b][1,4]dioxin-2-yl)methyl)piperidin-3-yl)-5,5-dimethylisothiazolidine 1,1-dioxide as solid.

¹H NMR (400 MHz, CDCl₃) δ ppm 1.41 (6H, d), 1.43-1.55 (1H, m), 1.59-1.78 (2H, m), 1.88-1.96 (1H, m), 2.06-2.17 (3H, m), 2.24 (1H, t), 2.58-2.71 (2H, m), 2.77-2.84 (1H, m), 3.04-3.11 (1H, m), 3.15-3.29 (2H, m), 3.60-3.69 (1H, m), 4.00 (1H, dd), 4.25-4.33 (2H, m), 6.79-6.89 (4H, m)

### EXAMPLE 163: 3-((S)-1-(((S)-2,3-Dihydrobenzo[b][1,4]dioxin-2-yl)methyl)piperidin-3-yl)oxazolidine-2,4-dione

### Step 1: N—((S)-1-(((S)-2,3-Dihydrobenzo[b][1,4]dioxin-2-yl)methyl)piperidin-3-yl)-2-hydroxyacetamide

To a solution of glycolic acid (0.234 g, 3.08 mmol) in DMF (5 ml) at rt was added TBTU (1.087 g, 3.38 mmol) and (S)-1-(((S)-2,3-dihydrobenzo[b][1,4]dioxin-2-yl)methyl)piperidin-3-amine (0,764 g, 3.08 mmol) in DMF (4 ml), followed by addition of N,N-diisopropylethylamine (DIPEA, 0.954 g, 7.38 mmol). The mixture was left stirring at rt overnight. The mixture was then quenched with water, extracted with EtOAc, and washed with brine. The extracts were dried (Na₂SO₄), and evaporated under vacuo. The crude product was purified by column chromatography with 4% MeOH in EtOAc, yielding 0.67 g of N—((S)-1-(((S)-2,3-dihydrobenzo[b][1,4]dioxin-2-yl)methyl)piperidin-3-yl)-2-hydroxyacetamide.

LC-MS, m/z=307.6 (M+1)⁺

### Step 2: 3-((S)-1-(((S)-2,3-Dihydrobenzo[b][1,4]dioxin-2-yl)methyl)piperidin-3-yl)oxazolidine-2,4-dione

The above product (0.583 g, 1.9 mmol) was dissolved in DMF (6 ml), to which was added N,N-carbonyldiimidazole (0.449 g, 2.77 mmol). The mixture was stirred at rt for 1 h, then heat up to 90° C. for 5 h. The reaction was monitored by LC-MS. The mixture was cooled to rt and diluted with EtOAc. Water was added and the mixture was extracted with EtOAc. Combined organic layers were washed with brine, dried (Na₂SO₄) and evaporated under vacuo. The product was purified by column chromatography with EtOAc:Hept (2:1), yielding 0.5175 g of 3-((S)-1-(((S)-2,3-dihydrobenzo[b][1,4]dioxin-2-yl)methyl)piperidin-3-yl)oxazolidine-2,4-dione.

¹HNMR (400 MHz, CDCl₃) δ ppm 1.70 (1H, m), 1.80 (2H, m), 2.10-2.20 (2H, m), 2.70-2.90 (5H, m), 4.0 (1H, m), 4.20 (1H, m), 4.30 (2H, m), 4.65 (2H, s), 6.85 (4H, m, ArH).

As already mentioned hereinbefore, the compounds of formula I show interesting pharmacological properties, namely they exhibit an improved selectivity for the alpha2C adrenoceptor subtype and/or an enhanced potency. Said properties are demonstrated with the pharmacological test presented below.

Experiment 1: Determination of alpha2A and alpha2C Antagonistic Activity In Vitro

Chinese hamster ovary (CHO) cells stably transfected with human alpha2A or alpha2C receptors (University of Turku, Finland) were cotransfected with the expression vector pCEP-Gα16 (Molecular Devices, CA, USA) were used in this experiment. The cells were maintained at 37° C. in a 5% CO₂/95% air atmosphere. The cells were cultured in HAM F-12 medium supplemented with 10% FCS, 25 mM HEPES, 100 IU/ml penicillin, 100 μg/ml streptomycin, 500 μg/ml geneticin and 240 μg/ml hygromycin B. The cells were subcultured twice weekly with 0.25% trypsin and 1 mM EDTA. The subculture ratio was 1:5-1:20. The growth medium was changed every 2 or 3 days. All cell culture reagents were from Gibco. The day before the experiment the cells were plated into black-walled, clear bottom 384-well plates at a density of 10,000 cells/well.

The growth medium was removed and the cells were incubated with the test compounds and the FLIPR Calcium 6 Assay reagent (Molecular Devices, CA, USA) for 2 h at 37° C. in dark. The test compounds (concentrations in cells 100 pM-10 μM) were dissolved in Probenecid-Ringer consisting of 150 mM NaCl, 3 mM KCl, 1.2 mM MgCl₂, 1 mM CaCl₂, 5 mM glucose, 20 mM HEPES and 2.5 mM probenecid (pH 7.4 adjusted with 1.0 M NaOH). The osmolarity was adjusted to 322 milliosmoles with Osmostat® OM-6020 osmometer (DIC Kyoto Daiichi Kagagu Co. Ltd, Japan). The changes in intracellular calcium were monitored using FLIPR Tetra high throughput cellular screening system (Molecular Devices, CA, USA) and displayed using ScreenWorks version 4.0 software. All experiments were performed at 37° C. For agonism measurements the test compounds dissolved in Probenecid-Ringer were applied by FLIPR Tetra at 15 s time point. In order to determine antagonism, the cells were stimulated either with 100 nM adrenaline or noradrenaline and the test compounds were added to the cells 2 h before the experiment with the FLIPR Calcium 6 Assay reagent. The IC₅₀ value for a given test compound was determined from dose-response curves, which ranged from 0.01 nM to 10 μM. Typically, there were four replicates at each concentration and six different dose levels. For example, if the number of plates from which results were obtained was three, 72 (4*6*3) wells were thus measured to construct dose-response relationship. The samples were excited at 485 nm and emission was detected at 525 nm with a 515 nm cut-off filter. The minimum fluorescence value subtracted from the maximum value for each well was used in the calculations. ScreenWorks version 4.0 software was used for analyzing the results. Fitting of the antagonist dose-response results was performed with the free Hill equation and the IC₅₀ values were fitted with IDBS XE software using model 200: y=(A+(B/(1+((x/C)̂D)))), where A is the curve maximum, B the curve minimum and C equals the EC50 value. D is slope factor (Hill). Kb was calculated with the Cheng-Prusoff equation Kb=A/((B/C)+1), where A is the IC₅₀ of antagonist, B the concentration of agonist and C the EC₅₀ of the agonist. The results are shown in Table 1.

TABLE 1
Alpha2A and alpha2C antagonistic activity in vitro.

| | Alpha 2A | | Alpha 2C | |
|---|---|---|---|---|
| | Adrenaline | | Adrenaline | |
| Compound | IC50 (nM) | Kb (nM) | IC50 (nM) | Kb (nM) |
| Compound of example 1 | >10000 | >449 | 0.320 | 0.030 |
| Compound of example 2 | 1334 | 60 | <0.100 | <0.010 |
| Compound of example 3 | 2682 | 120 | <0.100 | <0.010 |
| Compound of example 4 | >10000 | >449 | 1.770 | 0.180 |
| Compound of example 5 | 1303 | 58 | 0.130 | 0.010 |
| Compound of example 6 | 9871 | 443 | 672.330 | 67.720 |
| Compound of example 7 | 1728 | 78 | <0.100 | <0.010 |
| Compound of example 8 | >10000 | >449 | <0.100 | <0.010 |
| Compound of example 9 | 595 | 27 | <0.010 | <0.001 |
| Compound of example 10 | >10000 | >449 | 3.784 | 0.379 |
| Compound of example 11 | >10000 | >449 | 126.628 | 12.754 |
| Compound of example 12 | >10000 | >449 | 508.588 | 51.224 |
| Compound of example 13 | >10000 | >449 | 22.425 | 2.259 |
| Compound of example 14 | 5835 | 262 | 0.048 | 0.005 |
| Compound of example 15 | >10000 | >449 | 362.945 | 36.556 |
| Compound of example 16 | >10000 | >449 | 0.570 | 0.060 |
| Compound of example 17 | >10000 | >449 | 0.941 | 0.095 |
| Compound of example 18 | 8506 | 382 | 0.235 | 0.024 |
| Compound of example 19 | >10000 | >449 | 7.654 | 0.771 |
| Compound of example 20 | >10000 | >449 | 6.908 | 0.696 |
| Compound of example 21 | 1400 | 63 | 1.895 | 0.191 |
| Compound of example 22 | >10000 | >449 | <0.010 | <0.001 |
| Compound of example 23 | >10000 | >449 | 17.978 | 1.811 |
| Compound of example 24 | >10000 | >449 | 89.688 | 9.033 |
| Compound of example 25 | 535 | 24 | 19.380 | 1.950 |
| Compound of example 26 | >10000 | >449 | 27.890 | 2.810 |
| Compound of example 27 | >10000 | >449 | 20.755 | 2.090 |
| Compound of example 28 | >10000 | >449 | 5.435 | 0.547 |
| Compound of example 29 | 1611 | 72 | 0.838 | 0.083 |
| Compound of example 30 | >1000.000 | >45 | 1.110 | 0.110 |
| Compound of example 31 | >10000 | >449 | >10000 | >1007 |
| Compound of example 32 | 5207 | 234 | <0.010 | <0.001 |
| Compound of example 33 | 5807 | 261 | 1.340 | 0.135 |
| Compound of example 34 | 6865 | 308 | 0.020 | 0.002 |
| Compound of example 35 | 193 | 9 | <0.010 | <0.000 |
| Compound of example 36 | 1559 | 70 | 0.908 | 0.091 |
| Compound of example 37 | >10000 | >449 | <0.010 | <0.001 |
| Compound of example 38 | >10000 | >449 | 1.538 | 0.153 |
| Compound of example 39 | >10000 | >449 | 1.789 | 0.181 |
| Compound of example 40 | >10000 | >449 | 2.319 | 0.232 |
| Compound of example 41 | >10000 | >449 | 11.813 | 1.190 |
| Compound of example 42 | >10000 | >449 | 330.900 | 33.330 |
| Compound of example 43 | >10000 | >449 | <0.010 | <0.001 |
| Compound of example 44 | >10000 | >449 | 0.327 | 0.030 |
| Compound of example 45 | 1622 | 73 | <0.100 | <0.010 |
| Compound of example 46 | >1000.000 | >45 | 0.470 | 0.050 |
| Compound of example 47 | >10000 | >449 | 0.740 | 0.070 |
| Compound of example 48 | >10000 | >449 | 29.870 | 3.010 |
| Compound of example 49 | >10000 | >449 | 0.997 | 0.103 |
| Compound of example 50 | 1019 | 46 | <0.100 | <0.010 |
| Compound of example 51 | >10000 | >449 | 0.110 | 0.010 |
| Compound of example 52 | 1237 | 56 | <0.100 | <0.010 |
| Compound of example 52 | >10000 | >449 | 1.045 | 0.105 |
| Compound of example 54 | >10000 | >449 | 4.947 | 0.500 |
| Compound of example 55 | >10000 | >449 | 0.029 | 0.003 |
| Compound of example 56 | >10000 | >449 | 0.519 | 0.052 |
| Compound of example 57 | >10000 | >449 | 1.540 | 0.150 |
| Compound of example 58 | >10000 | >449 | 0.299 | 0.030 |
| Compound of example 59 | >10000 | >449 | <0.010 | <0.001 |
| Compound of example 60 | >10000 | >449 | <0.100 | <0.010 |
| Compound of example 61 | >10000 | >449 | 0.062 | 0.006 |
| Compound of example 62 | >10000 | >449 | <0.100 | <0.010 |
| Compound of example 63 | >10000 | >449 | <0.100 | <0.010 |
| Compound of example 64 | >10000 | >449 | 43.530 | 4.380 |
| Compound of example 65 | >10000 | >449 | <0.100 | <0.010 |
| Compound of example 66 | >10000 | >449 | <0.010 | <0.001 |
| Compound of example 67 | >10000 | >449 | 1.302 | 0.132 |
| Compound of example 68 | >10000 | >449 | 2.788 | 0.282 |
| Compound of example 69 | >10000 | >449 | 0.890 | 0.090 |
| Compound of example 70 | >10000 | >449 | 0.905 | 0.091 |
| Compound of example 71 | >10000 | >449 | 0.120 | 0.012 |
| Compound of example 72 | >10000 | >449 | 0.124 | 0.012 |
| Compound of example 73 | 185 | 8 | <0.100 | <0.010 |
| Compound of example 74 | 13 | 1 | <0.100 | <0.010 |
| Compound of example 75 | >10000 | >449 | 0.950 | 0.097 |
| Compound of example 76 | >10000 | >449 | 1769.002 | 178.173 |
| Compound of example 77 | >10000 | >449 | 10.463 | 1.054 |
| Compound of example 78 | >10000 | >449 | 256.380 | 25.820 |
| Compound of example 79 | >10000 | >449 | 5.397 | 0.544 |
| Compound of example 80 | >10000 | >449 | >10000 | >1007 |
| Compound of example 81 | >10000 | >449 | 652.750 | 65.740 |
| Compound of example 82 | >10000 | >449 | <0.100 | <0.010 |
| Compound of example 83 | >10000 | >449 | <0.100 | <0.010 |
| Compound of example 84 | 4333 | 195 | <0.100 | <0.010 |
| Compound of example 85 | >10000 | >449 | 1257.760 | 126.680 |
| Compound of example 86 | >10000 | >449 | <0.100 | <0.010 |
| Compound of example 87 | >10000 | >449 | <0.100 | <0.010 |
| Compound of example 88 | >10000 | >449 | <0.100 | <0.010 |
| Compound of example 89 | 9457 | 425 | <0.100 | <0.010 |
| Compound of example 90 | 11084 | 498 | <0.100 | <0.010 |
| Compound of example 91 | >10000 | >449 | <0.100 | <0.010 |
| Compound of example 92 | >10000 | >449 | 0.150 | 0.020 |
| Compound of example 93 | >10000 | >449 | 0.091 | 0.009 |
| Compound of example 94 | >10000 | >449 | <0.100 | <0.010 |
| Compound of example 95 | 9439 | 424 | 0.433 | 0.044 |
| Compound of example 96 | >10000 | >449 | 15.050 | 1.520 |
| Compound of example 97 | >10000 | >449 | 1.000 | 0.100 |
| Compound of example 98 | >10000 | >449 | 9.260 | 0.930 |
| Compound of example 99 | >10000 | >449 | 0.150 | 0.020 |
| Compound of example 100 | >10000 | >449 | 2.017 | 0.203 |
| Compound of example 101 | >10000 | >449 | 0.780 | 0.080 |
| Compound of example 102 | >10000 | >449 | 1.080 | 0.110 |
| Compound of example 103 | >10000 | >449 | 3.175 | 0.320 |
| Compound of example 104 | >10000 | >449 | 10.010 | 1.010 |
| Compound of example 105 | >10000 | >449 | 3.270 | 0.330 |
| Compound of example 106 | >10000 | >449 | 0.100 | 0.010 |
| Compound of example 107 | >10000 | >449 | 0.284 | 0.029 |
| Compound of example 108 | >10000 | >449 | 1.250 | 0.130 |
| Compound of example 109 | >10000 | >449 | 0.117 | 0.014 |
| Compound of example 110 | 8135 | 365 | <0.100 | <0.010 |
| Compound of example 111 | >10000 | >449 | 0.870 | 0.090 |
| Compound of example 112 | >10000 | >449 | 0.160 | 0.020 |
| Compound of example 113 | >10000 | >449 | 0.175 | 0.015 |
| Compound of example 114 | 2336 | 105 | <0.100 | <0.010 |
| Compound of example 115 | >10000 | >449 | 2.075 | 0.210 |
| Compound of example 116 | >10000 | >449 | 1.000 | 0.100 |
| Compound of example 117 | >10000 | >449 | 1.994 | 0.202 |
| Compound of example 118 | 1123 | 50 | <0.100 | <0.010 |
| Compound of example 119 | >10000 | >449 | 17.485 | 1.760 |
| Compound of example 120 | >10000 | >449 | 131.940 | 13.290 |
| Compound of example 121 | >10000 | >449 | 26.430 | 2.662 |
| Compound of example 122 | 1381 | 62 | 0.110 | 0.010 |
| Compound of example 123 | >10000 | >449 | 3.520 | 0.354 |
| Compound of example 124 | >10000 | >449 | 1.474 | 0.131 |
| Compound of example 125 | >10000 | >449 | 0.843 | 0.086 |
| Compound of example 126 | 3888 | 175 | 0.010 | 0.001 |
| Compound of example 127 | 8673 | 389 | 0.027 | 0.003 |
| Compound of example 128 | 8886 | 399 | 1.186 | 0.119 |
| Compound of example 129 | >10000 | >449 | 1.578 | 0.162 |
| Compound of example 130 | 6594 | 296 | 0.256 | 0.028 |
| Compound of example 131 | 5705 | 256 | 1.636 | 0.165 |
| Compound of example 132 | >10000 | >449 | 0.308 | 0.031 |
| Compound of examp1e133 | 807 | 36 | <0.010 | <0.001 |
| Compound of example 134 | >10000 | >449 | 0.796 | 0.080 |
| Compound of example 135 | >10000 | >449 | 443.705 | 44.690 |
| Compound of example 136 | >10000 | >449 | 0.599 | 0.060 |
| Compound of example 137 | >10000 | >449 | 5.504 | 0.554 |
| Compound of example 138 | >10000 | >449 | 4.347 | 0.438 |
| Compound of example 139 | 9827 | 441 | 0.113 | 0.012 |
| Compound of example 140 | >10000 | >449 | 2.189 | 0.220 |
| Compound of example 141 | >10000 | >449 | 0.446 | 0.045 |
| Compound of example 142 | >10000 | >449 | 0.846 | 0.085 |
| Compound of example 143 | >10000 | >449 | 1.383 | 0.140 |
| Compound of example 144 | >10000 | >449 | 2.530 | 0.255 |
| Compound of example 145 | 2785 | 125 | 0.850 | 0.090 |
| Compound of example 146 | 7187 | 323 | 1045.545 | 105.305 |
| Compound of example 147 | >10000 | >449 | 227.870 | 22.950 |
| Compound of example 148 | >10000 | >449 | <0.100 | <0.010 |
| Compound of example 149 | >10000 | >449 | 3216.840 | 324.000 |
| Compound of example 150 | 4476 | 201 | <0.100 | <0.010 |
| Compound of example 151 | 6155 | 276 | <0.100 | <0.010 |
| Compound of example 152 | 288 | 13 | 69.758 | 7.027 |
| Compound of example 153 | >10000 | >449 | 3584.128 | 360.991 |
| Compound of example 154 | >10000 | >449 | <0.100 | <0.010 |
| Compound of example 155 | >10000 | >449 | 56.039 | 5.644 |
| Compound of example 156 | 3584 | 161 | 0.012 | 0.001 |
| Compound of example 157 | >10000 | >449 | 1.628 | 0.164 |
| Compound of example 158 | >10000 | >449 | 3.432 | 0.346 |
| Compound of example 159 | >10000 | >449 | 0.010 | 0.001 |
| Compound of example 160 | >10000 | >449 | 0.150 | 0.016 |
| Compound of example 161 | >10000 | >449 | 0.294 | 0.030 |
| Compound of example 162 | >10000 | >449 | 6.130 | 0.620 |
| Compound of example 163 | >10000 | >449 | 2.080 | 0.207 |

In vivo effects of the compounds of formula I can be demonstrated with the pharmacological tests as described in WO 03/082866.

The compounds of formula I exhibit alpha2C antagonistic activity. The present disclosure thus provides compounds for use as a medicament. Compounds for use in the treatment of disorder, condition, or disease where an alpha2C antagonist is indicated to be useful are also provided. Furthermore, a method for the treatment of disorder, condition, or disease where an alpha2C antagonist is indicated to be useful is provided. In said method an effective amount of at least one compound of formula I is administered to a mammal, such as human, in need of such treatment. The use of the compounds of formula I for the manufacture of a medicament for the treatment of disorder, condition, or disease where an alpha2C antagonist is indicated to be useful is also provided.

In one embodiment of the invention the aforementioned disorder, condition, or disease where an alpha2C antagonist is indicated to be useful is a mental disorder propagated by stress, Parkinson's disease, depression, schizophrenia, attention deficit hyperactivity disorder, post-traumatic stress disorder, obsessive compulsive disorder, Tourette's syndrome, blepharospasm or other focal dystonias, temporal lobe epilepsy with psychosis, a drug-induced psychosis, Huntington's disease, a disorder caused by fluctuation of the levels of sex hormones, panic disorder, Alzheimer's disease, or mild cognitive impairment; for example, a mental disorder propagated by stress, Parkinson's disease, depression, schizophrenia, attention deficit hyperactivity disorder, obsessive compulsive disorder, or Alzheimer's disease; such as a mental disorder propagated by stress, depression, or schizophrenia. Representative examples of drug-induced psychoses include, but are not limited to, psychosis caused by chronic use of dopaminergic agents.

Representative examples of disorders caused by fluctuation of the levels of sex hormones include, but are not limited to, premenstrual syndrome and hot flashes.

The compounds of the present disclosure can be administered, for example, enterally, topically or parenterally by means of any pharmaceutical formulation useful for said administration and comprising at least one active compound of formula I in pharmaceutically acceptable and effective amounts together with pharmaceutically acceptable diluents, carriers and/or excipients known in the art. The manufacture of such pharmaceutical formulations is known in the art.

The therapeutic dose to be given to a subject in need of the treatment will vary depending on the compound being administered, the species, the age and the sex of the subject being treated, the particular condition being treated, as well as the route and method of administration, and is easily determined by a person skilled in the art. Accordingly, the typical dosage for oral administration is from 10 ng/kg to 100 mg/kg per day and for parenteral administration from 1 ng/kg to 10 mg/kg for an adult mammal.

The compounds of the present disclosure are given to the subject as such or in combination with one or more other active ingredients, each in its own composition or some or all of the active ingredients combined in a single composition, and/or suitable pharmaceutical excipients. Suitable pharmaceutical excipients include conventionally used excipients and formulation aids, such as fillers, binders, disintegrating agents, lubricants, solvents, gel forming agents, emulsifiers, stabilizers, colorants, and/or preservatives.

The compounds of the present disclosure are formulated into dosage forms using commonly known pharmaceutical manufacturing methods. The dosage forms can be, for example, tablets, capsules, granules, suppositories, emulsions, suspensions, or solutions. Depending on the route of administration and the galenic form, the amount of the active ingredient in a formulation can typically vary between 0.01% and 100% by weight.

A person skilled in the art will appreciate that the embodiments described herein can be modified without departing from the inventive concept. A person skilled in the art also understands that the present disclosure is not limited to the particular embodiments disclosed but is intended to also cover modifications of the embodiments that are within the scope of the present disclosure.

1. A compound of formula I, 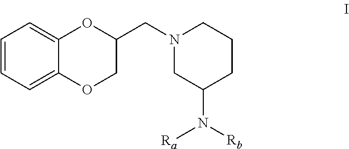
wherein;
Rₐ and R_{b} form, together with the nitrogen atom to which they are attached, a 5 or 6 membered saturated or unsaturated heterocyclic ring, containing, in addition to the nitrogen atom to which Rₐ and R_{b} are attached, 0, 1 or 2 ring heteroatom(s) each independently selected from N, O and S, wherein said heterocyclic ring is substituted with 1 substituent R₁, or said heterocyclic ring is substituted with 2 substituents R₁ and R₂, or said heterocyclic ring is substituted with 3 substituents R₁, R₂, and R₃, or said heterocyclic ring is substituted with 4 substituents R₁, R₂, R₃, and R₄, or said heterocyclic ring is substituted with 5 substituents R₁, R₂, R₃, R₄, and R₅;
R₁, R₂, R₃, R₄ and R₅ are independently oxo, (C₁-C₆)alkyl, (C₁-C₆)alkoxy(C₁-C₆)alkyl, (R₆)₂N—, (R₆)₂N—(C₁-C₆)alkyl, (R₆)₂N—(C═O)—(C₁-C₆)alkyl, (C₁-C₆)alkyl-(C═O)—NR₆—(C₁-C₆)alkyl, cyclo(C₃-C₆)alkyl, cyclo(C₃-C₆)alkyl(C₁-C₆)alkyl, phenyl, phenyl(C₁-C₆)alkyl, heterocyclyl, or heterocyclyl(C₁-C₆)alkyl, wherein said phenyl, cyclo(C₃-C₆)alkyl, or heterocyclyl is optionally substituted with 1 or 2 substituent(s) each independently being halogen, (C₁-C₆)alkoxy, or (C₁-C₆)alkyl-(C═O);
or two of R₃, R₄ and R₅, both attached to the same carbon ring atom form, together with the carbon ring atom to which they are attached, a 3 membered unsubstituted carbocyclic ring;
or two of R₁, R₂, R₃, R₄ and R₅, attached to the adjacent carbon ring atoms form, together with the carbon ring atoms to which they are attached, a phenyl ring, a 3 to 6 membered saturated or unsaturated carbocyclic ring, or a 5 or 6 membered saturated or unsaturated heterocyclic ring containing 1 or 2 ring heteroatom(s) each independently selected from N and S, wherein said phenyl ring, carbocyclic ring, or heterocyclic ring is unsubstituted, or said phenyl ring, carbocyclic ring, or heterocyclic ring is substituted with 1 substituent R₇, or said phenyl ring, carbocyclic ring, or heterocyclic ring is substituted with 2 substituents R₇ and R₈;
R₆ is H or (C₁-C₆)alkyl;
R₇ and R₈ are independently halogen, (C₁-C₆)alkyl, (C₁-C₆)alkoxy, (C₁-C₆)alkyl-S—, CN, or (C₁-C₆)alkyl-(C═O)—NR₆—(C₁-C₆)alkyl;
or R₇ and R₈, attached to the non-adjacent carbon ring atoms, form a bridge;
or a pharmaceutically acceptable salt or ester thereof.

2. The compound according to claim 1, wherein the compound is a compound of formula Ia,

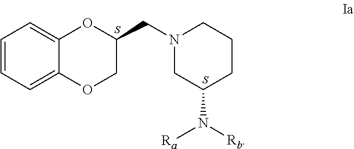

3. The compound according to claim 1, wherein;
Rₐ and R_{b} form, together with the nitrogen atom to which they are attached, a 5 or 6 membered saturated or unsaturated heterocyclic ring, containing, in addition to the nitrogen atom to which Rₐ and R_{b} are attached, 0, 1 or 2 ring heteroatom(s) each independently selected from N, O and S, wherein said heterocyclic ring is substituted with 1 substituent R₁, or said heterocyclic ring is substituted with 2 substituents R₁ and R₂, or said heterocyclic ring is substituted with 3 substituents R₁, R₂, and R₃, or said heterocyclic ring is substituted with 4 substituents R₁, R₂, R₃, and R₄, or said heterocyclic ring is substituted with 5 substituents R₁, R₂, R₃, R₄ and R₅;
R₁ is oxo,
R₂ is oxo, (C₁-C₆)alkyl, (C₁-C₆)alkoxy(C₁-C₆)alkyl, (R₆)₂N—(C₁-C₆)alkyl, (R₆)₂N—(C═O)—(C₁-C₆)alkyl, (C₁-C₆)alkyl-(C═O)—NR₆—(C₁-C₆)alkyl, cyclo(C₃-C₆)alkyl, cyclo(C₃-C₆)alkyl(C₁-C₆)alkyl, phenyl, phenyl(C₁-C₆)alkyl, heterocyclyl, or heterocyclyl(C₁-C₆)alkyl, wherein said phenyl, cyclo(C₃-C₆)alkyl, or heterocyclyl is optionally substituted with 1 or 2 substituent(s) each independently being halogen, (C₁-C₆)alkoxy, or (C₁-C₆)alkyl-(C═O);
R₃ is oxo, (C₁-C₆)alkyl, or phenyl;
R₄ is oxo or (C₁-C₆)alkyl;
R₅ is (C₁-C₆)alkyl;
or two of R₃, R₄ and R₅, both attached to the same carbon ring atom form, together with the carbon ring atom to which they are attached, a 3 membered unsubstituted carbocyclic ring;
or two of R₁, R₂, R₃, R₄ and R₅ attached to the adjacent carbon ring atoms form, together with the carbon ring atoms to which they are attached, a phenyl ring, a 3 to 6 membered saturated or unsaturated carbocyclic ring, or a 5 or 6 membered saturated or unsaturated heterocyclic ring containing 1 or 2 ring heteroatom(s) each independently selected from N and S, wherein said phenyl ring, carbocyclic ring, or heterocyclic ring is unsubstituted, or said phenyl ring, carbocyclic ring, or heterocyclic ring is substituted with 1 substituent R₇, or said phenyl ring, carbocyclic ring, or heterocyclic ring is substituted with 2 substituents R₇ and R₈;
R₆ is H or (C₁-C₆)alkyl;
R₇ is halogen, (C₁-C₆)alkyl, (C₁-C₆)alkoxy, (C₁-C₆)alkyl-S—, CN, or (C₁-C₆)alkyl-(C═O)—NR₆—(C₁-C₆)alkyl;
R₈ is halogen or (C₁-C₆)alkoxy;
or R₇ and R₈, attached to the non-adjacent carbon ring atoms, form a bridge.

4. The compound according to claim 1, wherein Rₐ and R_{b} form, together with the nitrogen atom to which they are attached any one of the following groups 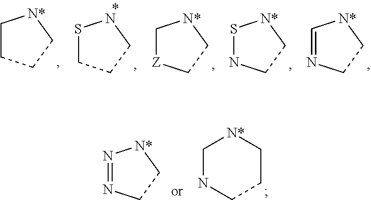
wherein;
Z is N or O; and
the atom marked with * is bonded to the parent molecular moiety, and the dotted line is a single or a double bond.

5. The compound according to claim 1, wherein Rₐ and R_{b} form, together with the nitrogen atom to which they are attached any one of the following groups 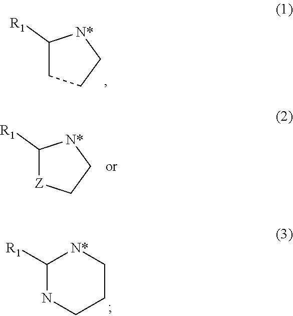
wherein;
group (1), (2), or (3) is optionally further substituted with R₂, R₃, and/or R₄;
Z is N or O;
R₁ is oxo;
R₂ is (C₁-C₆)alkyl, (R₆)₂N—(C═O)—(C₁-C₆)alkyl, (C₁-C₆)alkyl-(C═O)—NR₆—(C₁-C₆)alkyl, phenyl, or phenyl(C₁-C₆)alkyl, wherein said phenyl is optionally substituted with 1 substituent being halogen or (C₁-C₆)alkoxy;
R₃ is oxo, (C₁-C₆)alkyl, or phenyl;
R₄ is (C₁-C₆)alkyl;
R₆ is H or (C₁-C₆)alkyl;
the atom marked with * is bonded to the parent molecular moiety, and the dotted line is a single or a double bond.

6. The compound according to claim 1, wherein Rₐ and R_{b} form, together with the nitrogen atom to which they are attached any one of the following groups 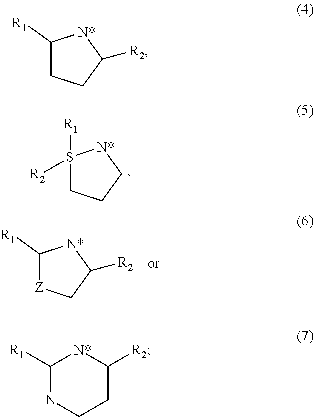
wherein;
group (4), (5), (6), or (7) is optionally further substituted with R₃, R₄, and/or R₅;
Z is N or O;
R₁ is oxo;
R₂ is oxo;
R₃ is (C₁-C₆)alkyl, (R₆)₂N—(C₁-C₆)alkyl, (R₆)₂N—(C═O)—(C₁-C₆)alkyl, cyclo(C₃-C₆)alkyl, cyclo(C₃-C₆)alkyl(C₁-C₆)alkyl, phenyl, phenyl(C₁-C₆)alkyl, heterocyclyl, or heterocyclyl(C₁-C₆)alkyl, wherein said phenyl, cyclo(C₃-C₆)alkyl, or heterocyclyl is optionally substituted with 1 or 2 substituent(s) each independently being halogen or (C₁-C₆)alkyl-(C═O);
R₄ is oxo or (C₁-C₆)alkyl;
R₅ is (C₁-C₆)alkyl;
R₆ is (C₁-C₆)alkyl;
or two of R₃, R₄ and R₅, both attached to the same carbon ring atom form, together with the carbon ring atom to which they are attached, a 3 membered unsubstituted carbocyclic ring; and the atom marked with * is bonded to the parent molecular moiety.

7. The compound according to claim 1, wherein Rₐ and R_{b} form, together with the nitrogen atom to which they are attached any one of the following groups 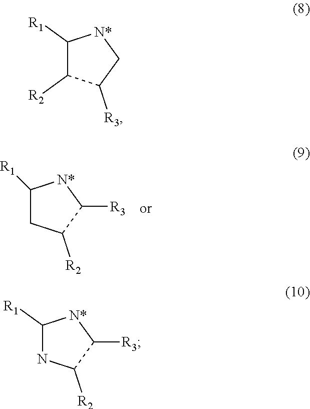
wherein;
group (8), (9), or (10) is optionally further substituted with R₄;
R₁ is oxo,
R₂ and R₃ form, together with the carbon ring atoms to which they are attached, a phenyl ring or a 5 or 6 membered unsaturated heterocyclic ring containing 1 or 2 ring heteroatom(s) each independently selected from N and S, wherein said phenyl ring or heterocyclic ring is unsubstituted, or said phenyl ring or heterocyclic ring is substituted with 1 substituent R₇, or said phenyl ring is substituted with 2 substituents R₇ and R₈;
R₄ is (C₁-C₆)alkyl or phenyl;
R₇ is halogen, (C₁-C₆)alkyl, (C₁-C₆)alkoxy, (C₁-C₆)alkyl-S—, CN, or (C₁-C₆)alkyl-(C═O)—NH—(C₁-C₆)alkyl;
R₈ is halogen or (C₁-C₆)alkoxy;
the atom marked with * is bonded to the parent molecular moiety, and the dotted line is a single or a double bond.

8. The compound according to claim 1, wherein Rₐ and R_{b} form, together with the nitrogen atom to which they are attached any one of the following groups 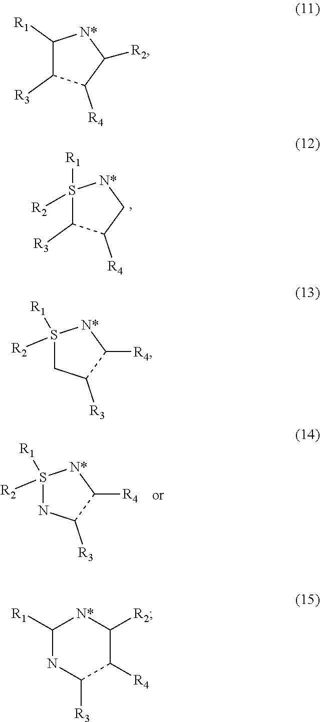
wherein;
group (11), (12), (13), (14), or (15) is optionally further substituted with R₅;
R₁ is oxo;
R₂ is oxo;
R₃ and R₄ form, together with the carbon ring atoms to which they are attached, a phenyl ring, a 3 to 6 membered saturated or unsaturated carbocyclic ring, or a 6 membered unsaturated heterocyclic ring containing 1 ring heteroatom being N, wherein said phenyl ring, carbocyclic ring, or heterocyclic ring is unsubstituted, or said phenyl ring is substituted with 1 substituent R₇, or said phenyl ring or carbocyclic ring is substituted with 2 substituents R₇ and R₈;
R₅ is phenyl;
R₇ is halogen or (C₁-C₆)alkoxy;
R₈ is (C₁-C₆)alkoxy;
or R₇ and R₈, attached to the non-adjacent carbon ring atoms, form a bridge;
the atom marked with * is bonded to the parent molecular moiety, and the dotted line is a single or a double bond.

9. The compound according to claim 1, wherein Rₐ and R_{b} form, together with the nitrogen atom to which they are attached any one of the following groups 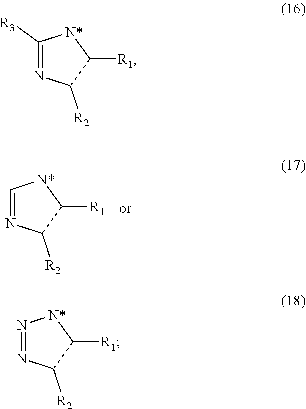
wherein;
R₁ and R₂ form, together with the carbon ring atoms to which they are attached, a phenyl ring, or a 6 membered saturated or unsaturated heterocyclic ring containing 1 or 2 ring heteroatom(s) each independently selected from N, wherein said phenyl ring, or heterocyclic ring is unsubstituted, or said phenyl ring, or heterocyclic ring is substituted with 1 substituent R₇, or said phenyl ring is substituted with 2 substituents R₇ and R₈;
R₃ is (C₁-C₆)alkyl, (C₁-C₆)alkoxy(C₁-C₆)alkyl, or (R₆)₂N—(C₁-C₆)alkyl;
R₆ is H or (C₁-C₆)alkyl;
R₇ is halogen or (C₁-C₆)alkoxy;
R₈ is halogen; and
the atom marked with * is bonded to the parent molecular moiety, and the dotted line is a single or a double bond.

10. The compound according to claim 1, wherein the compound is 1-((S)-1-(((S)-2,3-dihydrobenzo[b][1,4]dioxin-2-yl)methyl)piperidin-3-yl)-4,4-dimethylpyrrolidin-2-one, 1-((S)-1-(((S)-2,3-dihydrobenzo[b][1,4]dioxin-2-yl)methyl)piperidin-3-yl)-4,4-diphenyl-imidazolidin-2-one, 1-((S)-1-(((S)-2,3-dihydrobenzo[b][1,4]dioxin-2-yl)methyl)piperidin-3-yl)-4-phenylimidazolidin-2-one, (3R,4R)-1-((S)-1-(((S)-2,3-dihydrobenzo[b][1,4]dioxin-2-yl)methyl)piperidin-3-yl)-3,4-dimethylpyrrolidine-2,5-dione, 3-((S)-1-(((S)-2,3-dihydrobenzo[b][1,4]dioxin-2-yl)methyl)piperidin-3-yl)-5,5-diethyloxazolidine-2,4-dione, (R)-1-((S)-1-(((S)-2,3-dihydrobenzo[b][1,4]dioxin-2-yl)methyl)-piperidin-3-yl)-3-isopropyl-4-phenylimidazolidin-2-one, (S)-1-((S)-1-(((S)-2,3-dihydrobenzo[b][1,4]dioxin-2-yl)methyl)piperidin-3-yl)-3-isopropyl-4-phenylimidazolidin-2-one, (R)-1-((S)-1-(((S)-2,3-dihydrobenzo[b][1,4]dioxin-2-yl)methyl)piperidin-3-yl)-4-phenylimidazolidin-2-one, (S)-1-((S)-1-(((S)-2,3-dihydrobenzo[b][1,4]dioxin-2-yl)methyl)piperidin-3-yl)-4-phenyl-imidazolidin-2-one, 6-((S)-1-(((S)-2,3-dihydrobenzo[b][1,4]dioxin-2-yl)methyl)piperidin-3-yl)-6,7-dihydro-5H-pyrrolo[3,4-b]pyridin-5-one, 2-((S)-1-(((S)-2,3-dihydrobenzo[b][1,4]-dioxin-2-yl)methyl)piperidin-3-yl)-1H-pyrrolo[3,4-c]pyridin-3(2H)-one, 6-((S)-1-(((S)-2,3-dihydrobenzo[b][1,4]dioxin-2-yl)methyl)piperidin-3-yl)-2-(methylthio)-5H-pyrrolo[3,4-d]pyrimidin-7(6H)-one, 5-((S)-1-(((S)-2,3-dihydrobenzo[b][1,4]dioxin-2-yl)methyl)-piperidin-3-yl)-5,6-dihydro-4H-pyrrolo[3,4-d]thiazol-4-one, (R)-1-((S)-1-(((S)-2,3-dihydrobenzo[b][1,4]dioxin-2-yl)methyl)piperidin-3-yl)-3-methyl-3-phenylpyrrolidin-2-one, (S)-1-((S)-1-(((S)-2,3-dihydrobenzo[b][1,4]dioxin-2-yl)methyl)-piperidin-3-yl)-3-methyl-3-phenylpyrrolidin-2-one, 1-((S)-1-(((S)-2,3-dihydrobenzo[b][1,4]dioxin-2-yl)methyl)-piperidin-3-yl)-3-ethylimidazolidin-2-one, 2-(3-((S)-1-(((S)-2,3-dihydrobenzo[b][1,4]-dioxin-2-yl)methyl)-piperidin-3-yl)-2-oxoimidazolidin-1-yl)-N,N-dimethylacetamide, 5-tert-butyl-3-((S)-1-(((S)-2,3-dihydrobenzo[b][1,4]dioxin-2-yl)methyl)piperidin-3-yl)oxazolidin-2-one, 3-((S)-1-(((S)-2,3-dihydrobenzo[b][1,4]dioxin-2-yl)methyl)piperidin-3-yl)-5-isopropyloxazolidin-2-one, N-((3-((S)-1-(((S)-2,3-dihydrobenzo[b][1,4]dioxin-2-yl)methyl)-piperidin-3-yl)-2-oxooxazolidin-5-yl)methyl)acetamide, 3-((S)-1-(((S)-2,3-dihydrobenzo-[b][1,4]dioxin-2-yl)methyl)piperidin-3-yl)-5-(4-fluorophenyl)oxazolidin-2-one, 6-((S)-1-(((S)-2,3-dihydrobenzo[b][1,4]dioxin-2-yl)methyl)piperidin-3-yl)-2-(methylthio)-6,7-dihydro-5H-pyrrolo[3,4-d]pyrimidin-5-one, 6-((S)-1-(((S)-2,3-dihydrobenzo[b][1,4]dioxin-2-yl)methyl)piperidin-3-yl)-6,7-dihydro-5H-pyrrolo[3,4-d]pyrimidin-5-one, 5-((S)-1-(((S)-2,3-dihydrobenzo[b][1,4]dioxin-2-yl)methyl)piperidin-3-yl)-1-methyl-4,5-dihydropyrrolo-[3,4-c]pyrazol-6(1H)-one, 1-((S)-1-(((S)-2,3-dihydrobenzo[b][1,4]dioxin-2-yl)methyl)-piperidin-3-yl)-1,3-dihydrobenzo[c]isothiazole 2,2-dioxide, 1-((S)-1-(((S)-2,3-dihydrobenzo[b][1,4]dioxin-2-yl)methyl)piperidin-3-yl)indolin-2-one, 1-((S)-1-(((S)-2,3-dihydrobenzo[b][1,4]dioxin-2-yl)methyl)piperidin-3-yl)-4,6-difluoro-2-methyl-1H-benzo[d]imidazole, 1-((S)-1-(((S)-2,3-dihydrobenzo[b][1,4]dioxin-2-yl)methyl)piperidin-3-yl)-5,6-difluoro-2-methyl-1H-benzo[d]imidazole, 1-((S)-1-(((S)-2,3-dihydrobenzo[b][1,4]-dioxin-2-yl)methyl)piperidin-3-yl)pyrrolidin-2-one, 1-((S)-1-(((S)-2,3-dihydrobenzo[b][1,4]-dioxin-2-yl)methyl)piperidin-3-yl)-5-methylpyrrolidin-2-one, 1-((S)-1-(((S)-2,3-dihydrobenzo[b][1,4]dioxin-2-yl)methyl)piperidin-3-yl)-3-phenylpyrrolidin-2-one, 1-((S)-1-(((S)-2,3-dihydrobenzo[b][1,4]dioxin-2-yl)methyl)piperidin-3-yl)-4-phenylpyrrolidin-2-one, 1-((S)-1-(((S)-2,3-dihydrobenzo[b][1,4]dioxin-2-yl)methyl)piperidin-3-yl)pyrrolidine-2,5-dione, 2-((S)-1-(((S)-2,3-dihydrobenzo[b][1,4]dioxin-2-yl)methyl)piperidin-3-yl)-3a,4,7,7a-tetrahydro-1H-4,7-methanoisoindole-1,3(2H)-dione, 1-((S)-1-(((S)-2,3-dihydrobenzo-[b][1,4]dioxin-2-yl)methyl)piperidin-3-yl)-3-methyl-3-phenylpyrrolidine-2,5-dione, (R)-1-((S)-1-(((S)-2,3-dihydrobenzo[b][1,4]dioxin-2-yl)methyl)piperidin-3-yl)-3-methyl-3-phenylpyrrolidine-2,5-dione, (S)-1-((S)-1-(((S)-2,3-dihydrobenzo[b][1,4]dioxin-2-yl)methyl)piperidin-3-yl)-3-methyl-3-phenylpyrrolidine-2,5-dione, 3-((S)-1-(((S)-2,3-dihydrobenzo[b][1,4]dioxin-2-yl)methyl)piperidin-3-yl)-1-phenyl-3-azabicyclo[3.1.0]-hexane-2,4-dione, 1-((S)-1-(((S)-2,3-dihydrobenzo[b][1,4]dioxin-2-yl)methyl)piperidin-3-yl)-4-phenylpyrrolidine-2,3-dione, 1-((S)-1-(((S)-2,3-dihydrobenzo[b][1,4]dioxin-2-yl)methyl)piperidin-3-yl)-4-phenyl-1H-pyrrol-2(5H)-one, 1-((S)-1-(((S)-2,3-dihydrobenzo-[b][1,4]dioxin-2-yl)methyl)piperidin-3-yl)-4-methyl-1H-pyrrol-2(5H)-one, 1-((S)-1-(((S)-2,3-dihydrobenzo[b][1,4]dioxin-2-yl)methyl)piperidin-3-yl)-4-(4-fluorophenyl)-1H-pyrrol-2(5H)-one, 1-((S)-1-(((S)-2,3-dihydrobenzo[b][1,4]dioxin-2-yl)methyl)piperidin-3-yl)-4-methyl-3-phenyl-1H-pyrrol-2(5H)-one, 1-((S)-1-(((S)-2,3-dihydrobenzo[b][1,4]dioxin-2-yl)methyl)piperidin-3-yl)-4-(2-methoxyphenyl)-1H-pyrrol-2(5H)-one formate, 1-((S)-1-(((S)-2,3-dihydrobenzo[b][1,4]dioxin-2-yl)methyl)piperidin-3-yl)imidazolidin-2-one, 1-((S)-1-(((S)-2,3-dihydrobenzo[b][1,4]dioxin-2-yl)methyl)piperidin-3-yl)-4,4-dimethylimidazolidin-2-one, (R)-1-((S)-1-(((S)-2,3-dihydrobenzo[b][1,4]dioxin-2-yl)methyl)-piperidin-3-yl)-4-methylimidazolidin-2-one, 1-((S)-1-(((S)-2,3-dihydrobenzo[b][1,4]dioxin-2-yl)methyl)piperidin-3-yl)-5-methylimidazolidin-2-one, 1-((S)-1-(((S)-2,3-dihydrobenzo-[b][1,4]dioxin-2-yl)methyl)piperidin-3-yl)-3-phenylimidazolidin-2-one, 1-((S)-1-(((S)-2,3-dihydrobenzo[b][1,4]dioxin-2-yl)methyl)piperidin-3-yl)-3,4-dimethylimidazolidin-2-one, 1-benzyl-3-((S)-1-(((S)-2,3-dihydrobenzo[b][1,4]dioxin-2-yl)methyl)piperidin-3-yl)imidazolidin-2-one, 1-((S)-1-(((S)-2,3-dihydrobenzo[b][1,4]dioxin-2-yl)methyl)piperidin-3-yl)-3,4,4-trimethylimidazolidin-2-one, 1-((S)-1-(((S)-2,3-dihydrobenzo[b][1,4]dioxin-2-yl)methyl)piperidin-3-yl)-3-(1-phenylethyl)imidazolidin-2-one hydrochloride, 1-((S)-1-(((S)-2,3-dihydrobenzo[b][1,4]dioxin-2-yl)methyl)piperidin-3-yl)tetrahydropyrimidin-2(1H)-one, 1-((S)-1-(((S)-2,3-dihydrobenzo[b][1,4]dioxin-2-yl)methyl)piperidin-3-yl)-3-methyl-tetrahydropyrimidin-2(1H)-one, 2-((S)-1-(((S)-2,3-dihydrobenzo[b][1,4]dioxin-2-yl)methyl)piperidin-3-yl)-4-methoxyisoindolin-1-one, 2-((S)-1-(((S)-2,3-dihydrobenzo-[b][1,4]dioxin-2-yl)methyl)piperidin-3-yl)-4,5-difluoroisoindolin-1-one hydrochloride, 2-((S)-1-(((S)-2,3-dihydrobenzo[b][1,4]dioxin-2-yl)methyl)piperidin-3-yl)-4-fluoroisoindolin-1-one hydrochloride, 2-((S)-1-(((S)-2,3-dihydrobenzo[b][1,4]dioxin-2-yl)methyl)piperidin-3-yl)-5-fluoroisoindolin-1-one hydrochloride, 2-((S)-1-(((S)-2,3-dihydrobenzo[b][1,4]-dioxin-2-yl)methyl)piperidin-3-yl)-5-methylisoindolin-1-one formate, 5-chloro-2-((S)-1-(((S)-2,3-dihydrobenzo[b][1,4]dioxin-2-yl)methyl)piperidin-3-yl)isoindolin-1-one, 2-((S)-1-(((S)-2,3-dihydrobenzo[b][1,4]dioxin-2-yl)methyl)piperidin-3-yl)isoindoline-1,3-dione, 2-((S)-1-(((S)-2,3-dihydrobenzo[b][1,4]dioxin-2-yl)methyl)piperidin-3-yl)-5-fluoroisoindoline-1,3-dione, 2-((S)-1-(((S)-2,3-dihydrobenzo[b][1,4]dioxin-2-yl)methyl)-piperidin-3-yl)-4-fluoroisoindoline-1,3-dione hydrochloride, 4-chloro-2-((S)-1-(((S)-2,3-dihydrobenzo[b][1,4]dioxin-2-yl)methyl)piperidin-3-yl)isoindoline-1,3-dione hydrochloride, 2-((S)-1-(((S)-2,3-dihydrobenzo[b][1,4]dioxin-2-yl)methyl)piperidin-3-yl)-4-methoxy-isoindoline-1,3-dione, 2-((S)-1-(((S)-2,3-dihydrobenzo[b][1,4]dioxin-2-yl)methyl)piperidin-3-yl)-4,5-dimethoxyisoindoline-1,3-dione hydrochloride, 2-((S)-1-(((S)-2,3-dihydrobenzo-[b][1,4]dioxin-2-yl)methyl)piperidin-3-yl)-1-oxoisoindoline-5-carbonitrile, 2-((S)-1-(((S)-2,3-dihydrobenzo[b][1,4]dioxin-2-yl)methyl)piperidin-3-yl)-5,6-dimethoxyisoindolin-1-one, 2-((S)-1-(((S)-2,3-dihydrobenzo[b][1,4]dioxin-2-yl)methyl)piperidin-3-yl)-5-methoxyisoindolin-1-one, 2-((S)-1-(((S)-2,3-dihydrobenzo[b][1,4]dioxin-2-yl)methyl)-piperidin-3-yl)-6-methoxyisoindolin-1-one, N-((2-((S)-1-(((S)-2,3-dihydrobenzo[b][1,4]-dioxin-2-yl)methyl)piperidin-3-yl)-1-oxoisoindolin-5-yl)methyl)acetamide, 3-((S)-1-(((S)-2,3-dihydrobenzo[b][1,4]dioxin-2-yl)methyl)-piperidin-3-yl)quinazoline-2,4(1H,3H)-dione, 3-((S)-1-(((S)-2,3-dihydrobenzo[b][1,4]dioxin-2-yl)methyl)-piperidin-3-yl)-5-ethyl-1-methyl-5-phenylimidazolidine-2,4-dione, 3-((S)-1-(((S)-2,3-dihydrobenzo[b][1,4]dioxin-2-yl)methyl)piperidin-3-yl)-5-methyl-5-phenylimidazolidine-2,4-dione, 6-((S)-1-(((S)-2,3-dihydrobenzo[b][1,4]dioxin-2-yl)methyl)piperidin-3-yl)-5H-pyrrolo[3,4-b]pyridine-5,7(6H)-dione, 2-((S)-1-(((S)-2,3-dihydrobenzo[b][1,4]dioxin-2-yl)methyl)piperidin-3-yl)-2,3-dihydro-1H-pyrrolo[3,4-c]pyridin-1-one, 6-((S)-1-(((S)-2,3-dihydrobenzo[b][1,4]dioxin-2-yl)methyl)piperidin-3-yl)-5H-pyrrolo[3,4-b]pyridin-7(6H)-one, 6-((S)-1-(((S)-2,3-dihydrobenzo[b][1,4]dioxin-2-yl)methyl)piperidin-3-yl)-5H-pyrrolo[3,4-d]pyrimidin-7(6H)-one, 5-((S)-1-(((S)-2,3-dihydrobenzo[b][1,4]dioxin-2-yl)methyl)piperidin-3-yl)-4H-pyrrolo-[3,4-d]thiazol-6(5H)-one, 5-((S)-1-(((S)-2,3-dihydrobenzo[b][1,4]dioxin-2-yl)methyl)-piperidin-3-yl)-1-methyl-5,6-dihydropyrrolo[3,4-c]pyrazol-4(1H)-one, 2-((S)-1-(((S)-2,3-dihydrobenzo[b][1,4]dioxin-2-yl)methyl)piperidin-3-yl)-2,3-dihydrobenzo[d]isothiazole-1,1-dioxide, 1-((S)-1-(((S)-2,3-dihydrobenzo[b][1,4]dioxin-2-yl)methyl)piperidin-3-yl)-6-fluoro-1H-benzo[d]imidazole, 1-((S)-1-(((S)-2,3-dihydrobenzo[b][1,4]dioxin-2-yl)methyl)piperidin-3-yl)-6-fluoro-2-methyl-1H-benzo[d]imidazole, 1-((S)-1-(((S)-2,3-dihydrobenzo[b][1,4]dioxin-2-yl)methyl)piperidin-3-yl)-2-ethyl-6-fluoro-1H-benzo[d]-imidazole, 1-((S)-1-(((S)-2,3-dihydrobenzo[b][1,4]dioxin-2-yl)methyl)piperidin-3-yl)-6-fluoro-2-isopropyl-1H-benzo[d]imidazole, 1-((S)-1-(((S)-2,3-dihydrobenzo[b][1,4]dioxin-2-yl)methyl)piperidin-3-yl)-6-fluoro-1H-benzo[d]imidazol-2(3H)-one, 1-((S)-1-(((S)-2,3-dihydrobenzo[b][1,4]dioxin-2-yl)methyl)piperidin-3-yl)-5-fluoro-1H-benzo[d]imidazole, 1-((S)-1-(((S)-2,3-dihydrobenzo[b][1,4]dioxin-2-yl)methyl)piperidin-3-yl)-5-fluoro-1H-benzo[d]imidazol-2(3H)-one, 6-chloro-1-((S)-1-(((S)-2,3-dihydrobenzo[b][1,4]dioxin-2-yl)methyl)piperidin-3-yl)-1H-benzo[d]imidazole, 6-chloro-1-((S)-1-(((S)-2,3-dihydrobenzo-[b][1,4]dioxin-2-yl)methyl)piperidin-3-yl)-2-methyl-1H-benzo[d]imidazole, 5-chloro-1-((S)-1-(((S)-2,3-dihydrobenzo[b][1,4]dioxin-2-yl)methyl)piperidin-3-yl)-1H-benzo[d]-imidazole, 2-((S)-1-(((S)-2,3-dihydrobenzo[b][1,4]dioxin-2-yl)methyl)piperidin-3-yl)tetrahydrocyclopenta[c]pyrrole-1,3(2H,3aH)-dione hydrochloride, (3aR,7aS)-2-((S)-1-(((S)-2,3-dihydrobenzo[b][1,4]dioxin-2-yl)methyl)piperidin-3-yl)hexahydro-1H-isoindole-1,3(2H)-dione hydrochloride, 2-((S)-1-(((S)-2,3-dihydrobenzo[b][1,4]dioxin-2-yl)methyl)-piperidin-3-yl)-4,5,6,7-tetrahydro-1H-isoindole-1,3(2H)-dione hydrochloride, 3-((S)-1-(((S)-2,3-dihydrobenzo[b][1,4]dioxin-2-yl)methyl)piperidin-3-yl)-3-azabicyclo[3.1.0]hexane-2,4-dione, 1-((S)-1-(((S)-2,3-dihydrobenzo[b][1,4]dioxin-2-yl)methyl)-piperidin-3-yl)-3-ethylpyrimidine-2,4,6(1H,3H,5H)-trione, 1-((S)-1-(((S)-2,3-dihydrobenzo[b][1,4]dioxin-2-yl)methyl)-piperidin-3-yl)-3-methylpyrrolidin-2-one, 1-((S)-1-(((S)-2,3-dihydrobenzo-[b][1,4]dioxin-2-yl)methyl)piperidin-3-yl)-3-methylpyrrolidin-2-one diastereomer 1,1-((S)-1-(((S)-2,3-dihydrobenzo[b][1,4]dioxin-2-yl)methyl)piperidin-3-yl)-3-methylpyrrolidin-2-one diastereomer 2,1-((S)-1-(((S)-2,3-dihydrobenzo[b][1,4]dioxin-2-yl)methyl)piperidin-3-yl)-3,3-dimethylpyrrolidin-2-one, 1-((S)-1-(((S)-2,3-dihydrobenzo[b][1,4]dioxin-2-yl)methyl)piperidin-3-yl)-3-methylimidazolidin-2-one, 3-((S)-1-(((S)-2,3-dihydrobenzo-[b][1,4]dioxin-2-yl)methyl)piperidin-3-yl)imidazolidine-2,4-dione, 3-((S)-1-(((S)-2,3-dihydrobenzo[b][1,4]dioxin-2-yl)methyl)piperidin-3-yl)-1-methylimidazolidine-2,4-dione, 3-((S)-1-(((S)-2,3-dihydrobenzo[b][1,4]dioxin-2-yl)methyl)piperidin-3-yl)-1-isopropylimidazolidine-2,4-dione, 3-((S)-1-(((S)-2,3-dihydrobenzo[b][1,4]dioxin-2-yl)methyl)-piperidin-3-yl)-1-ethylimidazolidine-2,4-dione, 1-cyclopentyl-3-((S)-1-(((S)-2,3-dihydrobenzo[b][1,4]dioxin-2-yl)methyl)piperidin-3-yl)imidazolidine-2,4-dione, 3-((S)-1-(((S)-2,3-dihydrobenzo[b][1,4]dioxin-2-yl)methyl)piperidin-3-yl)-1-isobutylimidazolidine-2,4-dione, 1-(cyclopropylmethyl)-3-((S)-1-(((S)-2,3-dihydrobenzo[b][1,4]dioxin-2-yl)methyl)piperidin-3-yl)imidazolidine-2,4-dione, 2-(3-((S)-1-(((S)-2,3-dihydrobenzo-[b][1,4]dioxin-2-yl)methyl)piperidin-3-yl)-2,4-dioxoimidazolidin-1-yl)-N,N-dimethyl-acetamide, 3-((S)-1-(((S)-2,3-dihydrobenzo[b][1,4]dioxin-2-yl)methyl)piperidin-3-yl)-5,5-dimethylimidazolidine-2,4-dione, 3-((S)-1-(((S)-2,3-dihydrobenzo[b][1,4]dioxin-2-yl)methyl)piperidin-3-yl)-1,5,5-trimethylimidazolidine-2,4-dione, (R)-3-((S)-1-(((S)-2,3-dihydrobenzo[b][1,4]dioxin-2-yl)methyl)piperidin-3-yl)-5-methylimidazolidine-2,4-dione, (S)-3-((S)-1-(((S)-2,3-dihydrobenzo[b][1,4]dioxin-2-yl)methyl)piperidin-3-yl)-5-methylimidazolidine-2,4-dione, 3-((S)-1-(((S)-2,3-dihydrobenzo[b][1,4]dioxin-2-yl)methyl)-piperidin-3-yl)-5-phenylimidazolidine-2,4-dione, 3-((S)-1-(((S)-2,3-dihydrobenzo[b][1,4]-dioxin-2-yl)methyl)-piperidin-3-yl)-1,5-dimethylimidazolidine-2,4-dione, 3-((S)-1-(((S)-2,3-dihydrobenzo[b][1,4]dioxin-2-yl)methyl)-piperidin-3-yl)-1-isopropyl-5,5-dimethyl-imidazolidine-2,4-dione, 1-tert-butyl-3-((S)-1-(((S)-2,3-dihydrobenzo[b][1,4]dioxin-2-yl)methyl)piperidin-3-yl)imidazolidine-2,4-dione, 1-benzyl-3-((S)-1-(((S)-2,3-dihydrobenzo-[b][1,4]dioxin-2-yl)methyl)piperidin-3-yl)imidazolidine-2,4-dione, 1-cyclopropyl-3-((S)-1-(((S)-2,3-dihydrobenzo[b][1,4]dioxin-2-yl)methyl)piperidin-3-yl)imidazolidine-2,4-dione, 3-((S)-1-(((S)-2,3-dihydrobenzo[b][1,4]dioxin-2-yl)methyl)-piperidin-3-yl)-1-(oxetan-3-yl)imidazolidine-2,4-dione, 1-(3,3-difluorocyclobutyl)-3-((S)-1-(((S)-2,3-dihydrobenzo-[b][1,4]dioxin-2-yl)methyl)piperidin-3-yl)imidazolidine-2,4-dione, 6-((S)-1-(((S)-2,3-dihydrobenzo[b][1,4]dioxin-2-yl)methyl)-piperidin-3-yl)-4,6-diazaspiro[2.4]heptane-5,7-dione, 6-((S)-1-(((S)-2,3-dihydrobenzo[b][1,4]dioxin-2-yl)methyl)-piperidin-3-yl)-4-methyl-4,6-diazaspiro[2.4]heptane-5,7-dione, 2-(6-((S)-1-(((S)-2,3-dihydrobenzo[b][1,4]dioxin-2-yl)methyl)piperidin-3-yl)-5,7-dioxo-4,6-diazaspiro[2.4]heptan-4-yl)-N,N-dimethyl-acetamide, 1-((S)-1-(((S)-2,3-dihydrobenzo[b][1,4]dioxin-2-yl)methyl)-piperidin-3-yl)-3-ethylimidazolidine-2,4,5-trione, 1-cyclohexyl-3-((S)-1-(((S)-2,3-dihydrobenzo[b][1,4]-dioxin-2-yl)methyl)piperidin-3-yl)imidazolidine-2,4,5-trione, 1-cyclopentyl-3-((S)-1-(((S)-2,3-dihydrobenzo[b][1,4]dioxin-2-yl)methyl)piperidin-3-yl)imidazolidine-2,4,5-trione, 1-((S)-1-(((S)-2,3-dihydrobenzo[b][1,4]dioxin-2-yl)methyl)-piperidin-3-yl)-3-((R)-1-phenylethyl)imidazolidine-2,4,5-trione, 1-((S)-1-(((S)-2,3-dihydrobenzo[b][1,4]dioxin-2-yl)methyl)-piperidin-3-yl)-3-phenylimidazolidine-2,4,5-trione, 1-((S)-1-(((S)-2,3-dihydrobenzo[b][1,4]dioxin-2-yl)methyl)-piperidin-3-yl)-3-isopropylimidazolidine-2,4,5-trione, 1-benzyl-3-((S)-1-(((S)-2,3-dihydrobenzo[b][1,4]dioxin-2-yl)methyl)piperidin-3-yl)imidazolidine-2,4,5-trione, 1-((S)-1-(((S)-2,3-dihydrobenzo[b][1,4]dioxin-2-yl)methyl)-piperidin-3-yl)-3-propylimidazolidine-2,4,5-trione, 1-((S)-1-(((S)-2,3-dihydrobenzo[b][1,4]-dioxin-2-yl)methyl)-piperidin-3-yl)-3-((S)-1-phenylethyl)imidazolidine-2,4,5-trione, 1-tert-butyl-3-((S)-1-(((S)-2,3-dihydrobenzo[b][1,4]dioxin-2-yl)methyl)piperidin-3-yl)imidazolidine-2,4,5-trione, 1-((S)-1-(((S)-2,3-dihydrobenzo[b][1,4]dioxin-2-yl)methyl)-piperidin-3-yl)-3-(2-(dimethylamino)ethyl)imidazolidine-2,4,5-trione, 1-((S)-1-(((S)-2,3-dihydrobenzo[b][1,4]dioxin-2-yl)methyl)piperidin-3-yl)-3-(tetrahydro-2H-pyran-4-yl)imidazolidine-2,4,5-trione, 1-((S)-1-(((S)-2,3-dihydrobenzo[b][1,4]dioxin-2-yl)methyl)-piperidin-3-yl)-3-(piperidin-4-yl)imidazolidine-2,4,5-trione dihydrochloride, 1-((S)-1-(((S)-2,3-dihydrobenzo[b][1,4]dioxin-2-yl)methyl)piperidin-3-yl)-3-methylimidazolidine-2,4,5-trione, 1-(1-acetylpiperidin-4-yl)-3-((S)-1-(((S)-2,3-dihydrobenzo[b][1,4]dioxin-2-yl)-methyl)piperidin-3-yl)imidazolidine-2,4,5-trione, 1-((S)-1-(((S)-2,3-dihydrobenzo[b][1,4]-dioxin-2-yl)methyl)piperidin-3-yl)-3-(pyridin-4-ylmethyl)imidazolidine-2,4,5-trione, 1-((S)-1-(((S)-2,3-dihydrobenzo[b][1,4]dioxin-2-yl)methyl)piperidin-3-yl)-3-isobutylimidazolidine-2,4,5-trione, 1-((S)-1-(((S)-2,3-dihydrobenzo[b][1,4]dioxin-2-yl)methyl)-piperidin-3-yl)-3-(pyridin-2-ylmethyl)imidazolidine-2,4,5-trione, 1-((S)-1-(((S)-2,3-dihydrobenzo[b][1,4]dioxin-2-yl)methyl)piperidin-3-yl)-3-(pyridin-3-ylmethyl)-imidazolidine-2,4,5-trione, 1-((S)-1-(((S)-2,3-dihydrobenzo[b][1,4]dioxin-2-yl)methyl)piperidin-3-yl)-1H-benzo[d]imidazol-2(3H)-one, 1-((S)-1-(((S)-2,3-dihydrobenzo-[b][1,4]dioxin-2-yl)methyl)piperidin-3-yl)-1H-benzo[d][1,2,3]triazole, 1-((S)-1-(((S)-2,3-dihydrobenzo[b][1,4]dioxin-2-yl)methyl)piperidin-3-yl)-1,3-dihydrobenzo[c][1,2,5]-thiadiazole-2,2-dioxide, 1-((S)-1-(((S)-2,3-dihydrobenzo[b][1,4]dioxin-2-yl)methyl)-piperidin-3-yl)-3-methyl-1H-benzo[d]imidazol-2(3H)-one, 1-((S)-1-(((S)-2,3-dihydrobenzo-[b][1,4]dioxin-2-yl)methyl)piperidin-3-yl)-1H-benzo[d]imidazole, 1-((S)-1-(((S)-2,3-dihydrobenzo[b][1,4]dioxin-2-yl)methyl)piperidin-3-yl)-3-phenyl-1H-benzo[d]imidazol-2(3H)-one, 1-((S)-1-(((S)-2,3-dihydrobenzo[b][1,4]dioxin-2-yl)methyl)piperidin-3-yl)-2-methyl-1H-benzo[d]imidazole, 1-((S)-1-(((S)-2,3-dihydrobenzo[b][1,4]dioxin-2-yl)methyl)-piperidin-3-yl)-2-(methoxymethyl)-1H-benzo[d]imidazole, 1-(6-chloro-1-((S)-1-(((S)-2,3-dihydrobenzo[b][1,4]dioxin-2-yl)methyl)piperidin-3-yl)-1H-benzo[d]imidazol-2-yl)-N,N-dimethylmethanamine, 1-(6-chloro-1-((S)-1-(((S)-2,3-dihydrobenzo[b][1,4]dioxin-2-yl)methyl)piperidin-3-yl)-1H-benzo[d]imidazol-2-yl)-N-methylmethanamine, 1-((S)-1-(((S)-2,3-dihydrobenzo[b][1,4]dioxin-2-yl)methyl)piperidin-3-yl)-4-fluoro-1H-benzo[d]imidazol-2(3H)-one, 3-((S)-1-(((S)-2,3-dihydrobenzo[b][1,4]dioxin-2-yl)methyl)piperidin-3-yl)-5-methoxy-2-methyl-3H-imidazo[4,5-b]pyridine, 3-((S)-1-(((S)-2,3-dihydrobenzo[b][1,4]-dioxin-2-yl)methyl)-piperidin-3-yl)-2-methyl-3H-imidazo[4,5-b]pyridine, 1-((S)-1-(((S)-2,3-dihydrobenzo[b][1,4]dioxin-2-yl)methyl)-piperidin-3-yl)-2-methyl-1H-imidazo[4,5-b]-pyridine, 1-((S)-1-(((S)-2,3-dihydrobenzo[b][1,4]dioxin-2-yl)methyl)piperidin-3-yl)-2-methyl-1H-imidazo[4,5-c]pyridine, 3-((S)-1-(((S)-2,3-dihydrobenzo[b][1,4]dioxin-2-yl)-methyl)piperidin-3-yl)-2-ethyl-3H-imidazo[4,5-b]pyridine, 9-((S)-1-(((S)-2,3-dihydrobenzo-[b][1,4]dioxin-2-yl)methyl)piperidin-3-yl)-8-methyl-9H-purine, 9-((S)-1-(((S)-2,3-dihydrobenzo[b][1,4]dioxin-2-yl)methyl)-piperidin-3-yl)-9H-purine, 2-((S)-1-(((S)-2,3-dihydrobenzo[b][1,4]dioxin-2-yl)methyl)piperidin-3-yl)-5,5-dimethylisothiazolidine-1,1-dioxide, or 3-((S)-1-(((S)-2,3-dihydrobenzo[b][1,4]dioxin-2-yl)methyl)piperidin-3-yl)oxazolidine-2,4-dione.

11. (canceled)

12. (canceled)

13. (canceled)

14. A method for the treatment of a disorder, condition, or disease where an alpha2C antagonist is indicated to be useful, comprising administering to a mammal in need of such treatment an effective amount of a compound
of formula I, 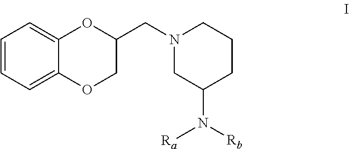
wherein;
Rₐ and R_{b} form, together with the nitrogen atom to which they are attached, a 5 or 6 membered saturated or unsaturated heterocyclic ring, containing, in addition to the nitrogen atom to which Rₐ and R_{b} are attached, 0, 1 or 2 ring heteroatom(s) each independently selected from N, O and S, wherein said heterocyclic ring is substituted with 1 substituent R₁, or said heterocyclic ring is substituted with 2 substituents R₁ and R₂, or said heterocyclic ring is substituted with 3 substituents R₁, R₂, and R₃, or said heterocyclic ring is substituted with 4 substituents R₁, R₂, R₃, and R₄, or said heterocyclic ring is substituted with 5 substituents R₁, R₂, R₃, R₄, and R₅;
R₁, R₂, R₃, R₄ and R₅ are independently oxo, (C₁-C₆)alkyl, (C₁-C₆)alkoxy(C₁-C₆)alkyl, (R₆)₂N—, (R₆)₂N—(C₁-C₆)alkyl, (R₆)₂N—(C═O)—(C₁-C₆)alkyl, (C₁-C₆)alkyl-(C═O)—NR₆—(C₁-C₆)alkyl, cyclo(C₃-C₆)alkyl, cyclo(C₃-C₆)alkyl(C₁-C₆)alkyl, phenyl, phenyl(C₁-C₆)alkyl, heterocyclyl, or heterocyclyl(C₁-C₆)alkyl, wherein said phenyl, cyclo(C₃-C₆)alkyl, or heterocyclyl is optionally substituted with 1 or 2 substituent(s) each independently being halogen, (C₁-C₆)alkoxy, or (C₁-C₆)alkyl-(C═O);
or two of R₃, R₄ and R₅, both attached to the same carbon ring atom form, together with the carbon ring atom to which they are attached, a 3 membered unsubstituted carbocyclic ring;
or two of R₁, R₂, R₃, R₄ and R₅, attached to the adjacent carbon ring atoms form, together with the carbon ring atoms to which they are attached, a phenyl ring, a 3 to 6 membered saturated or unsaturated carbocyclic ring, or a 5 or 6 membered saturated or unsaturated heterocyclic ring containing 1 or 2 ring heteroatom(s) each independently selected from N and S, wherein said phenyl ring, carbocyclic ring, or heterocyclic ring is unsubstituted, or said phenyl ring, carbocyclic ring, or heterocyclic ring is substituted with 1 substituent R₇, or said phenyl ring, carbocyclic ring, or heterocyclic ring is substituted with 2 substituents R₇ and R₈;
R₆ is H or (C₁-C₆)alkyl;
R₇ and R₈ are independently halogen, (C₁-C₆)alkyl, (C₁-C₆)alkoxy, (C₁-C₆)alkyl-S—, CN, or (C₁-C₆)alkyl-(C═O)—NR₆—(C₁-C₆)alkyl;
or R₇ and R₈, attached to the non-adjacent carbon ring atoms, form a bridge;
or a pharmaceutically acceptable salt or ester thereof.

15. The method according to claim 14, wherein the disorder, condition, or disease is a mental disorder propagated by stress, Parkinson's disease, depression, schizophrenia, attention deficit hyperactivity disorder, post-traumatic stress disorder, obsessive compulsive disorder, Tourette's syndrome, blepharospasm or other focal dystonias, temporal lobe epilepsy with psychosis, a drug-induced psychosis, Huntington's disease, a disorder caused by fluctuation of the levels of sex hormones, panic disorder, Alzheimer's disease, or mild cognitive impairment.

16. A pharmaceutical composition comprising at least one compound according to claim 1 and a pharmaceutically acceptable carrier, diluent, and/or excipient.

17. The pharmaceutical composition according to claim 16 wherein the composition comprises further another active ingredient.
